SEQUENCE LISTING (36 sequences)

SEQ ID NO: 1 - DNA, 2136 bp, Homo sapiens
gggaagaggcggagaacaatatggcggatggcgaggagccggagaagaaaagaaggagaatagaggagctgctggctgagaaaatggctgttgatggtgggtgtggggacactggagactgggaaggtcgctggaaccatgtaaagaagttcctcgagcgatctggacccttcacacaccctgatttcgaaccgagcactgaatctctccagttcttgttagatacatgtaaagttctagtcattggagctggcggcttaggatgtgagctcctgaaaaatctggccttgtctggttttagacagattcatgttatagatatggacactatagatgtttccaatctaaataggcagtttttatttaggcctaaagatattggaagacctaaggctgaagttgctgcagaatttctaaatgacagagttcctaattgcaatgtagttccacatttcaacaagattcaagattttaacgacactttctatcgacaatttcatattattgtatgtggactggactctatcatcgccagaagatggataaatggcatgctgatatctcttctaaattatgaagatggtgtcttagatccaagctccattgtccctttgatagatggggggacagaaggttttaaaggaaatgcccgggtgattctgcctggaatgactgcttgtatcgaatgcacgctggaactttatccaccacaggttaattttcccatgtgcaccattgcatctatgcccaggctaccagaacactgtattgagtatgtaaggatgttgcagtggcctaaggagcagccttttggagaaggggttccattagatggagatgatcctgaacatatacaatggattttccaaaaatccctagagagagcatcacaatataatattaggggtgttacgtataggctcactcaaggggtagtaaaaagaatcattcctgcagtagcttccacaaatgcagtcattgcagctgtgtgtgccactgaggtttttaaaatagccacaagtgcatacattcccttgaataattacttggtgtttaatgatgtagatgggctgtatacatacacatttgaagcagaaagaaaggaaaactgcccagcttgtagccagcttcctcaaaatattcagttttctccatcagctaaactacaggaggttttggattatctaaccaatagtgcttctctgcaaatgaaatctccagccatcacagccaccctagagggaaaaaatagaacactttacttacagtcggtaacctctattgaagaacgaacaaggccaaatctctccaaaacattgaaagaattggggcttgttgatggacaagaactggcggttgctgatgtcaccaccccacagactgtactattcaaacttcattttacttcttaaggaaaatctccacataatagaaaactcatggaaataatatactttgtggatgctaagaagttgaatcgatgtcatttttagcaatagtgttgccacgatttgtctttttttatataatgaaccactcttttttaactttgtaaccttcccttgaagacagaattttggtgttggtgcttgtaagcattttcattaataatatgagaaatgatacctggagagagagattatgagcaaatgtattgcttcttttagaggaggaagcatacaacctcttttgtgtgaattttgttattatggtcaaagaatgcattcctaagttttcatttgagtacccaaatacacaaaaggtgtccctttaaggaaaataaagaattaagttttaaataacattacattttacaatctgacatctggagtatattgaacataggctatttcttgatataacactcatttaattgtggccatccaaatgaatattattgcagaatttatcttgttcataatgatttgtaaatggtgttatagctgaatacctgtgcatgaaaatgggcaatattttcatctgtttacttgtagtgccatagaggccaatatgcacaatattaactaatgccaagacatggctgtttaaaaaatttaatgttcaaacagttatcactgatgcttttgcactatttattaataaaatcatatattgtgtaaaaaaaaaaaaaaaaaa

SEQ ID NO: 2 - protein, 463 aa, Homo sapiens
MADGEEPEKKRRRIEELLAEKMAVDGGCGDTGDWEGRWNHVKKFLERSGPFTHPDFEPSTESLQFLLDTCKVLVIGAGGLGCELLKNLALSGFRQIHVIDMDTIDVSNLNRQFLFRPKDIGRPKAEVAAEFLNDRVPNCNVVPHFNKIQDFNDTFYRQFHIIVCGLDSIIARRWINGMLISLLNYEDGVLDPSSIVPLIDGGTEGFKGNARVILPGMTACIECTLELYPPQVNFPMCTIASMPRLPEHCIEYVRMLQWPKEQPFGEGVPLDGDDPEHIQWIFQKSLERASQYNIRGVTYRLTQGVVKRIIPAVASTNAVIAAVCATEVFKIATSAYIPLNNYLVFNDVDGLYTYTFEAERKENCPACSQLPQNIQFSPSAKLQEVLDYLTNSASLQMKSPAITATLEGKNRTLYLQSVTSIEERTRPNLSKTLKELGLVDGQELAVADVTTPQTVLFKLHFTS

SEQ ID NO: 3 - protein, 534 aa, Homo sapiens
MAQLGKLLKEQKYDRQLRLWGDHGQEALESAHVCLINATATGTEILKNLVLPGIGSFTIIDGNQVSGEDAGNNFFLQRSSIGKNRAEAAMEFLQELNSDVSGSFVEESPENLLDNDPSFFCRFTVVVATQLPESTSLRLADVLWNSQIPLLICRTYGLVGYMRIIIKEHPVIESHPDNALEDLRLDKPFPELREHFQSYDLDHMEKKDHSHTPWIVIIAKYLAQWYSETNGRIPKTYKEKEDFRDLIRQGILKNENGAPEDEENFEEAIKNVNTALNTTQIPSSIEDIFNDDRCINITKQTPSFWILARALKEFVAKEGQGNLPVRGTIPDMIADSGKYIKLQNVYREKAKKDAAAVGNHVAKLLQSIGQAPESISEKELKLLCSNSAFLRVVRCRSLAEEYGLDTINKDEIISSMDNPDNEIVLYLMLRAVDRFHKQQGRYPGVSNYQVEEDIGKLKSCLTGFLQEYGLSVMVKDDYVHEFCRYGAAEPHTIAAFLGGAAAQEVIKIITKQFVIFNNTYIYSGMSQTSATFQL

SEQ ID NO: 4 - protein, 133 aa, Artificial Sequence
  consensus
ESKVLVVGAGGLGSEAAEALARAGVGKLTLVDKDTVELSNLNRQLLFREEDIGKPKAEVAKERLREINPEVEVEAVEERITEENLEELLKDVDLVVDALDNIEARLLLNDACVKKGIPLISAGVLGFKGQVVT

SEQ ID NO: 5 - protein, 42 aa, Artificial Sequence
  consensus
GVTECYECSPDPEEKSIPVCTLRSFPSEIEHCIEWARDLFEK

SEQ ID NO: 6 - protein, 9 aa, Artificial Sequence
  consensus
PXCTXXXXP

SEQ ID NO: 7 - protein, 65 aa, Artificial Sequence
  consensus
FDKDDDDHVDFVAAAANLRAENFGIKEKSRNEVKQIAGNIIPAIATTNAIVAAIVVLELIKVLEG

SEQ ID NO: 8 - protein, 84 aa, Artificial Sequence
  consensus
IEVSKSVTLEELIESLEERPELQLKKPSLTTAEKTLYMQSPPSLEEATRPNLSKKLKELVSDGQEIVVTDEKLPVSLKLRLKFK

SEQ ID NO: 9 - protein, 463 aa, Callithrix jacchus
MADGEEPEKKRRRIEELLAEKMAVDGGCGDSGDWEGRWNHVKKFLERSGPFTHPDFEPSTESLQFLLDTCKVLVIGAGGLGCELLKNLALSGFRQIHVIDMDTIDVSNLNRQFLFRPKDVGRPKAEVAAEFLNDRVPNCNVVPHFNKIQDFNDTFYRQFHIIVCGLDSIIARRWINGMLISLLNYEDGVLDPSSIIPLIDGGTEGFKGNARVILPGMTACIECTLELYPPQVNFPMCTIASMPRLPEHCIEYVRMLQWPKEQPFGEGVPLDGDDPEHIQWIFQKSLERASQYNIRGVTYRLTQGVVKRIIPAVASTNAVIAAVCATEVFKIATSAYIPLNNYLVFNDVDGLYTYTFEAERKENCPACSQLPQNIQFSPSAKLQEVLDYLTNSASLQMKSPAITATLEGKNRTLYLQSVTSIEERTRPNLSKTLKELGLVDGQELAVADVTTPQTVLFKLHFTS

SEQ ID NO: 10 - protein, 463 aa, Canis lupus familiaris
MADGEEPEKKRRRIEELLAEKMAVDGGCGDTGDWEGRWNHVKKFLERSGPFTHPDFEPSTESLQFLLDTCKVLVIGAGGLGCELLKNLALSGFRQIHVIDMDTIDVSNLNRQFLFRPKDVGRPKAEVAAEFLNDRVPNCNVVPHFNKIQDFNDTFYRQFHIIVCGLDSIIARRWINGMLISLLNYEDGVLDPSSIVPLIDGGTEGFKGNARVILPGMTACIECTLELYPPQVNFPMCTIASMPRLPEHCIEYVRILQWPKEQPFGEGVPLDGDDPDHIQWIFQKSLERASQYNIRGVTYRLTQGVVKRIIPAVASTNAVIAAVCATEVFKIATSAYIPLNNYLVFNDVDGLYTYTFEAERKENCPACSQLPQNIQFSPSAKLQEVLDYLTNSASLQMKSPAITATLEGKNRTLYLQSVTSIEERTRPNLSKTLKELGLVDGQELAVADVTTPQTVLFKLHFTS

SEQ ID NO: 11 - protein, 462 aa, Mus musculus
MADGEEPEKKRRRIEELLAEKMAVDGGCGDTGDWEGRWNHVKKFLERSGPFTHPDFEPSTESLQFLLDTCKVLVIGAGGLGCELLKNLALSGFRQIHVIDMDTIDVSNLNRQFLFRPKDVGRPKAEVAAEFLNDRVPNCNVVPHFNKIQDFNDTFYRQFHIIVCGLDSIIARRWINGMLISLLNYEDGVLDPSSIVPLIDGGTEGFKGNARVILPGMTACIECTLELYPPQVNFPMCTIASMPRLPEHCIEYVRMLQWPKEQPFGDGVPLDGDDPEHIQWIFQKSIERASQYNIRGVTYRLTQGVVKRIIPAVASTNAVIAAVCATEVFKIATSAYIPLNNYLVFNDVDGLYTYTFEAERKENCPACSQLPQNIQFSPSAKLQEVLDYLTNSASLQMKSPAITATLEGKNRTLYLQSVTSIEERTRPNLSKTLKELGLVDGQELAVADVTTPQTVLFKLHFT

SEQ ID NO: 12 - protein, 462 aa, Rattus norvegicus
MADGEEPEKKRRRIEELLAEKMAVDGGCGDTGDWEGRWNHVKKFLERSGPFTHPDFEPSTESLQFLLDTCKVLVIGAGGLGCELLKNLALSGFRQIHVIDMDTIDVSNLNRQFLFRPKDVGRPKAEVAAEFLNDRVPNCNVVPHFNKIQDFNDTFYRQFHIIVCGLDSIIARRWINGMLISLLNYEDGVLDPSSIVPLIDGGTEGFKGNARVILPGMTACIECTLELYPPQVNFPMCTIASMPRLPEHCIEYVRMLQWPKEQPFGDGVPLDGDDPEHIQWIFQKSVERASQYNIRGVTYRLTQGVVKRIIPAVASTNAVIAAVCATEVFKIATSAYIPLNNYLVFNDVDGLYTYTFEAERKENCPACSQLPQNIQFSPSAKLQEVLDYLTNSASLQMKSPAITATLEGKNRTLYLQSVTSIEERTRPNLSKTLKELGLVDGQELAVADVTTPQTVLFKLHFT

SEQ ID NO: 13 - protein, 463 aa, Bos taurus
MADGEEPEKKRRRIEELLAEKMAVDGGCGDTGDWEGRWNHVKKFLERSGPFTHPDFEPSTESLQFLLDTCKVLVIGAGGLGCELLKNLALSGFRQIHVIDMDTIDVSNLNRQFLFRPKDVGRPKAEVAAEFLNDRIPNCNVVPHFNKIQDFNDTFYRQFHIIVCGLDSIIARRWINGMLISLLNYEDGVLDPSSIVPLIDGGTEGFKGNARVILPGMTACIECTLELYPPQVNFPMCTIASMPRLPEHCIEYVRILQWPKEQPFGEGVPLDGDDPDHIQWIFQKALERASQYNIRGVTYRLTQGVVKRIIPAVASTNAVIAAVCATEVFKIATSAYIPLNNYLVFNDVDGLYTYTFEAERKENCPACSQLPQNIQFSPSAKLQEVLDYLTNSASLQMKSPAITATLEGKNRTLYLQSVTSIEERTRPNLSKTLKELGLVDGQELAVADVTTPQTVLFKLHFTS

SEQ ID NO: 14 - protein, 449 aa, Homo sapiens
MADGEEPMAVDGGCGDTGDWEGRWNHVKKFLERSGPFTHPDFEPSTESLQFLLDTCKVLVIGAGGLGCELLKNLALSGFRQIHVIDMDTIDVSNLNRQFLFRPKDIGRPKAEVAAEFLNDRVPNCNVVPHFNKIQDFNDTFYRQFHIIVCGLDSIIARRWINGMLISLLNYEDGVLDPSSIVPLIDGGTEGFKGNARVILPGMTACIECTLELYPPQVNFPMCTIASMPRLPEHCIEYVRMLQWPKEQPFGEGVPLDGDDPEHIQWIFQKSLERASQYNIRGVTYRLTQGVVKRIIPAVASTNAVIAAVCATEVFKIATSAYIPLNNYLVFNDVDGLYTYTFEAERKENCPACSQLPQNIQFSPSAKLQEVLDYLTNSASLQMKSPAITATLEGKNRTLYLQSVTSIEERTRPNLSKTLKELGLVDGQELAVADVTTPQTVLFKLHFTS

SEQ ID NO: 15 - protein, 449 aa, Sus scrofa
MSKGDQVMAVDGGCGDTGDWEGRWNHVKKFLERSGPFTHPDFEPSTESLQFLLETCKVLVIGAGGLGCELLKNLALSGFRQIHVIDMDTIDVSNLNRQFLFRPKDVGRPKAEVAAEFLNDRVPNCNVVPHFNKIQDFNDTFYRQFHIIVCGLDSIIARRWINGMLISLLNYEDGVLDPSSIVPLIDGGTEGFKGNARVILPGMTACIECTLELYPPQVNFPMCTIASMPRLPEHCIEYVRILQWPKEQPFGEGVPLDGDDPDHIQWIFQKSLERASQYNIRGVTYRLTQGVVKRIIPAVASTNAVIAAVCATEVFKIATSAYIPLNNYLVFNDVDGLYTYTFEAERKENCPACSQLPQNIQFSPSAKLQEVLDYLTNSASLQMKSPAITATLEGKNRTLYLQSVTSIEERTRPNLSKTLKELGLVDGQELAVADVTTPQTVLFKLHFTS

SEQ ID NO: 16 - protein, 461 aa, Xenopus laevis
MADAEEPEKKRRRIEELPDEMAVNGGCGETGDWEGRWNHVKKFLERSGPFTHPEFEPSNESLQFLLETCKLLVVGAGGLGCELLKNLALSGFRQIHVIDMDTIDVSNLNRQFLFRPKDVGRPKAEVAADFINARIPDCCVTPHFKKIQDFDETFYREFHIIVCGLDSIIARRWLNGMLMSLLNYEDSVLQQSTVIPLIDGGTEGFKGNSRVILPGMTACVECTLELYPPQINFPMCTIASMPRLPEHCIEYVRILQWPKEQPFGEGVQLDGDDPEHIEWIFTNSLERANQFNIRGVTYRLTQGVVKRIIPAVASTNAVIAAACATEVFKIATSAYIPLNNYLVFNDVDGLYSYTFEAEKKENCPACSQLPQNIQFPSSAKLQEVLDYLTNDTLQMKAPAITATLEGKNKTLYLQTVTSIEERTRPNLCRTLKELGLVDGQELAVADVTTPQTVLFKLHFTA

SEQ ID NO: 17 - protein, 442 aa, Salmo salar
MVVDGGSGDGGEWEGRWNHISTFLERPGPFTHPDFEPSTESLQFLLDTCKILVIGAGGLGCELLKDLALSGFRHIHVVDMDTIDVSNLNRQFLFRLKDVGRPKADIAADFINGRIPGCNVVPHFKKIQDFDESFYRQFHIIVCGLDSIIARRWMNGMLLSLLVYEDGVLDPSSIIPLIDGGTEGFKGNARVIFPGMTACIDCTLELYPPQINFPMCTIASMPRLPEHCVEYVRMLLWPKEKPFGDGVGLDADDPEHIQWVYQKSQERAAEFSITGVTYRLTQGVVKRIIPAVASTNAVIAAACATEVFKIASSAYIPLNNYMVFNDVDGLYTYTFEAERKENCSSCSQVPQDLHFSPSAKLQEVLDYLTENASLQMKSPAITATLEGKNKTLYLQTVASIEQRTRPNLSKSLKELGLLDGQELAVADVTTPQTVLFKLSFTS

SEQ ID NO: 18 - protein, 450 aa, Aedes aegypti
METTTTQTSDHQGKRWNHLRKILERSGPFCPPNFTASNETLEFLQNTCKILVIGAGGLGCELLKDMALMGFRDIHVIDMDTIELSNLNRQFLFRRADIGKSKAECAAAFVNARIPGCTVTPHFCKIQDFDAGFYRQFHIIVCGLDSIVARRWINGMLISMLEYEEDGSVDETSIIPLVDGGTEGFKGNARVILPGMTACIDCTLDLFPPQVTYPLCTIANTPRLPEHCIEYVKIIQWPKENPFGSDIGLDGDDPQHITWVYEKAQERANTFNITGLSYRLVQGVLKNIIPAVASTNAVIAAACATEVFKIASSCCEPLNNYMVFNDSDGIYTYTYEAEKKADCLACSQVPRPVDVVDPNTMTLQDLIQHLCDSAEFQMKSPGLTASINGKNKTLYMATVKSIEEATKGNLTQSLGELGLKDGQEIMVADVTNPNAILIKLKFQSNEVEMA

SEQ ID NO: 19 - protein, 449 aa, Anopheles gambiae str. PEST
MEVASPNNDHLSKRWNHLRKILERSGPLCHPYFVASSETLEFLLNTCKILVIGAGGLGCELLKDLALMGIRDIHVIDMDTIELSNLNRQFLFRRTDIGKSKAQCAAAFISARVPGCVVTPHFCKIQDFDSAFYRQFHIIVCGLDSIVARRWINGMMISMLEYEEDGSVDETSIIPFIDGGTEGFKGNARVILPGMTACIDCTLDLFPPQVNYPLCTIANTPRLPEHCIEYVKIIQWPKETPFGVDVALDGDDPQHVSWVYEKAQERANSFNITGLSYRLVQGVLKNIIPAVASTNAVIAAACATEVFKIASSCCEPSNNYMVFNDVDGIYTYTYEAEKRSDCLACSQVPRPVDIKDPNGMTLQDLIQLLCENPEFQMKSPGLTAVLEGKNKTLYMGTVKSIEEATKGNLTMSLSELGLKDGQEIMVADITTPNTILIKLKFQPNEVEMS

SEQ ID NO: 20 - protein, 445 aa, Pediculus humanus corporis
MATVASNGPLNRRWSHLQKILDRSGPFCHPQFEPSPDNLAFLQETCKILVIGAGGLGCELLKNLALMGFKLIHVIDMDIIELSNLNRQFLFRVKDIGLSKAQVAAKFINERVPGCKVIPHFGKIQDFDENFYSSFHIIVCGLDSVVARRWINGMLISLLRYNDNGELDESSTIPLIDGGTEGFKGNARVILPGINACIDCTLDLFPPQITYPLCTIANTPRLPEHCIEYVKEIQWPKENPWNVTLDGDDPNHLNWIYEKASERASQFNIKGINYRLVQGVVKNIIPAVASTNAVIAAACVTEVFKLATYCCLPLNNYMMFNNVSGVYTYTYEAERKPDCLSCSQITKILKLENSSLKLKDLIKILCDKPDYQMKNPGITAVVKGKNKTLYLPLVESIEKVTRSNLTKSLVDLGLEEGSEIMVADITTPKTLIFRLNFKSNDIEMI

SEQ ID NO: 21 - protein, 450 aa, Drosophila melanogaster
MSVHSPNPGLILQSKRFNGLRNILEREGPFCKDGFAASSENLEFLQTKCQVLIIGAGGLGCELLKDLALMGFGNLHVIDMDTIELSNLNRQFLFRRTDIGASKAECAARFINARVPTCRVTPHFKKIQDFDESFYQQFHLVVCGLDSIVARRWINGMLLSMLRYEEDGTIDTSSIVPMIDGGTEGFKGNARVILPGFTACIECTLDLFPPQVNYPLCTIANTPRLPEHCIEYVKIIQWEKQNPFGVPLDGDDPQHIGWIYERALERSNEFNITGVTYRLVQGVVKHIIPAVASTNAAIAAACALEVFKLATSCYDSMANYLNFNDLDGIYTYTYEAEKSENCLACSNTPQPLPIEDPNTTTLEDVIKLLCDSPRFQLKSPALTTVMKDGKRRTLYMSGVKSIEEATRKNLTQSLGELGLHDGQQLTVTDATSPSAMTLQLKYQSNEVEMV

SEQ ID NO: 22 - protein, 421 aa, Aspergillus niger CBS 513.88
MAATGAPQRWKHLYSVLTKSGPFSDEDWVPGQETISALESSKILVIGAGGLGCEILKNLALSGFKDIHVIDMDTIDISNLNRQFLFRQSDIGKPKAEVAAAFVERRVKGVKITPYVGKIQDKDEDYYMQFKIIVCGLDSIEARRWINSTLVGMVDFEDPESLKPLIDGGTEGFKGQARVILPTLSSCIECQLDMHAPRPAVPLCTIATIPRQPQHCIEWAHQIAWQEQRKDDAFDSDDMEHIGWVYNAALERAKQFNIPGVTFQMTQGVVKNIIPAIASTNAVIAAATTSEALKIATSCNPYLENYMMYAGEEGVYTYTFEAEKKPDCPVCGNLARKLTVNPNMTLEEFIETLGERPEAQLKKPSMRTEEKTLYQRFPPQLEEQTRANLKLKLKDLIEDGQEIAVSDPAYIIDFRFRLAFQ

SEQ ID NO: 23 - protein, 419 aa, Aspergillus fumigatus Af293
MAPLSSTSRWRHLYSVLSKPGPYSDEDWVPGPETISALESSKILGAGGLGCEILKNLALSGFKDIHVIDMDTIDISNLNRQFLFRQTDIGKPKAEVAASFVEKRVKGVKITPYVGKIQDKDEDYYMQFKIIVCGLDSIEARRWINSTLIGMVDPENPESLKPLIDGGTEGFKGQARVILPTLSSCIECQLDMHAPRPAVPLCTIATIPRQPQHCIEWAHQIAWQEKRKDDAFDSDDMEHISWVYNAALERANQFHIHGVTFQMTQGVVKNIIPAIASTNAVIAAATTSEALKIATSCNPYLENYMMYAGEDGVYTYTFEAEKKADCPVCGNLARKITVDPNMTLEEFIESLGERAEAQLKKPSMRTEEKTLYQRFPPQLEEQTRSNLKLKLKELVENGQEIAVSDPAYSIDFRFQLIFK

SEQ ID NO: 24 - protein, 425 aa, Schizosaccharomyces japonicus yFS275
MALSHLRKLLNNPGPFADAYDPEEATKAIQTTKILVVGAGGLGCEILVNLACLGFESIDVVDMDTIDLTNLNRQFLFRKKDVGQPKAQIAAEAIQRRMPNCRVTPIVSKVQDIPMDQLYTYGLVICGLDSVEARRWVNATLVSMVDDDDPQSLKALIDGGCEGFRGQARVILPTITSCYECSLDMLPSKKTYPICTIANKPRLLEHCVEWAYVLQWQAEQGEKDPSSEQIPFNPELPEHMDWLVRTASERAKEFNIPGVITHSSAQGIVKNIIPSVASTNAIIAAACCTEAFKLVTGCNPILDNYMMYTGDQGVYTYSFSLEKQKDCPVCGIEAVLLPVCGNEPLSAVVNRLKEKYRLSNPSLSLTPNSSSTPTRPLYYAAPPSLEASTRSNLSISMRELCQQCRSLTVTDTQLPVSMKVVLRWE

SEQ ID NO: 25 - protein, 444 aa, Schizosaccharomyces pombe 972h-
MPSSDVCKAGSHRHSGWIQSLKKPGPFNLDAPENPEETLKSAFSSKILIIGAGGLGCEILKDLALSGFRDLSVIDMDTIDITNLNRQFLFNESNIDEPKANVAASMIMKRIPSTVVTPFYGKIQDKTIEFYKEFKLIICGLDSVEARRWINSTLVAIAKTGDLIPLVDGGSEGLKGQARVIIPTITSCYECSLDMLTPKISYPICTLANTPRLPEHCVEWAYLLEWPRVFLNASVDSFSKQEVFEPLDGKNSNFEPDNIRHIDWLVKRSIERANKFQIPSSSINRFFVQGIVKRIIPAVASTNAIIAASCCNEALKILTESNPFLDNYMMYVGEDGAYTYTFNLEKRSDCPVCGVLSEVYDISASSTVTLKDILNHYSKSYNLQNPSVSTAAGTPLYLASPPALQVATSKNLSQPILSITSVDVNLVITDKNLSTSLSVQLREC

SEQ ID NO: 26 - protein, 299 aa, Saccharomyces cerevisiae S288c
MDCKILVLGAGGLGCEILKNLTMLSFVKQVHIVDIDTIELTNLNRQFLFCDKDIGKPKAQVAAQYVNTRFPQLEVVAHVQDLTTLPPSFYKDFQFIISGLDAIEPRRFINETLVKLTLESNYEICIPFIDGGTEGLKGHVKTIIPGITACWECSIDTLPSQQDTVPMCTIANNPRCIEHVVEYVSTIQYPDLNIESTADMEFLLEKCCERAAQFSISTEKLSTSFILGIIKSIIPSVSTTNAMVAATCCTQMVKIYNDLIDLENGNNFTLINCSEGCFMYSFKFERLPDCTVCSNSNSN

SEQ ID NO: 27 - protein, 640 aa, Homo sapiens
MALSRGLPRELAEAVAGGRVLVVGAGGIGCELLKNLVLTGFSHIDLIDLDTIDVSNLNRQFLFQKKHVGRSKAQVAKESVLQFYPKANIVAYHDSIMNPDYNVEFFRQFILVMNALDNRAARNHVNRMCLAADVPLIESGTAGYLGQVTTIKKGVTECYECHPKPTQRTFPGCTIRNTPSEPIHCIVWAKYLFNQLFGEEDADQEVSPDRADPEAAWEPTEAEARARASNEDGDIKRISTKEWAKSTGYDPVKLFTKLFKDDIRYLLTMDKLWRKRKPPVPLDWAEVQSQGEETNASDQQNEPQLGLKDQQVLDVKSYARLFSKSIETLRVHLAEKGDGAELIWDKDDPSAMDFVTSAANLRMHIFSMNMKSRFDIKSMAGNIIPAIATTNAVIAGLIVLEGLKILSGKIDQCRTIFLNKQPNPRKKLLVPCALDPPNPNCYVCASKPEVTVRLNVHKVTVLTLQDKIVKEKFAMVAPDVQIEDGKGTILISSEEGETEANNHKKLSEFGIRNGSRLQADDFLQDYTLLINILHSEDLGKDVEFEVVGDAPEKVGPKQAEDAAKSITNGSDDGAQPSTSTAQEQDDVLIVDSDEEDSSNNADVSEEERSRKRKLDEKENLSAKRSRIEQKEELDDVIALD

SEQ ID NO: 28 - protein, 1024 aa, Saccharomyces cerevisiae (strain ATCC 204508
MSSNNSGLSAAGEIDESLYSRQLYVLGKEAMLKMQTSNVLILGLKGLGVEIAKNVVLAGVKSMTVFDPEPVQLADLSTQFFLTEKDIGQKRGDVTRAKLAELNAYVPVNVLDSLDDVTQLSQFQVVVATDTVSLEDKVKINEFCHSSGIRFISSETRGLFGNTFVDLGDEFTVLDPTGEEPRTGMVSDIEPDGTVTMLDDNRHGLEDGNFVRFSEVEGLDKLNDGTLFKVEVLGPFAFRIGSVKEYGEYKKGGIFTEVKVPRKISFKSLKQQLSNPEFVFSDFAKFDRAAQLHLGFQALHQFAVRHNGELPRTMNDEDANELIKLVTDLSVQQPEVLGEGVDVNEDLIKELSYQARGDIPGVVAFFGGLVAQEVLKACSGKFTPLKQFMYFDSLESLPDPKNFPRNEKTTQPVNSRYDNQIAVFGLDFQKKIANSKVFLVGSGAIGCEMLKNWALLGLGSGSDGYIVVTDNDSIEKSNLNRQFLFRPKDVGKNKSEVAAEAVCAMNPDLKGKINAKIDKVGPETEEIFNDSFWESLDFVTNALDNVDARTYVDRRCVFYRKPLLESGTLGTKGNTQVIIPRLTESYSSSRDPPEKSIPLCTLRSFPNKIDHTIAWAKSLFQGYFTDSAENVNMYLTQPNFVEQTLKQSGDVKGVLESISDSLSSKPHNFEDCIKWARLEFEKKFNHDIKQLLFNFPKDAKTSNGEPFWSGAKRAPTPLEFDIYNNDHFHFVVAGASLRAYNYGIKSDDSNSKPNVDEYKSVIDHMIIPEFTPNANLKIQVNDDDPDPNANAANGSDEIDQLVSSLPDPSTLAGFKLEPVDFEKDDDTNHHIEFITACSNCRAQNYFIETADRQKTKFIAGRIIPAIATTTSLVTGLVNLELYKLIDNKTDIEQYKNGFVNLALPFFGFSEPIASPKGEYNNKKYDKIWDRFDIKGDIKLSDLIEHFEKDEGLEITMLSYGVSLLYASFFPPKKLKERLNLPITQLVKLVTKKDIPAHVSTMILEICADDKEGEDVEVPFITIHL

SEQ ID NO: 29 - protein, 1058 aa, Homo sapiens
MSSSPLSKKRRVSGPDPKPGSNCSPAQSVLSEVPSVPTNGMAKNGSEADIDEGLYSRQLYVLGHEAMKRLQTSSVLVSGLRGLGVEIAKNIILGGVKAVTLHDQGTAQWADLSSQFYLREEDIGKNRAEVSQPRLAELNSYVPVTAYTGPLVEDFLSGFQVVVLTNTPLEDQLRVGEFCHNRGIKLVVADTRGLFGQLFCDFGEEMILTDSNGEQPLSAMVSMVTKDNPGVVTCLDEARHGFESGDFVSFSEVQGMVELNGNQPMEIKVLGPYTFSICDTSNFSDYIRGGIVSQVKVPKKISFKSLVASLAEPDFVVTDFAKFSRPAQLHIGFQALHQFCAQHGRPPRPRNEEDAAELVALAQAVNARALPAVQQNNLDEDLIRKLAYVAAGDLAPINAFIGGLAAQEVMKACSGKFMPIMQWLYFDALECLPEDKEVLTEDKCLQRQNRYDGQVAVFGSDLQEKLGKQKYFLVGAGAIGCELLKNFAMIGLGCGEGGEIIVTDMDTIEKSNLNRQFLFRPWDVTKLKSDTAAAAVRQMNPHIRVTSHQNRVGPDTERIYDDDFFQNLDGVANALDNVDARMYMDRRCVYYRKPLLESGTLGTKGNVQVVIPFLTESYSSSQDPPEKSIPICTLKNFPNAIEHTLQWARDEFEGLFKQPAENVNQYLTDPKFVERTLRLAGTQPLEVLEAVQRSLVLQRPQTWADCVTWACHHWHTQYSNNIRQLLHNFPPDQLTSSGAPFWSGPKRCPHPLTFDVNNPLHLDYVMAAANLFAQTYGLTGSQDRAAVATFLQSVQVPEFTPKSGVKIHVSDQELQSANASVDDSRLEELKATLPSPDKLPGFKMYPIDFEKDDDSNFHMDFIVAASNLRAENYDIPSADRHKSKLIAGKIIPAIATTTAAVVGLVCLELYKVVQGHRQLDSYKNGFLNLALPFFGFSEPLAAPRHQYYNQEWTLWDRFEVQGLQPNGEEMTLKQFLDYFKTEHKLEITMLSQGVSMLYSFFMPAAKLKERLDQPMTEIVSRVSKRKLGRHVRALVLELCCNDESGEDVEVPYVRYTIR

SEQ ID NO: 30 - protein, 460 aa, Homo sapiens
MASREEVLALQAEVAQREEELNSLKQKLASALLAEQEPQPERLVPVSPLPPKAALSRDEILRYSRQLVLPELGVHGQLRLGTACVLIVGCGGLGCPLAQYLAAAGVGRLGLVDYDVVEMSNLARQVLHGEALAGQAKAFSAAASLRRLNSAVECVPYTQALTPATALDLVRRYDVVADCSDNVPTRYLVNDACVLAGRPLVSASALRFEGQITVYHYDGGPCYRCIFPQPPPAETVTNCADGGVLGVVTGVLGCLQALEVLKIAAGLGPSYSGSLLLFDALRGHFRSIRLRSRRLDCAACGERPTVTDLLDYEAFCGSSATDKCRSLQLLSPEERVSVTDYKRLLDSGAFHLLLDVRPQVEVDICRLPHALHIPLKHLERRDAESLKLLKEAIWEEKQGTQEGAAVPIYVICKLGNDSQKAVKILQSLSAAQELDPLTVRDVVGGLMAWAAKIDGTFPQY

SEQ ID NO: 31 - protein, 404 aa, Homo sapiens
MAESVERLQQRVQELERELAQERSLQVPRSGDGGGGRVRIEKMSSEVVDSNPYSRLMALKRMGIVSDYEKIRTFAVAIVGVGGVGSVTAEMLTRCGIGKLLLFDYDKVELANMNRLFFQPHQAGLSKVQAAEHTLRNINPDVLFEVHNYNITTVENFQHFMDRISNGGLEEGKPVDLVLSCVDNFEARMTINTACNELGQTWMESGVSENAVSGHIQLIIPGESACFACAPPLVVAANIDEKTLKREGVCAASLPTTMGVVAGILVQNVLKFLLNFGTVSFYLGYNAMQDFFPTMSMKPNPQCDDRNCRKQQEEYKKKVAALPKQEVIQEEEEIIHEDNEWGIELVSEVSEEELKNFSGPVPDLPEGITVAYTIPKKQEDSVTELTVEDSGESLEDLMAKMKNM

SEQ ID NO: 32 - protein, 1052 aa, Homo sapiens
MEGSEPVAAHQGEEASCSSWGTGSTNKNLPIMSTASVEIDDALYSRQRYVLGDTAMQKMAKSHVFLSGMGGLGLEIAKNLVLAGIKAVTIHDTEKCQAWDLGTNFFLSEDDVVNKRNRAEAVLKHIAELNPYVHVTSSSVPFNETTDLSFLDKYQCVVLTEMKLPLQKKINDFCRSQCPPIKFISADVHGIWSRLFCDFGDEFEVLDTTGEEPKEIFISNITQANPGIVTCLENHPHKLETGQFLTFREINGMTGLNGSIQQITVISPFSFSIGDTTELEPYLHGGIAVQVKTPKTVFFESLERQLKHPKCLIVDFSNPEAPLEIHTAMLALDQFQEKYSRKPNVGCQQDSEELLKLATSISETLEEKPDVNADIVHWLSWTAQGFLSPLAAAVGGVASQEVLKAVTGKFSPLCQWLYLEAADIVESLGKPECEEFLPRGDRYDALRACIGDTLCQKLQNLNIFLVGCGAIGCEMLKNFALLGVGTSKEKGMITVTDPDLIEKSNLNRQFLFRPHHIQKPKSYTAADATLKINSQIKIDAHLNKVCPTTETIYNDEFYTKQDVIITALDNVEARRYVDSRCLANLRPLLDSGTMGTKGHTEVIVPHLTESYNSHRDPPEEEIPFCTLKSFPAAIEHTIQWARDKFESSFSHKPSLFNKFWQTYSSAEEVLQKIQSGHSLEGCFQVIKLLSRRPRNWSQCVELARLKFEKYFNHKALQLLHCFPLDIRLKDGSLFWQSPKRPPSPIKFDLNEPLHLSFLQNAAKLYATVYCIPFAEEDLSADALLNILSEVKIQEFKPSNKVVQTDETARKPDHVPISSEDERNAIFQLEKAILSNEATKSDLQMAVLSFEKDDDHNGHIDFITAASNLRAKMYSIEPADRFKTKRIAGKIIPAIATTTATVSGLVALEMIKVTGGYPFEAYKNCFLNLAIPIVVFTETTEVRKTKIRNGISFTIWDRWTVHGKEDFTLLDFINAVKEKYGIEPTMVVQGVKMLYVPVMPGHAKRLKLTMHKLVKPTTEKKYVDLTVSFAPDIDGDEDLPGPPVRYYFSHDTD

SEQ ID NO: 33 - protein, 1012 aa, Homo sapiens
MDALDASKLLDEELYSRQLYVLGSPAMQRIQGARVLVSGLQGLGAEVAKNLVLMGVGSLTLHDPHPTCWSDLAAQFLLSEQDLERSRAEASQELLAQLNRAVQVVVHTGDITEDLLLDFQVVVLTAAKLEEQLKVGTLCHKHGVCFLAADTRGLVGQLFCDFGEDFTVQDPTEAEPLTAAIQHISQGSPGILTLRKGANTHYFRDGDLVTFSGIEGMVELNDCDPRSIHVREDGSLEIGDTTTFSRYLRGGAITEVKRPKTVRHKSLDTALLQPHVVAQSSQEVHHAHCLHQAFCALHKFQHLHGRPPQPWDPVDAETVVGLARDLEPLKRTEEEPLEEPLDEALVRTVALSSAGVLSPMVAMLGAVAAQEVLKAISRKFMPLDQWLYFDALDCLPEDGELLPSPEDCALRGSRYDGQIAVFGAGFQEKLRRQHYLLVGAGAIGCELLKVFALVGLGAGNSGGLTVVDMDHIERSNLSRQFLFRSQDVGRPKAEVAAAAARGLNPDLQVIPLTYPLDPTTEHIYGDNFFSRVDGVAAALDSFQARRYVAARCTHYLKPLLEAGTSGTWGSATVFMPHVTEAYRAPASAAASEDAPYPVCTVRYFPSTAEHTLQWARHEFEELFRLSAETINHHQQAHTSLADMDEPQTLTLLKPVLGVLRVRPQNWQDCVAWALGHWKLCFHYGIKQLLRHFPPNKVLEDGTPFWSGPKQCPQPLEFDTNQDTHLLYVLAAANLYAQMHGLPGSQDWTALRELLKLLPQPDPQQMAPIFASNLELASASAEFGPEQQKELNKALEVWSVGPPLKPLMFEKDDDSNFHVDFVVAAASLRCQNYGIPPVNRAQSKRIVGQIIPAIATTTAAVAGLLGLELYKVVSGPRPRSAFRHSYLHLAENYLIRYMPFAPAIQTFHHLKWTSWDRLKVPAGQPERTLESLLAHLQEQHGLRVRILLHGSALLYAAGWSPEKQAQHLPLRVTELVQQLTGQAPAPGQRVLVLELSCEGDDEDTAFPPLHYEL

SEQ ID NO: 34 - protein, 703 aa, Homo sapiens
MAAATGDPGLSKLQFAPFSSALDVGFWHELTQKKLNEYRLDEAPKDIKGYYYNGDSAGLPARLTLEFSAFDMSAPTPARCCPAIGTLYNTNTLESFKTADKKLLLEQAANEIWESIKSGTALENPVLLNKFLLLTFADLKKYHFYYWFCYPALCLPESLPLIQGPVGLDQRFSLKQIEALECAYDNLCQTEGVTALPYFLIKYDENMVLVSLLKHYSDFFQGQRTKITIGVYDPCNLAQYPGWPLRNFLVLAAHRWSSSFQSVEVVCFRDRTMQGARDVAHSIIFEVKLPEMAFSPDCPKAVGWEKNQKGGMGPRMVNLSECMDPKRLAESSVDLNLKLMCWRLVPTLDLDKVVSVKCLLLGAGTLGCNVARTLMGWGVRHITFVDNAKISYSNPVRQPLYEFEDCLGGGKPKALAAADRLQKIFPGVNARGFNMSIPMPGHPVNFSSVTLEQARRDVEQLEQLIESHDVVFLLMDTRESRWLPAVIAASKRKLVINAALGFDTFVVMRHGLKKPKQQGAGDLCPNHPVASADLLGSSLFANIPGYKLGCYFCNDVVAPGDSTRDRTLDQQCTVSRPGLAVIAGALAVELMVSVLQHPEGGYAIASSSDDRMNEPPTSLGLVPHQIRGFLSRFDNVLPVSLAFDKCTACSSKVLDQYEREGFNFLAKVFNSSHSFLEDLTGLTLLHQETQAAEIWDMSDDETI

SEQ ID NO: 35 - protein, 249 aa, Escherichia coli (strain K12)
MAELSDQEMLRYNRQIILRGFDFDGQEALKDSRVLIVGLGGLGCAASQYLASAGVGNLTLLDFDTVSLSNLQRQTLHSDATVGQPKVESARDALTRINPHIAITPVNALLDDAELAALIAEHDLVLDCTDNVAVRNQLNAGCFAAKVPLVSGAAIRMEGQITVFTYQDGEPCYRCLSRLFGENALTCVEAGVMAPLIGVIGSLQAMEAIKMLAGYGKPASGKIVMYDAMTCQFREMKLMRNPGCEVCGQ

SEQ ID NO: 36 - protein, 251 aa, Escherichia coli (strain K12)
MNDRDFMRYSRQILLDDIALDGQQKLLDSQVLIIGLGGLGTPAALYLAGAGVGTLVLADDDDVHLSNLQRQILFTTEDIDRPKSQVSQQRLTQLNPDIQLTALQQRLTGEALKDAVARADVVLDCTDNMATRQEINAACVALNTPLITASAVGFGGQLMVLTPPWEQGCYRCLWPDNQEPERNCRTAGVVGPVVGVMGTLQALEAIKLLSGIETPAGELRLFDGKSSQWRSLALRRASGCPVCGGSNADPV

(19) United States
(12) Patent Application Publication
(10) Pub. No.: US 2015/0030601 A1
(43) Pub. Date: Jan. 29, 2015

(54) E1 ENZYME MUTANTS AND USES THEREOF

(71) Applicant: MILLENNIUM PHARMACEUTICALS, INC., Cambridge, MA (US)

(72) Inventors: Benjamin Stone Amidon, Arlington, MA (US); James E. Brownell, Winchester, MA (US); James M. Gavin, South Easton, MA (US); Erik M. Koenig, North Reading, MA (US); Michael D. Sintchak, Winchester, MA (US); Peter G. Smith, Arlington, MA (US)

(73) Assignee: Millennium Pharmaceuticals, Inc., Cambridge, MA (US)

(21) Appl. No.: 14/349,843

(22) PCT Filed: Oct. 5, 2012
(86) PCT No.: PCT/US2012/058983

§ 371 Date: Apr. 4, 2014

Related U.S. Application Data
(60) Provisional application No. 61/544,843, filed on Oct. 7, 2011.
(60) Provisional application No. 61/596,420, filed on Feb. 8, 2012.

(51) Int. Cl. C12Q 1/68; G01N 33/573; C07K 16/40; C12N 9/02

(52) U.S. Cl. CPC C12Q 1/6883; C12N 9/0008; G01N 33/573; C07K 16/40; G01N 2500/04; C12Y 102/02; G01N 2333/90203; C12Q 2600/158; C12Q 2600/106
USPC 424/139.1; 435/191; 435/7.4; 536/23.2; 530/387.9; 435/320.1; 435/325; 435/6.11; 435/184

(57) ABSTRACT

The invention provides isolated nucleic acids molecules, designated UBA3, UAE, or UBA6, or other E1 enzyme variant nucleic acid molecules, which encode novel E1 enzyme variant proteins. The invention also provides antisense nucleic acid molecules, recombinant expression vectors containing UBA3, UAE, or UBA6, or other E1 enzyme variant nucleic acid molecules, host cells into which the expression vectors have been introduced, and nonhuman transgenic animals in which a UBA3, UAE, or UBA6, or other E1 enzyme variant gene has been introduced or disrupted. The invention still further provides isolated UBA3, UAE, or UBA6, or other E1 enzyme variant proteins, fusion proteins, antigenic peptides and anti-UBA3, UAE, or UBA6, or other E1 enzyme variant antibodies. The invention provides methods to identify agents that inhibit UBA3, UAE, or UBA6, or other E1 enzyme variant expression or activity. Diagnostic and therapeutic methods utilizing compositions of the invention are also provided.

### RELATED APPLICATIONS

This application claims priority to U.S. Provisional Application No. 61/596,420 filed on Feb. 8, 2012 and U.S. Provisional Application No. 61/544,843 filed on Oct. 7, 2011. The entire contents of the foregoing applications are incorporated herein by reference.

### SEQUENCE LISTING

This application contains a Sequence Listing which is submitted herewith in electronically readable format. The Sequence Listing file was created on Oct. 3, 2012, is named “sequencelisting.txt,” and is 135 kb (138,289 bytes). The entire contents of the Sequence Listing in this sequencelisting.txt file are incorporated herein by this reference.

### BACKGROUND

The post-translational modification of proteins by ubiquitin-like molecules (ubls) is a regulatory process within cells, playing roles in controlling many biological processes including cell division, cell signaling and the immune response. Ubls are small proteins that are covalently attached through the action of a coordinated series of enzymes to a lysine on a target protein via an isopeptide linkage with a C-terminal glycine of the ubl. The ubiquitin-like molecule alters the molecular surface of the target protein and can affect such properties as protein-protein interactions, enzymatic activity, stability and cellular localization of the target.

Developing therapies that modulate the series of enzymes that attach ubls to target proteins has provided opportunities to interfere with a variety of biochemical pathways involved in maintaining the integrity of cell division and cell signaling. As such, inhibition of these enzymes, and the resultant inhibition of downstream effects of ubl-conjugation, represents a method of interfering with the integrity of cell division, cell signaling, and several aspects of cellular physiology which are important for disease mechanisms. Some of this interference can lead to apoptotic death of tumor cells.

Chemotherapy, such as for treating cancer, commonly involves the administration of one or more cytotoxic or cytostatic drugs to a patient. A goal of cancer chemotherapy is to eradicate a tumor comprising transformed cells from the body of the individual, or to suppress or to attenuate growth of the tumor. Another goal of cancer chemotherapy is stabilization (clinical management) of the afflicted individual's health status. Although some tumors may initially respond to chemotherapy, in many instances the initial chemotherapeutic treatment regimen becomes less effective or ceases to impede tumor growth. Phenotypic or genotypic properties allow some tumor cells to resist the effects of a chemotherapeutic drug. The occurrence of amino acid substitutions has been described as a common form of resistance for cancer drugs such as tyrosine kinase inhibitors including imitanib, gefitinib and erlotinib (Shah et al., 2002, Kobayashi et al., 2005, Pao et al., 2005). More recent examples include amino acid substitutions in the anaplastic lymphoma kinase (ALK) following crizotinib therapy that occurred in lung cancer patients harboring an EML4-ALK translocation (Choi et al., 2010). Mutations in ALK that reduced sensitivity to crizotinib were originally described in pre-clinical studies using models of NPM-ALK translocations which led to their prediction that they may occur in EML4-ALK cancers (Lu et al., 2009). These data confirm that the enzyme targets of these drugs “drive” the cancer and that the activity of the inhibitors is through inhibition of the target.

Identification of variations which enable cells of tumors or pathogenic organisms to resist chemotherapy can aid in the development of therapies to address the resistance.

### SUMMARY

The present invention relates to the field of E1 enzymes, and variants exhibiting a reduced sensitivity to particular agents. The invention relates to E1 enzyme variants which exhibit a decreased sensitivity to an E1 enzyme inhibitor, and to methods to identify inhibitors which overcome the decreased sensitivity. The present invention also relates to isolated E1 enzyme variants that comprise at least one nucleotide mutation in the E1 enzyme gene, including variants wherein said nucleotide mutations result in at least one amino acid mutation, such as an amino acid substitution, in the E1 enzyme protein. In one aspect the E1 enzyme is NEDD8-activating enzyme (NAE, comprised of APPBP1/NAEα and UBA3/NAEβ). In another aspect, the E1 enzyme is UBA1. In another aspect, the E1 enzyme is UBA6.

The present invention also relates to isolated UBA3 variants that comprise at least one nucleotide mutation in the UBA3 gene, including variants wherein said nucleotide mutations result in at least one mutation, e.g. an amino acid substitution in the UBA3 protein. The invention relates to UBA3 variants which exhibit a decreased sensitivity to an NAE inhibitor, such as a 1-substituted methyl sulfamate (See FIG. 1). In addition, the present invention relates to the field of diagnosing the susceptibility of a tumor sample to inhibitors and to a method and/or assay for the detection of mutations in the UBA3 gene.

In one aspect, the UBA3, UAE, or UBA6, or other E1 enzyme variants comprise nucleotide sequences which are expressed in drug resistant tumor cells (drug resistance sequences). Expression of the resistance sequences, e.g., UBA3, UAE, or UBA6, or other E1 enzyme variant sequences is either increased (up-regulated sequences) or decreased (down-regulated sequences) in a particular tumor cell when compared to expression in a control cell (i.e., non-drug resistant cell) or a less drug resistant tumor cell. Drug resistance sequences include nucleic acids and polypeptides that are useful in, for example, diagnostic methods related to identification of drug resistant cells (e.g., cancer cells). Resistance sequences (i.e., resistance genes, resistance mRNAs, resistance cDNAs, resistance polypeptides, and resistance proteins or fragments of any of the foregoing) are also useful in screening methods directed to the identification of compounds that can modulate (increase or decrease) the drug resistance of a particular cell type or multiple cell types.

Isolated nucleic acids corresponding to UBA3, UAE, or UBA6, or other E1 enzyme variant nucleic acid sequences are provided. For example, the isolated polynucleic acids comprise at least one nucleotide mutation in the UBA3, UAE, or UBA6, or other E1 enzyme gene. A nucleotide mutation further can result in at least one amino acid mutation, e.g., substitution and/or deletion in the UBA3, UAE, or UBA6, or other E1 enzyme protein. A nucleotide mutation in a polynucleic acid comprising a UBA3 nucleotide mutation can be expressed to produce a polypeptide comprising at least a fragment of a UBA3 variant protein and can lead to a reduced sensitivity to an NAE inhibitor, such as a 1-substituted methyl sulfamate. Additionally, amino acid sequences corresponding to the variant polynucleotides are encompassed. The nucleotide sequence of a wild type cDNA encoding human UBA3 is shown in SEQ ID NO:1, and the amino acid sequence of a wild type human UBA3 polypeptide is shown in SEQ ID NO:2. In particular, the present invention provides for isolated nucleic acid molecules comprising variant UBA3 nucleotide sequences encoding variant UBA3 polypeptides, e.g., variations of the amino acid sequence shown in SEQ ID NO: 2. Further provided are expression products from these isolated polynucleic acids and to UBA3 polypeptides or fragments thereof having a variant amino acid sequence encoded by a nucleic acid molecule described herein.

Nucleic acid molecules and polypeptides substantially homologous to the nucleotide and amino acid sequences set forth in the sequence listings are encompassed by the present invention. Additionally, fragments and substantially homologous fragments of the nucleotide and amino acid sequences are provided.

In a related aspect, the invention further provides nucleic acid constructs which include a UBA3, UAE, or UBA6, or other E1 enzyme variant nucleic acid molecule described herein. In some embodiments, the nucleic acid molecules of the invention are operatively linked to native or heterologous regulatory sequences. The present invention also provides vectors and cultured host cells for recombinant expression of the nucleic acid molecules described herein, as well as methods of making such vectors and host cells and for using them for production of the polypeptides or peptides of the invention by recombinant techniques. In a related aspect, the invention provides UBA3, UAE, or UBA6, or other E1 enzyme variant polypeptides or fragments operatively linked to non-UBA3, UAE, or UBA6, or other E1 enzyme variant polypeptides to form fusion proteins.

The UBA3, UAE, or UBA6, or other E1 enzyme variant molecules of the present invention are useful for modulating cell growth, cell-cycle proliferation and cellular signal transduction. The UBA3 variant molecules are useful for the diagnosis and treatment of a disorder wherein there is aberrant cell growth and proliferation, cell-cycle progression or aberrant signal transduction, including resistance to NAE modulators. Accordingly, in one aspect, this invention provides isolated nucleic acid molecules encoding UBA3, UAE, or UBA6, or other E1 enzyme variant proteins or biologically active portions thereof, as well as nucleic acid fragments suitable as primers or hybridization probes for the detection or isolation of UBA3, UAE, or UBA6, or other E1 enzyme variant-encoding nucleic acids. In another aspect, isolated nucleic acid molecules that are antisense to UBA3, UAE, or UBA6, or other E1 enzyme variant-encoding nucleic acid molecules are provided. In addition, a UBA3, UAE, or UBA6, or other E1 enzyme variant nucleic acid, fragment or complement thereof can be incorporated into a pharmaceutical composition, which optionally includes a pharmaceutically acceptable carrier.

Another aspect of this invention features isolated or recombinant UBA3 variant proteins and polypeptides, and biologically active or antigenic fragments thereof that are useful, e.g., as reagents or targets in assays applicable to treatment and diagnosis of NAE-associated or other disorders. In another embodiment, the invention provides UBA3 polypeptides having a UBA3 variant activity. In certain embodiments, polypeptides are UBA3 variant proteins including at least one ATP binding pocket, NEDD8 binding pocket, ThiF family domain, UBA_e1_thiolCys domain, UBACT domain or E2_bind domain, and, optionally, having a UBA3 activity, e.g., a UBA3 activity as described herein. In some embodiments, UBA3 variant proteins and polypeptides possess at least one biological activity possessed by wild type UBA3 proteins. In other embodiments, a UBA3 variant protein has a change in the activity of one or more domains compared to wild type UBA3 protein. A UBA3 variant protein can have an altered domain such that the activity of the domain is decreased, reduced or eliminated. In addition, a UBA3 variant protein (a protein encoded by a UBA3 variant gene) or biologically active portions thereof can be incorporated into a pharmaceutical composition, which optionally includes pharmaceutically acceptable carriers.

Antibodies and antibody fragments that react with, e.g., specifically or selectively bind, the UBA3, UAE, or UBA6, or other E1 enzyme variant polypeptides and fragments are provided. The antibodies or fragments thereof can distinguish a UBA3, UAE, or UBA6, or other E1 enzyme variant polypeptide from a wild type UBA3, UAE, or UBA6, or other E1 enzyme protein.

In another aspect, the invention provides methods of screening for compounds that modulate the expression or activity of the UBA3, UAE, or UBA6, or other E1 enzyme variant polypeptides or nucleic acids. In general, screening for compounds that modulate the expression involves measuring UBA3, UAE, or UBA6, or other E1 enzyme variant expression in the presence and absence of a test compound and identifying those compounds which alter the UBA3, UAE, or UBA6, or other E1 enzyme variant expression. The UBA3, UAE, or UBA6, or other E1 enzyme variant expression that is measured can be expression of a UBA3, UAE, or UBA6, or other E1 enzyme variant nucleic acid or a UBA3, UAE, or UBA6, or other E1 enzyme variant polypeptide. In general, a method for identifying a compound that modulates UBA3, UAE, or UBA6, or other E1 enzyme variant activity entails measuring a biological activity of the polypeptide in the presence and absence of a test compound and identifying those compounds which alter the activity of the polypeptide. In particular, UBA3, UAE, or UBA6, or other E1 enzyme variants or polynucleic acids or expression products or compositions of the present invention are used in a process for the selection of at least one drug that modulates variant UBA3, UAE, or UBA6, or other E1 enzyme expression or activity.

The invention features methods for identifying a compound which binds to a UBA3, UAE, or UBA6, or other E1 enzyme variant polypeptide. These methods include the steps of contacting a UBA3, UAE, or UBA6, or other E1 enzyme variant polypeptide with a test compound and then determining whether the polypeptide binds to the test compound. In various embodiments of these methods, the binding of the test compound to the UBA3, UAE, or UBA6, or other E1 enzyme variant polypeptide is detected using an assay which measures direct binding of the test compound to the polypeptide or indirect binding using a competition binding assay.

In another aspect, the present invention provides a method for detecting the presence of resistance activity or expression in a biological sample by contacting the biological sample with an agent capable of detecting an indicator of a UBA3, UAE, or UBA6, or other E1 enzyme variant sequence activity such that the presence of the activity is detected in the biological sample. For example, the invention includes a method for detecting the presence of a UBA3, UAE, or UBA6, or other E1 enzyme variant polypeptide in a sample. This method features the steps of contacting the sample with a compound which selectively binds to the polypeptide and then determining whether the compound binds to a polypeptide in the sample. In some cases, the compound which binds to the polypeptide is an antibody. In another aspect, the present invention provides a method for detecting the presence of UBA3, UAE, or UBA6, or other E1 enzyme variant activity or expression in a biological sample by contacting the biological sample with an agent capable of detecting an indicator of UBA3, UAE, or UBA6, or other E1 enzyme variant activity such that the presence of the UBA3, UAE, or UBA6, or other E1 enzyme variant activity is detected in the biological sample. The invention also features methods for detecting the presence of a UBA3, UAE, or UBA6, or other E1 enzyme variant mRNA (an mRNA encoding a UBA3, UAE, or UBA6, or other E1 enzyme variant protein in a sample). This method includes the steps of contacting the sample with a nucleic acid probe or primer which selectively hybridizes to a UBA3, UAE, or UBA6, or other E1 enzyme variant mRNA; and then determining whether the nucleic acid probe or primer binds to a nucleic acid molecule in the sample.

In a further aspect, the invention provides assays for determining the presence or absence of a genetic alteration in a UBA3, UAE, or UBA6, or other E1 enzyme polypeptide or nucleic acid molecule, including for disease diagnosis. The present invention also provides a diagnostic assay for identifying the presence or absence of a genetic lesion or mutation characterized by at least one of: (i) aberrant modification or mutation of a gene encoding a UBA3, UAE, or UBA6, or other E1 enzyme protein; (ii) mis-regulation of a gene encoding a UBA3, UAE, or UBA6, or other E1 enzyme protein; (iii) aberrant RNA splicing; and (iv) aberrant post-translational modification of a UBA3, UAE, or UBA6, or other E1 enzyme protein, wherein a wild-type form of the gene encodes a protein with a normal UBA3, UAE, or UBA6, or other E1 enzyme activity.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a UBA3, UAE, or UBA6, or other E1 enzyme variant molecule. In one embodiment, the capture probe is a nucleic acid, e.g., a probe complementary to a UBA3, UAE, or UBA6, or other E1 enzyme variant nucleic acid sequence. In another embodiment, the capture probe is a polypeptide, e.g., an antibody specific for UBA3, UAE, or UBA6, or other E1 enzyme variant polypeptides. Also featured is a method of analyzing a sample by contacting the sample to the aforementioned array and detecting binding of the sample to the array.

Also within the invention are kits that include a compound which selectively binds to a UBA3, UAE, or UBA6, or other E1 enzyme variant polypeptide or nucleic acid and instructions for use. Such kits can be used to determine whether a particular cell type or cells within a biological sample, e.g., a sample of cells obtained from a patient, are drug resistant.

Also within the invention is a method of determining whether a cell has a drug-resistant phenotype by measuring the expression or activity of a UBA3, UAE, or UBA6, or other E1 enzyme variant sequence (e.g., an mRNA or a polypeptide) in the cell and comparing this expression or activity to expression or activity in a control cell. Increased expression or activity of an up-regulated UBA3, UAE, or UBA6, or other E1 enzyme variant sequence or its product in the cell compared to the control cell indicates that the cell has a drug-resistant phenotype. Decreased expression or activity of a down-regulated UBA3, UAE, or UBA6, or other E1 enzyme variant sequence, its product or genes in its pathway in the cell compared to the control cell indicates that the cell has a drug-resistant phenotype.

In one embodiment of this method, drug resistance is determined by measuring a UBA3 variant sequence (e.g., measuring an up-regulated or down-regulated UBA3 variant protein such as SEQ ID NO:2 with a mutation described herein, e.g., using an antibody that binds an epitope characterized by the mutated residue). In another embodiment, UBA3 variant sequence expression is measured by quantifying mRNA encoding a UBA3 variant protein or the copy number of the gene encoding the UBA3 variant protein. In another embodiment UBA3 variant sequence activity is measured using any assay which can quantify a biological activity of a UBA3 variant protein.

In yet another aspect the invention features a method for determining whether a subject has or is at risk of developing a drug resistant tumor, the method including measuring the expression of a UBA3, UAE, or UBA6, or other E1 enzyme variant sequence or the activity of a UBA3, UAE, or UBA6, or other E1 enzyme variant protein.

In another aspect, the invention provides a method for modulating the activity of a UBA3, UAE, or UBA6, or other E1 enzyme variant protein comprising contacting a cell with an agent that modulates (inhibits or stimulates) activity of the UBA3, UAE, or UBA6, or other E1 enzyme variant protein or expression such that UBA3 variant activity or expression in the cell is modulated, e.g., using the compounds identified in the screens described herein. This method includes the steps of contacting the polypeptide or a cell expressing the polypeptide with a compound which binds to the polypeptide in a sufficient concentration to modulate the activity of the polypeptide. In one embodiment, the agent is an antibody that specifically binds to a UBA3, UAE, or UBA6, or other E1 enzyme variant protein. In another embodiment, the agent modulates UBA3, UAE, or UBA6, or other E1 enzyme variant expression by modulating transcription of a gene encoding a UBA3, UAE, or UBA6, or other E1 enzyme variant protein, splicing of a UBA3, UAE, or UBA6, or other E1 enzyme variant mRNA, or translation of a UBA3, UAE, or UBA6, or other E1 enzyme variant mRNA. In yet another embodiment, the agent is a nucleic acid molecule having a nucleotide sequence that is antisense to the coding strand of a UBA3, UAE, or UBA6, or other E1 enzyme variant mRNA or a gene encoding a UBA3, UAE, or UBA6, or other E1 enzyme variant protein.

The invention also includes a method for modulating the drug resistance of a cell by modulating UBA3, UAE, or UBA6, or other E1 enzyme variant expression or activity within the cell. Thus in one embodiment, the drug resistance of a cell is reduced by contacting the cell with a molecule (e.g., an antisense nucleic acid molecule or siRNA) that reduces the expression of an up-regulated UBA3, UAE, or UBA6, or other E1 enzyme variant sequence within the cell. In another embodiment, drug resistance of a cell is reduced by contacting the cell with a molecule which increases the expression of a down-regulated UBA3, UAE, or UBA6, or other E1 enzyme variant sequence within the cell.

In one embodiment, the methods of the present invention are used to treat a subject having, or suspected of having, a disorder (e.g., a drug-resistant cancer or infection) characterized by aberrant UBA3, UAE, or UBA6, or other E1 enzyme variant sequence expression (e.g., of a protein or nucleic acid) or activity by administering to the subject an agent, e.g., a therapeutically effective amount of a compound, which is a UBA3, UAE, or UBA6, or other E1 enzyme variant modulator. In one embodiment, the UBA3, UAE, or UBA6, or other E1 enzyme variant modulator is a UBA3, UAE, or UBA6, or other E1 enzyme variant protein. In another embodiment the UBA3, UAE, or UBA6, or other E1 enzyme variant modulator is a UBA3, UAE, or UBA6, or other E1 enzyme variant nucleic acid molecule, e.g., a molecule that alters the expression of a UBA3, UAE, or UBA6, or other E1 enzyme variant sequence. In other embodiments, the UBA3, UAE, or UBA6, or other E1 enzyme variant modulator is a peptide, peptidomimetic, or other small molecule. Examples of disorders involving aberrant or deficient NAE function or expression include, but are not limited to, cellular proliferative and/or differentiative disorders, infections, e.g., parasitic infections, immune e.g., inflammatory, disorders or neurodegenerative disorders.

Another aspect of the present invention is a method of improving effectiveness of chemotherapy for a mammal having a disorder associated with the presence of drug-resistant neoplastic cells. In this method, a chemotherapeutic drug and a molecule that reduces expression of an up-regulated UBA3, UAE, or UBA6, or other E1 enzyme variant sequence or increases expression of a down-regulated UBA3, UAE, or UBA6, or other E1 enzyme variant sequence can be co-administered to a mammal.

The agents tested in the present methods can be a single agent or a combination of agents. For example, the present methods can be used to determine whether a single chemotherapeutic agent, such as an NAE inhibitor, can be used to treat a cancer or whether a combination of two or more agents can be used.

The invention also features a method for treating a drug resistant tumor in a patient, the method comprising administering to said subject an amount of a resistance sequence antagonist or agonist effective to reduce drug resistance of said tumor in the patient. In another aspect, the invention features the use of an inhibitor of expression of a resistance sequence, or pharmaceutically acceptable salt thereof, or a pharmaceutical composition containing either entity, for the manufacture of a medicament for the treatment of a drug resistant tumor in a patient.

Unless otherwise defined, all technical and scientific terms used herein have the same meaning as commonly understood by one or ordinary skill in the art to which this invention belongs. Although methods and materials similar or equivalent to those described herein can be used in the practice or testing of the present invention, examples of methods and materials are described herein. All publications, patent applications, patents, and other references mentioned herein are incorporated by reference in their entirety. In addition, the materials, methods, and examples are illustrative only and are not intended to be limited. The details of one or more embodiments of the invention are set forth in the accompanying drawings and the description below. Other features, objects and advantages of the invention will be apparent from the detailed description, sequence listing, drawings and from the claims.

### DRAWINGS

FIG. 1. General structure of 1-substituted methyl sulfamate. G¹ is —O— or —CH₂—; G² is —H or —OH; G³ is —H or —OH; G⁴ is —NH—, —O— or a covalent bond; and G⁵ is substituted heteroaryl.

FIG. 2. General pathway for modifying substrate proteins with ubiquitin or ubiquitin-like proteins (using the ubiquitin (Ub) pathway as an example). E1: activating enzyme uses ATP to activate the Ub C-terminal glycine; E2: conjugating enzyme accepts Ub from E1 thru a transthiolation reaction; E3: ligase collaborates with E2 to transfer Ub to a lysine residue in a substrate protein. Ubl can be a mono-addition to a substrate or a poly-Ubl chain.

FIG. 3. Multiple alignment of human UBA3 SEQ ID NO:2 (isoform 1) with homologous proteins in multiple species. Sequences were retrieved from the results of a BLAST search of SEQ ID NO:2 and aligned by Clustal W (version 1.83, run through software by GenomeQuest, Inc., Westborough, Mass.). Sequences are: marmoset, SEQ ID NO:9; dog, SEQ ID NO:10; mouse, SEQ ID NO:11; rat, SEQ ID NO:12; cattle, SEQ ID NO:13; human UBA3 isoform 2, SEQ ID NO:14; pig, SEQ ID NO:15; African frog, SEQ ID NO:16; salmon, SEQ ID NO:17; yellow fever mosquito, SEQ ID NO:18; malaria mosquito, SEQ ID NO:19; body louse, SEQ ID NO:20; fruit fly, SEQ ID NO:21; black mold, SEQ ID NO:22; lung mold, SEQ ID NO:23; yeast j., SEQ ID NO:24; yeast p., SEQ ID NO:25; yeast c., SEQ ID NO:26. Percent identity of the BLAST alignment with SEQ ID NO:2 is depicted in the first portion of the alignment. Small letters above the alignment mark residues which mutate for resistance in UBA3 SEQ ID NO:2: a=A171, b=G201, c=E204, d=G205, e=N209, f=R211, g=Y228, h=P229, i=V305, j=P311, k=A314, l=C324.

FIG. 4. Multiple alignment of enzymes with structural or mechanistic similarity to UBA3 in the region comprising residues homologous to the region around residue 171 of SEQ ID NO:2. ̂ marks positions of residues that are conserved among all enzymes; * marks the position of A171T of UBA3. Key to sequences: UBA3: residues 165 to 182 of SEQ ID NO:2; SAE2: residues 115 to 132 of SEQ ID NO:27; yUAE: residues 542 to 559 of SEQ ID NO:28; hUAE: residues 574 to 591 of SEQ ID NO:29; UBA4: residues 179 to 196 of SEQ ID NO:30; UBA5: residues 181 to 198 of SEQ ID NO:31; UBA6: residues 567 to 584 of SEQ ID NO:32; UBA7: residues 538 to 555 of SEQ ID NO:33; ATG7: residues 474 to 491 of SEQ ID NO:34; moeb: residues 128 to 145 of SEQ ID NO:35; thif: residues 125 to 142 of SEQ ID NO:36.

FIG. 5. MLN4924 resistant cell line clones. HCT-116 cells and resistant clones were treated with DMSO or various concentrations of MLN4924 for 96 hours and cell viability was assessed with ATPlite assay.

FIG. 6. MLN4924 resistant cell line clones. A. Calu-6 and B. NCI-H460 cells and resistant clones were treated with DMSO or various concentrations of MLN4924 for 96 hours and cell viability was assessed with ATPlite assay.

FIG. 7. HCT-116 cells and resistant clones were treated with DMSO or various concentrations of A. bortezomib, B. doxorubicin or C. SN-38 for 96 hours and cell viability was assessed with ATPlite assay.

FIG. 8. Treatment of cells with efflux inhibitors. A. HCT-116 WT, B. HCT-116.1 A171T and C. HCT-116.4 C324Y cells were treated with DMSO or various concentrations of MLN4924 for 96 hours and cell viability was assessed with ATPlite assay. Co-incubation studies were performed with various inhibitors of drug efflux at their highest non toxic concentration. Diypridamole (10 μM), MK571 (10 μM), GF918 (1 μM), KO143 (1 μM) or LY5979 (5 μM) were added at the same time as DMSO or MLN4924 and incubated for 96 hours and cell viability assessed with ATPlite assay.

FIG. 9. Structure of UBA3. A. Schematic representation of location and frequency of NAEβ mutations detected in cells and xenografts. Expanded sequences are amino acid residues 148 to 171 and 201 to 229 of SEQ ID NO:2. B) Crystal structure of NAE with NEDD8-MLN4924 adduct bound (PDB entry 3GZN, Brownell et al., 2010) highlighting UBA3 mutations.

FIG. 10. Western blots of pathway proteins of MLN4924-treated cells. A. HCT-116 WT, B. HCT-116.1 A171T, C. HCT-116.5 G201V. Cells were treated with DMSO or various concentrations of MLN4924 for 24 hours. Western blots were probed for NEDD8-cullin, NEDD8-NAEβ, NEDD8-Ubc12, NEDD8-MLN4924 adduct, CDT1, NRF2 and tubulin.

FIG. 11. Cell cycle analysis. A. HCT-116 WT, B. HCT-116.1 A171T and C. HCT-116.5 G201V cells were treated with DMSO (top) or 1 μM MLN4924 (bottom) for 24 hours after which cells were stained with Propidium Iodide and cell cycle analysis performed.

FIG. 12. Western blots of CRL substrates in treated cells. A. Calu-6 WT, B. Calu-6.1 N209K, C. NCI-H460 and D. NCI-H460.2 A171D cells were treated with DMSO or various concentrations of MLN4924 for 24 hours. Western blots were probed for NEDD8-cullin, NEDD8-NAEβ, NEDD8-Ubc12, NEDD8-MLN4924 adduct, CDT1, NRF2 and tubulin.

FIG. 13. Resistant activity in HCT-116 xenografts. A. Immunocompromised nude rats bearing HCT-116 xenografts were administered 180 mg/kg MLN4924 on days 1, 4, 8, 11 of a 21 day cycle for 3 cycles. Tumors were harvested at the end of treatment for analysis and the mutational status of NAEβ determined. B. A tumor containing an Alanine 171 to threonine mutation was re-established in nude rats and treated with 180 mg/kg MLN4924 on days 1, 4, 8, 11 of a 21 day schedule. The response of WT (parental) tumors is included on the graph for comparison. Nude rats bearing HCT-116 parental or A171T xenografts were administered a single dose of 180 mg/kg MLN4924 and tumors were excised at the indicated times and measured for NEDD8-cullin conjugate levels C., Cdt-1 levels D., cleaved caspase-3 levels E. and NEDD8-adduct levels F.

FIG. 14. Acute Myelogenous Leukemia and Diffuse Large B-cell Lymphoma xenografts. A. CB.17 SCID mice bearing THP-1 AML xenografts were administered 90 mg/kg BID on days 1, 4, 8, 11, 15, 18 of a 21-day cycle for up to 5 cycles. Tumors were harvested at the end of treatment for analysis and the mutational status of NAEβ determined. B. A tumor containing an Alanine 171 to threonine mutation was re-established in CB.17 SCID mice and treated with 90 mg/kg BID MLN4924 on days 1, 4, 8, 11, 15, 18 of a 21-day cycle. The response of WT (parental) tumors is included on the graph for comparison. CB.17 SCID mice bearing THP-1 parental or A171T xenografts were administered a single dose of 90 mg/kg MLN4924 and tumors were excised at the indicated times and measured for C. NEDD8-cullin conjugate levels, D. Cdt-1 levels and E. cleaved caspase-3 levels. F. CB.17 SCID mice bearing OCI-Ly10 DLBCL xenografts were administered 90 mg/kg BID on days 1, 4, 8, 11, 15, 18 of a 21-day cycle for up to 5 cycles. Tumors were harvested at the end of treatment for analysis and the mutational status of NAEβ determined. G. A tumor containing a glutamic acid 204 to glycine mutation was re-established in CB.17 SCID mice and treated with 90 mg/kg BID MLN4924 on days 1, 4, 8, 11, 15, 18 of a 21-day cycle. The response of WT (parental) tumors is included on the graph for comparison.

FIG. 15. Inhibitor recovery of UBA3 mutants. NAEβ mutants were inhibited with MLN4294 or compound 1, purified and complexes were added to transthiolation reaction containing 1 mM ATP to measure recovery of enzyme activity. A. WT UBA3, B. A171T UBA3, C. N209K UBA3, D. E204K UBA3, E. G201V UBA3.

FIG. 16. Recovery of pathway activity in cells with UBA3 mutants. HCT-116 A. WT, B. A171T or C. G201V cells were treated with 1 or 10 uM MLN4924 for one hour, compound was washed out and cells incubated in drug free media and harvested at the indicated times. Protein lysates were probed by Western blotting for NEDD8-cullin, NEDD8-NAEβ, NEDD8-Ubc12, NEDD8-MLN4924 adduct, CDT1, NRF2 and tubulin.

FIG. 17. Recovery of pathway activity in cells with UBA3 mutants. Immunoprecipiation assays were performed with a NAEβ antibody and resultant isolates probed with NAEβ, NAE1 and NEDD8-MLN4924 adduct antibody. Flow through from the immunoprecipitation assays were probed with NEDD8-MLN4924 adduct antibody.

FIG. 18. Recovery of pathway activity in cells with UBA3 mutants. HCT-116 A. WT, B. A171T or C. G201V cells were treated with various concentrations of Compound 1 for four hours and protein lysates were probed by Western blotting for Ubc10, ubiquitin K48 chains, NEDD8-cullin, NEDD8-NAEβ, NEDD8-Ubc12, CDT1 and tubulin.

### DETAILED DESCRIPTION

Some tumor cells possess variant genes that are not susceptible to inhibitory effects of a therapeutic regimen. Alternatively, treatment of some tumor cells with chemotherapeutic agents results in mutations and enzyme gene variants which confer resistance of tumor cell progeny to the agent. These variant genes allow the tumor cells or their progeny to survive and/or proliferate and the tumor to persist or grow despite therapeutic intervention.

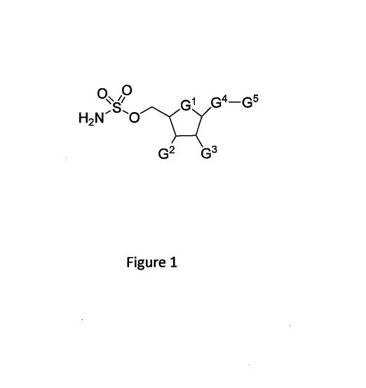

The invention relates to monitoring the emergence or presence of NAE variants exhibiting a reduced sensitivity to particular agents, and screening for and/or developing and/or designing other agents having properties suitable for making them useful in new therapeutic regimes. In accordance with the present invention, the inventors have identified variants of NAE with mutations in the UBA3 gene which reduce the sensitivity of NAE to an NAE inhibitor, such as a 1-substituted methyl sulfamate (FIG. 1). In some embodiments, the variants are sensitive to one or more other including, but not limited to a proteasome inhibitor, e.g., bortezomib, an anthracycline, e.g., doxorubicin or a topoisomerase I inhibitor, e.g., the irinotecan metabolite, SN-38.

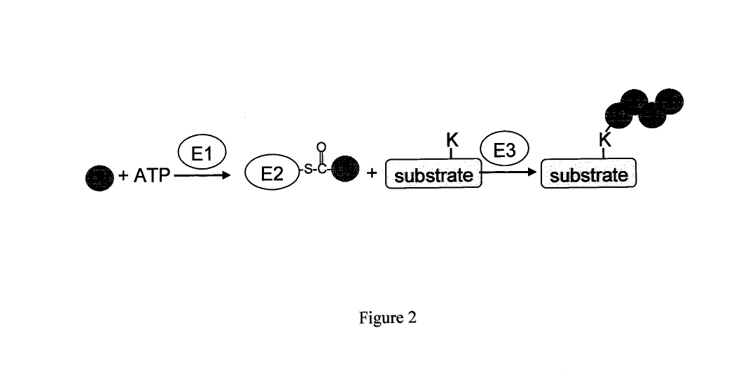

Ubiquitin and other ubls are activated by a specific enzyme (an E1 enzyme) which catalyzes the formation of an acyl-adenylate intermediate with the C-terminal glycine of the ubl (see FIG. 2). The activated ubl is then transferred to a catalytic cysteine residue within the E1 enzyme through formation of a thioester bond intermediate. The E1-ubl intermediate and an E2 associate, resulting in a thioester exchange wherein the ubl is transferred to the active site cysteine of the E2. The ubl is then conjugated to the target protein, either directly or in conjunction with an E3 ligase, through isopeptide bond formation with the amino group of a lysine side chain in the target protein. The ubl named Neural precursor cell-Expressed Developmentally Downregulated 8 (NEDD8) is activated by the heterodimer NEDD8-activating enzyme (NAE, also known as APPBP1-UBA3, UBE1C (ubiquitin-activating enzyme E1C)) and is transferred to one of two E2 conjugating enzymes (ubiquitin carrier protein 12 (UBC12) and UBC17), ultimately resulting in ligation of NEDD8 to cullin proteins by the cullin-RING subtype of ubiquitin (E3) ligases. A function of neddylation is the activation of cullin-based ubiquitin ligases involved in the turnover of many cell cycle and cell signaling proteins, including p27 and I-κB. See Pan et al., Oncogene 23:1985-97 (2004). Inhibition of NAE can disrupt cullin-RING ligase-mediated protein turnover and can lead to apoptotic death in cells, e.g., tumor cells or cells of a pathogenic organism, e.g. a parasite.

E1 activating enzymes function at the first step of ubl conjugation pathways; thus, inhibition of an E1 activating enzyme will specifically modulate the downstream biological consequences of the ubl modification. NAE is a heterodimeric E1 activating enzyme of regulatory and catalytic subunits. NAE1 (NAEα, amyloid beta precursor protein-binding protein 1, AppBp1, predominant isoform has GenBank Accession No. NM_{—}003905, GenPept NP_{—}003896, SEQ ID NO:3) is the regulatory subunit and UBA3 (ubiquitin activating enzyme 3, NAEβ, the longer isoform is GenBank Accession No. NM_{—}003968, SEQ ID NO:1, GenPept Accession No. NP_{—}003959, SEQ ID NO:2) is the catalytic subunit. NAE catalyzes the attachment of NEDD8 to UBC12. UBC12 then transfers NEDD8 to the cullin-based ubiquitin ligases for transfer onto protein substrates.

NAE catalyzes the attachment of NEDD8 to UBC12 by first catalyzing the combination of NEDD8 and ATP to create a NEDD8-AMP intermediate (plus free pyrophosphate). AMP then leaves as NEDD8 forms a thioester link with C237 of UBA3, SEQ ID NO:2. When a second NEDD8 is combined into a NEDD8-AMP intermediate on UBA3, UBA3 then transfers the first NEDD8 onto UBC12.

As used herein, the term “E1,” “E1 enzyme,” or “E1 activating enzyme” refers to any one of a family of related ATP-dependent activating enzymes involved in activating or promoting ubiquitin or ubiquitin-like (collectively “ubl”) conjugation to target molecules. E1 activating enzymes function through an adenylation/thioester intermediate formation to transfer of the appropriate ubl to the respective E2 conjugating enzyme through a transthiolation reaction. The resulting activated ubl-E2 promotes ultimate conjugation of the ubl to a target protein. A variety of cellular proteins that play a role in cell signaling, cell cycle, and protein turnover are substrates for ubl conjugation which is regulated through E1 activating enzymes (e.g., NAE, UAE, SAE). Unless otherwise indicated by context, the term “E1 enzyme” is meant to refer to any E1 activating enzyme protein, including, without limitation, NEDD8 activating enzyme (NAE (APPBP1/Uba3)), ubiquitin activating enzyme (UAE (Uba1)), sumo activating enzyme (SAE (Aos1/Uba2)), UBA4, UBA5, UBA6, UBA7, ATG7, or ISG15 activating enzyme (Ube1L).

The term “E1 enzyme inhibitor” or “inhibitor of E1 enzyme” is used to signify a compound having a structure as defined herein, which is capable of interacting with an E1 enzyme and inhibiting its enzymatic activity. Inhibiting E1 enzymatic activity means reducing the ability of an E1 enzyme to activate ubiquitin like (ubl) conjugation to a substrate peptide or protein (e.g., ubiquitination, neddylation, sumoylation). In various embodiments, such reduction of E1 enzyme activity is at least about 50%, at least about 75%, at least about 90%, at least about 95%, or at least about 99%. In various embodiments, the concentration of E1 enzyme inhibitor required to reduce an E1 enzymatic activity is less than about 1 μM, less than about 500 nM, less than about 100 nM, less than about 50 nM, or less than about 10 nM.

As used herein, the term “NAE inhibitor” refers to 1-substituted methyl sulfamate, including MLN4924. Langston S. et al. U.S. patent application Ser. No. 11/700,614, which is hereby incorporated by reference in its entirety and whose PCT application was published as WO07/092213, discloses compounds which are effective inhibitors of E1 activating enzymes, e.g., NAE. The compounds are useful for inhibiting E1 activity in vitro and in vivo and are useful for the treatment of disorders of cell proliferation, e.g., cancer, and other disorders associated with E1 activity, such as pathogenic infections and neurodegenerative disorders. One class of compounds described in Langston et al. are 4-substituted ((1S,2S,4R)-2-hydroxy-4-{7H-pyrrolo[2,3-d]pyrimidin-7-yl}cyclopentyl)methyl sulfamates.

In some embodiments, such inhibition is selective, i.e., the E1 enzyme inhibitor reduces the ability of one or more E1 enzymes (e.g., NAE, UAE, or SAE) to promote ubl conjugation to substrate peptide or protein at a concentration that is lower than the concentration of the inhibitor that is required to produce another, unrelated biological effect. In some such embodiments, the E1 enzyme inhibitor reduces the activity of one E1 enzyme at a concentration that is lower than the concentration of the inhibitor that is required to reduce enzymatic activity of a different E1 enzyme. In other embodiments, the E1 enzyme inhibitor also reduces the enzymatic activity of another E1 enzyme, such as one that is implicated in regulation of pathways involved in cancer (e.g., NAE and UAE).

MLN4924 (((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulphamate) disrupts cullin-RING ligase-mediated protein turnover leading to apoptotic death in human tumor cells by perturbation of cellular protein homeostasis (Soucy et al. (2009) Nature 458:732-736). The evaluation of MLN4924 in cellular and tumor xenograft studies has revealed two distinct mechanisms of action. The first is the induction of DNA re-replication, DNA damage and cell death through MLN4924-mediated dysregulation of the CRL1^{SKP2} and CRL4^{DDB1} substrate Cdt-1 (Milhollen et al., 2011). It has been shown that p53 status does not impact the induction of DNA re-replication but may make cells more prone to undergo apoptosis or senescence depending on the appropriate genetic background (Milhollen et al., 2011, Lin et al., 2010a and Lin et al., 2010b). The second mechanism is the inhibition of NF-κB pathway activity in NF-κB dependent Diffuse Large B-Cell Lymphomas primarily through dysregulation of CRL1^{βTRCP} mediated turnover of phosphorylated IκBα (Milhollen et al., 2010). In addition, pre-clinical models of Acute Myelogenous Leukemia (AML) are sensitive to MLN4924 inhibition in both cell lines and primary patient blasts through mechanisms related to Cdt-1 dysregulation, NF-κB inhibition and induction of reactive oxygen species (Swords et al., 2010).

MLN4924 is a mechanism-based inhibitor of NAE and creates a covalent NEDD8-MLN4924 adduct catalyzed by the enzyme (Brownell et al. (2010) Mol. Cell 37:102-111). The NEDD8-MLN4924 adduct resembles NEDD8 adenylate, the first intermediate in the NAE reaction cycle, but cannot be further utilized in subsequent intraenzyme reactions. The stability of the NEDD8-MLN4924 adduct within the NAE active site blocks enzyme activity, thereby accounting for the potent inhibition of the NEDD8 pathway by MLN4924.

Herein is described the emergence of mutations in the NAEβ (UBA3) subunit of NAE in cell lines and xenograft models of cancer following selection pressure with MLN4924. In some embodiments, the mutations are heterozygous. In other embodiments, the mutations can be broadly classified into two classes which impact the ATP or NEDD8 binding regions of UBA3 (NAEβ). Biochemical studies show that both classes of mutations can reduce compound potency by a mechanism such as by slowing the rate of NEDD8-MLN4924 adduct formation and by promoting a faster dissociation of the adduct. Evidence in cultured cells and xenografts shows a reduction in pathway inhibition, lower amounts of UBA3 (NAEβ)-bound NEDD8-MLN4924 adduct and faster recovery of pathway activity following inhibition. A framework is provided for the design of E1 enzyme inhibitors, such as NAE inhibitors, e.g., 1-substituted methyl sulfamates, MLN4924 analogs with activity against the mutant enzymes. The framework provides the potential for re-treatment of patients that initially respond to E1 enzyme inhibitors, such as NAE inhibitors, e.g., a 1-substituted methyl sulfamate, MLN4924, therapy but ultimately relapse and thus overcome resistance mediated by mutations in NAEβ.

One aspect of the invention is related to isolated NAE variants that comprise at least one nucleotide mutation in the UBA3 gene. A nucleotide mutation further can result in at least one amino acid mutation, e.g. an amino acid substitution, in a UBA3 protein. A nucleotide mutation can lead to a reduced sensitivity to the NAE inhibitor, such as a 1-substituted methyl sulfamate. A UBA3 variant can comprise at least one nucleotide mutation that results in at least one amino acid mutation, e.g. an amino acid substitution, of the alanine at residue 171 of the UBA3 protein of SEQ ID NO:2. In one embodiment, this alanine can be mutated into a threonine (A171T). In another embodiment, this alanine can be mutated into an aspartate (A171D). In yet another embodiment, this alanine can be mutated into a valine (A171V). In other embodiments, this alanine can be mutated into a glutamate (A171E or a serine (A171S). A UBA3 variant can comprise at least one nucleotide mutation which results in at least one amino acid mutation, e.g. an amino acid substitution of the glycine at residue 201 of the UBA3 protein of SEQ ID NO:2. In one embodiment, this glycine can be mutated into a valine (G201V). A UBA3 variant can comprise at least one nucleotide mutation which results in at least one amino acid mutation, e.g. an amino acid substitution of the glutamate at residue 204 of the UBA3 protein of SEQ ID NO:2. In one embodiment, this glutamate can be mutated into a lysine (E204K). In another embodiment, this glutamate can be mutated into a glycine (E204G). A UBA3 variant can comprise at least one nucleotide mutation which results in at least one amino acid mutation, e.g. an amino acid substitution of the glycine at residue 205 of the UBA3 protein of SEQ ID NO:2. In one embodiment, this glycine can be mutated into a cysteine (G205C). A UBA3 variant can comprise at least one nucleotide mutation which results in at least one amino acid mutation, e.g. an amino acid substitution of the asparagine at residue 209 of the UBA3 protein of SEQ ID NO:2. In one embodiment, this asparagine can be mutated into a lysine (N209K). In another embodiment, this asparagine can be mutated into an aspartate (N209D). A UBA3 variant can comprise at least one nucleotide mutation which results in at least one amino acid mutation, e.g. an amino acid substitution of the arginine at residue 211 of the UBA3 protein of SEQ ID NO:2. In one embodiment, this arginine can be mutated into a glutamine (R211Q). A UBA3 variant can comprise at least one nucleotide mutation which results in at least one amino acid mutation, e.g. an amino acid substitution of the tyrosine at residue 228 of the UBA3 protein of SEQ ID NO:2. In one embodiment, this tyrosine can be mutated into a histidine (Y228H). In another embodiment, this tyrosine can be mutated into a cysteine (Y228C). A UBA3 variant can comprise at least one nucleotide mutation which results in at least one amino acid mutation, e.g. an amino acid substitution of the proline at residue 229 of the UBA3 protein of SEQ ID NO:2. In one embodiment, this proline can be mutated into a glutamine (P229Q). A UBA3 variant can comprise at least one nucleotide mutation which results in at least one amino acid mutation, e.g. an amino acid substitution of the valine at residue 305 of the UBA3 protein of SEQ ID NO:2. In one embodiment, this valine can be mutated into an alanine (V305A). A UBA3 variant can comprise at least one nucleotide mutation which results in at least one amino acid mutation, e.g. an amino acid substitution of the proline at residue 311 of the UBA3 protein of SEQ ID NO:2. In one embodiment, this proline can be mutated into a serine (P311S). In one embodiment, this proline can be mutated into a threonine (P311T). A UBA3 variant can comprise at least one nucleotide mutation which results in at least one amino acid mutation, e.g. an amino acid substitution of the alanine at residue 314 of the UBA3 protein of SEQ ID NO:2. In one embodiment, this alanine can be mutated into a proline (A314P). A UBA3 variant can comprise at least one nucleotide mutation which results in at least one amino acid mutation, e.g. an amino acid substitution of the cysteine at residue 324 of the UBA3 protein of SEQ ID NO:2. In one embodiment, this cysteine can be mutated into a tyrosine (C324Y). A UBA3 variant can comprise at least one nucleotide mutation which results in at least one amino acid mutation, e.g. an amino acid substitution of the cysteine at residue 249 of the UBA3 protein of SEQ ID NO:2. In one embodiment, this cysteine can be mutated into a tyrosine (C249Y). A UBA3 variant according to the invention can exhibit a decreased sensitivity to a NAE inhibitor, such as a 1-substituted methyl sulfamate.

The at least one nucleotide mutation in a UBA3 variant gene can comprise a mutated nucleotide in a GCC codon at 531 to 533 of SEQ ID NO:1, so that it does not code for alanine. The at least one nucleotide mutation in a UBA3 variant gene can comprise a mutated nucleotide 531, 532 and/or 533 of SEQ ID NO:1. In one embodiment, nucleotide 531 can be adenine, thymine or cytosine. A mutation that changes the guanine at nucleotide 531 to adenine can cause amino acid 171 of SEQ ID NO:2 to be threonine in a UBA3 variant instead of alanine in wild type UBA3. A mutation that changes the guanine at nucleotide 531 to thymine can cause amino acid 171 of SEQ ID NO:2 to be serine in a UBA3 variant instead of alanine in wild type UBA3. In another embodiment, nucleotide 532 can be guanine, adenine or thymine. A mutation that changes the cytosine at nucleotide 532 to adenine and the cytosine at nucleotide 533 to adenine or guanine can cause amino acid 171 of SEQ ID NO:2 to be glutamate in a UBA3 variant instead of alanine in wild type UBA3.

The at least one nucleotide mutation in a UBA3 variant gene can comprise a mutated nucleotide in a GGG codon at 621 to 623 of SEQ ID NO:1. The at least one nucleotide mutation in a UBA3 variant gene can comprise a mutated nucleotide 621, 622 and/or 623 of SEQ ID NO:1. In one embodiment, nucleotide 621 can be adenine, thymine or cytosine. A mutation that changes the guanine at nucleotide 621 to thymine can cause amino acid 201 of SEQ ID NO:2 to be valine in a UBA3 variant instead of glycine in wild type UBA3. In another embodiment, nucleotide 622 can be adenine, thymine or cytosine. A mutation that changes the guanine at nucleotide 622 to cytosine can cause amino acid 201 of SEQ ID NO:2 to be alanine in a UBA3 variant instead of glycine in wild type UBA3.

The at least one nucleotide mutation in a UBA3 variant gene can comprise a mutated nucleotide in a GAA codon at 630 to 632 of SEQ ID NO:1. The at least one nucleotide mutation in a UBA3 variant gene can comprise a mutated nucleotide 630, 631 and/or 632 of SEQ ID NO:1. In one embodiment, nucleotide 630 can be adenine, thymine or cytosine. A mutation that changes the guanine at nucleotide 630 to adenine can cause amino acid 204 of SEQ ID NO:2 to be lysine in a UBA3 variant instead of glutamate in wild type UBA3. In another embodiment, nucleotide 631 can be guanine, cytosine or thymine. A mutation that changes the adenine at nucleotide 631 to guanine can cause amino acid 204 of SEQ ID NO:2 to be glycine in a UBA3 variant instead of glutamate in wild type UBA3. In another embodiment, nucleotide 632 can be thymine or cytosine. A mutation that changes the adenine at nucleotide 632 to cytosine can cause amino acid 204 of SEQ ID NO:2 to be aspartate in a UBA3 variant instead of glutamate in wild type UBA3.

The at least one nucleotide mutation in a UBA3 variant gene can comprise a mutated nucleotide in a GGT codon at 633 to 635 of SEQ ID NO:1. The at least one nucleotide mutation in a UBA3 variant gene can comprise a mutated nucleotide 633, 634 and/or 635 of SEQ ID NO:1. In one embodiment, nucleotide 633 can be adenine, thymine or cytosine. A mutation that changes the guanine at nucleotide 633 to thymine can cause amino acid 205 of SEQ ID NO:2 to be cysteine in a UBA3 variant instead of glycine in wild type UBA3. In another embodiment, nucleotide 634 can be adenine, cytosine or thymine. A mutation that changes the guanine at nucleotide 634 to adenine can cause amino acid 204 of SEQ ID NO:2 to be aspartate in a UBA3 variant instead of glycine in wild type UBA3.

The at least one nucleotide mutation in a UBA3 variant gene can comprise a mutated nucleotide in a AAT codon at 645 to 647 of SEQ ID NO:1. The at least one nucleotide mutation in a UBA3 variant gene can comprise a mutated nucleotide 645, 646 and/or 647 of SEQ ID NO:1. In one embodiment, nucleotide 645 can be guanine, thymine or cytosine. A mutation that changes the adenine at nucleotide 645 to guanine can cause amino acid 209 of SEQ ID NO:2 to be aspartate in a UBA3 variant instead of asparagine in wild type UBA3. In another embodiment, nucleotide 646 can be adenine, thymine or cytosine. A mutation that changes the guanine at nucleotide 646 to cytosine can cause amino acid 209 of SEQ ID NO:2 to be alanine in a UBA3 variant instead of asparagine in wild type UBA3. In another embodiment, nucleotide 647 can be adenine or guanine. A mutation that changes the thymine at nucleotide 647 to adenine or guanine can cause amino acid 209 of SEQ ID NO:2 to be lysine in a UBA3 variant instead of asparagine in wild type UBA3.

The at least one nucleotide mutation in a UBA3 variant gene can comprise a mutated nucleotide in a CGG codon at 651 to 653 of SEQ ID NO:1. The at least one nucleotide mutation in a UBA3 variant gene can comprise a mutated nucleotide 651, 652 and/or 653 of SEQ ID NO:1. In one embodiment, nucleotide 651 can be guanine or thymine. A mutation that changes the cytosine at nucleotide 651 to guanine can cause amino acid 211 of SEQ ID NO:2 to be glycine in a UBA3 variant instead of arginine in wild type UBA3. In another embodiment, nucleotide 652 can be adenine, thymine or cytosine. A mutation that changes the guanine at nucleotide 652 to adenine can cause amino acid 211 of SEQ ID NO:2 to be glutamine in a UBA3 variant instead of arginine in wild type UBA3.

The at least one nucleotide mutation in a UBA3 variant gene can comprise a mutated nucleotide in a TAT codon at 702 to 704 of SEQ ID NO:1. The at least one nucleotide mutation in a UBA3 variant gene can comprise a mutated nucleotide 702, 703 and/or 704 of SEQ ID NO:1. In one embodiment, nucleotide 702 can be guanine, adenine or cytosine. A mutation that changes the adenine at nucleotide 702 to cytosine can cause amino acid 228 of SEQ ID NO:2 to be histidine in a UBA3 variant instead of tyrosine in wild type UBA3. In another embodiment, nucleotide 703 can be guanine, thymine or cytosine. A mutation that changes the adenine at nucleotide 703 to guanine can cause amino acid 228 of SEQ ID NO:2 to be cysteine in a UBA3 variant instead of tyrosine in wild type UBA3.

The at least one nucleotide mutation in a UBA3 variant gene can comprise a mutated nucleotide in a CCA codon at 705 to 707 of SEQ ID NO:1. The at least one nucleotide mutation in a UBA3 variant gene can comprise a mutated nucleotide 705, 706 and/or 707 of SEQ ID NO:1. In one embodiment, nucleotide 705 can be adenine, guanine or thymine. A mutation that changes the cytosine at nucleotide 705 to adenine can cause amino acid 229 of SEQ ID NO:2 to be threonine in a UBA3 variant instead of proline in wild type UBA3. In another embodiment, nucleotide 706 can be adenine, thymine or guanine. A mutation that changes the cytosine at nucleotide 706 to adenine can cause amino acid 229 of SEQ ID NO:2 to be glutamine in a UBA3 variant instead of proline in wild type UBA3.

The at least one nucleotide mutation in a UBA3 variant gene can comprise a mutated nucleotide in a GTA codon at 933 to 935 of SEQ ID NO:1. The at least one nucleotide mutation in a UBA3 variant gene can comprise a mutated nucleotide 933, 934 and/or 935 of SEQ ID NO:1. In one embodiment, nucleotide 933 can be adenine, cytosine or thymine. A mutation that changes the guanine at nucleotide 933 to adenine can cause amino acid 305 of SEQ ID NO:2 to be isoleucine in a UBA3 variant instead of valine in wild type UBA3. In another embodiment, nucleotide 934 can be adenine, guanine or cytosine. A mutation that changes the thymine at nucleotide 934 to cytosine can cause amino acid 305 of SEQ ID NO:2 to be alanine in a UBA3 variant instead of valine in wild type UBA3.

The at least one nucleotide mutation in a UBA3 variant gene can comprise a mutated nucleotide in a CCT codon at 951 to 953 of SEQ ID NO:1. The at least one nucleotide mutation in a UBA3 variant gene can comprise a mutated nucleotide 951, 952 and/or 953 of SEQ ID NO:1. In one embodiment, nucleotide 951 can be adenine, guanine or thymine. A mutation that changes the cytosine at nucleotide 951 to adenine can cause amino acid 311 of SEQ ID NO:2 to be threonine in a UBA3 variant instead of proline in wild type UBA3. A mutation that changes the cytosine at nucleotide 951 to thymine can cause amino acid 311 of SEQ ID NO:2 to be senile in a UBA3 variant instead of proline in wild type UBA3. In another embodiment, nucleotide 952 can be adenine, thymine or guanine. A mutation that changes the cytosine at nucleotide 952 to adenine can cause amino acid 311 of SEQ ID NO:2 to be glutamine in a UBA3 variant instead of proline in wild type UBA3.

The at least one nucleotide mutation in a UBA3 variant gene can comprise a mutated nucleotide in a GCT codon at 960 to 962 of SEQ ID NO:1. The at least one nucleotide mutation in a UBA3 variant gene can comprise a mutated nucleotide 960, 961, and/or 962 of SEQ ID NO:1. In one embodiment, nucleotide 960 can be adenine, cytosine or thymine. A mutation that changes the guanine at nucleotide 960 to cytosine can cause amino acid 314 of SEQ ID NO:2 to be proline in a UBA3 variant instead of alanine in wild type UBA3. A mutation that changes the cytosine at nucleotide 961 to adenine can cause amino acid 314 of SEQ ID NO:2 to be aspartate in a UBA3 variant instead of alanine in wild type UBA3. In another embodiment, nucleotide 962 can be adenine, cytosine or guanine.

The at least one nucleotide mutation in a UBA3 variant gene can comprise a mutated nucleotide in a TGT codon at 765 to 767 of SEQ ID NO:1. The at least one nucleotide mutation in a UBA3 variant gene can comprise a mutated nucleotide 765, 766 and/or 767 of SEQ ID NO:1. In one embodiment, nucleotide 765 can be guanine, adenine or cytosine. A mutation that changes the thymine at nucleotide 765 to adenine can cause amino acid 249 of SEQ ID NO:2 to be serine in a UBA3 variant instead of cysteine in wild type UBA3. In another embodiment, nucleotide 766 can be adenine, thymine or cytosine. A mutation that changes the guanine at nucleotide 766 to adenine can cause amino acid 249 of SEQ ID NO:2 to be tyrosine in a UBA3 variant instead of cysteine in wild type UBA3. In another embodiment, nucleotide 767 can be guanine. A mutation that changes the thymine at nucleotide 767 to guanine can cause amino acid 249 of SEQ ID NO:2 to be tryptophan in a UBA3 variant instead of cysteine in wild type UBA3.

The at least one nucleotide mutation in a UBA3 variant gene can comprise a mutated nucleotide in a TGT codon at 989 to 991 of SEQ ID NO:1. The at least one nucleotide mutation in a UBA3 variant gene can comprise a mutated nucleotide 989, 990 and/or 991 of SEQ ID NO:1. In one embodiment, nucleotide 989 can be guanine, adenine or cytosine. A mutation that changes the thymine at nucleotide 989 to adenine can cause amino acid 324 of SEQ ID NO:2 to be serine in a UBA3 variant instead of cysteine in wild type UBA3. In another embodiment, nucleotide 990 can be adenine, thymine or cytosine. A mutation that changes the guanine at nucleotide 990 to adenine can cause amino acid 324 of SEQ ID NO:2 to be tyrosine in a UBA3 variant instead of cysteine in wild type UBA3. In another embodiment, nucleotide 991 can be guanine. A mutation that changes the thymine at nucleotide 991 to guanine can cause amino acid 324 of SEQ ID NO:2 to be tryptophan in a UBA3 variant instead of cysteine in wild type UBA3.

The at least one nucleotide mutation in a UBA3 variant gene can comprise mutated genotypic patterns at sites which do not result in an amino acid change. Such a mutation can be “silent” in the protein structure, but have an effect on the nucleotide structure. In one embodiment, a UBA3 variant can comprise at least one nucleotide mutation which does not result in at least one amino acid mutation, e.g. an amino acid substitution. A mutation in a UBA3 variant that changes the thymine at nucleotide 962 to cytosine does not cause amino acid 314 of SEQ ID NO:2 to change from alanine. In this embodiment, the mutation can result in an altered expression of the UBA3 protein, or can be accompanied by a mutation in another part of the NAE heterodimer, such as in NAE1 or another amino acid in UBA3.

The at least one nucleotide mutation in a UBA3 variant gene can further comprise mutated genotypic patterns at other sites of NAE, e.g., sites which do not confer resistance to E1 enzyme inhibitors, such as NAE inhibitors, e.g., 1-substituted methyl sulfamate (e.g., MLN4924). The further mutation can be silent or can be accompanied by a mutation in another part of the NAE heterodimer, such as in NAE1.

Thus, the at least one nucleotide mutation in a UBA3 variant nucleic acid is selected from the group consisting of nucleotide 531, 532, 533, 621, 622, 623, 630, 631, 632, 633, 634, 635, 645, 646, 647, 651, 652, 653, 702, 703, 704, 705, 706, 707, 765, 766, 767, 933, 934, 935, 951, 952, 953, 960, 961, 962, 989, 990, and 991 of SEQ ID NO:1. In one embodiment, the at least one nucleotide mutation in a UBA3 variant nucleic acid does not comprise a change at nucleotide 765, 766 and/or 767 of SEQ ID NO:1. In this embodiment, the at least one nucleotide mutation in a UBA3 variant nucleic acid is selected from the group consisting of nucleotide 531, 532, 533, 621, 622, 623, 630, 631, 632, 633, 634, 635, 645, 646, 647, 651, 652, 653, 702, 703, 704, 705, 706, 707, 933, 934, 935, 951, 952, 953, 960, 961, 962, 989, 990, and 991 of SEQ ID NO:1. The at least one nucleotide mutation in a UBA3 variant nucleic acid can result in an amino acid change at a residue selected from the group consisting of amino acid residue 171, 201, 204, 205, 209, 211, 228, 229, 249, 305, 311, 314 and 324 of SEQ ID NO:2. In one embodiment, the at least one nucleotide mutation in a UBA3 variant nucleic acid does not comprise a change at amino acid 249 of SEQ ID NO:2. In this embodiment, the at least one amino acid change is selected from the group consisting of a change in residue 171, 201, 204, 205, 209, 211, 228, 229, 305, 311, 314 and 324 of SEQ ID NO:2.

In additional embodiments, the present invention extends to isolated UBA3 variants that comprise two or more nucleotide mutations in SEQ ID NO:1 selected from the group consisting of nucleotide 531, 532, 533, 621, 622, 623, 630, 631, 632, 633, 634, 635, 645, 646, 647, 651, 652, 653, 702, 703, 704, 705, 706, 707, 765, 766, 767, 933, 934, 935, 951, 952, 953, 960, 961, 962, 989, 990, and 991 of SEQ ID NO:1 to result in two or more amino acid substitutions in the UBA3 protein. For example, a change at residue 171 of SEQ ID NO:2 can be accompanied by a change at an additional residue, which may or may not contribute to the resistance. In an embodiment, the change in UBA3 is heterozygous, i.e., present in only one UBA3 allele in a cell. In another embodiment, the change in UBA3 is homozygous. In other embodiments, a combination of changes can be any two or more than two amino acids selected from the group consisting of a change at residue 171, 201, 204, 205, 209, 211, 228, 229, 249, 305, 311, 314 and 324 of SEQ ID NO:2. A combination of changes alternatively can comprise at least one change on one allele of UBA3 in a cell, and another change in another allele of UBA3 in the cell. In such a cell, each variation is heterozygous, but no wild type UBA3 is present in the cell.

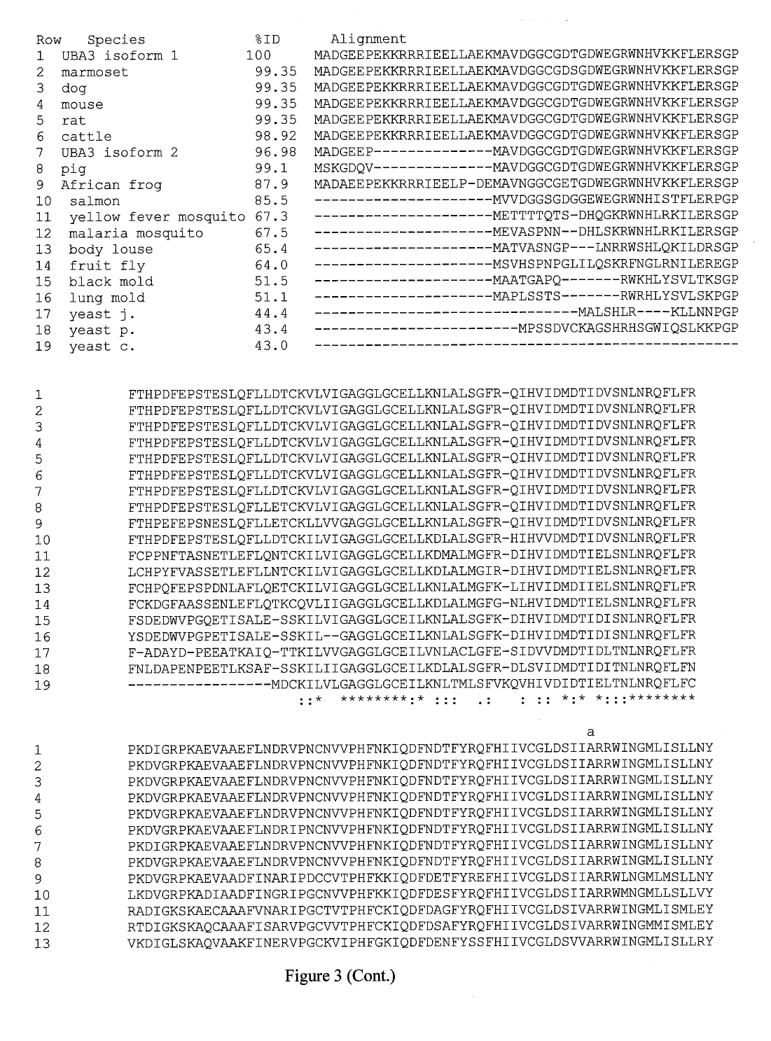

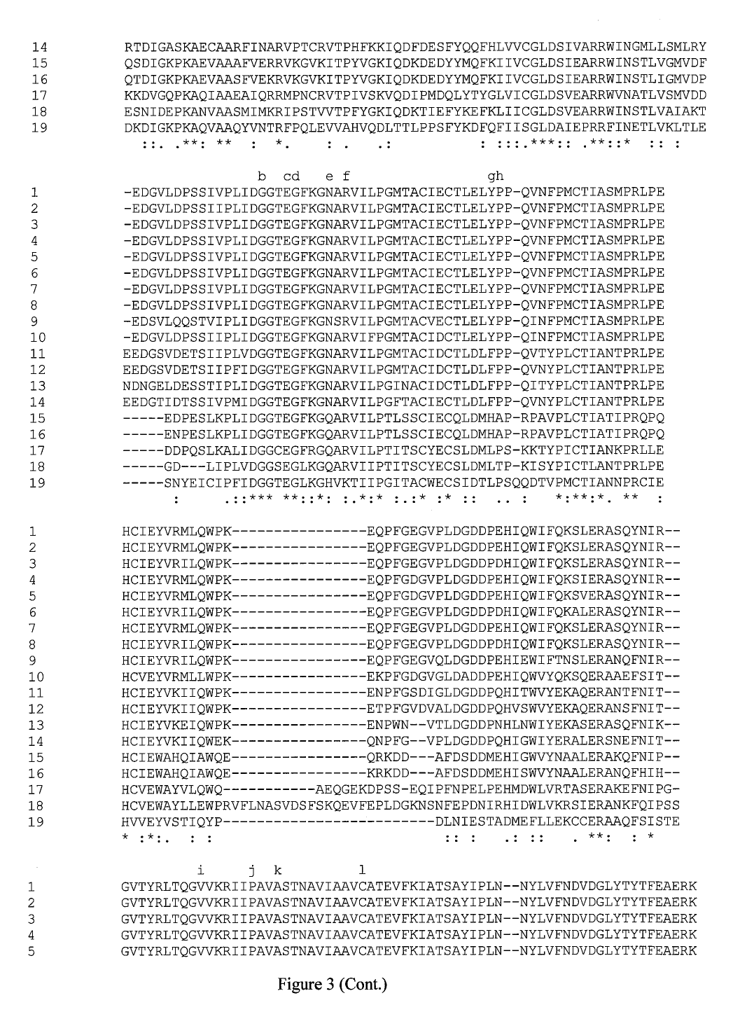

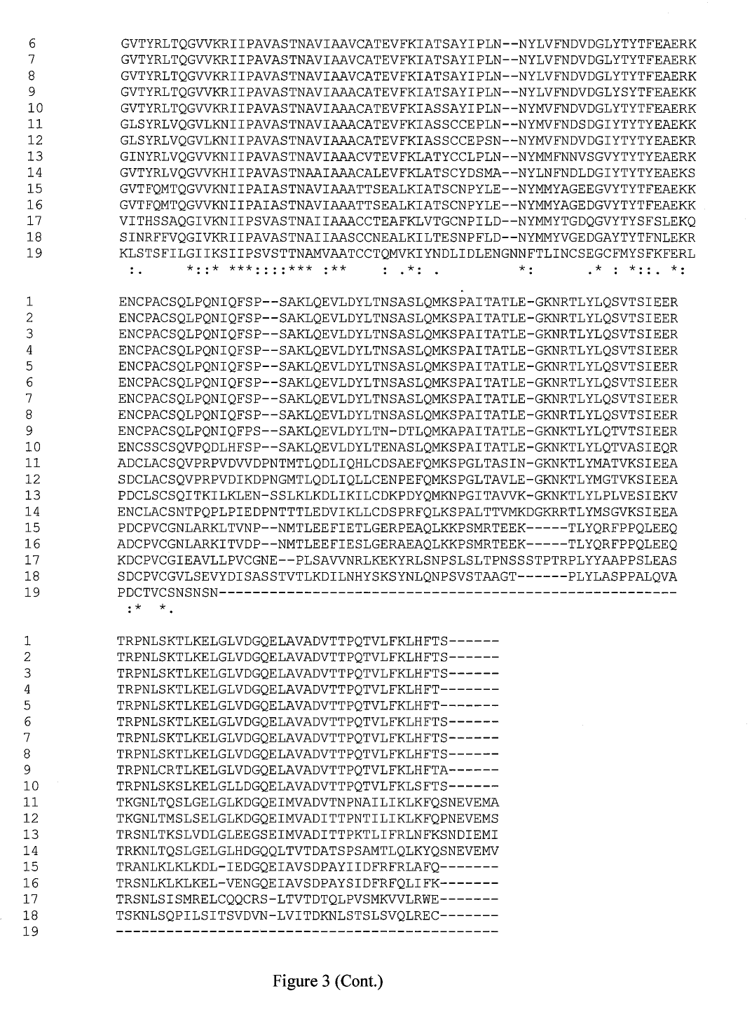

UBA3 is highly conserved among phyla and species. The alignment of FIG. 3 shows that amino acid residues 171, 201, 204, 209, 228, 229, 305 and 324 of SEQ ID NO:2 are conserved among mammals (human, marmoset, dog, rat, mouse, cow, pig), birds and fish (which were overall not less than 85% identical to SEQ ID NO:2). Residues 201, 204, 205 and 311 are conserved not only among the species listed above, but also for invertebrates, molds and yeast (which were overall not less than 40% identical to SEQ ID NO:2). Only one yeast species in FIG. 2 has a residue other than alanine at the location corresponding to A171, a residue other than arginine at the location corresponding to R211, a residue other than cysteine at the location corresponding to C249 or a residue other than alanine at the location corresponding to A314 of SEQ ID NO:2. Among the sequences with a residue other than asparagine at the corresponding residue as N209, there are only conserved substitutions (glutamine, in molds and two yeast species; histidine in a third yeast species). There is conserved substitution of tyrosine at the residue corresponding to Y228 in invertebrates, and only the molds show a substitution for cysteine at the residue corresponding to C324. Accordingly, resistance mediated by the human UBA3 variants described herein can be contemplated for multiple phyla and species, e.g., vertebrate, e.g., mammals, and invertebrate, such as primate (e.g., human, marmoset), rodents (mouse, rat), ungulates (cattle), (fish) (frog) (bird invertebrates (mosquito, louse) (mold) (yeast). Assays for inhibitors, such as chemotherapies that overcome resistance to drugs targeting those enzymes can use variants at the respective residues of the enzymes in FIG. 3. Uses for domesticated animals in FIG. 3 can be related to treatment of diseases, such as cancer. Assays for inhibitors that overcome resistance to drugs targeting the rodent and invertebrate enzymes in FIG. 3 can be used for developing pest control products.

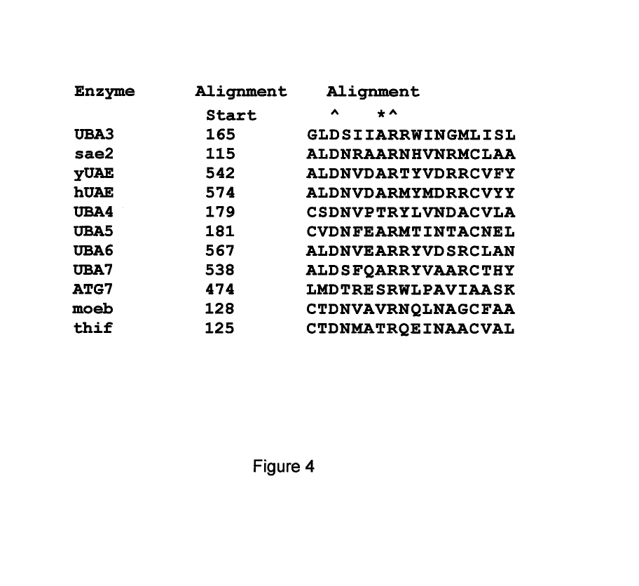

Alanine at position 171 is conserved in additional enzymes or subunits of dimeric enzymes (see FIG. 4), including E1 activating enzymes such as Sumo-activating enzyme (SAE2) (SEQ ID NO:27), UBA1 (SEQ ID NO:28 (yeast), SEQ ID NO:29 (human)), MOCS3 (UBA4, SEQ ID NO:30), UBA5 (SEQ ID NO:31), UBA6 (SEQ ID NO:32), UBA7 (SEQ ID NO:33), and ATG7 (SEQ ID NO:34) and other enzymes, such as the bacterial enzymes moeb (SEQ ID NO:35) and thif (SEQ ID NO:36), which are structurally and mechanistically related to E1 enzymes, i.e., they have an active site cysteine and make a thioester bond with a substrate in the course of their activitiy. Alanine 171 of human UBA3 corresponds to alanine at residue 121 in SAE2 (SEQ ID NO:27), alanine at residue 548 in yeast UBA1 (SEQ ID NO:28), alanine at residue 580 in human UBA1 (SEQ ID NO:29), threonine at residue 185 in MOCS3 (UBA4, SEQ ID NO:30), alanine at residue 187 in UBA5 (SEQ ID NO:31), alanine at residue 573 in UBA6 (SEQ ID NO:32), alanine at residue 544 in UBA7 (SEQ ID NO:33), serine at residue 480 in ATG7 (SEQ ID NO:34), valine at residue 134 in moeb (SEQ ID NO:35) and threonine at residue 131 in thif (SEQ ID NO:36). Assays for inhibitors, such as chemotherapies that overcome resistance to drugs targeting those enzymes can use variants at that residue which corresponds to A171 of SEQ ID NO:2, which is residue A121 in SEQ ID NO:27, A548 in SEQ ID NO:28, A580 in SEQ ID NO:29, T185 in SEQ ID NO:30, A187 in SEQ ID NO:31, A573 in SEQ ID NO:32, A544 in SEQ ID NO:33, S480 in SEQ ID NO:34, V134 in SEQ ID NO:35 and T131 in SEQ ID NO:36. Assays for inhibitors, such as antibiotics that overcome resistance to drugs targeting those enzymes can use variants at that residue, which is residue V134 in SEQ ID NO:35 and T131 in SEQ ID NO:36.

One aspect of the invention is related to isolated UBA1 variants that comprise at least one nucleotide mutation in the UBA1 gene. A nucleotide mutation further can result in at least one amino acid mutation, e.g. an amino acid substitution in a UBA1 protein. A nucleotide mutation in a UBA1 gene can lead to a reduced sensitivity to an E1 enzyme inhibitor, such as an NAE inhibitor, such as a 1-substituted methyl sulfamate. A UBA1 variant can comprise at least one nucleotide mutation that results in at least one amino acid mutation, e.g. an amino acid substitution of the alanine at residue 580 of the UBA1 protein of SEQ ID NO:29. In one embodiment, this alanine can be mutated into a threonine (A580T). In another embodiment, this alanine can be mutated into an aspartate (A580D).

Another aspect of the invention is related to isolated UBA6 variants that comprise at least one nucleotide mutation in the UBA6 gene. A nucleotide mutation further can result in at least one amino acid mutation, e.g. an amino acid substitution in a UBA6 protein. A nucleotide mutation in a UBA6 gene can lead to a reduced sensitivity to an E1 enzyme inhibitor, such as an NAE inhibitor, such as a 1-substituted methyl sulfamate. A UBA6 variant can comprise at least one nucleotide mutation that results in at least one amino acid mutation, e.g. an amino acid substitution of the alanine at residue 573 of the UBA6 protein of SEQ ID NO:32. In one embodiment, this alanine can be mutated into a threonine (A573T). In another embodiment, this alanine can be mutated into an aspartate (A573D).

Human UBA3 can result from translation of the open reading frame (about bases 21 to 1412) of SEQ ID NO:1 and contains the following regions or other structural features (for general information regarding PFAM identifiers, PS prefix and PF prefix domain identification numbers, refer to Sonnhammer et al. (1997) Protein 28:405-420; Pfam: Finn et al. (2010) Nuc. Ac. Res. 38:D211-222 and the website maintained by the Wellcome Trust Sanger Institute, Hinxton, Cambridge, UK; ProSite: Sigrist et al. (2010) Nuc. Ac. Res. 38:161-166 and the website for the ExPASy proteomics server maintained by the Swiss Institute of Bioinformatics, Lausanne, Switzerland): a ThiF domain (PFAM Accession Number PF00899) located at about amino acid residues 69 to 211 of SEQ ID NO:2; a UBA_e1_thiolCys domain (PFAM Accession Number PF10585) located at about amino acid residues 216 to 262 of SEQ ID NO:2; a UBACT domain (PFAM Accession Number PF02134) located at about amino acid residues 270 to 334 of SEQ ID NO:2; a E2_bind domain (PFAM Accession Number PF08825) located at about amino acid residues 374 to 462 of SEQ ID NO:2; a ProSite ubiquitin-activating enzyme signature sequence (Prosite PD0000463) at about amino acid residues 235 to 243 of SEQ ID NO:2. A catalytic cysteine residue, e.g., for NEDD8 binding, can be residue 237 of SEQ ID NO:2. ATP binding pocket, including about amino acid residues 148 to about 171 of SEQ ID NO:2; NEDD8 binding pocket, residues about amino acid residues 201 to about 229 of SEQ ID NO:2; Catalytic Cys domain, about residues 229 to 309 of SEQ ID NO:2.

In one embodiment, the at least one mutation in a UBA3 variant can be in the ATP binding pocket. Thus, this ATP binding pocket mutation can affect binding of a molecule, e.g., a NAE inhibitor, such as a 1-substituted methyl sulfamate, e.g., MLN4924 or a nucleotide, e.g., ATP, ADP, ATPyS, deoxy-ATP, AMP-PNP, AMP-amidate and/or hydrolysis of a molecule, e.g., a nucleotide, e.g., ATP, ADP, ATPγS, deoxy-ATP. Alternatively, the ATP binding pocket mutation can affect binding of one NAE inhibitor, such as a 1-substituted methyl sulfamate, e.g., MLN4924, but not affect the binding of another NAE inhibitor, such as adenosine sulfamate or a compound such as a 1-substituted methyl sulfamate which has two, three or four fold or less difference between the IC50 for the variant than wild type UBA3. In some embodiments a UBA3 variant can be resistant to some compounds in Table 13 but not to other compounds in Table 13.

In another embodiment, the at least one mutation in a UBA3 variant can be in the NEDD8 binding pocket. Thus, this NEDD8 binding pocket mutation can affect binding of the ubl, e.g., NEDD8 to UBA3 and/or thioester formation of NEDD8 with UBA3, and/or adenylation of NEDD8, and/or thioester formation of a 1-substituted methyl sulfamate, e.g., adenosine sulfamate or MLN4924 with NEDD8, and/or affect the binding of the NEDD8-to-NAE inhibitor, e.g., NEDD8-to-1-substituted methyl sulfamate adduct. In another embodiment, the at least one mutation in a UBA3 variant can be in the region for binding to NAE1. Thus, this mutation can interfere with heterodimer formation and subsequent enzyme activity.

As used herein, the term “ThiF domain” includes an amino acid sequence of about 135 to 145 amino acid residues in length and having a significant alignment of the sequence to the ThiF domain consensus sequence of SEQ ID NO:4. The ThiF domain can mediate ATP binding to UBA3. The ThiF domain (HMM) has been assigned the PFAM Accession Number PF00899 and can be found at about amino acid residues 69 to about 211 of SEQ ID NO:2.

As used herein, the term “UBA_e1_thiolCys domain” includes an amino acid sequence of about 40 to 50 amino acid residues in length and having a significant alignment of the sequence to the UBA_e1_thiolCys domain (HMM) consensus of SEQ ID NO:5. A UBA_e1_thiolCys domain can mediate NEDD8 binding and thioester linkage and has the ubiquitin-activating enzyme signature sequence (Prosite PDO000463) at about amino acid residues 235 to 243 of SEQ ID NO:2. This signature sequence comprises the active site cysteine at about amino acid residue 237 of SEQ ID NO:2. A UBA_e1_thiolCys domain can include a Prosite ubiquitin-activating enzyme signature sequence PS00865 (P-[LIVMG]-C-T-[LIVM]-[KRHA]-x-[FTNM]-P, SEQ ID NO:6), or sequences homologous thereto. In the above signature sequence, and other motifs or sequences described herein, the standard IUPAC one-letter code for the amino acids is used. Each element in the pattern is separated by a dash (-); square brackets ([ ]) indicate the particular residues that are accepted at that position; and x indicates that any residue is accepted at that position. The UBA_e1_thiolCys domain (HMM) has been assigned the PFAM Accession Number PF10585 and can be found at about amino acid residues 216 to about 260 of SEQ ID NO:2.

In three dimensional space, the UBA3 ATP binding site can involve residues about 99 to 124 and about 146 to 174 of SEQ ID NO:2. In three dimensional space, the UBA3 NEDD8 C-terminus (residues 71-76) binding site can involve residues about 78 to 81, 165 to 172, 201 to 229 and 310 to 344 of SEQ ID NO:2.

As used herein, the term “UBACT domain” includes an amino acid sequence of about 60 to 70 amino acid residues in length and having a significant alignment of the sequence to the UBACT domain (HMM) consensus of SEQ ID NO:7. A UBACT domain can mediate NEDD8 binding. The UBACT domain (HMM) has been assigned the PFAM Accession Number PF02134 and can be found at about amino acid residues 270 to about 334 of SEQ ID NO:2.

As used herein, the term “E2_bind domain” or “E2 binding domain” includes an amino acid sequence of about 80 to 95 amino acid residues in length and having a significant alignment of the sequence to the E2_bind domain consensus sequence of SEQ ID NO:8. An E2_bind domain can mediate association of UBA3 with an E2 enzyme, e.g., UBC12 or UBE2F. The E2_bind domain of UBA3, e.g., human UBA3 polypeptide can be located about the C-terminus of SEQ ID NO:2. The E2_bind domain (HMM) has been assigned the PFAM Accession Number PF08825 and can be found at about amino acid residues 374 to about 462 of SEQ ID NO:2.

The UBA3 variant proteins can have one or more of the following UBA3 activities: (1) the ability to bind a nucleotide (e.g. adenosine triphosphate (ATP), adenosine 5′-diphosphate (ADP), adenosine 5′[γ-thio]triphosphate (ATPγS), deoxy-ATP), Adenosine 5′-(β,γ-imido)triphosphate (AMP-PNP), AMP-amidate, 1-substituted methyl sulfamates, e.g., MLN4924; (2) the ability to hydrolyze a nucleotide (e.g. ATP, ADP, ATPγS, deoxy-ATP); (3) the ability to bind pyrophosphate (PPi), (4) the ability to bind NEDD8 or a NEDD8-adenylate analog (e.g., adenosyl-phospho-NEDD8 (APN)); (5) the ability to adenylate NEDD8; (6) the ability to form a covalent thioester bond with NEDD8; (7) the ability to bind an E2 enzyme, e.g., UBC12 or UBE2F; (8) the ability to catalyze the transthiolation of NEDD8 to an E2 (e.g., UBC12); (9) the ability to bind NAE1; (10) the ability to tightly bind a NAE inhibitor-NEDD8 adduct.

Alternatively, a UBA3 variant protein can be unable or have decreased ability to perform one or more of the UBA3 activities. In one embodiment, a UBA3 variant protein can have a decreased ability to bind and/or form a MLN4924-NEDD8 adduct than than the ability of wild type UBA3 to bind the adduct. Examples of NAE variants with this variant function are variants with mutations in the ATP binding pocket, e.g., varying at or near alanine 171 of SEQ ID NO:2, and/or the NEDD8 binding cleft, e.g., varying at or near glycine 201, glutamate 204, asparagine 209, and/or cysteine 324 of SEQ ID NO:2. In another embodiment, a UBA3 variant protein, e.g., varying at or near tyrosine 228, can have a decreased ability to clamp the C-terminus of NEDD8 into the adenylation domain, to result in reduced ability to adenylate NEDD8. In another embodiment, a UBA3 variant protein, e.g., varying at or near cysteine 249 of SEQ ID NO:2, can have a decreased ability to form a heterodimer with NAE1.

Reference to “decreased” or “reduced” sensitivity in relation to a UBA3 variant includes and encompasses a complete or substantial resistance to the E1 inhibitor as well as partial resistance relative to wild-type sensitivity to the inhibitor. The level of decrease in the ability of a UBA3 variant to perform a UBA3 activity can be 2-fold, 3-fold, 4-fold, 5-fold, 6-fold, 7-fold, 10-fold, 15-fold, 20-fold, 35-fold, 50-fold, 100-fold or more compared to wild type UBA3. The activity, e.g., IC50 (the concentration of inhibitor required to cause a 50% decrease in reaction rate), of a NAE inhibitor, e.g., MLN4924 can be reduced 2-fold, 3-fold, 4-fold, 5-fold, 6-fold, 7-fold, 10-fold, 15-fold, 20-fold, 35-fold, 50-fold, 100-fold or more on an E1 enzyme, e.g., NAE, comprising a UBA3 variant compared an E1 enzyme, e.g., NAE comprising wild type UBA3. In another embodiment, it can take 2 times, 3 times 4 times, 5 times, 6 times, 7 times, 10 times, 15 times, 20 times, 35 times, 50 times, 100 times or more NAE inhibitor, e.g., MLN4924 to kill a cell, e.g., a tumor cell, comprising a UBA3 variant than to kill a cell, e.g., a tumor cell, comprising only, e.g., homozygous for, wild type UBA3.

A UBA3 activity also can be an indirect activity, e.g., a cellular signaling activity mediated by interaction of the neddylated protein with a cullin ring ligase. For example, a UBA3 variant can have one or more of the following indirect activities: 1) the ability to mediate turnover of substrates of the cullin ring ligase; 2) the ability to participate in protein homeostasis; and 3) the ability to support tumor cell survival. An indirect UBA3 activity can be inhibited or decreased in the presence of an E1 enzyme inhibitor, e.g., an NAE inhibitor, e.g., MLN4924. A UBA3 variant can have an indirect UBA3 activity in the presence of an E1 enzyme inhibitor, e.g., an NAE inhibitor, e.g., MLN4924.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

All protein accession numbers provided herein refer to the Entrez Protein database maintained by the National Center for Biotechnology Information (NCBI), Bethesda, Md. or the UniProt database maintained by the Uniprot Consortium (European Bioinformatics Institute (Hinxton, Cambridge UK), Swiss Bioinformatics Institute (Geneva, CH) and Protein Information Resource (Washington, D.C.)).

The phrase “one or more mutations” or “at least one mutation” as used herein, refers to a number of mutations that equals from one to the maximum number of mutations possible based on the number of variant nucleotides or amino acid residues described herein, provided that the conditions of stability and codon feasibility are met. Unless otherwise indicated, an optionally mutated position in a nucleic acid or amino acid sequence may have a mutation at each mutabable position of the sequence, and the UBA3 variants may be either the same or different. As used herein, the term “independently selected” means that the same or different values may be selected for multiple instances of a given variable in a single variant.

“Hybridization” is the process wherein substantially homologous complementary nucleotide sequences anneal to each other. The hybridization process can occur entirely in solution, i.e. both complementary nucleic acids are in solution. Tools in molecular biology relying on such a process include PCR, subtractive hybridization and DNA sequence determination. The hybridization process can also occur with one of the complementary nucleic acids immobilized to a matrix such as magnetic beads, Sepharose beads or any other resin or type of beads. Tools in molecular biology relying on such a process include the isolation of poly (A⁺) mRNA. The hybridization process can furthermore occur with one of the complementary nucleic acids immobilized to a solid support such as a nitrocellulose or nylon membrane, a glass slide or fused silica (quartz) slide (the latter known as nucleic acid arrays or microarrays or as nucleic acid chips), a gold film, a polypyrrole film, an optical fiber or in e.g. a polyacrylamide gel or a microplate well. Tools in molecular biology relying on such a process include RNA and DNA gel blot analysis, colony hybridization, plaque hybridization, reverse hybridization and microarray hybridization. In order to allow hybridization to occur, the nucleic acid molecules are generally thermally, chemically (e.g. by NaOH) or electrochemically denatured to melt a double strand into two single strands and/or to remove hairpins or ‘Molecular Beacons’ probes (single dual-labeled) or other secondary structures from single stranded nucleic acids.

As used herein, the term “hybridizes under low stringency, medium stringency, high stringency, or very high stringency conditions” describes conditions for hybridization and washing. Guidance for performing hybridization reactions can be found in Current Protocols in Molecular Biology (1989) John Wiley & Sons, N.Y., 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and 4) very high stringency hybridization conditions are 0.5M sodium phosphate, 7% SDS at 65° C., followed by one or more washes at 0.2×SSC, 1% SDS at 65° C. In some embodiments, very high stringency conditions are used unless otherwise specified.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding a full length protein, for example a mammalian UBA3 protein, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a UBA3 variant protein having less than about 30%, 20%, 10% or 5% (by dry weight), of non-UBA3 variant protein (also referred to herein as a “contaminating protein”), or of chemical precursors or non-UBA3 variant chemicals. When the UBA3 variant protein or biologically active portion thereof is recombinantly produced, it also can be substantially free of culture medium, i.e., culture medium represents less than about 20%, less than about 10%, or less than about 5% of the volume of the protein preparation. The invention includes isolated or purified preparations of at least 0.01, 0.1, 1.0, and 10 milligrams in dry weight.

The invention further encompasses nucleic acid molecules that differ from the nucleotide sequence shown in SEQ ID NO:1. Such differences can be due to degeneracy of the genetic code (and result in a nucleic acid which encodes the same UBA3 variant proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:2.

A “non-essential” amino acid residue is a residue that can be altered from the wild-type sequence of UBA3 or UBA3 variant (e.g., the sequence of SEQ ID NO:2) without abolishing or without substantially altering a biological activity, whereas an “essential” amino acid residue results in such a change. For example, amino acid residues that are conserved among the polypeptides of the present invention, e.g., those residues in FIGS. 3 and 4 which show the highest degree of conservation, i.e., high identity among species, are essential, and are critical for one or more of enzyme activities, e.g., the activities of UBA3. The amino acid residues which show a high or moderately high identity among species are residues which are important for enzymatic activities, but may not essential. Mutations in such residues can impair or reduce one or more activities of UBA3, however one or more activities of the mutated enzyme still functions, in some embodiments to an altered e.g., reduced, degree. Such residues can be the residues which are mutated in UBA3 variants which resist inhibitors that are designed for wild type enzymatic structures and mechanisms. In some embodiments, residues which are essential or important for enzymatic activity, e.g., a UBA3 activity, can be found in one or more domain of UBA3, e.g., the ATP binding domain, the ATP binding pocket, the NEDD8 binding pocket, the ThiF domain, the UBA_e1_thiolCys domain, the UBACT domain, the E2_bind domain and whose residues are predicted to be conserved, i.e., generally unamenable to alteration. Non-essential amino acids show high variation among species and are likely to be between structural features associated with enzymatic mechanism, e.g., between the domains, e.g., ATP binding domain, the ATP binding pocket, the NEDD8 binding pocket, the ThiF domain, the UBA_e1_thiolCys domain, the UBACT domain, the E2_bind domain, than residues which vary. Mutations to non-essential amino acid residues can be “silent” and not affect a UBA3 activity.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a UBA3 variant protein is replaced with another amino acid residue from the same side chain family. Alternatively, in another embodiment, mutations can be introduced randomly along all or part of a UBA3 variant coding sequence, such as by saturation mutagenesis, and the resultant mutant proteins, e.g., proteins with at least one amino acid substitution, can be screened for UBA3 biological activity to identify mutants that retain activity. Following mutagenesis of SEQ ID NO:1, the encoded protein can be expressed recombinantly and the activity of the protein can be determined.

As used herein, a “biologically active portion” of a UBA3 variant protein includes a fragment of a UBA3 variant protein which participates in an interaction between a UBA3 variant molecule and a non-UBA3 variant molecule. Biologically active portions of a UBA3 variant protein include peptides comprising amino acid sequences sufficiently homologous to or derived from the amino acid sequence of the UBA3 variant protein, e.g., the amino acid sequence shown in SEQ ID NO:2, with one or more variations described herein which include fewer amino acids than the full length UBA3 variant protein, and exhibit at least one activity of a UBA3 variant protein. Typically, biologically active portions comprise a domain or motif with at least one activity of the UBA3 variant protein, e.g., the ability to bind a nucleotide, the ability to hydrolyze a nucleotide, the ability to bind NEDD8, the ability to adenylate NEDD8, the ability to form a covalent thioester bond with NEDD8, the ability to bind an E2 enzyme, the ability to catalyze the transthiolation of NEDD8 to an E2 or the ability to bind NAE1. A biologically active portion of a UBA3 variant protein can be a polypeptide which is, for example, 10, 25, 50, 100, 200 or more amino acids in length. Biologically active portions of a UBA3 variant protein can be used as targets for developing agents which modulate a UBA3 variant mediated activity, e.g., the ability to bind a nucleotide, the ability to hydrolyze a nucleotide, the ability to bind NEDD8, the ability to adenylate NEDD8, the ability to form a covalent thioester bond with NEDD8, the ability to bind an E2 enzyme, the ability to catalyze the transthiolation of NEDD8 to an E2 or the ability to bind NAE1 in the presence of an E1 enzyme inhibitor, e.g., an NAE inhibitor, e.g., 1-methyl sulfamate (e.g., MLN4924).

Calculations of homology or sequence identity (the terms “homology” and “identity” are used interchangeably herein) between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In an embodiment, the length of a reference sequence aligned for comparison purposes is at least 30%, at least 40%, at least 50%, at least 60%, or at least 70%, 80%, 90%, 100% of the length of the reference sequence (e.g., when aligning a second sequence to the UBA3 variant amino acid sequence of SEQ ID NO:2 having 463 amino acid residues, at least 139, at least 185, at least 232, at least 278, at least 324, 370, or 417 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch (1970) J. Mol. Biol. 48:444-453 algorithm which has been incorporated into the GAP program in the GCG software package (available at Accelrys Inc., San Diego, Calif.), using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. One set of parameters are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller ((1989) CABIOS, 4:11-17) which has been incorporated into the ALIGN program (version 2.0), using a PAM120 weight residue table, a gap length penalty of 12 and a gap penalty of 4.

To identify conserved amino acids among more than two sequences, a multiple alignment program can be useful. One multiple alignment program is the Clustal program (first described in 1988 (Higgins and Sharp Gene 73:237-244) and refined in different versions over the years (see, e.g., Thompson et al. (1994) Nucleic Acids Research 22:4673-4680). The conserved residues in can be seen in the same positions in each row and can be seen as identical in columns associated with their positions in the multiple alignment. FIG. 3 herein is an example of a Clustal alignment.

The nucleic acid and protein sequences described herein can be used as a “query sequence” to perform a search against public databases to, for example, identify other family members or related sequences. Such searches can be performed using the NBLAST and)(BLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to UBA3 variant nucleic acid molecules of the invention. BLAST protein searches can be performed with the XBLAST program, score=50, wordlength=3 to obtain amino acid sequences homologous to UBA3 variant protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g.,)(BLAST and NBLAST) can be used. (See the website maintained by the National Center for Biotechnology Information, Bethesda, Md.)

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or 99% identity to SEQ ID NO:1 are termed substantially identical.

“Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

A “drug-resistant phenotype” refers to a cellular phenotype which is associated with increased survival after exposure to a particular dose of a compound, e.g., an NAE inhibitor, such as a 1-substituted methyl sulfamate, e.g., MLN4924, compared to a cell that does not have this phenotype. A “drug-resistant cell” refers to a cell that exhibits this phenotype. Drug resistance can occur as multi-drug resistance (multiple drug resistance) in which a cell population or tumor becomes relatively resistant to a drug to which it has been exposed as well as to other drugs to which it has not been exposed.

“Subject”, as used herein, can refer to a mammal, e.g., a primate, a human, or to an experimental or animal, e.g., non-human primate, mouse, rat, rabbit or disease model, e.g., an immunocompromised animal with a tumor xenograft. The subject can also be a non-human animal, e.g., a horse, cow, goat, or other domestic animal.

A “purified preparation of cells”, as used herein, refers to, in the case of plant or animal cells, an in vitro preparation of cells and not an entire intact plant or animal. In the case of cultured cells or microbial cells, it consists of a preparation of at least 10% or at least 50% of the subject cells.

### Isolated Nucleic Acid Molecules

In one aspect, the invention provides an isolated or purified, nucleic acid molecule that encodes a E1 enzyme variant polypeptide described herein, e.g., a full length UBA3, UAE, UBA6, or other E1 enzyme variant protein or a fragment thereof, e.g., a biologically active portion of a UBA3, UAE, or UBA6, or other E1 enzyme variant protein. Also included is a nucleic acid fragment suitable for use as a hybridization probe, which can be used, e.g., to identify a nucleic acid molecule encoding a variant polypeptide of the invention, such as a UBA3, UAE or UBA6 variant. Further included are nucleic acid fragments suitable for use as primers, e.g., PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs.

An “isolated” nucleic acid molecule is one which is separated from other nucleic acid molecules which are present in the natural source of the nucleic acid. In one embodiment, an “isolated” nucleic acid is free of sequences (such as protein encoding sequences) which naturally flank the nucleic acid (i.e., sequences located at the 5′ and 3′ ends of the nucleic acid) in the genomic DNA of the organism from which the nucleic acid is derived. For example, in various embodiments, an isolated resistance nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. An isolated nucleic acid molecule can have undergone at least one purification step away from naturally occurring body fluid and/or tissue or that it is not present in its native environment. Alternatively, the variants may be maintained in isolated body fluid and/or tissue or may be in a polynucleic acid form. Typically, this means that the UBA3 variant or polynucleic acid is free of at least one of the host proteins and/or host nucleic acids. In general, the isolated UBA3 variant or polynucleic acid is present in an in vitro environment. “Isolated” does not mean that the UBA3 variant or polynucleic acid must be purified or homogeneous, although such preparations do fall within the scope of the term. “Isolated” simply means raised to a degree of purity, to the extent required excluding product of nature and accidental anticipations from the scope of the claims. “Isolated” is meant to include any biological material taken either directly from a subject, e.g. human being or animal, or after purifying (enrichment). “Biological material” may be e.g. expectorations of any kind, broncheolavages, blood, skin tissue, biopsies, sperm, lymphocyte blood culture material, colonies, liquid cultures, faecal samples, urine, etc. “Biological material” may also be artificially transfected, e.g., recombinant, cell cultures or the liquid phase thereof.

In one embodiment, an isolated nucleic acid molecule of the invention includes a variant of the nucleotide sequence shown in SEQ ID NO:1, or a portion of any of this nucleotide sequence. In one embodiment, the nucleic acid molecule includes sequences encoding the human UBA3 variant protein (i.e., variant of “the coding region” of SEQ ID NO:1, as bases 21 to 1412 of SEQ ID NO:1), as well as 5′ untranslated sequences (nucleotides 1 to 20 of SEQ ID NO:1) and 3′ untranslated sequences (nucleotides 1413 to 2136 of SEQ ID NO:1). Alternatively, the nucleic acid molecule can include only the coding region of a variant of SEQ ID NO:1 (e.g., with at least one nucleotide mutation as described herein) and, e.g., no flanking sequences which normally accompany the subject sequence. In another embodiment, the nucleic acid molecule encodes a sequence corresponding to a variant fragment of the variant protein, comprising at least one nucleotide mutation that results in at least one amino acid mutation, e.g. an amino acid substitution in the variant UBA3 as described herein. In an embodiment, the isolated nucleic acid molecule has about 85%, about 90%, about 95%, about 96%, about 97%, about 98% or about 99% identity to SEQ ID NO:1 and comprises at least one nucleotide mutation that results in at least one amino acid mutation, e.g. an amino acid substitution in the variant UBA3 as described herein. In such an embodiment, variation in addition to at least one mutation described herein can encode a non-essential amino acid residue.

Another aspect of the invention relates to fragments of the above-mentioned expression products, which fragments comprise the described amino acid mutations e.g., substitutions leading to a reduced sensitivity to a 1-substituted methyl sulfamate and/or other E1 enzyme inhibitor.

In an embodiment, a nucleic acid fragment can include a sequence comprising at least one mutation, e.g., encoding a variant domain, region, or functional site described herein, e.g., encoding a variant amino acid residue, e.g., an amino acid mutation, e.g. an amino acid substitution. Examples of such variant fragments include a variant ThiF domain about amino acid 69 to about 211 of SEQ ID NO:2, or a fragment thereof, e.g., comprising a variant ATP binding site about 99 to 124 and about 146 to 174 of SEQ ID NO:2 or comprising a variant ATP binding pocket from about amino acid 148 to about 171 of SEQ ID NO:2, a fragment comprising a variant NEDD8 binding pocket from about amino acid 201 to about 229 of SEQ ID NO:2 or a fragment comprising the variant UBACT domain about amino acid residue 270 to about 334 of SEQ ID NO:2. Nucleic acid fragments can encode a specific domain or site described herein or fragments thereof, particularly fragments thereof which are at least 10, 20, 30, 40, 60, 80, 100, 120 or 140 amino acids in length. Such nucleic acid fragments can encode variant amino acid residues whose presence in a UBA3 variant leads to resistance to an E1 enzyme inhibitor. Fragments also include nucleic acid sequences encoding specific amino acid sequences described above or fragments thereof. Nucleic acid fragments should not to be construed as encompassing those fragments that may have been disclosed prior to the invention.

A nucleic acid molecule of use in the present invention, e.g., a nucleic acid molecule having a variant of the nucleotide sequence of SEQ ID NO:1, or a complement thereof, can be isolated using standard molecular biology techniques and the sequence information provided herein. Using all of the nucleic acid sequence of SEQ ID NO:1, or portion thereof comprising a mutated nucleotide, or a complement of any of these nucleotide sequences, as a hybridization probe, UBA3 variant nucleic acid molecules can be isolated using standard hybridization and cloning techniques (e.g., as described in Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989).

A nucleic acid of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis, e.g., as described in the Examples.

In another embodiment, an isolated nucleic acid molecule of the invention includes a nucleic acid molecule which is a complement of a variant of the nucleotide sequence shown in SEQ ID NO:1 or a complement of a portion of the variant nucleotide sequence. In other embodiments, the nucleic acid molecule of the invention is sufficiently complementary to the nucleotide sequence shown in SEQ ID NO:1 such that it can hybridize, under conditions known in the art or described herein, to the variant nucleotide sequence shown in SEQ ID NO:1, thereby forming a stable duplex.

The UBA3 variant nucleotide sequences encoding a resistance UBA3 protein (i.e., a resistance gene) described herein allow for the generation of probes and primers designed for use in identifying and/or cloning resistance homologues in other cell types, e.g., from other tissues, as well as resistance nucleic acid homologues and orthologs from other species, such as mammals, insects or fungi. The probe/primer typically comprises substantially purified oligonucleotide which is at least 5 or 10, or less than 200, less than 100, or less than 50, bases in length. The oligonucleotide typically comprises a region of nucleotide sequence that hybridizes under stringent conditions to at least about 12, about 20, about 25, about 50, 75, 100, 125, 150, 175, 200, or more consecutive nucleotides of the sense or anti-sense sequence of SEQ ID NO:1, e.g., a portion comprising a mutated nucleotide. Furthermore, examples of oligonucleotides capable of discriminating, in an UBA3 polynucleic acid or a fragment thereof, sequences encoding mutated amino acid residues have been provided in Tables 2 and 3.

Probes based on a resistance nucleotide sequence can be used to detect transcripts or genomic sequences encoding the same or identical proteins. The probe can comprise a labeled group attached thereto, e.g., a radioisotope, a fluorescent compound, an enzyme, an enzyme co-factor a chemiluminescent agent, a colorimetric, phosphorescent or infrared dye or a surface-enhanced Raman label or plasmon resonant particle (PRP). Such probes can be used as a part of a diagnostic test kit for identifying UBA3 variants and orthologs of the resistance protein of the present invention, identifying cells or tissue which mis-express a resistance sequence, such as by measuring a level of a resistance protein-encoding nucleic acid in a sample of cells from a subject, e.g., detecting resistance mRNA levels or determining whether a genomic resistance gene has been mutated or deleted.

Oligonucleotides can be made in vitro by means of a nucleotide sequence amplification method. If such an amplified oligonucleotide is double-stranded, conversion to a single-stranded molecule can be achieved by a suitable exonuclease given that the desired single-stranded oligonucleotide is protected against said exonuclease activity. Alternatively, oligonucleotides are derived from recombinant plasmids containing inserts including the corresponding nucleotide sequences, if need be by cleaving the latter out from the cloned plasmids upon using the adequate nucleases and recovering them, e.g. by fractionation according to molecular weight. The oligonucleotides according to the present invention can also be synthetic, i.e. be synthesized chemically, for instance by applying the conventional phospho-triester or phosphoramidite chemistry, e.g., using an automated DNA synthesizer. Oligonucleotides can further be synthesized in situ on a glass slide via solid-phase oligonucleotide synthesis or via photolitographic synthesis.

Nucleic acids of the invention can be chosen for having codons, which are tailored for a particular expression system. E.g., the nucleic acid can be one in which at least one codon, or at least 10%, or 20% of the codons has been altered such that the sequence is optimized for expression in E. coli, yeast, human, insect, or CHO cells.

An isolated nucleic acid molecule encoding a resistance polypeptide can be created by introducing one or more nucleotide mutations, such as substitutions, additions or deletions into the nucleotide sequence of a resistance nucleic acid (variant of SEQ ID NO:1) such that one or more amino acid mutations, such as substitutions, additions or deletions are introduced into the encoded protein. Mutations can be introduced by standard techniques, such as site-directed mutagenesis and PCR-mediated mutagenesis. In some embodiments, mutations result in amino acid changes described herein. In other embodiments, conservative amino acid substitutions are made at one or more predicted amino acid residues described as causing resistance when mutated.

### Antisense Nucleic Acid Molecules, Ribozymes and Modified UBA3 Variant Nucleic Acid Molecules

In another aspect, the invention features an isolated nucleic acid molecule which is antisense to an E1 enzyme variant, e.g., UBA3, UAE, or UBA6, or other E1 enzyme variant described herein. An “antisense” nucleic acid can include a nucleotide sequence which is complementary to a “sense” nucleic acid encoding a protein, e.g., complementary to the coding strand of a double-stranded cDNA molecule or complementary to an mRNA sequence. An antisense nucleic acid can be complementary to an entire UBA3 variant coding strand, or to only a portion thereof (e.g., a portion comprising a mutated nucleotide described herein). In another embodiment, the antisense nucleic acid molecule is antisense to a “noncoding region” of the coding strand of a nucleotide sequence encoding a UBA3 variant (e.g., the 5′ and 3′ untranslated regions).

An antisense nucleic acid can be designed such that it is complementary to the entire coding region of a UBA3 variant mRNA, or the antisense nucleic acid can be an oligonucleotide which is complementary to only a portion of the coding or noncoding region of UBA3 variant mRNA. For example, the antisense oligonucleotide can be complementary to the region surrounding the translation start site of UBA3 variant mRNA, e.g., between the −10 and +10 regions of the target gene nucleotide sequence of interest. In another example, an antisense nucleic acid can be complementary to a portion of SEQ ID NO:1 comprising a mutation described herein. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a UBA3 variant protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically or selectively bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule can be placed under the control of a strong pol II or pol III promoter.

Nucleic acids complementary to UBA3 variant mRNA can be designed, e.g., to interfere with UBA3 variant expression. Such nucleic acids can employ the RNA interference (RNAi) method of post-transcriptional gene regulation. RNAi is induced by short double stranded RNA (dsRNA) molecules (Fire et al. (1998) Nature 391:806-811). Examples of dsRNA molecules are short hairpin RNAs (shRNAs), short interfering RNAs (siRNAs) and microRNAs (miRNAs). These short dsRNA molecules cause the destruction of messenger RNAs (mRNAs) which share sequence homology with the siRNA to within one nucleotide resolution (Elbashir S M et al. (2001), Genes Dev, 15: 188-200). It is believed that the siRNA and the targeted mRNA bind to an RNA-induced silencing complex (RISC), which cleaves the targeted mRNA and can recycle the siRNA. The siRNA's comprise a sense RNA strand and a complementary antisense RNA strand. There can be a 3′ overhang, i.e. at least one unpaired nucleotide extending about 1 to 6 nucleotides from the 3′-end of one or both RNA strand. Techniques for designing siRNAs are widely available, such as on websites maintained by providers of siRNA reagents, such as Dharmacon division of Thermo Scientific (Lafayette, Colo.) or Ambion division of Applied Biosystems (Austin Tex.). An siRNA targeting UBA3 variant expression is complementary to a portion of SEQ ID NO:1, comprising a mutation at a base selected from the group consisting of nucleotide 531, 532, 533, 621, 622, 623, 630, 631, 632, 633, 634, 635, 645, 646, 647, 651, 652, 653, 702, 703, 704, 705, 706, 707, 765, 766, 767, 933, 934, 935, 951, 952, 953, 960, 961, 962, 989, 990, and 991. In some embodiments, a siRNA is about 15 nucleotides to about 50 nucleotides in length, about 19 to about 30 nucleotides in length, or about 20 to 25 nucleotides in length. siRNAs can be administered, e.g., to a subject with a tumor or a parasitic infection, directly, e.g. delivered as siRNA molecules in a form that can enter the cell comprising the target mRNA, e.g., in liposomes or conjugated to a membrane soluble molecule or to a molecule which is internalized into endosomes, or they can be provided in a form, e.g., a vector or encapsulated in a viral agent, which can be expressed in the cell and cleaved to form the siRNA.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a UBA3 variant-encoding nucleic acid can include one or more sequences complementary to the nucleotide sequence of a UBA3 variant cDNA disclosed herein (i.e., a variant of SEQ ID NO:1), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a UBA3 variant-encoding mRNA. See, e.g., Cech et al. U.S. Pat. No. 4,987,071; and Cech et al. U.S. Pat. No. 5,116,742. Alternatively, UBA3 variant mRNA can be used to select a catalytic RNA having a specific ribonuclease activity from a pool of RNA molecules. See, e.g., Bartel and Szostak (1993) Science 261:1411-1418.

UBA3 variant gene expression can be inhibited by targeting nucleotide sequences complementary to the regulatory region of the UBA3 variant (e.g., the UBA3 variant promoter and/or enhancers) to form triple helical structures that prevent transcription of the UBA3 variant gene in target cells. See generally, Helene (1991) Anticancer Drug Des. 6:569-84; Helene (1992) Ann. N.Y. Acad. Sci. 660:27-36; and Maher (1992) Bioassays 14:807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

A UBA3 variant nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. 93: 14670-675.

PNAs of UBA3 variant nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of UBA3 variant nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup et al. (1996) supra)); or as probes or primers for DNA sequencing or hybridization (Hyrup et al. (1996) supra; Perry-O'Keefe supra).

The invention also includes molecular beacon oligonucleotide primer and probe molecules having at least one region which is complementary to a UBA3 variant nucleic acid of the invention, two complementary regions one having a fluorophore and one a quencher such that the molecular beacon is useful for quantitating the presence of the UBA3 variant nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

### Isolated E1 Enzyme Variant Polypeptides

In another aspect, the invention provides E1 enzyme variant, e.g., UBA3, UAE, or UBA6, or other E1 enzyme variant polypeptides. In one embodiment, a UBA3 variant polypeptide of the present invention can have an amino acid sequence substantially identical to variants of the amino acid sequence of SEQ ID NO:2. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 75% identity, 85% identity, 90% identity, 91%, 92%, 93%, 94%, 95%, 96%, 97%, 98% or 99% identity to a variant of SEQ ID NO:2 described herein are termed substantially identical.

In another aspect, the invention features an isolated UBA3, UAE, or UBA6, or other E1 enzyme variant protein, or fragment thereof. In one embodiment, the isolated UBA3 variant protein, or fragment thereof can be a biologically active portion, e.g., comprising one or more than one portion of UBA3 such as a binding site or a domain, e.g., a ThiF domain, a UBA_e1_thiolCys domain, a ubiquitin-activating enzyme signature sequence, a ATP binding site, a UBACT domain, a NEDD8 binding cleft or site, or an E2 bind domain. An example of a use of a biologically active portion of a UBA3 variant protein is a biochemical assay which isolates one or more than one particular function for analysis. Examples of such isolated biochemical assays are a nucleotide-, e.g. ATP-, binding assay, a nucleotide hydrolysis assay, a pyrophosphate binding assay, a pyrophosphate exchange assay, a NEDD8 binding assay, a NEDD8 adenylation assay, a NEDD8 thioester bond assay, an E2 enzyme binding assay, an assay to detect or measure transthiolation of NEDD8 to an E2 enzyme, a NAE1 binding assay, or an assay which measures the tightness of binding or on-off kinetics of binding to a NAE inhibitor-NEDD8 adduct. In another embodiment, an isolated UBA3 variant protein, or fragment thereof can be an about 8 to about 150 amino acid consecutive sequence portion, e.g., 8, 10, 15, 20, 50, 70, 100 or more amino acids as immunogens or antigens to raise or test (or more generally to bind) anti-UBA3 variant antibodies. In these embodiments, the polypeptide comprising the UBA3 portion or fragment can comprise a variant residue, including a variant residue which results in reduced sensitivity or resistance to an E1 enzyme inhibitor. A UBA3 variant protein or fragment thereof can be isolated from cells or tissue sources using standard protein purification techniques. A UBA3 variant protein or fragment thereof can be produced by recombinant DNA techniques or synthesized chemically.

Polypeptides of the invention include those which arise as a result of the existence of multiple genes, alternative transcription events, alternative RNA splicing events, and alternative translational and post-translational events. In an embodiment, the isolated polypeptide has about 85%, about 90%, about 95%, about 96%, about 97%, about 98% or about 99% identity to SEQ ID NO:2 and comprises at least one amino acid mutation, e.g. an amino acid substitution as described herein. In such an embodiment, variation in addition to at least one amino acid mutation, e.g. an amino acid substitution described herein can be a mutation, e.g. an amino acid substitution for a non-essential amino acid residue. The polypeptide can be expressed in systems, e.g., cultured cells, which result in substantially the same post-translational modifications present when the polypeptide is expressed in a native cell, or in systems which result in the alteration or omission of post-translational modifications, e.g., glycosylation or cleavage, present in a native cell.

The present invention extends to expression products that comprise at least one amino acid mutation, e.g. an amino acid substitution and/or deletion in the UBA3, UAE, or UBA6, or other E1 enzyme gene. For example, the invention relates to expression products comprising an amino acid mutation, e.g. an amino acid substitution described herein. The present invention also covers expression products that comprise besides the mutations or substitutions described herein, further amino acid mutations, e.g. amino acid substitutions in UBA3, UAE, or UBA6, or other E1 enzyme. Covered are expression products that comprise at least two amino acid mutations, e.g., substitutions in UBA3, UAE, or UBA6, or other E1 enzyme described herein. In some embodiments are expression products comprising one substitution of the alanine at amino acid residue 171 of SEQ ID NO:2 and at least one additional substitution described herein.

### E1 Enzyme Variant Chimeric or Fusion Proteins

In another aspect, the invention provides E1 enzyme variant, e.g., UBA3, UAE, or UBA6, or other E1 enzyme variant chimeric or fusion proteins. As used herein, a UBA3, UAE, or UBA6, or other E1 enzyme variant “chimeric protein” or “fusion protein” includes a UBA3, UAE, or UBA6, or other E1 enzyme variant polypeptide or variant portion thereof linked to a non-variant polypeptide. A “non-variant polypeptide” refers to a polypeptide having an amino acid sequence corresponding to a protein or a portion of a protein which does not comprise a mutated, e.g., substituted, amino acid residue, such as a variant amino acid residue described herein. A non-variant polypeptide can be a protein, e.g., a protein or a selectable portion thereof which is different from the UBA3, UAE, or UBA6, or other E1 enzyme variant or variant portion thereof and which is derived from the same or a different organism. A non-variant polypeptide can be a portion of an E1 enzyme which does not comprise an amino acid mutation, e.g., substitution, e.g., a portion of a wild type UBA3, UAE, or UBA6, or other E1 enzyme. In such an embodiment, a portion, e.g., a binding site or a domain, of an E1 variant enzyme comprising an amino acid mutation, e.g. an amino acid substitution can be fused to a different portion, e.g., a binding site or a domain, of a wild type E1 enzyme. In an embodiment, a UBA3, UAE, or UBA6, or other E1 enzyme variant fusion protein includes at least one or more than one biologically active portion of a UBA3, UAE, or UBA6, or other E1 enzyme variant protein. The non-variant polypeptide can be fused to the N-terminus or C-terminus of the UBA3, UAE, or UBA6, or other E1 enzyme variant polypeptide.

The fusion protein can include a moiety which has a high affinity for a ligand as the non-variant polypeptide. For example, the fusion protein can be a GST-UBA3, UAE, or UBA6, or other E1 enzyme variant fusion protein in which the UBA3, UAE, or UBA6, or other E1 enzyme variant sequences are fused to the C-terminus of the GST sequences. Such fusion proteins can facilitate the purification of recombinant UBA3, UAE, or UBA6, or other E1 enzyme variant. Alternatively, the fusion protein can be a UBA3, UAE, or UBA6, or other E1 enzyme variant protein containing a heterologous signal sequence, such as using the secretory sequence of gp67 baculovirus envelope protein, melittin and human placental alkaline phosphatase, prokaryotic secretory signal for phoA or protein A at its N-terminus. In certain host cells (e.g., mammalian host cells), expression and/or secretion of UBA3, UAE, or UBA6, or other E1 enzyme variant can be increased through use of a heterologous signal sequence.

An E1 enzyme variant fusion protein can include, e.g., as the non-variant polypeptide, all or a part of a serum protein, e.g., a portion of an immunoglobulin (e.g., IgG, IgA, or IgE), e.g., an Fc region and/or the hinge C1 and C2 sequences of an immunoglobulin or human serum albumin.

In another embodiment, the fusion protein can further comprise a heterologous epitope tag. Examples of a heterologous epitope tag include: a His₆ tag, a FLAG tag, a c-myc tag, glutathione-S-transferase (GST) tag, a hemagglutinin (HA) tag, a T7 gene 10 tag, a V5 tag, an HSV tag, and a VSV-G tag.

The UBA3, UAE, or UBA6, or other E1 enzyme variant fusion proteins of the invention can be incorporated into pharmaceutical compositions and administered to a subject in vivo. The UBA3, UAE, or UBA6, or other E1 enzyme variantfusion proteins can be used to affect the bioavailability of a UBA3, UAE, or UBA6, or other E1 enzyme variant substrate. UBA3, UAE, or UBA6, or other E1 enzyme variant fusion proteins can be useful therapeutically for the treatment of disorders caused by, for example, (i) aberrant modification or mutation of a gene encoding a UBA3, UAE, or UBA6, or other E1 enzyme protein; (ii) mis-regulation of the UBA3, UAE, or UBA6, or other E1 enzyme variant gene; and (iii) aberrant post-translational modification of a UBA3, UAE, or UBA6, or other E1 enzyme variant protein.

Moreover, the UBA3, UAE, or UBA6, or other E1 enzyme variant-fusion proteins of the invention can be used as immunogens to produce anti-UBA3, UAE, or UBA6, or other E1 enzyme variant antibodies in a subject, to purify UBA3, UAE, or UBA6, or other E1 enzyme variant ligands and in screening assays to identify molecules which inhibit the interaction of UBA3, UAE, or UBA6, or other E1 enzyme variant with a UBA3, UAE, or UBA6, or other E1 enzyme variant substrate or E1 enzyme inhibitor.

Expression vectors are commercially available that already encode a fusion moiety (e.g., a GST polypeptide). A UBA3, UAE, or UBA6, or other E1 enzyme variant-encoding nucleic acid can be cloned into such an expression vector such that the fusion moiety is linked in-frame to the UBA3, UAE, or UBA6, or other E1 enzyme variant protein.

### Variants of E1 Enzyme Proteins

Variants of an E1 enzyme proteins can be identified by screening combinatorial libraries of mutants, e.g., truncation mutants, of a E1 enzyme protein for agonist or antagonist activity. In some embodiments variants can result from adding or deleting a cysteine residue or a residue which is glycosylated.

Libraries of fragments e.g., N terminal, C terminal, or internal fragments, of a Ei enzyme variant protein coding sequence can be used to generate a diverse population of fragments for screening and subsequent selection of variants of an E1 enzyme protein. For example, a library of coding sequence fragments can be generated by treating a double stranded PCR fragment of a resistance protein coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of a resistance protein.

Methods for screening gene products of combinatorial libraries made by point mutations or truncation, and for screening cDNA libraries for gene products having a selected property are known in the art. Recursive ensemble mutagenesis (REM), a technique which enhances the frequency of functional mutants in the libraries, can be used in combination with the screening assays to identify E1 enzyme variants (Arkin and Yourvan (1992) Proc. Natl. Acad. Sci. USA 89:7811-7815; Delgrave et al. (1993) Protein Engineering 6:327-331).

Cell based assays can be exploited to analyze a diverse E1 enzyme variant library. For example, a library of expression vectors comprising an E1 enzyme variant protein or biologically active portion thereof can be transfected into a cell line, e.g., a cell line, which ordinarily responds to E1 enzyme activity in a substrate-dependent manner. The transfected cells are then contacted with a substrate and the effect of the expression of the mutant on signaling by the E1 enzyme substrate can be detected, e.g., measuring cellular signaling activity mediated by interaction of the transthiolated, e.g., ubiquitinated, sumoylated or neddylated protein with an E3 ligase, e.g., cullin ring ligase. For example, activity of an E1 enzyme variant, e.g., a UBA3 variant can be identified and measured by: 1) the ability to mediate turnover of substrates of the cullin ring ligase; 2) the ability to participate in protein homeostasis; and 3) the ability to support tumor cell survival, or more specifically by measuring E2 enzyme binding, or NAE1 binding. Plasmid DNA can then be recovered from the cells which score for inhibition, or alternatively, potentiation of signaling by the E1 enzyme substrate in the presence of an E1 enzyme inhibitor, e.g., MLN4924, and the individual clones further characterized.

In another aspect, the invention features a method of making a UBA3, UAE, or UBA6, or other E1 enzyme variant polypeptide, e.g., a peptide having a non-wild type activity, e.g., an antagonist, agonist, or super agonist of a naturally occurring UBA3, UAE, or UBA6, or other E1 enzyme variant polypeptide, e.g., a naturally occurring UBA3, UAE, or UBA6, or other E1 enzyme variant polypeptide. The method includes altering the sequence of a UBA3, UAE, or UBA6, or other E1 enzyme variant polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a UBA3, UAE, or UBA6, or other E1 enzyme variant polypeptide a biological activity of a naturally occurring UBA3, UAE, or UBA6, or other E1 enzyme variant polypeptide. The method includes altering the sequence, e.g., by substitution or deletion of one or more residues, of a UBA3, UAE, or UBA6, or other E1 enzyme variant polypeptide, e.g., altering the sequence of a non-conserved region, or a domain or residue described herein, and testing the altered polypeptide for the desired activity.

### Anti-E1 Enzyme Variant Antibodies

In another aspect, the invention provides an anti-UBA3, UAE, or UBA6, or other E1 enzyme variant antibody. The term “antibody” herein is used in the broadest sense and specifically covers full length monoclonal antibodies, immunoglobulins, polyclonal antibodies, multispecific antibodies (e.g. bispecific antibodies) formed from at least two full length antibodies, and individual antigen binding fragments, including dAbs, scFv, Fab, F(ab)′₂, Fab′, including human, humanized, chimeric and antibodies from non-human species and recombinant antigen binding forms such as monobodies and diabodies. The antibody can have effector function and can fix complement. The antibody can be coupled to a toxin, detectable label or imaging agent. Chimeric, humanized, or completely human antibodies are useful for applications which include repeated administration, e.g., therapeutic treatment of human patients, and some diagnostic applications.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

A full-length UBA3, UAE, or UBA6, or other E1 enzyme variant protein or, antigenic peptide fragment of UBA3, UAE, or UBA6, or other E1 enzyme variant can be used as an immunogen or can be used to identify anti-UBA3, UAE, or UBA6, or other E1 enzyme variant antibodies made with other immunogens, e.g., cells, cell lysates, membrane preparations, and the like. The antigenic peptide of UBA3, UAE, or UBA6, or other E1 enzyme variant should include at least 8 amino acid residues of the E1 enzyme variant amino acid sequence e.g., SEQ ID NO:2, 29 or 32 and encompasses an epitope of the UBA3, UAE, or UBA6, or other E1 enzyme variant. In some embodiments, the antigenic peptide includes at least 10 amino acid residues, at least 15 amino acid residues, at least 20 amino acid residues, or at least 30 amino acid residues and comprises a mutated, e.g., substituted amino acid residue, e.g., a residue that confers decreased sensitivity or resistance to an E1 Enzyme inhibitor, as described herein. In some embodiments, the antigenic peptide comprises amino acid residue 171, 201, 204, 205, 209, 211, 228, 229, 249, 305, 311, 314 or 324 of SEQ ID NO:2, amino acid residue 121 in SEQ ID NO:27, amino acid residue 548 in SEQ ID NO:28, amino acid residue 580 in SEQ ID NO:29, amino acid residue 185 in SEQ ID NO:30, amino acid residue 187 in SEQ ID NO:31, amino acid residue 573 in SEQ ID NO:32, amino acid residue 544 in SEQ ID NO:33, amino acid residue 480 in SEQ ID NO:34, amino acid residue 134 in SEQ ID NO:35 or amino acid residue 131 in SEQ ID NO:36, wherein the amino acid residue is a substitute for the wild type residue in the respective SEQ ID NO.

Alternatively, fragments of UBA3, UAE, or UBA6, or other E1 enzyme variant which includes a variant domain or binding site, e.g. a ThiF domain, a UBA_e1_thiolCys domain, a ubiquitin-activating enzyme signature sequence, a ATP binding site, a UBACT domain, a ubiquitin or NEDD8 binding cleft or site, or an E2_bind domain can be used to make an antibody against the variant region of the UBA3, UAE, or UBA6, or other E1 enzyme variant protein. In some embodiments, a domain or binding site including an amino acid mutation, e.g. an amino acid substitution as described herein are useful as immunogens to generate antibodies in a UBA3 variant and can include about amino acid residues 69 to about 211, about amino acid residues 235 to 243, about amino acid residues 216 to about 260, residues about 99 to 124 and about 146 to 174, about 78 to 81, 165 to 172, 201 to 229 and 310 to 344, about amino acid residues 270 to about 334, or about amino acid residues 374 to about 462 of SEQ ID NO:2.

In some embodiments, epitopes encompassed by the antigenic peptide are regions of UBA3, UAE, or UBA6, or other E1 enzyme variant located on the surface of the protein, e.g., hydrophilic regions, as well as regions with high antigenicity. For example, an Emini surface probability analysis of the human UBA3, UAE, or UBA6, or other E1 enzyme variant protein sequence can be used to indicate the regions that have a particularly high probability of being localized to the surface of the UBA3, UAE, or UBA6, or other E1 enzyme variant protein and are thus likely to constitute surface residues useful for targeting antibody production.

In some embodiments, an antibody to a UBA3, UAE, or UBA6, or other E1 enzyme variant can selectively bind to a portion of a UBA3, UAE, or UBA6, or other E1 enzyme comprising an amino acid mutation, e.g. an amino acid substitution described herein. For example, the antibody binding would be selective for a UBA3, UAE, or UBA6, or other E1 enzyme comprising the mutated, e.g., substituted residue and not a UBA3, UAE, or UBA6, or other E1 enzyme polypeptide comprising the wild type residue. In some embodiments an antibody to a UBA3, UAE, or UBA6, or other E1 enzyme variant can bind a UBA3 variant with a thr, asp, val, glu or ser at residue 171 of SEQ ID NO:2, a UBA3 variant with a val at residue 201 of SEQ ID NO:2, a UBA3 variant with a lys or gly at residue 204 of SEQ ID NO:2, a UBA3 variant with a cys at residue 205 of SEQ ID NO:2, a UBA3 variant with a lys or asp at residue 209 of SEQ ID NO:2, a UBA3 variant with a gln at residue 211 of SEQ ID NO:2, a UBA3 variant with an his at residue 228 of SEQ ID NO:2, a UBA3 variant with a gln at residue 229 of SEQ ID NO:2, a UBA3 variant with an ala at residue 305 of SEQ ID NO:2, a UBA3 variant with an ser or thr at residue 311 of SEQ ID NO:2, a UBA3 variant with a pro at residue 314 of SEQ ID NO:2, a UBA3 variant with with a tyr at residue 324 of SEQ ID NO:2, a UBA3 variant with a tyr at residue 249 of SEQ ID NO:2, a UAE variant with a thr at residue 280 of SEQ ID NO:29 or a UBA6 variant with a thr or asp at residue 573 of SEQ ID NO:32.

In some embodiments, an antibody can be made by immunizing an animal, e.g., mouse, rat, chicken, rabbit, sheep or goat, with a purified UBA3, UAE, or UBA6, or other E1 enzyme variant antigen, or a fragment thereof, e.g., a fragment comprising a mutated, e.g., substituted amino acid residue described herein, a membrane associated antigen, tissue, e.g., a crude tissue preparations, whole cells, e.g., living cells, lysed cells, or cell fractions, e.g., cytosol fractions, nuclear fractions or membrane fractions. Antibodies reactive with, or specific or selective for, any of these regions described above, or other regions or domains described herein are provided. In the case of antibodies directed against small peptides such as fragments of a protein of the invention, said peptides are generally coupled to a carrier protein before immunization of animals. Such protein carriers include keyhole limpet hemocyanin (KLH), bovine serum albumin (BSA), ovalbumin and Tetanus toxoid.

Chimeric, humanized and primatized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559).

A humanized or complementarity determining region (CDR)-grafted antibody will have at least one or two, but generally all three recipient CDR's (of heavy and or light immuoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to a UBA3, UAE, or UBA6, or other E1 enzyme variant or a fragment thereof. In one embodiment, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, e.g., 90%, 95%, 99% or higher identical thereto.

As used herein, the term “consensus sequence” refers to the sequence formed from the most frequently occurring amino acids (or nucleotides) in a family of related sequences (See e.g., Winnaker, (1987) From Genes to Clones (Verlagsgesellschaft, Weinheim, Germany). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison (1985) Science 229:1202-1207, by Oi et al. (1986) BioTechniques 4:214, and by Queen et al. U.S. Pat. Nos. 5,585,089, 5,693,761 and 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a UBA3, UAE, or UBA6, or other E1 enzyme variant polypeptide or variant fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. (1986) Nature 321:552-525; Verhoeyan et al. (1988) Science 239:1534; Beidler et al. (1988) J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. In some embodiments, humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Useful locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In some embodiments, completely human antibodies are generated for therapeutic treatment of human patients. The term “human antibody” includes an antibody that possesses a sequence that is derived from a human germ-line immunoglobulin sequence. See, for example, Lonberg and Huszar (1995) Int. Rev. Immunol. 13:65-93); and U.S. Pat. Nos. 5,625,126; 5,633,425; 5,569,825; 5,661,016; and 5,545,806. Examples of human antibodies include an antibody derived from transgenic mice having human immunoglobulin genes (e.g., XENOMOUSE genetically engineered mice (Abgenix, Fremont, Calif.), HUMAB-MOUSE®, KIRIN TC MOUSE™ transchromosome mice, KMMOUSE® (MEDAREX, Princeton, N.J.)), human phage display libraries, human myeloma cells, or human B cells.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

The anti-UBA3, UAE, or UBA6, or other E1 enzyme variant antibody can be a single chain antibody. A single-chain antibody (scFV) can be engineered as described in, for example, Colcher et al. (1999) Ann. N Y Acad. Sci. 880:263-80; and Reiter (1996) Clin. Cancer Res. 2:245-52. The single chain antibody can be dimerized or multimerized to generate multivalent antibodies having specificities for different epitopes of the same target UBA3, UAE, or UBA6, or other E1 enzyme variant protein.

In an embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, auristatins (see, e.g., Doronina et al., Nature Biotech., 21: 778-784 (2003); Hamblen et al, Clin. Cancer Res., 10: 7063-7070 (2004); Carter and Senter, Cancer J., 14 154-169 (2008); U.S. Pat. Nos. 7,498,298; 6,884,869; 7,091,186; 7,837,980; 7,659,241; or US Patent Publication No. 20080300192), maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol, auristatins and maytansinoids). Radioactive ions include, but are not limited to iodine, yttrium and praseodymium.

The conjugates of the invention can be used for modifying a given biological response; the therapeutic moiety is not to be construed as limited to classical chemical therapeutic agents. For example, the therapeutic moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

An anti-UBA3, UAE, or UBA6, or other E1 enzyme variant antibody (e.g., monoclonal antibody) can be used to isolate a UBA3, UAE, or UBA6, or other E1 enzyme variant by standard techniques, such as affinity chromatography or immunoprecipitation. Moreover, an anti-UBA3, UAE, or UBA6, or other E1 enzyme variant antibody can be used to detect UBA3, UAE, or UBA6, or other E1 enzyme variant protein (e.g., in a cellular lysate or cell supernatant) in order to evaluate the abundance and pattern of expression of the protein. Anti-UBA3, UAE, or UBA6, or other E1 enzyme variant antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorothazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Tools useful in relying on antibodies against a UBA3, UAE, or UBA6, or other E1 enzyme variant protein include protein gel blot analysis, screening of expression libraries allowing gene identification, protein quantitative methods including ELISA (enzyme-linked immunosorbent assay), RIA (radio-immuno-assay) and LIA (line immuno-assay), immunoaffinity purification of proteins, immunoprecipitation of proteins and immunolocalization of proteins.

Antibodies which bind only a native UBA3, UAE, or UBA6, or other E1 enzyme variant protein, only denatured or otherwise non-native UBA3, UAE, or UBA6, or other E1 enzyme variant protein, or which bind both, are within the invention. Antibodies with linear or conformational epitopes are within the invention. Conformational epitopes sometimes can be identified by identifying antibodies which bind to native but not denatured UBA3, UAE, or UBA6, or other E1 enzyme variant protein.

### Recombinant Expression Vectors, Host Cells and Genetically Engineered Cells

In another aspect, the invention includes, vectors, e.g., expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a UBA3, UAE, or UBA6, or other E1 enzyme variant nucleic acid in a form suitable for expression of the nucleic acid in a host cell. In some embodiments, the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. Expression may furthermore be transient expression or stable expression or, alternatively, controllable expression. Controllable expression comprises inducible expression, e.g. using a tetracyclin-regulatable promoter, a stress-inducible (e.g. human hsp70 gene promoter), a methallothionine promoter, a glucocorticoid promoter or a progesterone promoter. Promoters further can include HBV promoters such as the core promoter and heterologous promoters such as the cytomegalovirus (CMV) immediate early (IE) promoter. The expression vectors of the invention can be introduced into host cells to thereby produce proteins or polypeptides, including fusion proteins or polypeptides, encoded by nucleic acids as described herein (e.g., UBA3, UAE, or UBA6, or other E1 enzyme variant proteins, fusion proteins, and the like).

The recombinant expression vectors of the invention can be designed for expression of UBA3, UAE, or UBA6, or other E1 enzyme variant proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in UBA3, UAE, or UBA6, or other E1 enzyme variant activity assays, (e.g., direct assays or competitive assays described in detail below), or to generate antibodies specific or selective for UBA3, UAE, or UBA6, or other E1 enzyme variant proteins. In one embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in host bacteria with an impaired capacity to proteolytically cleave the recombinant protein (Gottesman (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The UBA3, UAE, or UBA6, or other E1 enzyme variant expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub et aL, (1986) Reviews—Trends in Genetics 1:1.

A vector, or an expression vector, may furthermore be capable of autonomous replication in a host cell or may be an integrative vector, i.e. a vector completely or partially, and stably, integrating in the genome of a host cell. Integration of any first DNA fragment, e.g. a vector or a fragment thereof, in any other second DNA fragment, e.g. the genome of a host cell, can be reversed if said first DNA fragment is flanked e.g. by site-specific recombination sites or by repeat sequences typical for transposons. Alternatively, said site-specific recombination sites or transposon-repeat sequences are comprised in said second DNA fragment and are flanking said first DNA fragment. Another aspect the invention provides a host cell which includes a nucleic acid molecule described herein, e.g., a UBA3, UAE, or UBA6, or other E1 enzyme variant nucleic acid molecule within a recombinant expression vector or a UBA3, UAE, or UBA6, or other E1 enzyme variant nucleic acid molecule containing sequences which allow it to homologously recombine into a specific site of the host cell's genome. The terms “host cell” and “recombinant host cell” are used interchangeably herein. Such terms refer not only to the particular subject cell but also to the progeny or potential progeny of such a cell. Because certain modifications can occur in succeeding generations due to either mutation or environmental influences, such progeny may not, in fact, be identical to the parent cell, but are still included within the scope of the term as used herein.

A host cell can be any prokaryotic or eukaryotic cell, e.g. a cell in culture. For example, a UBA3, UAE, or UBA6, or other E1 enzyme variant protein can be expressed in bacterial cells such as E. coli, insect cells, yeast or mammalian cells (such as Chinese hamster ovary (CHO) cells or CV-1 origin, SV-40 (COS) cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including comprise heat-shock mediated transformation (e.g. of E. coli), conjugative DNA transfer, calcium phosphate or calcium chloride co-precipitation, DEAE-dextran-mediated transfection, lipofection, or electroporation direct introduction by e.g. microinjection or particle bombardment, or introduction by means of a virus, virion or viral particle.

A host cell of the invention can be used to produce (i.e., express) a UBA3, UAE, or UBA6, or other E1 enzyme variant protein. Accordingly, the invention further provides methods for producing a UBA3, UAE, or UBA6, or other E1 enzyme variant protein using the host cells of the invention. In one embodiment, the method includes culturing the host cell of the invention (into which a recombinant expression vector encoding a UBA3, UAE, or UBA6, or other E1 enzyme variant protein has been introduced) in a suitable medium such that a UBA3, UAE, or UBA6, or other E1 enzyme variant protein is produced. The method can employ a selectable marker to enrich for cells comprising the UBA3, UAE, or UBA6, or other E1 enzyme variant. In another embodiment, the method further includes isolating a UBA3, UAE, or UBA6, or other E1 enzyme variant protein from the medium or the host cell.

In another aspect, the invention features, a cell or purified preparation of cells which include a UBA3, UAE, or UBA6, or other E1 enzyme variant transgene, or which otherwise express or misexpress UBA3, UAE, or UBA6, or other E1 enzyme variant. The cell preparation can consist of human or non-human cells, e.g., rodent cells, e.g., mouse or rat cells, rabbit cells, or pig cells. In some embodiments, the cell or cells include a UBA3, UAE, or UBA6, or other E1 enzyme variant transgene, e.g., a heterologous form of a UBA3, UAE, or UBA6, or other E1 enzyme variant, e.g., a gene derived from humans (in the case of a non-human cell). The UBA3, UAE, or UBA6, or other E1 enzyme variant transgene can be misexpressed, e.g., overexpressed or underexpressed. A cell comprising the UBA3, UAE, or UBA6, or other E1 enzyme variant gene can be used in culture or used in an animal host, such as mouse, guinea pig or rat e.g., in a graft (e.g., a tumor graft). Location of the cell comprising UBA3, UAE, or UBA6, or other E1 enzyme variant in the host can be intraperitoneal, subcutaneous or disseminated, e.g., after injection into the bloodstream.

In other embodiments, the cell or cells include a gene which misexpresses an endogenous UBA3, UAE, or UBA6, or other E1 enzyme variant, e.g., a gene the expression of which is disrupted, e.g., a knockout. Such cells can serve as a model for studying disorders which are related to misexpressed UBA3, UAE, or UBA6, or other E1 enzyme variant alleles or for use in drug screening, e.g., to identify E1 inhibitors that overcome the lower sensitivity or resistance conferred by the variant.

In another aspect, the invention feature a human cell, e.g., a hematopoietic stem cell, transformed with nucleic acid which encodes a subject UBA3, UAE, or UBA6, or other E1 enzyme variant polypeptide.

Also provided are cells, such as human cells, e.g., human hematopoietic or fibroblast cells, in which an endogenous UBA3, UAE, or UBA6, or other E1 enzyme variant is under the control of a regulatory sequence that does not normally control the expression of the endogenous UBA3, UAE, or UBA6, or other E1 enzyme variant gene. The expression characteristics of an endogenous gene within a cell, e.g., a cell line or microorganism, can be modified by inserting a heterologous DNA regulatory element into the genome of the cell such that the inserted regulatory element is operably linked to the endogenous UBA3, UAE, or UBA6, or other E1 enzyme variant gene. For example, an endogenous UBA3, UAE, or UBA6, or other E1 enzyme variant gene which is “transcriptionally silent,” e.g., not normally expressed, or expressed only at very low levels, can be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

### Transgenic Animals

The invention provides non-human transgenic animals. Such animals are useful for studying the function and/or activity of a UBA3, UAE, or UBA6, or other E1 enzyme variant protein and for identifying and/or evaluating modulators of UBA3, UAE, or UBA6, or other E1 enzyme variant activity. As used herein, a “transgenic animal” is a non-human animal, such as a mammal, e.g., a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which can be integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous UBA3, UAE, or UBA6, or other E1 enzyme variant gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention in order to direct expression of a UBA3, UAE, or UBA6, or other E1 enzyme variant protein to particular cells. A transgenic founder animal can be identified based upon the presence of a UBA3, UAE, or UBA6, or other E1 enzyme variant transgene in its genome and/or expression of UBA3, UAE, or UBA6, or other E1 enzyme variant mRNA in tissues or cells of the animals. A transgenic founder animal can then be used to breed additional animals carrying the transgene. Moreover, transgenic animals carrying a transgene encoding a UBA3, UAE, or UBA6, or other E1 enzyme variant protein can further be bred to other transgenic animals carrying other transgenes.

UBA3, UAE, or UBA6, or other E1 enzyme variant proteins or polypeptides can be expressed in transgenic animals or plants, e.g., a nucleic acid encoding the protein or polypeptide can be introduced into the genome of an animal. In some embodiments the nucleic acid is placed under the control of a tissue specific promoter, e.g., a milk- or egg-specific promoter, and recovered from the milk or eggs produced by the animal. Suitable animals are mice, pigs, cows, goats, and sheep.

The invention also includes a population of cells from a transgenic animal, as discussed, e.g., below.

### Uses

The nucleic acid molecules, proteins, protein homologs, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a UBA3, UAE, or UBA6, or other E1 enzyme variant protein (e.g., via a recombinant expression vector in a host cell in gene therapy applications), to detect a UBA3, UAE, or UBA6, or other E1 enzyme variant mRNA (e.g., in a biological sample) or a genetic alteration in a UBA3, UAE, or UBA6, or other E1 enzyme variant gene, and to modulate UBA3, UAE, or UBA6, or other E1 enzyme variant activity, as described further below. The UBA3, UAE, or UBA6, or other E1 enzyme variant proteins can be used to treat disorders characterized by insufficient or excessive production of a E1 enzyme substrate or production of UBA3, UAE, or UBA6, or other E1 enzyme variant inhibitors. In addition, the UBA3, UAE, or UBA6, or other E1 enzyme variant proteins can be used to screen for naturally occurring UBA3, UAE, or UBA6, or other E1 enzyme variant substrates, to screen for drugs or compounds which modulate UBA3, UAE, or UBA6, or other E1 enzyme variant activity, as well as to treat disorders characterized by insufficient or excessive production of UBA3, UAE, or UBA6, or other E1 enzyme variant protein or production of UBA3, UAE, or UBA6, or other E1 enzyme variant protein forms which have decreased, aberrant or unwanted activity compared to E1 enzyme wild type protein (e.g., for UBA3, the ability to bind a nucleotide, the ability to hydrolyze a nucleotide, the ability to bind NEDD8, the ability to adenylate NEDD8, the ability to form a covalent thioester bond with NEDD8, the ability to bind an E2 enzyme, the ability to catalyze the transthiolation of NEDD8 to an E2 or the ability to bind NAE1 or expression). Moreover, the anti-UBA3, UAE, or UBA6, or other E1 enzyme variant antibodies of the invention can be used to detect and isolate UBA3, UAE, or UBA6, or other E1 enzyme variant proteins, regulate the bioavailability of UBA3, UAE, or UBA6, or other E1 enzyme variant proteins, modulate UBA3, UAE, or UBA6, or other E1 enzyme variant activity or treat disorders related to UBA3, UAE, or UBA6, or other E1 enzyme variant function or expression.

A method of evaluating a compound for the ability to interact with, e.g., bind, a subject UBA3, UAE, or UBA6, or other E1 enzyme variant polypeptide is provided. The method includes: contacting the compound with the subject UBA3, UAE, or UBA6, or other E1 enzyme variant polypeptide; and evaluating ability of the compound to interact with, e.g., to bind or form a complex with the subject UBA3, UAE, or UBA6, or other E1 enzyme variant polypeptide. This method can be performed in vitro, e.g., in a cell-free system, or in vivo, e.g., in a two-hybrid interaction trap assay or in a disease model. This method can be used to identify naturally occurring molecules which interact with subject UBA3, UAE, or UBA6, or other E1 enzyme variant polypeptide. It can also be used to find natural or synthetic inhibitors of subject UBA3, UAE, or UBA6, or other E1 enzyme variant polypeptide. Screening methods are discussed in more detail below.

### Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to UBA3, UAE, or UBA6, or other E1 enzyme variant proteins, have a stimulatory or inhibitory effect on, for example, UBA3, UAE, or UBA6, or other E1 enzyme variant expression or UBA3, UAE, or UBA6, or other E1 enzyme variant activity, or have a stimulatory or inhibitory effect on, for example, the expression or activity of a UBA3, UAE, or UBA6, or other E1 enzyme variant substrate or proteins in the E1 enzyme pathway, e.g., in the NAE pathway. Compounds thus identified can be used to modulate the activity of target gene products (e.g., UBA3, UAE, or UBA6, or other E1 enzyme variant genes) in a therapeutic protocol, to elaborate the biological function of the target gene product, or to identify compounds that disrupt UBA3, UAE, or UBA6, or other E1 enzyme variant gene interactions.

In one embodiment, the assay can identify compounds which bind UBA3 more tightly than MLN4924. In other embodiments, the assay can identify compounds which bind to a UBA3 variant with at least one amino acid difference from SEQ ID NO:2 selected from the group consisting of amino acid residue 171, 201, 204, 205, 209, 211, 228, 229, 249, 305, 311, 314 and 324 of SEQ ID NO:2.

In some embodiments, the assay can identify compounds which modulate one or more activity of a E1 enzyme variant, e.g., an E1 enzyme with a mutation, e.g. an amino acid substitution, e.g., as described herein. For some examples, the activity of an E1 enzyme variant, e.g., a UBA3 variant can be: the ability to mediate turnover of substrates of the E3 ligase, e.g., cullin ring ligase; the ability to participate in protein homeostasis; the ability to support tumor cell survival; the ability to bind a nucleotide, the ability to hydrolyze a nucleotide, the ability to bind a pyrophosphate, the ability to bind a UBL, the ability to adenylate a UBL, the ability to form a thioester bond with a UBL, the ability to bind an E2 enzyme, the ability to transthiolate a UBL to an E2 enzyme, or an assay which measures the tightness or on-off kinetics of binding to an E1 enzyme inhibitor-UBL adduct. Assays which can identify compounds which modulate one or more activity of a UBA3 variant include the ability to bind a nucleotide, the ability to hydrolyze a nucleotide, the ability to bind NEDD8, the ability to adenylate NEDD8, the ability to form a covalent thioester bond with NEDD8, the ability to bind an E2 enzyme, the ability to catalyze the transthiolation of NEDD8 to an E2, the ability to bind NAE1, the ability to mediate turnover of substrates of the cullin ring ligase, the ability to participate in protein homeostasis, and/or the ability to support tumor cell survival. In some embodiments, there can be a comparison of the activity of the UBA3 variant in the presence of the test agent with the activity in the presence of an E1 enzyme inhibitor, e.g., an NAE inhibitor, e.g., MLN4924, to which the UBA3 variant has resistance. Assays for UBA3 variant activities can be performed by the methods described in the Examples.

In one embodiment, the invention provides assays for screening candidate or test compounds which are substrates of a UBA3, UAE, or UBA6, or other E1 enzyme variant protein or polypeptide or a biologically active portion thereof. In another embodiment, the invention provides assays for screening candidate or test compounds which bind to or modulate the activity of a UBA3, UAE, or UBA6, or other E1 enzyme variant protein or polypeptide or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909-13; Erb et al. (1994) Proc. Natl. Acad. Sci. USA 91:11422-426; Zuckermann et al. (1994). J. Med Chem. 37:2678-85; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233-51.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

Assays UBA3, UAE, or UBA6, or other E1 enzyme variant can be cell-based assays. Cell-based assays can measure turnover of substrates of the E3 ligase; the maintenance of protein homeostasis; the number of surviving tumor cells, the amount of interaction between the E1 enzyme and the E2 enzyme, or the amount of signaling by the E1 enzyme substrate e.g., cellular signaling activity mediated by interaction of the transthiolated, e.g., ubiquitinated, sumoylated or neddylated protein with an E3 ligase. Assays can be in vitro assays.

In one embodiment, an assay is a cell-based assay in which a cell which expresses a UBA3 variant protein or biologically active portion thereof is contacted with a test compound, and the ability of the test compound to modulate UBA3 variant activity is determined. Determining the ability of the test compound to modulate UBA3 variant activity can be accomplished by monitoring, for example, the ability to bind a nucleotide, the ability to hydrolyze a nucleotide, the ability to bind NEDD8, the ability to adenylate NEDD8, the ability to form a covalent thioester bond with NEDD8, the ability to bind an E2 enzyme, the ability to catalyze the transthiolation of NEDD8 to an E2, the ability to bind NAE1, the ability to mediate turnover of substrates of the cullin ring ligase, the ability to participate in protein homeostasis, and/or the ability to support tumor cell survival. The assay can be performed in a cell-based system, e.g., with optical or radioactive detection and quantification methods or in a system that lyses the cells after time for reaction and then assays cell contents. In some embodiments, there can be a comparison of the activity of the UBA3 variant in the presence of the test agent with the activity in the presence of an E1 enzyme inhibitor, e.g., an NAE inhibitor, e.g., MLN4924, to which the UBA3 variant has resistance. The cell, for example, can be of mammalian origin, e.g., human. In other embodiments, the assay can determine the ability of the test compound to modulate a variant of an enzyme structurally or mechanistically similar to UBA3 in a drug resistant parasitic cell.

The ability of the test compound to modulate UBA3, UAE, or UBA6, or other E1 enzyme variant binding to a compound, e.g., a UBA3, UAE, or UBA6, or other E1 enzyme variant substrate, or to bind to UBA3, UAE, or UBA6, or other E1 enzyme variant can also be evaluated. This can be accomplished, for example, by coupling the compound, e.g., the substrate, with a radioisotope or enzymatic label such that binding of the compound, e.g., the substrate, to UBA3, UAE, or UBA6, or other E1 enzyme variant can be determined by detecting the labeled compound, e.g., substrate, in a complex. Alternatively, UBA3, UAE, or UBA6, or other E1 enzyme variant could be coupled with a radioisotope or enzymatic label to monitor the ability of a test compound to modulate UBA3, UAE, or UBA6, or other E1 enzyme variant binding to a UBA3, UAE, or UBA6, or other E1 enzyme variant substrate in a complex. For example, compounds (e.g., UBA3, UAE, or UBA6, or other E1 enzyme variant substrates) can be labeled with ¹²⁵I, ¹⁴C, ³⁵S or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a UBA3, UAE, or UBA6, or other E1 enzyme variant substrate) to interact with UBA3, UAE, or UBA6, or other E1 enzyme variant with or without the labeling of any of the interactants can be evaluated. For example, a microphysiometer can be used to detect the interaction of a compound with UBA3, UAE, or UBA6, or other E1 enzyme variant without the labeling of either the compound or the UBA3, UAE, or UBA6, or other E1 enzyme variant. McConnell et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and UBA3, UAE, or UBA6, or other E1 enzyme variant.

In yet another embodiment, a cell-free assay is provided in which a polypeptide, e.g., a UBA3, UAE, or UBA6, or other E1 enzyme variant protein or biologically active portion thereof is contacted with a test compound and the ability of the test compound to bind to the UBA3, UAE, or UBA6, or other E1 enzyme variant protein or biologically active portion thereof is evaluated. In some embodiments, biologically active portions of the UBA3, UAE, or UBA6, or other E1 enzyme variant proteins to be used in assays of the present invention include fragments which participate in interactions with non-UBA3, UAE, or UBA6, or other E1 enzyme variant molecules, e.g., fragments with high surface probability scores. In embodiments wherein a compound is being measured in a cell free assay of a polypeptide comprising a UBA3 variant or biologically active portion thereof comprising a mutation, e.g. an amino acid substitution described herein, the assay can measure the ability of the polypeptide to bind a nucleotide, the ability to hydrolyze a nucleotide, the ability to bind NEDD8, the ability to adenylate NEDD8, the ability to form a covalent thioester bond with NEDD8, the ability to bind an E2 enzyme, the ability to catalyze the transthiolation of NEDD8 to an E2, or the ability to bind NAE 1. In an embodiment, a cell-free assay is a pyrophosphate exchange assay. In another embodiment, a cell-free assay analyzes the binding kinetics of a UBL-test compound, e.g., UBL-E1 enzyme inhibitor (e.g., NEDD8-MLN4924, NEDD8-Compound 1, NEDD8-adenosine sulfamate, ubiquitin-MLN4924, ubiquitin-Compound 1, or ubiquitin-adenosine sulfamate), adduct on and off the variant UBA3, UAE, or UBA6, or other E1 enzyme. Such assays also can test these characteristics on wild type E1 enzyme.

Soluble and/or membrane-bound forms of isolated proteins (e.g., UBA3, UAE, or UBA6, or other E1 enzyme variant proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON® X-100, TRITON® X-114, THESIT®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture, e.g., a composition of the target gene protein, e.g., a UBA3, UAE, or UBA6, or other E1 enzyme variant or a biologically active portion thereof, e.g., comprising a variant residue and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

A heterogeneous time-resolved fluorescence (HTRF) assay can be used to measure UBA3 variant activity. Examples of HTRF assays and how to configure HTRF assays for kinase phosphorylation assays can be performed according to the instructions of CisBio International (Bagnols-sur-Ceze Cedex, France). HTRF is an alternative to radiometric methods comprising two steps, a kinase reaction step, and a detection step. In a reaction step, the enzyme of interest is combined with a substrate, (e.g., ATP), and optionally a compound to be tested. In the detection step, a solution of europium cryptate-labeled antibody and fluorophore-conjugated streptavidin (e.g. SA-XL665, CisBio International, France), or anti-tag-XL665 conjugate for a fusion protein. Fluoresence resonance energy transfer occurs when the two fluorescent tracers are brought in close proximity of one another. The resultant signal (e.g. XL-665-specific signal) is proportional to the amount or level of phosphorylation of the kinase substrate. The ratio of the emissions from the two tracers can be calculated to make a measurement independent of test compound interference.

In another embodiment, determining the ability of the UBA3, UAE, or UBA6, or other E1 enzyme variant protein to bind to a target molecule can be accomplished using real-time Biomolecular Interaction Analysis (BIA) (see, e.g., Sjolander and Urbaniczky (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. In some embodiments, the target gene product can be anchored onto a solid surface, and the test compound, (which is not anchored), can be labeled, either directly or indirectly, with detectable labels discussed herein.

It may be desirable to immobilize either UBA3, UAE, or UBA6, or other E1 enzyme variant, a biologically active portion thereof comprising a mutation, e.g. an amino acid substitution, an anti-UBA3, UAE, or UBA6, or other E1 enzyme variant antibody or its target molecule to facilitate separation of complexed from uncomplexed forms of one or both of the proteins, as well as to accommodate automation of the assay. Binding of a test compound to a UBA3, UAE, or UBA6, or other E1 enzyme variant protein, or interaction of a UBA3, UAE, or UBA6, or other E1 enzyme variant protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/UBA3, UAE, or UBA6, or other E1 enzyme variant fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or UBA3, UAE, or UBA6, or other E1 enzyme variant protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of UBA3, UAE, or UBA6, or other E1 enzyme variant binding or activity determined using standard techniques.

Other techniques for immobilizing either a UBA3, UAE, or UBA6, or other E1 enzyme variant protein or a target molecule on matrices include using conjugation of biotin and streptavidin. Biotinylated UBA3, UAE, or UBA6, or other E1 enzyme variant protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific or selective for the immobilized component (the antibody, in turn, can be directly labeled or indirectly labeled with, e.g., a labeled anti-Ig antibody).

In one embodiment, this assay is performed utilizing antibodies reactive with UBA3, UAE, or UBA6, or other E1 enzyme variant protein or target molecules but which do not interfere with binding of the UBA3, UAE, or UBA6, or other E1 enzyme variant protein to its target molecule. Such antibodies can be derivatized to the wells of the plate, and unbound target or UBA3, UAE, or UBA6, or other E1 enzyme variant protein trapped in the wells by antibody conjugation. Methods for detecting such complexes, in addition to detecting complexes when a UBA3, UAE, or UBA6, or other E1 enzyme variant protein comprises an epitope tag, e.g., a heterologous epitope tag selected from the group consisting of: a His₆ tag, a FLAG tag, a c-myc tag, glutathione-S-transferase (GST) tag, a hemagglutinin (HA) tag, a T7 gene 10 tag, a V5 tag, an HSV tag, and a VSV-G tag, include immunodetection of complexes using antibodies reactive with the UBA3 variant protein or target molecule, as well as enzyme-linked assays which rely on detecting an enzymatic activity associated with the UBA3, UAE, or UBA6, or other E1 enzyme variant protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas and Minton (1993) Trends Biochem Sci 18:284-7); chromatography (gel filtration chromatography, ion-exchange chromatography); electrophoresis (see, e.g., Ausubel et al., eds. (1999) Current Protocols in Molecular Biology, J. Wiley, New York); and immunoprecipitation (see, for example, Ausubel et al., eds. (1999) Current Protocols in Molecular Biology, J. Wiley, New York). Such resins and chromatographic techniques are known to one skilled in the art (see, e.g., Heegaard (1998) J Mol Recognit 11:141-8; Hage and Tweed (1997) J Chromatogr B Biomed Sci Appl. 699:499-525). Further, fluorescence energy transfer can also be conveniently utilized, as described herein, to detect binding without further purification of the complex from solution.

In an embodiment, the assay includes contacting a polypeptide comprising a UBA3, UAE, or UBA6, or other E1 enzyme variant protein or biologically active portion thereof, e.g., comprising a mutation, e.g. an amino acid substitution with a known compound which binds to the polypeptide to form an assay mixture, contacting the assay mixture with a test compound, and determining the ability of the test compound to interact with a UBA3, UAE, or UBA6, or other E1 enzyme variant protein, wherein determining the ability of the test compound to interact with a UBA3, UAE, or UBA6, or other E1 enzyme variant protein includes determining the ability of the test compound to preferentially bind to UBA3, UAE, or UBA6, or other E1 enzyme variant or biologically active portion thereof, or to modulate the activity of a target molecule, as compared to the known compound.

The target gene products of the invention can, in vivo, interact with one or more cellular or extracellular macromolecules, such as proteins, such as NAE1, a UBL or an E2 enzyme. For the purposes of this discussion, such cellular and extracellular macromolecules are referred to herein as “binding partners.” Compounds that disrupt such interactions or disrupt E1 enzyme, e.g., NAE pathway gene activities can be useful in regulating the activity of the target gene product. Such compounds can include, but are not limited to molecules such as antibodies, peptides, and small molecules. Target genes/products for use in this embodiment can be the UBA3, UAE, or UBA6, or other E1 enzyme variant genes herein identified. In an alternative embodiment, the invention provides methods for determining the ability of the test compound to modulate the activity of a UBA3, UAE, or UBA6, or other E1 enzyme variant protein through modulation of the activity of a downstream effector of a E1 enzyme, e.g., a UBA3 variant target molecule, e.g., the activity of an E3 enzyme such as cullin ring ligase. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene, e.g., UBA3, UAE, or UBA6, or other E1 enzyme variant products and a substrate or binding partner, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific or selective for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific or selective for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific or selective for one of the binding components to anchor any complexes formed in solution, and a labeled antibody specific or selective for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the UBA3, UAE, or UBA6, or other E1 enzyme variant proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with UBA3, UAE, or UBA6, or other E1 enzyme variant (“UBA3-binding proteins”, “UAE-binding proteins”, or “UBA6-binding proteins”, or other “E1 enzyme variant-binding proteins” or “UBA3-bp”, “UAE-bp”, “UBA6-bp”, or other “E1 enzyme variant-bp”) and are involved in UBA3, UAE, or UBA6, or other E1 enzyme variant activity. Such UBA3, UAE, or UBA6, or other E1 enzyme variant-bps can be activators or inhibitors of signals by the UBA3, UAE, or UBA6, or other E1 enzyme variant proteins or UBA3, UAE, or UBA6, or other E1 enzyme variant targets as, for example, downstream elements of a UBA3, UAE, or UBA6, or other E1 enzyme variant-mediated signaling pathway.

The two-hybrid system is based on the modular nature of most transcription factors, which consist of separable DNA-binding and activation domains. Briefly, the assay utilizes two different DNA constructs. In one construct, the gene that codes for a UBA3, UAE, or UBA6, or other E1 enzyme variant protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: UBA3, UAE, or UBA6, or other E1 enzyme variant protein can be the fused to the activator domain.) If the “bait” and the “prey” proteins are able to interact, in vivo, forming a UBA3 variant-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the UBA3, UAE, or UBA6, or other E1 enzyme variant protein.

In another embodiment, modulators of UBA3, UAE, or UBA6, or other E1 enzyme variant expression are identified. For example, a cell or cell free mixture is contacted with a candidate compound and the expression of UBA3, UAE, or UBA6, or other E1 enzyme variant mRNA or protein evaluated relative to the level of expression of UBA3, UAE, or UBA6, or other E1 enzyme variant mRNA or protein in the absence of the candidate compound. When expression of UBA3, UAE, or UBA6, or other E1 enzyme variant mRNA or protein is greater in the presence of the candidate compound than in its absence, the candidate compound is identified as a stimulator of UBA3, UAE, or UBA6, or other E1 enzyme variant mRNA or protein expression. Alternatively, when expression of UBA3, UAE, or UBA6, or other E1 enzyme variant mRNA or protein is less (statistically significantly less) in the presence of the candidate compound than in its absence, the candidate compound is identified as an inhibitor of UBA3, UAE, or UBA6, or other E1 enzyme variant mRNA or protein expression. The level of UBA3, UAE, or UBA6, or other E1 enzyme variant mRNA or protein expression can be determined by methods described herein for detecting UBA3, UAE, or UBA6, or other E1 enzyme variant mRNA or protein. The modulation can be direct modulation by inhibition of a UBA3, UAE, or UBA6, or other E1 enzyme variant nucleic acid, e.g., by binding a UBA3, UAE, or UBA6, or other E1 enzyme variant nucleic acid. In such enbodiments, the modulator can be an antisense nucleic acid, an RNAi or an siRNA.

The invention also includes a method of identifying a compound that modulates the drug resistance of a cell by first contacting the cell with a test compound and then measuring and comparing expression of a resistance sequence, e.g., a UBA3, UAE, or UBA6, or other E1 enzyme variant, in the cell exposed to the compound to expression of the resistance sequence in a control cell not exposed to the compound. The compound is identified as modulator of drug resistance when the level of expression of the resistance sequence in the cell exposed to the compound differs from the level of expression of the resistance sequence in a cell not exposed to the compound. In one embodiment of this method, the cell has a drug-resistant phenotype. In another embodiment, the cell is a mammalian cell, e.g. a tumor cell. In another embodiment, the cell is of or from a parasitic organism. This method may also include an optional step of measuring the drug resistance of the cell in the presence of the identified modulator of drug resistance. The compounds modulating resistance that are identified in the foregoing methods are also included within the invention.

The invention features a method for determining whether a test compound modulates the drug resistance of a cell, the method including: a) measuring the level of expression of a resistance sequence (e.g., a resistance protein encoded by an endogenous or heterologous UBA3, UAE, UBA6 or other E1 enzyme gene) in a cell in the presence of a test compound; b) measuring the level of expression of the resistance sequence in the cell in the absence of the test compound; and c) identifying the compound as a modulator of drug resistance of the cell if the level of expression of the resistance sequence in the cell in the presence of the test compound differs from the level of expression of the resistance sequence in the cell in the absence of the test compound.

In another aspect, the invention pertains to a combination of two or more of the assays described herein. For example, a modulating agent can be identified using a cell-based or a cell free assay, and the ability of the agent to modulate the activity of a UBA3, UAE, or UBA6, or other E1 enzyme variant protein can be confirmed in vivo, e.g., in an animal such as an an immunocompromised rodent harboring a xenograft of a tumor comprising or able to generate a UBA3, UAE, or UBA6, or other E1 enzyme variant nucleic acid or protein. In another example, a modulating agent can be identified using a model for pathogenic infection by an organism resistant to an E1 enzyme inhibitor, e.g., an NAE inhibitor.

Related to this aspect, the invention features a method for determining whether a test compound modulates the drug resistance of a cell, the method including: a) incubating a composition comprising resistance protein, e.g., a UBA3, UAE, or UBA6, or other E1 enzyme variant, or a portion thereof which performs at least one UBA3, UAE, or UBA6, or other E1 enzyme variant activity, in the presence of a test compound; b) determining whether the test compound binds to the resistance protein; c) selecting a test compound which binds to the resistance protein; d) administering the test compound selected in step c) to a non-human mammal having drug resistant cells; e) determining whether the test compound alters the drug resistance of the cells in the non-human mammal; and f) identifying the test compound as a modulator of drug resistance of the cell if the compound alters the drug resistance of the cells in step e).

The invention further features a method for determining whether a test cell, e.g., a cell from a biological sample, has a drug-resistant phenotype, the method including: a) measuring the expression of a resistance sequence in the test cell; b) comparing the expression of the resistance sequence measured in step a) to the expression of the resistance sequence in a control cell not having a drug-resistant phenotype; and c) determining that the test cell has a drug resistant phenotype if the expression of the resistance sequence in the test cell is greater than the expression of the resistance sequence in the control cell when the resistance sequence is an up-regulated sequence. In another embodiment of this aspect of the invention, the test cell of step (c) may have a drug resistant phenotype if the expression of the resistance sequence in the test cell is lower than the expression of the resistance sequence in the control cell when the resistance sequence is a down-regulated sequence.

In another aspect the invention features a method of determining whether a test cell, e.g., a cell from a biological sample, has a drug-resistant phenotype, the method including: a) measuring the activity of a resistance sequence in the test cell; b) comparing the activity of the resistance sequence measured in step a) to the activity of the resistance sequence in a control cell not having a drug-resistant phenotype; and c) determining that the test cell has a drug resistant phenotype if the activity of the resistance sequence in the test cell is greater than the activity of the resistance sequence in the control cell when the resistance sequence is an up-regulated sequence. In another embodiment, the test cell of step (c) has a drug resistant phenotype if the activity of the resistance sequence in the test cell is less than the activity of the resistance sequence in the control cell when the resistance sequence is a down-regulated sequence.

This invention further pertains to novel agents identified by the above-described screening assays. Accordingly, it is within the scope of this invention to further use an agent identified as described herein (e.g., a UBA3, UAE, or UBA6, or other E1 enzyme variant modulating agent, an antisense UBA3, UAE, or UBA6, or other E1 enzyme variant nucleic acid molecule, a UBA3, UAE, or UBA6, or other E1 enzyme variant-specific antibody, or a UBA3, UAE, or UBA6, or other E1 enzyme variant-binding partner) in an appropriate animal model to determine the efficacy, toxicity, side effects, or mechanism of action, of treatment with such an agent. Furthermore, novel agents identified by the above-described screening assays can be used for treatments as described herein.

### Detection Assays

Further aspects of the present invention are methods for detecting the presence of an UBA3 variant in a biological sample; and/or for detecting resistance to an E1 enzyme inhibitor, e.g., an NAE inhibitor, e.g., MLN4924 by an UBA3 variant present in a biological sample; and/or for detecting the presence of a UBA3 variant nucleic acid which varies from SEQ ID NO:1 at one or more bases selected from the group consisting of nucleotide 531, 532, 533, 621, 622, 623, 630, 631, 632, 633, 634, 635, 645, 646, 647, 651, 652, 653, 702, 703, 704, 705, 706, 707, 765, 766, 767, 933, 934, 935, 951, 952, 953, 960, 961, 962, 989, 990, and 991 of SEQ ID NO:1 in a biological sample. In other embodiments, an assay can detect the presence of a UAE variant which varies from SEQ ID NO:29 at amino acid 580 or the presence of a UBA6 variant which varies from SEQ ID NO:32 at amino acid 573.

Portions or fragments of the nucleic acid sequences identified herein can be used as polynucleotide reagents. For example, these sequences can be used to: (i) map their respective genes on a chromosome e.g., to locate gene regions associated with genetic disease or to associate UBA3, UAE, or UBA6, or other E1 enzyme variant with a disease; (ii) identify an individual from a minute biological sample (tissue typing); and (iii) aid in forensic identification of a biological sample. These applications are described in the subsections below.

### Tissue Typing

UBA3, UAE, or UBA6, or other E1 enzyme variant sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the UBA3, UAE, or UBA6, or other E1 enzyme variant nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 can provide positive individual identification with a panel of perhaps 10 to 1,000 primers which each yield a noncoding amplified sequence of 100 bases. If predicted coding sequences are used, a more appropriate number of primers for positive individual identification would be 500-2,000.

If a panel of reagents from UBA3, UAE, or UBA6, or other E1 enzyme variant nucleotide sequences described herein is used to generate a unique identification database for an individual, those same reagents can later be used to identify tissue from that individual. Using the unique identification database, positive identification of the individual, living or dead, can be made from extremely small tissue samples.

### Predictive Medicine

The present invention also pertains to the field of predictive medicine in which diagnostic assays, prognostic assays, and monitoring clinical trials are used for prognostic (predictive) purposes to thereby treat an individual.

Methods of assessing expression are useful, especially undesirable expression, of a cellular resistance sequence. Undesirable expression (e.g., increased expression of an up-regulated sequence or decreased expression of a down-regulated sequence) may indicate the presence, persistence or reappearance of drug-resistant (e.g., resistant to an E1 enzyme inhibitor, e.g., an NAE inhibitor, e.g., a 1-methyl sulfamate, e.g., MLN4924) tumor cells in an individual's tissue. More generally, aberrant expression may indicate the occurrence of a deleterious or disease-associated phenotype contributed to by the expression of a resistance sequence.

Generally, the invention provides a method of determining if a subject is at risk for a disorder related to a lesion in or the misexpression of a gene which encodes a UBA3, UAE, or UBA6, or other E1 enzyme variant.

Such disorders include, e.g., a disorder associated with the misexpression of UBA3, UAE, or UBA6, or other E1 enzyme variant gene; a cellular proliferative and/or differentiative disorder, an infection, e.g., a parasitic infection, an immune e.g., inflammatory, disorder or neurodegenerative disorder.

In yet another aspect the invention features a method for determining whether a subject has or is at risk of developing a drug resistant tumor, the method including: a) measuring the expression of UBA3, UAE, or UBA6, or other E1 enzyme variant sequence (e.g., mRNA encoding a UBA3, UAE, or UBA6, or other E1 enzyme variant protein) in a biological sample obtained from the subject (using, e.g., a nucleic acid molecule that hybridizes to the mRNA); b) comparing the expression of the mRNA measured in step a) to the expression of the mRNA in a biological sample obtained from a control subject not having a drug resistant tumor, e.g., wild type UBA3, UAE, or UBA6, or other E1 enzyme expression; and c) determining that the patient has or is at risk of developing a drug resistant tumor if the expression of the mRNA in the biological sample obtained from the patient is higher than the expression of the mRNA in the biological sample obtained from the control subject when the UBA3, UAE, or UBA6, or other E1 enzyme variant mRNA is an up-regulated sequence. In another embodiment, the patient has or is at risk of developing a drug resistant tumor if the expression of the UBA3, UAE, or UBA6, or other E1 enzyme variant mRNA in the biological sample obtained from the patient is lower than the expression of the mRNA in the biological sample obtained from the control subject when the mRNA is a down-regulated sequence.

In still another aspect the invention features a method for determining whether a subject has or is at risk of developing a drug resistant tumor, the method including: a) measuring the activity of a UBA3, UAE, or UBA6, or other E1 enzyme variant sequence in a biological sample obtained from the subject (using, e.g., an agent that binds to the NAEβ variant protein); b) comparing the activity of the UBA3, UAE, or UBA6, or other E1 enzyme variant measured in step a) to the expression of the UBA3, UAE, or UBA6, or other E1 enzyme wild type sequence in a biological sample obtained from a control subject not having a drug resistant tumor; and c) determining that the patient has or is at risk of developing a drug resistant tumor if the activity of the resistance sequence in the biological sample obtained from the patient is higher than the activity of the resistance sequence in the biological sample obtained from the control subject when the resistance sequence is an up-regulated sequence. In another embodiment, the patient has or is at risk of developing a drug resistant tumor if the activity of the resistance sequence in the biological sample obtained from the patient is lower than the activity of the resistance sequence in the biological sample obtained from the control subject when the resistance sequence is a down-regulated sequence.

The method includes one or more of the following:

detecting, in a tissue of the subject, the presence or absence of a mutation which affects the expression of the UBA3, UAE, or UBA6, or other E1 enzyme variant gene, or detecting the presence or absence of a mutation in a region which controls the expression of the gene, e.g., a mutation in the 5′ control region;

detecting, in a tissue of the subject, the presence or absence of a mutation which alters the structure of the UBA3, UAE, or UBA6, or other E1 enzyme gene;

detecting, in a tissue of the subject, the misexpression of the UBA3, UAE, or UBA6, or other E1 enzyme variant gene, at the mRNA level, e.g., detecting a non-wild type level of an UBA3, UAE, or UBA6, or other E1 enzyme variant mRNA;

detecting, in a tissue of the subject, the misexpression of the gene, at the protein level, e.g., detecting a non-wild type level of a UBA3, UAE, or UBA6, or other E1 enzyme variant polypeptide.

An exemplary method for detecting the presence or absence of a resistance sequence in a biological sample involves obtaining a biological sample (such as from a body site implicated in a possible diagnosis of diseased or malignant tissue) from a test subject and contacting the biological sample with a compound or an agent capable of detecting the resistance sequence (e.g., mRNA, genomic DNA, polypeptide) such that the presence of the resistance sequence is detected in the biological sample. The presence and/or relative abundance (e.g., compared to a normal tissue or non-drug resistant tumor of the same type) of the resistance sequence indicates aberrant or undesirable expression of a cellular resistance gene, and correlates with the occurrence in situ of cells having a drug-resistant phenotype.

In some embodiments the method includes: ascertaining the existence of at least one of the following changes among the nucleotides in the UBA3 gene of SEQ ID NO:1: a deletion of one or more nucleotides from the UBA3 gene; an insertion of one or more nucleotides into the gene; a point mutation, e.g., a substitution of one or more nucleotides of the gene; a gross chromosomal rearrangement of the gene, e.g., a translocation or inversion; an alteration in the level of a messenger RNA transcript of a UBA3, UAE, or UBA6, or other E1 enzyme variant gene; aberrant modification of a UBA3, UAE, or UBA6, or other E1 enzyme variant gene, such as of the methylation pattern of the genomic DNA; the presence of a non-wild type splicing pattern of a messenger RNA transcript of a UBA3, UAE, or UBA6, or other E1 enzyme variant gene; a non-wild type level of a UBA3, UAE, or UBA6, or other E1 enzyme variant-protein; allelic loss of a UBA3, UAE, or UBA6, or other E1 enzyme wild type gene, and 10) inappropriate post-translational modification of a UBA3, UAE, or UBA6, or other E1 enzyme variant-protein. In some embodiments, the method includes detecting a UBA3 variant nucleic acid which varies from SEQ ID NO:1 at one or more bases selected from the group consisting of nucleotide 531, 532, 533, 621, 622, 623, 630, 631, 632, 633, 634, 635, 645, 646, 647, 651, 652, 653, 702, 703, 704, 705, 706, 707, 765, 766, 767, 933, 934, 935, 951, 952, 953, 960, 961, 962, 989, 990, and 991 of SEQ ID NO:1.

For example, detecting the genetic lesion can include: (i) providing a probe/primer including an oligonucleotide containing a region of nucleotide sequence which hybridizes to a sense or antisense sequence from SEQ ID NO:1, or naturally occurring mutants thereof or 5′ or 3′ flanking sequences naturally associated with the UBA3 variant gene; (ii) exposing the probe/primer to nucleic acid of the tissue; and detecting, by hybridization, e.g., in situ hybridization, of the probe/primer to the nucleic acid, the presence or absence of the genetic lesion. Exemplary reagents that can detect a UBA3 variant nucleic acid can be found in the Examples.

In some embodiments detecting the misexpression includes ascertaining the existence of at least one of: an alteration in the level of a messenger RNA transcript of the UBA3 variant gene; the presence of a non-wild type splicing pattern of a messenger RNA transcript of the gene; or a non-wild type level of UBA3 variant.

Methods of the invention can be used prenatally or to determine if a subject's offspring will be at risk for a disorder.

In some embodiments the method includes determining the structure of a UBA3 variant gene, an abnormal structure being indicative of risk for the disorder.

In some embodiments the method includes contacting a sample from the subject with an antibody to the UBA3, UAE, or UBA6, or other E1 enzyme variant protein. In other embodiments, the method includes contacting a sample from the subject with a nucleic acid which hybridizes specifically with the UBA3, UAE, or UBA6, or other E1 enzyme gene. These and other embodiments are discussed below.

### Diagnostic and Prognostic Assays

In the context of cancer treatment, the expression level of a UBA3, UAE, or UBA6, or other E1 enzyme variant sequence may be used to: 1) determine if a cancer, particularly a drug resistant cancer, can be treated by an agent or combination of agents; 2) determine if a cancer is responding to treatment with an agent or combination of agents; 3) select an appropriate agent or combination of agents for treating a cancer; 4) monitor the effectiveness of an ongoing treatment; and 5) identify new cancer treatments (either single agent or combination of agents). In particular, a UBA3, UAE, or UBA6, or other E1 enzyme variant sequence may be used as a marker (surrogate and/or direct) to determine appropriate therapy, to monitor clinical therapy and human trials of a drug being tested for efficacy, and in developing new agents and therapeutic combinations.

Accordingly, the present invention provides methods for determining whether an agent, e.g., a chemotherapeutic agent such as E1 enzyme inhibitor, e.g., an NAE inhibitor, e.g., an 1-methyl sulfamate, e.g., MLN4924, will be effective in reducing the growth rate of cancer cells comprising the steps of: a) obtaining a sample of cancer cells; b) determining the level of expression in the cancer cells of a resistance sequence; and c) identifying that an agent will be effective when the resistance sequence is expressed at a level not associated with drug resistance (e.g., an up-regulated resistance sequence is not expressed or is expressed at relatively low level compared to a non-drug resistant cancer cell; a down-regulated resistance sequence may be expressed at a relatively high level). Alternatively, in step (c), an agent can be identified as being relatively ineffective for treating the cancer when a resistance sequence is expressed at a level associated with resistance to that agent (e.g., an up-regulated resistance sequence at a relatively high level compared to a non-drug resistant cell or a down-regulated resistance sequence can be expressed at a relatively low level).

As used herein, an agent is said to reduce the rate of growth of cancer cells when the agent can reduce at least 50%, at least 75%, or at least 95% of the growth of the cancer cells. Such inhibition can further include a reduction in survivability and an increase in the rate of death of the cancer cells. The amount of agent used for this determination will vary based on the agent selected. Typically, the amount will be a predefined therapeutic amount.

The presence, level, or absence of UBA3, UAE, or UBA6, or other E1 enzyme variant protein or nucleic acid in a biological sample can be evaluated by obtaining a biological sample from a test subject and contacting the biological sample with a compound or an agent capable of detecting UBA3, UAE, or UBA6, or other E1 enzyme variant protein or nucleic acid (e.g., mRNA, genomic DNA) that encodes UBA3, UAE, or UBA6, or other E1 enzyme variant protein such that the presence of UBA3, UAE, or UBA6, or other E1 enzyme variant protein or nucleic acid is detected in the biological sample. The term “biological sample” includes tissues, cells and biological fluids isolated from a subject, as well as tissues, cells and fluids present within a subject. In one embodiment, the biological sample contains protein molecules from the test subject. Alternatively, the biological sample can contain mRNA molecules from the test subject or genomic DNA molecules from the test subject. A biological sample can be a peripheral blood leukocyte sample isolated by conventional means from a subject. A biological sample can be serum. A biological sample can be a tumor sample. A biological sample can comprise a lymphocyte, an infected cell or a parasite obtained from the subject. The level of expression of the UBA3, UAE, or UBA6, or other E1 enzyme variant gene can be measured in a number of ways, including, but not limited to: measuring the mRNA encoded by the UBA3, UAE, or UBA6, or other E1 enzyme variant genes; measuring the amount of protein encoded by the UBA3, UAE, or UBA6, or other E1 enzyme variant genes; or measuring the activity of the protein encoded by the UBA3, UAE, or UBA6, or other E1 enzyme variant genes.

In another embodiment, the methods further involve obtaining a control biological sample from a control subject or from a non-diseased site from the test subject, contacting the control sample with a compound or agent capable of detecting a resistance protein, mRNA, or genomic DNA, such that the presence of the resistance protein, mRNA or genomic DNA is detected in the biological sample, and comparing the presence of the resistance protein, mRNA or genomic DNA in the control sample with the presence of the resistance protein, mRNA or genomic DNA in the test sample. In another embodiment, the methods further contacting a control sample with a compound or agent capable of detecting UBA3, UAE, or UBA6, or other E1 enzyme variant mRNA, or genomic DNA, and comparing the presence of UBA3, UAE, or UBA6, or other E1 enzyme variant mRNA or genomic DNA in the control sample with the presence of UBA3, UAE, or UBA6, or other E1 enzyme variant mRNA or genomic DNA in the test sample.

The level of mRNA corresponding to the UBA3, UAE, or UBA6, or other E1 enzyme variant gene in a cell can be determined both by in situ and by in vitro formats.

The isolated mRNA can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length UBA3 variant nucleic acid, such as the nucleic acid of SEQ ID NO:1, or a portion thereof, such as an oligonucleotide of at least 7, 15, 30, 50, 100, 250 or 500 nucleotides in length and sufficient to specifically hybridize under stringent conditions to UBA3 variant mRNA or genomic DNA. Other suitable probes for use in the diagnostic assays are described herein, including the Examples.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by the UBA3, UAE, or UBA6, or other E1 enzyme variant genes.

The level of mRNA in a sample that is encoded by one of UBA3, UAE, or UBA6, or other E1 enzyme variant can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the UBA3, UAE, or UBA6, or other E1 enzyme variant gene being analyzed.

With “physical detection methods” is meant in the present context methods of nucleotide sequence polymorphism detection that require one or more physical processes for detection although not excluding the enzymatic process of prior PCR amplification of the target DNA sequence comprising one or more nucleotide sequence polymorphisms. Examples of physical processes include electrophoresis, chromatography, spectrometry, optical signal sensing and spectroscopy.

A variety of methods can be used to determine the level of protein encoded by UBA3, UAE, or UBA6, or other E1 enzyme variant. In general, these methods include contacting an agent that selectively binds to the protein, such as an antibody with a sample, to evaluate the level of protein in the sample. In an embodiment, the antibody bears a detectable label. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect UBA3, UAE, or UBA6, or other E1 enzyme variant protein in a biological sample in vitro as well as in vivo. In vitro techniques for detection of UBA3, UAE, or UBA6, or other E1 enzyme variant protein include enzyme linked immunosorbent assays (ELISAs), immunoprecipitations, immunofluorescence, enzyme immunoassay (EIA), radioimmunoassay (RIA), and Western blot analysis. In vivo techniques for detection of UBA3, UAE, or UBA6, or other E1 enzyme variant protein include introducing into a subject a labeled anti-UBA3, UAE, or UBA6, or other E1 enzyme variant antibody. For example, the antibody can be labeled with a radioactive marker whose presence and location in a subject can be detected by standard imaging techniques. An antibody that can be used for these methods can selectively bind an amino acid mutation, e.g., substitution in a UBA3, UAE, or UBA6, or other E1 enzyme. For example, the antibody would recognize a UBA3, UAE, or UBA6, or other E1 enzyme comprising a mutated, e.g., substituted residue and not a UBA3, UAE, or UBA6, or other E1 enzyme polypeptide comprising the wild type residue. In some embodiments an antibody for use in these methods can bind a UBA3 variant protein comprising a mutation from wild type UBA3, e.g. selected from the group consisting of an amino acid residue which does not equal the residue at 171, 201, 204, 205, 209, 211, 228, 229, 249, 305, 311, 314 and 324 of SEQ ID NO:2.

In another embodiment, the methods further include contacting the control sample with a compound or agent capable of detecting UBA3, UAE, or UBA6, or other E1 enzyme variant protein, and comparing the presence of UBA3, UAE, or UBA6, or other E1 enzyme variant protein in the control sample with the presence of UBA3, UAE, or UBA6, or other E1 enzyme variant protein in the test sample.

The invention also includes kits for detecting the presence of UBA3, UAE, or UBA6, or other E1 enzyme variant in a biological sample. Such kits can be used to determine if a subject is suffering from or is at increased risk of developing a disorder associated with aberrant expression of a resistance (e.g., the presence of a drug resistant cancer). For example, the kit can include a compound or agent capable of detecting UBA3, UAE, or UBA6, or other E1 enzyme variant protein or mRNA in a biological sample; and a means for determining the amount of resistance sequence is above or below a normal level, e.g., a standard. The compound or agent can be packaged in a suitable container. The kit can further comprise instructions for using the kit to detect UBA3, UAE, or UBA6, or other E1 enzyme variant protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

In yet another alternative, the UBA3, UAE, or UBA6, or other E1 enzyme variant can be detected phenotypically, i.e. said UBA3, UAE, or UBA6, or other E1 enzyme variant may display a unique pattern of E1 inhibitor sensitivity not shared with UBA3, UAE, or UBA6, or other E1 enzyme variant comprising wild type residues. Phenotypic detection of the UBA3, UAE, or UBA6, or other E1 enzyme variant includes e.g. the steps of determining the sensitivity of an activity of an UBA3, UAE, or UBA6, or other E1 enzyme variant from a wild type UBA3, UAE, or UBA6, or other E1 enzyme present in a biological sample to a panel of E1 enzyme inhibitors.

For oligonucleotide-based kits, the kit can include: (1) an oligonucleotide, e.g., a detectably labeled oligonucleotide, which hybridizes to a nucleic acid sequence encoding a polypeptide corresponding to a UBA3, UAE, or UBA6, or other E1 enzyme variant or (2) a pair of primers useful for amplifying a nucleic acid molecule corresponding to a UBA3, UAE, or UBA6, or other E1 enzyme variant. The kit can also include a buffering agent, a preservative, or a protein stabilizing agent. The kit can also include components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

Furthermore embodied are said oligonucleotide-based kits wherein said oligonucleotide or oligonucleotides are attached or immobilized to a solid support. Other embodiments thereto include said kits comprising: a. a means for obtaining a target UBA3, UAE, or UBA6, or other E1 enzyme variant polynucleic acid present in said biological sample and/or obtaining the nucleotide sequence thereof; b. when appropriate, at least one oligonucleotide pair suitable for amplification of a target UBA3, UAE, or UBA6, or other E1 enzyme variant polynucleic acid according to the invention; c. when appropriate, a means for denaturing nucleic acids; d. when appropriate, at least one oligonucleotide according to the invention; e. when appropriate, an enzyme capable of modifying a double stranded or single stranded nucleic acid molecule; f. when appropriate, a hybridization buffer, or components necessary for producing said buffer; g. when appropriate, a wash solution, or components necessary for producing said solution; h. when appropriate, a means for detecting partially or completely denatured polynucleic acids and/or a means for detecting hybrids formed in the preceding hybridization and/or a means for detecting enzymatic modifications of nucleic acids; i. when appropriate, a means for attaching an oligonucleotide to a known location on a solid support; j. a means for inferring from the partially or completely denatured polynucleic acids and/or from the hybrids and/or from the enzymatic modifications, all detected in (h), and/or from the nucleotide sequence obtained in (a), the presence of said UBA3, UAE, or UBA6, or other E1 enzyme variant in said biological sample.

In general, inferring, from a nucleic acid sequence, the presence of a mutation, e.g. a substitution, at nucleotide Y (Y is number as indicated) in a variant sequence encoding a mutated, e.g., a substituted, amino acid X (X is amino acid as indicated) is meant any technique or method to (i) localize in said nucleic acid sequence a codon comprising a mutated, e.g., substituted nucleotide Y, (ii) to translate said codon comprising the mutated, e.g., substituted nucleotide Y into the amino acid encoded by the codon, and (iii) to conclude from (ii) if the amino acid encoded by said codon comprising mutated, e.g., substituted nucleotide Y is the same as or is different as the codon encoding said amino acid X. Said techniques can include methods wherein (i) to (iii) all are performed manually and/or computationally. Said techniques may include aligning and/or comparing an obtained nucleic acid sequence with a set of nucleic acid sequences contained within a database. Said techniques may furthermore include the result of (i) to (iii) being presented in the form of a report wherein said report can be in paper form, in electronic form or on a computer readable carrier or medium. Said techniques may furthermore include the searching of (nucleic acid and/or amino acid) sequence databases and/or the creation of (nucleic acid and/or amino acid) sequence alignments, the results of which may or may not be included in said report.

The diagnostic methods described herein can identify subjects having, or at risk of developing, a disease or disorder associated with misexpressed or aberrant or unwanted UBA3, UAE, or UBA6, or other E1 enzyme variant expression or activity. As used herein, the term “unwanted” includes an unwanted phenomenon involved in a biological response such as pain or deregulated cell proliferation.

One embodiment comprises identification of a disease or disorder associated with aberrant or unwanted UBA3, UAE, or UBA6, or other E1 enzyme variant expression or activity, e.g., resistance to an E1 enzyme inhibitor. A test sample is obtained from a subject and UBA3, UAE, or UBA6, or other E1 enzyme variant protein or nucleic acid (e.g., mRNA or genomic DNA) is evaluated, wherein the level, e.g., the presence or absence, of UBA3, UAE, or UBA6, or other E1 enzyme variant protein or nucleic acid is diagnostic for a subject having or at risk of developing a disease or disorder associated with aberrant or unwanted UBA3, UAE, or UBA6, or other E1 enzyme variant expression or activity. As used herein, a “test sample” refers to a biological sample obtained from a subject of interest, including a biological fluid (e.g., serum), cell sample, e.g., a sample comprising tumor cells, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted UBA3, UAE, or UBA6, or other E1 enzyme variant expression or activity. For example, such methods can be used to determine whether a subject can be effectively treated with an E1 enzyme inhibitor.

An alteration can be detected without a probe/primer in a polymerase chain reaction, such as anchor PCR or RACE PCR, or, alternatively, in a ligation chain reaction (LCR), the latter of which can be particularly useful for detecting point mutations in the UBA3, UAE, or UBA6, or other E1 enzyme variant-gene. This method can include the steps of collecting a sample of cells from a subject, isolating nucleic acid (e.g., genomic, mRNA or both) from the sample, contacting the nucleic acid sample with one or more primers which specifically hybridize to a UBA3, UAE, or UBA6, or other E1 enzyme variant gene under conditions such that hybridization and amplification of the UBA3, UAE, or UBA6, or other E1 enzyme variant gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a UBA3, UAE, or UBA6, or other E1 enzyme variant gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in UBA3, UAE, or UBA6, or other E1 enzyme variant can be identified by hybridizing a sample and control nucleic acids, e.g., DNA or RNA, two dimensional arrays, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin et al. (1996) Human Mutation 7: 244-255; Kozal et al. (1996) Nature Medicine 2: 753-759). For example, genetic mutations in UBA3, UAE, or UBA6, or other E1 enzyme variant can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the UBA3, UAE, or UBA6, or other E1 enzyme variant gene and detect mutations by comparing the sequence of the sample UBA3, UAE, or UBA6, or other E1 enzyme variant with the corresponding wild-type (control) sequence. Automated sequencing procedures can be utilized when performing the diagnostic assays (Naeve et al. (1995) Biotechniques 19:448-53), including sequencing by mass spectrometry. Descriptions of some sequencing methods can be found in the Examples.

Other methods for detecting mutations in the UBA3, UAE, or UBA6, or other E1 enzyme variant gene include methods in which protection from cleavage agents is used to detect mismatched bases in RNA/RNA or RNA/DNA heteroduplexes (Myers et al. (1985) Science 230:1242; Cotton et al. (1988) Proc. Natl Acad Sci USA 85:4397; Saleeba et al. (1992) Methods Enzymol. 217:286-295)

In still another embodiment, the mismatch cleavage reaction employs one or more proteins that recognize mismatched base pairs in double-stranded DNA (so called “DNA mismatch repair” enzymes) in defined systems for detecting and mapping point mutations in UBA3, UAE, or UBA6, or other E1 enzyme variant cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in UBA3, UAE, or UBA6, or other E1 enzyme variant genes. For example, single strand conformation polymorphism (SSCP) can be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control UBA3, UAE, or UBA6, or other E1 enzyme variant nucleic acids will be denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In an embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl Acad. Sci USA 86:6230).

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification can also be performed using Taq ligase for amplification (Barany (1991) Proc. Natl. Acad. Sci USA 88:189-93). In such cases, ligation will occur only if there is a perfect match at the 3′ end of the 5′ sequence making it possible to detect the presence of a known mutation at a specific site by looking for the presence or absence of amplification.

The methods described herein can be performed, for example, by utilizing pre-packaged diagnostic kits comprising at least one probe nucleic acid or antibody reagent described herein, which can be conveniently used, e.g., in clinical settings to diagnose patients exhibiting symptoms or family history of a disease or illness involving a UBA3, UAE, or UBA6, or other E1 enzyme variant gene.

The UBA3, UAE, or UBA6, or other E1 enzyme variant molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject, e.g., whether an E1 enzyme inhibitor is producing an inhibitory effect. For example, a pharmacodynamic marker can be indicative of the concentration of the drug in a biological tissue, in that the marker is either expressed or transcribed or not expressed or transcribed in that tissue in relationship to the level of the drug. In this fashion, the distribution or uptake of the drug can be monitored by the pharmacodynamic marker. Similarly, the presence or quantity of the pharmacodynamic marker can be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug can be sufficient to activate multiple rounds of marker (e.g., a UBA3, UAE, or UBA6, or other E1 enzyme variant marker) transcription or expression, the amplified marker can be in a quantity which is more readily detectable than the drug itself. Also, the marker can be more easily detected due to the nature of the marker itself; for example, using the methods described herein, anti-UBA3, UAE, or UBA6, or other E1 enzyme variant antibodies can be employed in an immune-based detection system for a UBA3, UAE, or UBA6, or other E1 enzyme variant protein marker, or UBA3, UAE, or UBA6, or other E1 enzyme variant-specific radiolabeled probes can be used to detect a UBA3, UAE, or UBA6, or other E1 enzyme variant mRNA marker. Furthermore, the use of a pharmacodynamic marker can offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am. J. Health-Syst. Pharm. 56 Suppl. 3: S16-S20.

The UBA3, UAE, or UBA6, or other E1 enzyme variant molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, can be selected. For example, based on the presence or quantity of RNA, or protein (e.g., UBA3, UAE, or UBA6, or other E1 enzyme variant protein or RNA) for specific tumor markers in a subject, a drug or course of treatment can be selected that is optimized for the treatment of the specific tumor likely to be present in the subject. In some embodiments, the presence of a UBA3, UAE, or UBA6, or other E1 enzyme variant, e.g., comprising a mutation, e.g., a substitution described herein, in a sample from a subject, would suggest that the subject is resistant or at risk of developing resistant to treatment by an E1 inhibitor, such as MLN4924. Similarly, the presence or absence of a specific sequence mutation in UBA3, UAE, or UBA6, or other E1 enzyme DNA can correlate with an E1 enzyme inhibitor resistance. The use of pharmacogenomic markers therefore permits the application of the most appropriate treatment for each subject without having to administer the therapy.

The invention also features a method for monitoring the effect of an anti-tumor treatment on a patient, the method including: a) measuring the expression of a UBA3, UAE, or UBA6, or other E1 enzyme variant resistance sequence in a tumor sample obtained from the patient (using, e.g., a nucleic acid molecule that hybridizes to the resistance mRNA); b) comparing the expression of the UBA3, UAE, or UBA6, or other E1 enzyme variant resistance sequence measured in step a) to the expression of the UBA3, UAE, or UBA6, or other E1 enzyme variant resistance sequence in a control sample of cells; and c) determining that the anti-tumor treatment should be discontinued or modified if the expression of the UBA3, UAE, or UBA6, or other E1 enzyme variant resistance sequence in the tumor sample is higher than the expression of UBA3, UAE, or UBA6, or other E1 enzyme variant resistance sequence in the control sample of cells when the resistance sequence is an up-regulated sequence. In another embodiment, the anti-tumor treatment should be discontinued or modified as in step (c) if the expression of the UBA3, UAE, or UBA6, or other E1 enzyme variant resistance sequence in the tumor sample is lower than the expression of the resistance sequence in the control sample of cells when the UBA3, UAE, or UBA6, or other E1 enzyme variant resistance sequence is a down-regulated sequence.

The invention also features a method for monitoring the effect of an anti-tumor treatment on a patient, the method including: a) measuring the activity of a UBA3, UAE, or UBA6, or other E1 enzyme variant resistance sequence in a tumor sample obtained from the patient (using, e.g., an agent that binds to the UBA3, UAE, or UBA6, or other E1 enzyme variant resistance protein); b) comparing the activity of the UBA3, UAE, or UBA6, or other E1 enzyme variant resistance sequence measured in step a) to the activity of the UBA3, UAE, or UBA6, or other E1 enzyme variant resistance sequence in a control sample of cells; and c) determining that the anti-tumor treatment should be discontinued or modified if the activity of the UBA3, UAE, or UBA6, or other E1 enzyme variant resistance sequence in the tumor sample is higher than the activity of the resistance sequence in the control sample of cells when the UBA3, UAE, or UBA6, or other E1 enzyme variant resistance sequence is an up-regulated sequence. In another embodiment, it is determined that the anti-tumor treatment should be discontinued or modified as in step (c) if the activity of the UBA3, UAE, or UBA6, or other E1 enzyme variant resistance sequence in the tumor sample is lower than the activity of the UBA3, UAE, or UBA6, or other E1 enzyme variant resistance sequence in the control sample of cells when the UBA3, UAE, or UBA6, or other E1 enzyme variant resistance sequence is a down-regulated sequence.

### Pharmacogenomics

The UBA3, UAE, or UBA6, or other E1 enzyme variant molecules of the present invention, as well as agents, or modulators which have a stimulatory or inhibitory effect on UBA3, UAE, or UBA6, or other E1 enzyme variant activity (e.g., UBA3, UAE, or UBA6, or other E1 enzyme variant gene expression) as identified by a screening assay described herein can be administered to individuals to treat (prophylactically or therapeutically) E1 enzyme inhibitor resistance associated with amino acid mutations, e.g., amino acid substitutions such as those described herein. In conjunction with such treatment, pharmacogenomics (i.e., the study of the relationship between an individual's genotype and that individual's response to a foreign compound or drug) can be considered. Thus, another aspect of the invention provides methods for tailoring an individual's prophylactic or therapeutic treatment with either the UBA3, UAE, or UBA6, or other E1 enzyme variant molecules of the present invention or UBA3, UAE, or UBA6, or other E1 enzyme variant modulators according to that individual's drug response genotype. For example, differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a E1 inhibitor, such as an inhibitor which overcomes resistance, as well as tailoring the dosage and/or therapeutic regimen of treatment with an E1 inhibitor.

Monitoring the influence of agents (e.g., drugs, compounds) on the expression or activity of a UBA3, UAE, or UBA6, or other E1 enzyme variant nucleic acid or protein (e.g., the ability to modulate the drug-resistant phenotype of a cell) can be applied not only in basic drug screening, but also in clinical trials. For example, the effectiveness of an agent determined by a screening assay to decrease UBA3, UAE, or UBA6, or other E1 enzyme variant gene expression, protein levels, or downregulate UBA3, UAE, or UBA6, or other E1 enzyme variant activity, can be monitored in clinical trials of subjects exhibiting increased UBA3, UAE, or UBA6, or other E1 enzyme variant gene expression, protein levels, or upregulated UBA3, UAE, or UBA6, or other E1 enzyme variant activity. In such assays or clinical trials, the expression or activity of a UBA3, UAE, or UBA6, or other E1 enzyme variant gene, and in some cases, other genes that have been implicated in, for example, a UBA3, UAE, or UBA6, or other E1 enzyme variant-associated disorder can be used as a “read out” or markers of the phenotype of a particular cell.

For example, and not by way of limitation, genes, including a resistance gene, that are modulated in cells by treatment with an E1 enzyme inhibitor (e.g., compound, drug or small molecule) which modulates activity of a resistance sequence, i.e., overcomes resistance (e.g., identified in a screening assay or otherwise described herein) can be identified. Thus, to study the effect of agents on cellular proliferation disorders, for example, in a clinical trial, cells can be isolated and RNA prepared and analyzed for the levels of expression of a resistance sequence and other sequences (nucleic acid or polypeptide) implicated in the disorder. The levels of expression (i.e., a gene expression pattern) can be quantified by Northern blot analysis or RT-PCR, as described herein, or alternatively by measuring the amount of protein produced, by one of the methods as described herein, or by measuring the levels of activity of a resistance sequence or other sequence including a genes encoding such sequences. In this way, the gene expression pattern can serve as a marker, indicative of the physiological response of the cells to the agent. Accordingly, this response state may be determined before, and at various points during, treatment of the individual with the agent.

In an embodiment, the present invention provides a method for monitoring the effectiveness of treatment of a subject with an agent (e.g., an agonist, antagonist, peptidomimetic, protein, peptide, nucleic acid, small molecule, or other drug candidate, e.g., an E1 enzyme inhibitor, e.g., an NAE inhibitor, identified by the screening assays described herein) comprising the steps of (i) obtaining a pre-administration sample from a subject prior to administration of the agent; (ii) detecting the level of expression of a resistance sequence (e.g., protein, mRNA, or genomic DNA) in the preadministration sample; (iii) obtaining one or more post-administration samples from the subject; (iv) detecting the level of expression or activity of the resistance sequence in the post-administration samples; (v) comparing the level of expression or activity of the resistance sequence in the pre-administration sample with the resistance sequence in the post administration sample or samples; and (vi) altering the administration of the agent to the subject accordingly. For example, increased administration of the agent may be desirable to decrease the expression or activity of an up-regulated resistance sequence beyond what was detected in the post administration sample, i.e., to increase the effectiveness of the agent.

In some embodiments, cancer cells include acute myelogenous leukemia cells or melanoma cells. Cancer cells include, but are not limited to, carcinomas, such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, seminoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; leukemias and lymphomas such as granulocytic leukemia, monocytic leukemia, lymphocytic leukemia, malignant lymphoma, plasmocytoma, reticulum cell sarcoma, or Hodgkins disease; and tumors of the nervous system including glioma, meningioma, medulloblastoma, schwannoma or epidymoma.

The source of the cancer cells used in the methods of the invention will be based on how the method of the present invention is being used. For example, if the method is being used to determine whether a patient's cancer can be treated with an agent, or a combination of agents, then the source of cancer cells can be cancer cells obtained from a cancer biopsy from the patient. Alternatively, a cancer cell line of similar type to that being treated can be assayed. For example if breast cancer is being treated, then a breast cancer cell line can be used. If the method is being used to monitor the effectiveness of a therapeutic protocol, then a tissue sample, e.g., a sample comprising tumor cells, lymphocytes, neural tissue or pathogen- or parasite-infected cells, can be obtained from the patient being treated. If the method is being used to identify new therapeutic agents or combinations, then any cancer cells, e.g., cells of a cancer cell line, can be used.

A skilled artisan can readily select and obtain the appropriate cancer cells that are used in the present method. For example, the HCT-116, Calu-6 and NCI-H460 cancer cell lines, used in the examples, can be made used. For cancer cells obtained from a patient, standard biopsy methods, such as a needle biopsy, can be employed.

In the methods of the present invention, the level or amount of expression of a resistance sequence is determined. As used herein, the level or amount of expression refers to the absolute level of expression of an resistance mRNA or the absolute level of expression of a resistance protein (i.e., whether or not expression is occurring in the cancer cells).

As an alternative to making determinations based on the absolute expression level of selected genes, determinations may be based on the normalized expression levels. Expression levels are normalized by correcting the absolute expression level of a sensitivity or resistance sequence by comparing its expression to the expression of a sequence that is not a sensitivity or resistance sequence, e.g., a sequence encoded by housekeeping gene that is constitutively expressed. Suitable genes for normalization include housekeeping genes such as the actin gene. This normalization allows one to compare the expression level in one sample, e.g., a patient sample, to another sample, e.g., a non-cancer sample, or between samples from different sources. Alternatively, the expression level can be provided as a relative expression level. To determine a relative expression level of a gene, the level of expression of the gene is determined for 10 or more samples, or 50 or more samples, prior to the determination of the expression level for the sample in question. The mean expression level of the gene assayed in the larger number of samples is determined and this is used as a baseline expression level for the gene in question. The expression level of the gene determined for the test sample (absolute level of expression) is then divided by the mean expression value obtained for that gene. This provides a relative expression level and aids in identifying extreme cases of sensitivity or resistance. In embodiments measuring expression in tumor cells, the normalization samples used will be from similar tumors or from non-cancerous cells of the same tissue origin as the tumor in question. The choice of the cell source is dependent on the use of the relative expression level data. For example, using tumors of similar types for obtaining a mean expression score allows for the identification of extreme cases of sensitivity or resistance. Using expression found in normal tissues as a mean expression score aids in validating whether the gene assayed is tumor specific (versus normal cells).

Also within the invention is a method for increasing drug resistance in a cell by altering the level of expression of a resistance sequence by administering a compound that alters the expression of the resistance sequence. For example, drug resistance may be increased by increasing the expression of an up-regulated sequence in the cell. Decreasing expression of a down-regulated sequence can increase drug resistance. Such methods are useful for the protection of non-neoplastic cells during chemotherapy.

The compounds useful for this invention are inhibitors of E1 enzyme activity. In particular, the compounds are designed to be inhibitors of NAE, UAE, and/or SAE. Inhibitors are meant to include compounds which reduce the promoting effects of E1 enzymes in ubl conjugation to target proteins (e.g., reduction of ubiquitination, neddylation, sumoylation), reduce intracellular signaling mediated by ubl conjugation, and/or reduce proteolysis mediated by ubl conjugation (e.g., inhibition of cullin-dependent ubiquitination and proteolysis (e.g., the ubiquitin-proteasome pathway)). Thus, the compounds of this invention may be assayed for their ability to inhibit the E1 enzyme in vitro or in vivo, or in cells or animal models according to methods provided in further detail herein, or methods known in the art. The compounds may be assessed for their ability to bind or mediate E1 enzyme activity directly, e.g., in a pyrophosphate exchange assay. Alternatively, the activity of compounds may be assessed through indirect cellular assays, or assays of downstream effects of E1 activation to assess inhibition of downstream effects of E1 inhibition (e.g., inhibition of cullin-dependent ubiquitination and proteolysis). For example, activity may be assessed by detection of ubl-conjugated substrates (e.g., ubl-conjugated E2s, neddylated cullins, ubiquitinated substrates, sumoylated substrates); detection of downstream protein substrate stabilization (e.g., stabilization of p27, stabilization of IκB); detection of inhibition of UPP activity; detection of downstream effects of protein E1 inhibition and substrate stabilization (e.g., reporter assays, e.g., NFκB reporter assays, p27 reporter assays). Assays for assessing activities are described below in the Experimental section and/or are known in the art.

### Pharmaceutical Compositions

The nucleic acid and polypeptides, fragments thereof, as well as anti-UBA3, UAE, or UBA6, or other E1 enzyme variant antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

The term “pharmaceutically acceptable carrier” is used herein to refer to a material that is compatible with a recipient subject, such as a mammal, e.g., a human, and is suitable for delivering an active agent to the target site without terminating the activity of the agent.

A pharmaceutical composition is formulated to be compatible with its intended route of administration. Examples of routes of administration include parenteral, e.g., intravenous, intradermal, subcutaneous, oral, transdermal (e.g. topical), transmucosal (e.g., inhalation of aerosol or absorption of eye drop), rectal administration or via an implanted reservoir. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride, dextrose or polyalcohols such as manitol, sorbitol. pH can be adjusted with acids or bases, such as hydrochloric acid or sodium hydroxide. The parenteral preparation can be enclosed in ampoules, disposable syringes or multiple dose vials made of glass or plastic.

The pharmaceutical compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain stabilizers, pH modifiers, surfactants, lyoprotectants, solubilizing agents, bioavailability modifiers and combinations of these.

According to one embodiment, the compositions of this invention are formulated for pharmaceutical administration to a mammal, such as a human being or domesticated animal. The term “parenteral” as used herein includes subcutaneous, intravenous, intraperitoneal, intramuscular, intra-articular, intra-synovial, intrasternal, intrathecal, intrahepatic, intralesional and intracranial injection or infusion techniques. The formulations of the invention may be designed to be short-acting, fast-releasing, or long-acting. Still further, compounds can be administered in a local rather than systemic means, such as administration (e.g., by injection) at a tumor site.

Pharmaceutical formulations may be prepared as liquid suspensions or solutions using a liquid, such as, but not limited to, an oil, water, an alcohol, and combinations of these. Solubilizing agents such as cyclodextrins may be included. Pharmaceutically suitable surfactants, suspending agents, or emulsifying agents, may be added for oral or parenteral administration. Suspensions may include oils, such as but not limited to, peanut oil, sesame oil, cottonseed oil, corn oil and olive oil or polyoxyethylated versions thereof. Suspension preparation may also contain a long-chain alcohol diluent or dispersant, such as carboxymethyl cellulose or similar dispersing agents or esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, and polyol (for example, glycerol, propylene glycol, and propylene glycol) and suitable mixtures thereof. Ethers, such as but not limited to, poly(ethyleneglycol), petroleum hydrocarbons such as mineral oil and petrolatum; and water may also be used in suspension formulations. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In injectable formats, the composition must be sterile and should be fluid to the extent that easy syringability exists. It should be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation can be vacuum drying and freeze-drying which yields a powder of the active ingredient plus any additional desired ingredient from a previously sterile-filtered solution thereof.

The pharmaceutical compositions of this invention may be orally administered in any orally acceptable dosage form including, but not limited to, capsules, e.g., gelatin capsules, tablets, troches, aqueous suspensions or solutions. When aqueous suspensions are required for oral use, the active ingredient is combined with emulsifying and suspending agents. If desired, certain sweetening, flavoring or coloring agents may also be added. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Lubricating agents, such as magnesium stearate, are also typically added. Coatings may be used for a variety of purposes; e.g., to mask taste, to affect the site of dissolution or absorption, or to prolong drug action. Coatings may be applied to a tablet or to granulated particles for use in a capsule.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Alternatively, the pharmaceutical compositions of this invention may be administered in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The pharmaceutical compositions of this invention are particularly useful in therapeutic applications relating to disorders as described herein (e.g., proliferation disorders, e.g., cancers, inflammatory, neurodegenerative disorders or parasitic infections). The composition can be formulated for administration to a patient having or at risk of developing or experiencing a recurrence of the relevant disorder being treated through resistance to an E1 enzyme inhibitor. The term “patient”, as used herein, means an animal, a mammal, or a human. In some embodiments, pharmaceutical compositions of the invention are those formulated for oral, intravenous, or subcutaneous administration. However, any of the above dosage forms containing a therapeutically effective amount of a compound of the invention are well within the bounds of routine experimentation and therefore, well within the scope of the instant invention. In certain embodiments, the pharmaceutical composition of the invention may further comprise another therapeutic agent. Such other therapeutic agent can be one normally administered to patients with the disorder, disease or condition being treated.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

By “therapeutically effective amount” is meant an amount of compound or composition sufficient, upon single or multiple dose administration, to cause a detectable decrease in E1 enzyme variant activity and/or the severity of the disorder or disease state being treated. “Therapeutically effective amount” is also intended to include an amount sufficient to treat a cell, prolong or prevent advancement of the disorder or disease state being treated (e.g., prevent additional resistant tumor growth of a cancer, prevent inflammatory or parasite resistance), ameliorate, alleviate, relieve, or improve a subject's symptoms of the a disorder beyond that expected in the absence of such treatment. The amount of E1 enzyme inhibitor required will depend on the particular compound of the composition given, the type of disorder being treated, the route of administration, and the length of time required to treat the disorder. It should also be understood that a specific dosage and treatment regimen for any particular patient will depend upon a variety of factors, including the activity of the specific compound employed, the age, body weight, general health, sex, and diet of the patient, time of administration, rate of excretion, drug combinations, the judgment of the treating physician, and the severity of the particular disease being treated. In certain aspects where the inhibitor is administered in combination with another agent, the amount of additional therapeutic agent present in a composition of this invention typically will be no more than the amount that would normally be administered in a composition comprising that therapeutic agent as the only active agent. In some embodiments, the amount of additional therapeutic agent will range from about 50% to about 100% of the amount normally present in a composition comprising that agent as the only therapeutically active agent.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. In some embodiments, compounds exhibit high therapeutic indices. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds can lie within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, about 0.01 to 25 mg/kg body weight, about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, between 2 to 8 weeks, between about 3 to 7 weeks, or for about 4, 5, or 6 weeks. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody, unconjugated or conjugated as described herein, can include a single treatment or can include a series of treatments.

For antibodies, the dosage can be 0.1 to 20 mg/kg of body weight (generally 3 mg/kg to 10 mg/kg). If the antibody is to act in the brain, a dosage of 50 mg/kg to 100 mg/kg can be appropriate. Generally, partially human antibodies and fully human antibodies have a longer half-life within the human body than other antibodies. Accordingly, lower dosages and less frequent administration can be possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention also encompasses agents which modulate expression. An agent can, for example, be a small molecule. For example, such small molecules include, but are not limited to, amino acids, amino acid analogs, polynucleotides, polynucleotide analogs, dsRNA molecules, nucleotides, nucleotide analogs, organic or inorganic compounds (i.e., including heteroorganic and organometallic compounds) having a molecular weight less than about 10,000 grams per mole, e.g., 5, 000, 1,000 or 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### Methods of Treatment

The present invention provides for both prophylactic and therapeutic methods of treating a subject at risk of (or susceptible to) a disorder or having a disorder associated with aberrant or unwanted UBA3, UAE, or UBA6, or other E1 enzyme variant expression or activity, such as resistance or reduced sensitivity to an E1 enzyme inhibitor. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes, dsRNA molecules and antisense oligonucleotides, e.g., an agent which can overcome resistance to E1 enzyme inhibition. In some embodiments, examples of an agents which can overcome resistance can include agents identified in a screening assay described herein, agents listed in Table 13 as about 4-fold or less IC50 ratio of A271T/WT, an E1 enzyme inhibitor that when in adduct form with a UBL (e.g., NEDD8-MLN4924 adduct) can bind tightly to a variant E1 enzyme, or an adenosine-sulfamate-like inhibitor without a large N6-substitution (i.e., a bulky group, e.g., indane, off an amino substituent of the heteroaryl (e.g., purine))).

With regards to both prophylactic and therapeutic methods of treatment, such treatments can be specifically tailored or modified, based on knowledge obtained from the field of pharmacogenomics. Pharmacogenomics allows a clinician or physician to target prophylactic or therapeutic treatments to patients who will most benefit from the treatment and not to provide this treatment to patients who will experience toxic drug-related side effects or will not respond or will be resistant to treatment.

In one aspect, the invention provides a method for preventing in a subject, a disease or condition associated with an aberrant or unwanted UBA3, UAE, or UBA6, or other E1 enzyme variant expression or activity, by administering to the subject a UBA3, UAE, or UBA6, or other E1 enzyme variant or an agent which modulates UBA3, UAE, or UBA6, or other E1 enzyme variant expression or at least one UBA3, UAE, or UBA6, or other E1 enzyme variant activity. Subjects at risk for a disease which is caused or contributed to by aberrant or unwanted UBA3, UAE, or UBA6, or other E1 enzyme variant expression or activity can be identified by, for example, any or a combination of diagnostic or prognostic assays as described herein. Administration of a prophylactic agent can occur prior to the manifestation of symptoms characteristic of the UBA3, UAE, or UBA6, or other E1 enzyme variant aberrance, such that a disease or disorder is prevented or, alternatively, delayed in its progression. Depending on the type of UBA3, UAE, or UBA6, or other E1 enzyme variant aberrance, for example, a UBA3, UAE, or UBA6, or other E1 enzyme variant agonist or UBA3, UAE, or UBA6, or other E1 enzyme variant antagonist agent can be used for treating the subject. The appropriate agent can be determined based on screening assays described herein.

Another aspect of the invention pertains to methods of modulating resistance nucleic acid or protein expression or activity for therapeutic purposes. For example, the effectiveness of chemotherapy is “potentiated” (enhanced) by restoring or improving vulnerability of the transformed cells to the cytotoxic effects of a chemotherapeutic drug that otherwise would be less effective by reducing the expression of a resistance sequence in the cells. The modulatory method of the invention involves contacting a cell with an agent that modulates one or more of the activities of resistance protein activity associated with the cell. An agent that modulates a resistance protein activity can be an agent as described herein, such as a nucleic acid or a protein, a naturally-occurring cognate ligand of a resistance protein, a peptide, a resistance peptidomimetic, or other small molecule. In one embodiment, the agent stimulates one or more of the biological activities of a resistance protein. Examples of such stimulatory agents include active resistance protein and a nucleic acid molecule encoding a resistance protein that has been introduced into the cell. Such agents are particularly useful for increasing expression or activity of a down-regulated resistance nucleic acid or protein in a drug resistant cell. In another embodiment, the agent inhibits one or more of the biological activities of a resistance protein. Examples of such inhibitory agents include antisense resistance nucleic acid molecules, dsRNA molecules and anti-resistance protein antibodies. These modulatory methods can be performed in vitro (e.g., by culturing the cell with the agent) or, alternatively, in vivo (e.g., by administering the agent to a subject). Such methods are particularly useful for decreasing expression or activity of an up-regulated resistance nucleic acid or protein in a drug resistant cell. As such, the present invention provides methods of treating an individual afflicted with a disease or disorder characterized by aberrant expression or activity of a resistance sequence molecule. In one embodiment, the method involves administering an agent (e.g., an agent identified by a screening assay or otherwise described herein), or combination of agents that modulates (e.g., up-regulates or down-regulates) resistance expression or activity. In another embodiment, the method involves administering a resistance sequence molecule (e.g., a nucleic acid or a protein) as therapy to compensate for reduced or aberrant resistance expression or activity.

One embodiment of the invention relates to a method of inhibiting or decreasing E1 enzyme activity in a sample comprising contacting the sample with a compound of this invention, or composition comprising a compound of the invention. The sample, as used herein, includes, without limitation, sample comprising purified or partially purified E1 enzyme, cultured cells or extracts of cell cultures; biopsied cells or fluid obtained from a mammal, or extracts thereof; and body fluid (e.g., blood, serum, saliva, urine, feces, semen, tears) or extracts thereof. Inhibition of E1 enzyme activity in a sample may be carried out in vitro or in vivo, in cellulo, or in situ.

In another embodiment, the invention provides a method for treating a patient having a disorder, a symptom of a disorder, at risk of developing or experiencing a recurrence of a disorder, comprises administering to the patient a compound or pharmaceutical composition according to the invention. Treating can be to cure, heal, alleviate, relieve, alter, remedy, ameliorate, palliate, improve or affect the disorder, the symptoms of the disorder or the predisposition toward the disorder. While not wishing to be bound by theory, treating is believed to cause the inhibition of growth, ablation, or killing of a cell or tissue in vitro or in vivo, or otherwise reduce capacity of a cell or tissue (e.g., an aberrant cell, a diseased tissue) to mediate a disorder, e.g., a disorder as described herein (e.g., a proliferative disorder, e.g., a cancer, an inflammatory disorder or a parasitic infection). As used herein, “inhibiting the growth” or “inhibition of growth” of a cell or tissue (e.g., a proliferative cell, tumor tissue) refers to slowing, interrupting, arresting or stopping its growth and metastases and does not necessarily indicate a total elimination of growth.

The invention also features a method of treating a mammal suspected of having a disorder associated with the presence of drug-resistant cells. This method includes the steps of determining whether a mammal has a disorder associated with the presence of drug-resistant cells (e.g., drug-resistant cancer or parasitic infection), and administering to the mammal a compound that sufficiently alters activity or expression of, e.g., an up-regulated resistance sequence, so that the drug resistance of the cells associated with the disorder is modulated (i.e., reduced). In the case of a down-regulated resistance sequence, the compound administered to the mammal increases activity or expression of the sequence thereby modulating (i.e., reducing) drug resistance.

The invention also features a method of increasing the effectiveness of a chemotherapeutic compound in a patient suffering from a disorder associated with the presence of drug-resistant neoplastic, pathogenic or parasitic cells, the method including: a) administering a chemotherapeutic compound to the patient; and b) administering a compound which reduces the expression of an up-regulated resistance sequence in the patient. The invention further features a method of increasing the effectiveness of a chemotherapeutic compound in a patient suffering from a disorder associated with the presence of drug-resistant neoplastic cells, the method including: a) administering a chemotherapeutic compound to the patient; and b) administering a compound which reduces the expression of a down-regulated resistance sequence in the patient.

The invention features a method of treating a mammal suspected of having a disorder associated with the presence of drug-resistant cells, the method including administering to the mammal a compound that reduces the activity or expression of a resistance sequence in the drug-resistant cells, the reduction being sufficient to reduce the drug resistance of the drug resistant cells when the resistance sequence is an up-regulated resistance sequence. In another embodiment, the invention features a method of treating a mammal suspected of having a disorder associated with the presence of drug-resistant cells, the method including administering to the mammal a compound that increases the activity or expression of a resistance sequence in the drug-resistant cells, the reduction being sufficient to reduce the drug resistance of the drug resistant cells when the resistance sequence is a down-regulated resistance sequence.

Disease applications include those disorders in which inhibition of E1 enzyme activity is detrimental to survival and/or expansion of diseased cells or tissue (e.g., cells are sensitive to E1 inhibition; inhibition of E1 activity disrupts disease mechanisms; reduction of E1 activity stabilizes protein which are inhibitors of disease mechanisms; reduction of E1 activity results in inhibition of proteins which are activators of disease mechanisms). Disease applications are also intended to include any disorder, disease or condition which requires effective cullin and/or ubiquitination activity, which activity can be regulated by diminishing E1 enzyme activity (e.g., NAE, UAE, UBA6 activity).

For example, methods of the invention are useful in treatment of disorders involving cellular proliferation, including, but not limited to, disorders which require an effective cullin-dependent ubiquitination and proteolysis pathway (e.g., the ubiquitin proteasome pathway) for maintenance and/or progression of the disease state. The methods of the invention are useful in treatment of disorders mediated via proteins (e.g., NFκB activation, p27^{Kip} activation, p21^{WAF/CIP1} activation, p53 activation) which are regulated by E1 activity (e.g., NAE activity, UAE activity, SAE activity). Relevant disorders include proliferative disorders, including cancers and inflammatory disorders (e.g., rheumatoid arthritis, inflammatory bowel disease, asthma, chronic obstructive pulmonary disease (COPD), osteoarthritis, dermatosis (e.g., atopic dermatitis, psoriasis), vascular proliferative disorders (e.g., atherosclerosis, restenosis) autoimmune diseases (e.g., multiple sclerosis, tissue and organ rejection)); as well as inflammation associated with infection (e.g., immune responses), neurodegenerative disorders (e.g., Alzheimer's disease, Parkinson's disease, motor neurone disease, neuropathic pain, triplet repeat disorders, astrocytoma, and neurodegeneration as result of alcoholic liver disease), ischemic injury (e.g., stroke), and cachexia (e.g., accelerated muscle protein breakdown that accompanies various physiological and pathological states, (e.g., nerve injury, fasting, fever, acidosis, HIV infection, cancer affliction, and certain endocrinopathies)).

The compounds and pharmaceutical compositions of the invention are particularly useful for the treatment of cancer. As used herein, the term “cancer” (also used interchangeably with the terms, “hyperproliferative” and “neoplastic”) refers to a cellular disorder characterized by uncontrolled or disregulated cell proliferation, decreased cellular differentiation, inappropriate ability to invade surrounding tissue, and/or ability to establish new growth at ectopic sites. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes, but is not limited to, solid tumors and liquid or bloodborne tumors, i.e., a cell suspension in blood or other body fluid. The term “cancer” encompasses diseases of skin, tissues, organs, bone, cartilage, blood, and vessels. The term “cancer” further encompasses primary and metastatic cancers.

In some embodiments, the cancer is a solid tumor. Non-limiting examples of solid tumors that can be treated by the methods of the invention include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma.

In some other embodiments, the cancer is a hematologic malignancy. Non-limiting examples of hematologic malignancy include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma; T-cell lymphoma; multiple myeloma (MM); Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes.

In some embodiments, the compound or composition of the invention is used to treat a patient having or at risk of developing or experiencing a recurrence in a cancer selected from the group consisting of colorectal cancer, ovarian cancer, lung cancer, breast cancer, gastric cancer, prostate cancer, and pancreatic cancer. In certain embodiments, the cancer is selected from the group consisting of lung cancer, colorectal cancer, ovarian cancer and a hematologic cancer.

Depending on the particular disorder or condition to be treated, in some embodiments, the E1 enzyme inhibitor of the invention is administered in conjunction with additional therapeutic agent or agents. In some embodiments, the additional therapeutic agent(s) is one that is normally administered to patients with the disorder or condition being treated. As used herein, additional therapeutic agents that are normally administered to treat a particular disorder or condition are known as “appropriate for the disorder or condition being treated.”

Further, poly- or oligo-nucleotide, e.g., antisense, dsRNA, or ribozyme molecules that inhibit expression of the resistance gene can also be used in accordance with the invention to reduce the level of resistance gene expression, thus effectively reducing the level of resistance gene activity. Still further, triple helix molecules can be utilized in reducing the level of target gene activity. For example, a poly- or oligo-nucleotide pharmaceutical composition (or a cocktail composition comprising a poly- or oligo-nucleotide targeted to an up-regulated resistance sequence in combination with one or more other poly- or oligo-nucleotides) can be administered to the individual sufficiently in advance of administration of the chemotherapeutic drug to allow the poly- or oligo-nucleotide composition to permeate the individual's tissues, especially tissue comprising the transformed cells to be eradicated; to be internalized by transformed cells; and to disrupt resistance (e.g., up-regulated) sequence expression (e.g., disruption of expression of a resistance mRNA and/or resistance protein production).

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, wild type nucleic acid molecules that encode and express wild type UBA3, UAE, or UBA6, or other E1 enzyme gene polypeptides exhibiting normal target gene activity can be introduced into cells via gene therapy method.

Another method by which nucleic acid molecules can be utilized in treating or preventing a disease characterized by UBA3, UAE, or UBA6, or other E1 enzyme variant expression is through the use of aptamer molecules specific for UBA3, UAE, or UBA6, or other E1 enzyme variant protein. Aptamers are nucleic acid molecules having a tertiary structure which permits them to specifically or selectively bind to protein ligands (see, e.g., Osborne et al. (1997) Curr. Opin. Chem Biol. 1: 5-9; and Patel (1997) Curr Opin Chem Biol 1:32-46). Since nucleic acid molecules can in many cases be more conveniently introduced into target cells than therapeutic protein molecules can be, aptamers offer a method by which UBA3, UAE, or UBA6, or other E1 enzyme variant protein activity can be specifically decreased without the introduction of drugs or other molecules which can have pluripotent effects.

Antibodies can be generated that are both specific for target gene product and that reduce target gene product activity. Such antibodies can, therefore, by administered in instances whereby negative modulatory techniques are appropriate for the treatment of UBA3, UAE, or UBA6, or other E1 enzyme variant disorders. For a description of antibodies, see the Antibody section above.

In circumstances wherein injection of an animal or a human subject with a UBA3, UAE, or UBA6, or other E1 enzyme variant protein or epitope for stimulating antibody production is harmful to the subject, it is possible to generate an immune response against UBA3, UAE, or UBA6, or other E1 enzyme variant through the use of anti-idiotypic antibodies (see, for example, Herlyn (1999) Ann Med 31:66-78; and Bhattacharya-Chatterjee and Foon (1998) Cancer Treat Res. 94:51-68). If an anti-idiotypic antibody is introduced into a mammal or human subject, it should stimulate the production of anti-anti-idiotypic antibodies, which should be specific to the UBA3, UAE, or UBA6, or other E1 enzyme variant protein. Vaccines directed to a disease characterized by UBA3, UAE, or UBA6, or other E1 enzyme variant expression can also be generated in this fashion.

In instances where the target antigen is intracellular and whole antibodies are used, internalizing antibodies can be useful. Lipofectin or liposomes can be used to deliver the antibody or a fragment of the Fab region that binds to the target antigen into cells. Embodiments using fragments of the antibody can use the smallest inhibitory fragment that binds to the target antigen. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate UBA3, UAE, or UBA6, or other E1 enzyme variant disorders. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds can lie within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays can utilize antibody mimics and/or “biosensors” that have been created through molecular imprinting techniques. The compound which is able to modulate UBA3, UAE, or UBA6, or other E1 enzyme variant activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al (1996) Current Opinion in Biotechnology 7:89-94 and in Shea (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis et al (1993) Nature 361:645-647. Through the use of isotope-labeling, the “free” concentration of compound which modulates the expression or activity of UBA3, UAE, or UBA6, or other E1 enzyme variant can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz et al (1995) Analytical Chemistry 67:2142-2144.

When used in combination therapy, the E1 inhibitor of the invention may be administered with the other therapeutic agent in a single dosage form or as a separate dosage form. When administered as a separate dosage form, the other therapeutic agent may be administered prior to, at the same time as, or following administration of the E1 inhibitor of the invention.

In some embodiments, the E1 enzyme inhibitor of the invention is administered in conjunction with a therapeutic agent selected from the group consisting of cytotoxic agents, radiotherapy, and immunotherapy appropriate for treatment of proliferative disorders and cancer. Non-limiting examples of cytotoxic agents suitable for use in combination with the E1 enzyme inhibitors of the invention include: antimetabolites, including, e.g., capecitibine, gemcitabine, 5-fluorouracil or 5-fluorouracil/leucovorin, fludarabine, cytarabine, mercaptopurine, thioguanine, pentostatin, and methotrexate; topoisomerase inhibitors, including, e.g., etoposide, teniposide, camptothecin, topotecan, irinotecan, doxorubicin, and daunorubicin; vinca alkaloids, including, e.g., vincristine and vinblastin; taxanes, including, e.g., paclitaxel and docetaxel; platinum agents, including, e.g., cisplatin, carboplatin, and oxaliplatin; antibiotics, including, e.g., actinomycin D, bleomycin, mitomycin C, adriamycin, daunorubicin, idarubicin, doxorubicin and pegylated liposomal doxorubicin; alkylating agents such as melphalan, chlorambucil, busulfan, thiotepa, ifosfamide, carmustine, lomustine, semustine, streptozocin, decarbazine, and cyclophosphamide; including, e.g., CC-5013 and CC-4047; protein tyrosine kinase inhibitors, including, e.g., imatinib mesylate and gefitinib; proteasome inhibitors, including, e.g., bortezomib, thalidomide and related analogs; antibodies, including, e.g., trastuzumab, rituximab, cetuximab, and bevacizumab; mitoxantrone; dexamethasone; prednisone; and temozolomide.

Other examples of agents the inhibitors of the invention may be combined with include anti-inflammatory agents such as corticosteroids, TNF blockers, Il-1 RA, azathioprine, cyclophosphamide, and sulfasalazine; immunomodulatory and immunosuppressive agents such as cyclosporine, tacrolimus, rapamycin, mycophenolate mofetil, interferons, corticosteroids, cyclophosphamide, azathioprine, methotrexate, and sulfasalazine; antibacterial and antiviral agents; and agents for Alzheimer's treatment such as donepezil, galantamine, memantine and rivastigmine.

### OTHER EMBODIMENTS

In another aspect, the invention features a method of analyzing a plurality of capture probes. The method is useful, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence, wherein the capture probes are from a cell or subject which expresses UBA3, UAE, or UBA6, or other E1 enzyme variant or from a cell or subject in which a UBA3, UAE, or UBA6, or other E1 enzyme variant mediated response has been elicited; contacting the array with a UBA3, UAE, or UBA6, or other E1 enzyme variant nucleic acid (e.g., purified), a UBA3, UAE, or UBA6, or other E1 enzyme variant polypeptide (e.g., purified), or an anti-UBA3, UAE, or UBA6, or other E1 enzyme variant antibody, and thereby evaluating the plurality of capture probes. Binding, e.g., in the case of a nucleic acid, hybridization with a capture probe at an address of the plurality, is detected, e.g., by a signal generated from a label attached to the UBA3, UAE, or UBA6, or other E1 enzyme variant nucleic acid, polypeptide, or antibody.

The capture probes can be a set of nucleic acids from a selected sample, e.g., a sample of nucleic acids derived from a control or non-stimulated tissue or cell.

The method can include contacting the UBA3, UAE, or UBA6, or other E1 enzyme variant nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of UBA3, UAE, or UBA6, or other E1 enzyme variant. Such methods can be used to diagnose a subject, e.g., to evaluate risk for a disease or disorder, to evaluate suitability of a selected treatment for a subject, to evaluate whether a subject has a disease or disorder characterized by resistance to an E1 enzyme inhibitor.

In another aspect, the invention features, a method of analyzing UBA3, UAE, or UBA6, or other E1 enzyme variant, e.g., analyzing structure, function, or relatedness to other nucleic acid or amino acid sequences. The method includes: providing a UBA3, UAE, or UBA6, or other E1 enzyme variant nucleic acid or amino acid sequence; comparing the UBA3, UAE, or UBA6, or other E1 enzyme variant sequence with one or more or a plurality of sequences from a collection of sequences, e.g., a nucleic acid or protein sequence database; to thereby analyze UBA3, UAE, or UBA6, or other E1 enzyme variant.

The method can include evaluating the sequence identity between a UBA3, UAE, or UBA6, or other E1 enzyme variant sequence and a database sequence. The method can be performed by accessing the database at a second site, e.g., over the internet. Examples of databases include GenBank™ (National Center for Biotechnology Information) and SwissProt (Swiss Bioinformatics Institute).

In another aspect, the invention features, a set of oligonucleotides, useful, e.g., for identifying SNP's, or identifying specific alleles of UBA3, UAE, or UBA6, or other E1 enzyme variant. The set includes a plurality of oligonucleotides, each of which has a different nucleotide at an interrogation position, e.g., an SNP or the site of a mutation. In an embodiment, the oligonucleotides of the plurality are identical in sequence with one another (except for differences in length). The oligonucleotides can be provided with differential labels, such that an oligonucleotide which hybridizes to one allele provides a signal that is distinguishable from oligonucleotides which hybridizes to a second allele.

The sequences of UBA3, UAE, or UBA6, or other E1 enzyme variant molecules are provided in a variety of mediums to facilitate use thereof. A sequence can be provided as a manufacture, other than an isolated nucleic acid or amino acid molecule, which contains a UBA3, UAE, or UBA6, or other E1 enzyme variant molecule. Such a manufacture can provide a nucleotide or amino acid sequence, e.g., an open reading frame, in a form which allows examination of the manufacture using means not directly applicable to examining the nucleotide or amino acid sequences, or a subset thereof, as they exist in nature or in purified form.

A UBA3, UAE, or UBA6, or other E1 enzyme variant nucleotide or amino acid sequence can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as compact disc and CD-ROM; electrical storage media such as RAM, ROM, EPROM, EEPROM, and the like; and general hard disks and hybrids of these categories such as magnetic/optical storage media. The medium is adapted or configured for having thereon UBA3, UAE, or UBA6, or other E1 enzyme variant sequence information of the present invention.

As used herein, the term “electronic apparatus” is intended to include any suitable computing or processing apparatus of other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as personal digital assistants (PDAs), cellular phones, pagers, and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the UBA3, UAE, or UBA6, or other E1 enzyme variant sequence information.

A variety of data storage structures are available to a skilled artisan for creating a computer readable medium having recorded thereon a UBA3, UAE, or UBA6, or other E1 enzyme variant nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

By providing the UBA3, UAE, or UBA6, or other E1 enzyme variant nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif.

The present invention therefore provides a medium for holding instructions for performing a method for determining whether a subject has a UBA3, UAE, or UBA6, or other E1 enzyme variant-associated disease or disorder or a pre-disposition to a UBA3, UAE, or UBA6, or other E1 enzyme variant-associated disease or disorder, wherein the method comprises the steps of determining UBA3, UAE, or UBA6, or other E1 enzyme variant sequence information associated with the subject and based on the UBA3, UAE, or UBA6, or other E1 enzyme variant sequence information, determining whether the subject has a UBA3, UAE, or UBA6, or other E1 enzyme variant-associated disease or disorder and/or recommending a particular treatment for the disease, disorder, or pre-disease condition.

The present invention further provides in an electronic system and/or in a network, a method for determining whether a subject has a UBA3, UAE, or UBA6, or other E1 enzyme variant-associated disease or disorder or a pre-disposition to a disease associated with a UBA3, UAE, or UBA6, or other E1 enzyme variant, wherein the method comprises the steps of determining UBA3, UAE, or UBA6, or other E1 enzyme variant sequence information associated with the subject, and based on the UBA3, UAE, or UBA6, or other E1 enzyme variant sequence information, determining whether the subject has a UBA3, UAE, or UBA6, or other E1 enzyme variant-associated disease or disorder or a pre-disposition to a UBA3, UAE, or UBA6, or other E1 enzyme variant-associated disease or disorder, and/or recommending a particular treatment for the disease, disorder, or pre-disease condition. The method may further comprise the step of receiving phenotypic information associated with the subject and/or acquiring from a network phenotypic information associated with the subject.

The present invention also provides in a network, a method for determining whether a subject has a UBA3, UAE, or UBA6, or other E1 enzyme variant-associated disease or disorder or a pre-disposition to a UBA3, UAE, or UBA6, or other E1 enzyme variant-associated disease or disorder, said method comprising the steps of receiving UBA3, UAE, or UBA6, or other E1 enzyme variant sequence information from the subject and/or information related thereto, receiving phenotypic information associated with the subject, acquiring information from the network corresponding to UBA3, UAE, or UBA6, or other E1 enzyme variant and/or corresponding to a UBA3, UAE, or UBA6, or other E1 enzyme variant-associated disease or disorder, and based on one or more of the phenotypic information, the UBA3, UAE, or UBA6, or other E1 enzyme variant information (e.g., sequence information and/or information related thereto), and the acquired information, determining whether the subject has a UBA3, UAE, or UBA6, or other E1 enzyme variant-associated disease or disorder or a pre-disposition to a UBA3, UAE, or UBA6, or other E1 enzyme variant-associated disease or disorder. The method may further comprise the step of recommending a particular treatment for the disease, disorder, or pre-disease condition.

The present invention also provides a business method for determining whether a subject has a UBA3, UAE, or UBA6, or other E1 enzyme variant-associated disease or disorder or a pre-disposition to a UBA3, UAE, or UBA6, or other E1 enzyme variant-associated disease or disorder, said method comprising the steps of receiving information related to UBA3, UAE, or UBA6, or other E1 enzyme variant (e.g., sequence information and/or information related thereto), receiving phenotypic information associated with the subject, acquiring information from the network related to UBA3, UAE, or UBA6, or other E1 enzyme variant and/or related to a UBA3, UAE, or UBA6, or other E1 enzyme variant-associated disease or disorder, and based on one or more of the phenotypic information, the UBA3, UAE, or UBA6, or other E1 enzyme variant information, and the acquired information, determining whether the subject has a UBA3, UAE, or UBA6, or other E1 enzyme variant-associated disease or disorder or a pre-disposition to a UBA3, UAE, or UBA6, or other E1 enzyme variant-associated disease or disorder. The method may further comprise the step of recommending a particular treatment for the disease, disorder, or pre-disease condition.

The invention also includes an array comprising a UBA3, UAE, or UBA6, or other E1 enzyme variant sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be UBA3, UAE, or UBA6, or other E1 enzyme variant. This allows a profile to be developed showing a battery of genes specifically expressed in one or more tissues.

In addition to such qualitative information, the invention allows the quantification of gene expression. Thus, not only tissue specificity, but also the level of expression of a battery of genes in the tissue if ascertainable. Thus, genes can be grouped on the basis of their tissue expression per se and level of expression in that tissue. This is useful, for example, in ascertaining the relationship of gene expression in that tissue. Thus, one tissue can be perturbed and the effect on gene expression in a second tissue can be determined. In this context, the effect of one cell type on another cell type in response to a biological stimulus can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a UBA3, UAE, or UBA6, or other E1 enzyme variant-associated disease or disorder, progression of UBA3, UAE, or UBA6, or other E1 enzyme variant-associated disease or disorder, and processes, such a cellular transformation associated with the UBA3, UAE, or UBA6, or other E1 enzyme variant-associated disease or disorder.

The array is also useful for ascertaining the effect of the expression of a gene on the expression of other genes in the same cell or in different cells (e.g., acertaining the effect of UBA3, UAE, or UBA6, or other E1 enzyme variant expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including a UBA3, UAE, or UBA6, or other E1 enzyme variant) that could serve as a molecular target for diagnosis or therapeutic intervention.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a UBA3, UAE, or UBA6, or other E1 enzyme variant sequence which includes recording the sequence on a computer readable matrix. In an embodiment, the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features a method of analyzing a sequence. The method includes: providing a UBA3, UAE, or UBA6, or other E1 enzyme variant sequence, or record, in computer readable form; comparing a second sequence to the UBA3, UAE, or UBA6, or other E1 enzyme variant sequence; thereby analyzing a sequence. Comparison can include comparing to sequences for sequence identity or determining if one sequence is included within the other, e.g., determining if the UBA3, UAE, or UBA6, or other E1 enzyme variant sequence includes a sequence being compared. In an embodiment, the UBA3, UAE, or UBA6, or other E1 enzyme variant or second sequence is stored on a first computer, e.g., at a first site and the comparison is performed, read, or recorded on a second computer, e.g., at a second site. E.g., the UBA3, UAE, or UBA6, or other E1 enzyme variant or second sequence can be stored in a public or proprietary database in one computer, and the results of the comparison performed, read, or recorded on a second computer. In an embodiment, the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In order that this invention be more fully understood, the following preparative and testing examples are set forth. These examples are for the purpose of illustration only and are not to be construed as limiting the scope of the invention in any way.

### Examples

### Example 1

### Clonal Selection of Tumor Cell Lines that are Resistant to MLN4924

Cell line cultures were maintained using appropriate cell culture media as recommended by ATCC and as previously reported (Soucy et al, 2009, Milhollen et al., 2010). To derive resistant cell lines HCT-116, Calu-6 and NCI-H460 cells were incubated with high concentrations of MLN4924 (≧EC₉₀ concentrations, 1 μM for HCT-116, Calu-6 and 10 μM for NCI-H460) for four days after which remaining cells were removed, plated as single cell clones and cultured in drug-free media. Cell viability assays completed using 96 hours ATPlite assay (PerkinElmer) as previously reported (Soucy et al., 2009).

Drug Efflux Inhibitor Experiments

Diypridamole (Sigma), MK571 (Tocris), GF918, KO143 (Tocris) and LY5979 were obtained from commercial sources indicated. The highest non-toxic concentration of each agent was determined in ATPlite viability assays and used in co-incubation assays with a dose titration of MLN4924.

Quantitative RT-PCR

cDNA synthesis and quantitative RT-PCR was performed using ABI Gene Expression Assays, reagents, and ABI PRISM® 7900HT Sequence Detection Systems (Applied Biosystems, Foster City, Calif.) using the following cycle conditions: hold at 50° C. for 2 minutes for AmpErase UNG activation, then 95.0° C. for 10 minutes to activate DNA polymerase then run 40 two-part cycles of 95.0° C. for 15 seconds and 60.0° C. for 1 minute. The dCt was calculated by using the average Ct of control genes B2M (Hs99999907_m1) and RPLPO (Hs99999902_m1). Relative mRNA expression quantitation was derived using the Comparative Ct Method (Applied Biosystems). mRNA expression fold change values were generated from a comparison of each parental cell line and corresponding resistant clones.

Results

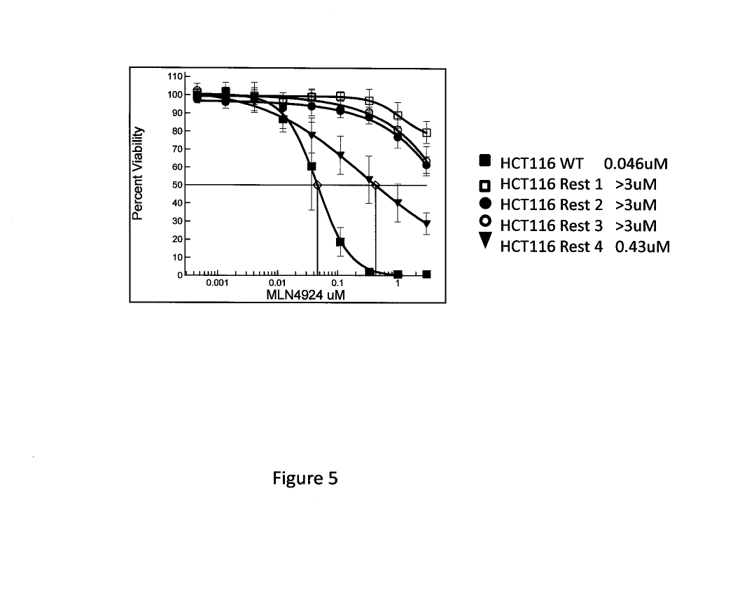

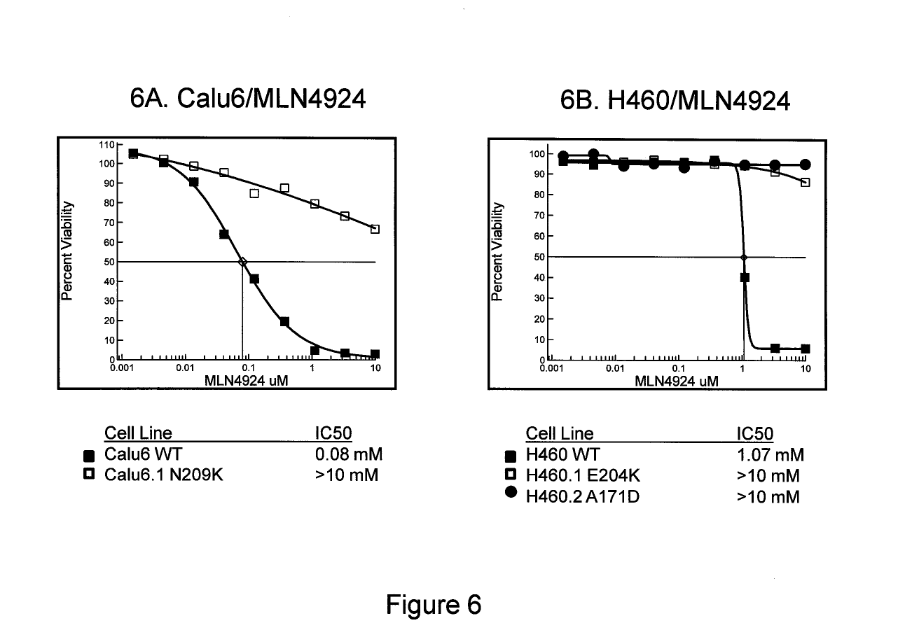

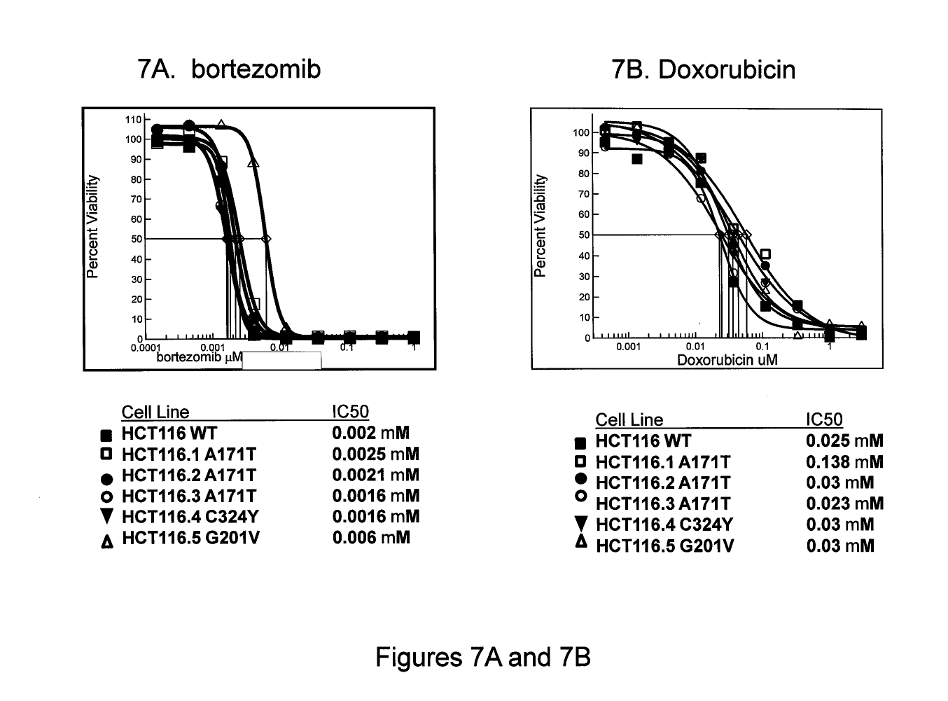

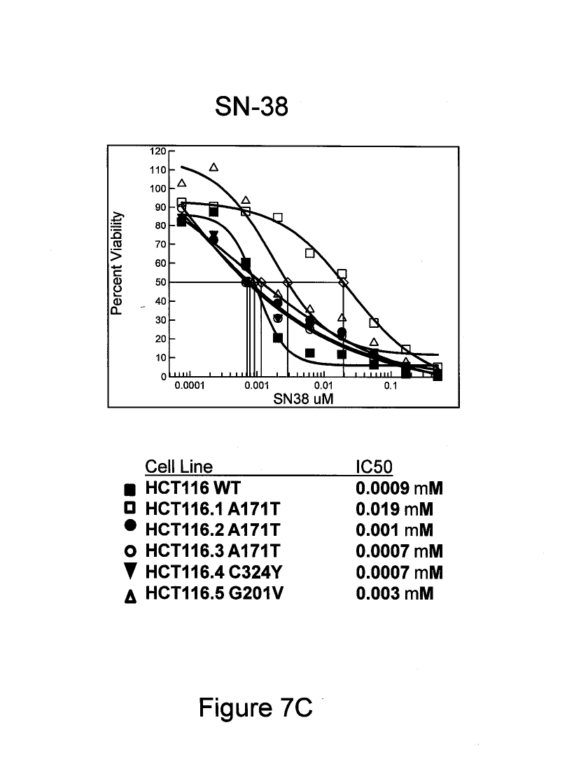

Three solid tumor cell lines (HCT-116 colorectal, NCI-H460 lung, Calu-6 lung) that have been shown to undergo DNA re-replication in response to NAE inhibition were chosen to study potential mechanisms of resistance to MLN4924 (Soucy et al., 2009). These cell lines have differential sensitivity to MLN4924 induced cytotoxicity with EC₅₀ values (cell viability assay) of 46, 80 and 1070 nM for HCT-116, Calu-6 and NCI-H460 cells respectively. Exposure to high concentrations of MLN4924 (≧EC₉₀ concentrations) for four days results in almost complete cell kill. Five resistant clones were obtained. One HCT-116 clone was found to be 10-fold less sensitive to MLN4924 and four had EC₅₀ values greater than 10 μM (FIG. 5). Similarly, two NCI-H460 and one Calu-6 clones were isolated and found to have EC₅₀ values of >10 μM (FIGS. 6A-B). The MLN4924 resistant HCT-116 cell clones remained sensitive to other chemotherapies (bortezomib, SN-38 and doxorubicin, FIGS. 7A-C) suggesting the resistance mechanism is not shared with other agents.

To determine whether changes in the NEDD8 pathway may explain the resistance to MLN4924 cells were evaluated for changes in gene transcript levels or the presence of DNA mutations in NAEβ, NAE1, NEDD8 or Ubc12, the primary E2 for neddylation. No substantial changes were found in mRNA levels of neddylation pathway genes (See Table 1).

TABLE 1
mRNA measurements of genes known to be involved
in drug efflux and NEDD8 pathway conjugation.

| | HCT- | HCT- | HCT- | HCT- | HCT- | Calu- | NCI- | NCI- |
|---|---|---|---|---|---|---|---|---|
| | 116.1 | 116.2 | 116.3 | 116.4 | 116.5 | 6.1 | H460.1 | H460.2 |
| Gene | A171T | A171T | A171T | C324Y | G201V | N209K | E204K | A171D |
| ABCB1 | 3.14 | 5.00 | 7.31 | 6.97 | 0.17 | 0.24 | 7.47 | 0.11 |
| ABCA12 | 3.96 | 2.18 | 2.54 | 0.48 | 1.55 | 0.00 | 0.00 | 0.00 |
| ABCG2 | 1.32 | 2.31 | 2.24 | 2.11 | 0.52 | 1.52 | 1.05 | 2.87 |
| ABCC6 | 2.66 | 1.26 | 2.16 | 2.14 | 0.74 | 0.67 | 1.11 | 1.51 |
| ABCA4 | 2.08 | 1.45 | 2.13 | 1.88 | 1.06 | 2.56 | 0.72 | 1.61 |
| ABCA1 | 0.84 | 1.42 | 2.10 | 1.91 | 0.06 | 0.56 | 1.32 | 0.82 |
| ABCC3 | 1.13 | 1.22 | 2.08 | 1.29 | 1.27 | 1.93 | 1.04 | 1.25 |
| ABCG1 | 1.00 | 1.42 | 2.04 | 1.91 | 0.29 | 3.00 | 0.81 | 0.44 |
| ABCG4 | 1.50 | 1.61 | 1.42 | 1.79 | 1.24 | 1.62 | 1.18 | 1.97 |
| ABCC2 | 0.61 | 0.62 | 1.28 | 0.78 | 1.22 | 4.84 | 0.97 | 1.59 |
| ABCC5 | 0.87 | 0.91 | 1.09 | 1.06 | 0.84 | 0.94 | 1.87 | 3.24 |
| ABCA11 | 1.21 | 1.15 | 1.07 | 1.08 | 2.26 | 1.09 | 0.87 | 1.46 |
| ABCA7 | 1.24 | 0.81 | 0.96 | 0.89 | 2.01 | 1.11 | 1.07 | 1.23 |
| ABCA10 | 0.67 | 0.34 | 0.77 | 0.76 | 0.00 | 0.68 | 1.04 | 1.13 |
| ABCC11 | 0.42 | 0.74 | 0.76 | 0.78 | 1.00 | 0.20 | 1.96 | 0.93 |
| ABCA2 | 0.77 | 0.57 | 0.69 | 0.70 | 1.10 | 0.89 | 0.97 | 1.50 |
| ABCA5 | 0.74 | 0.46 | 0.65 | 0.54 | 0.72 | 0.88 | 1.20 | 1.32 |
| ABCA3 | 1.06 | 0.70 | 0.58 | 1.29 | 1.13 | 1.24 | 1.02 | 1.72 |
| ABCA6 | 0.59 | 0.97 | 0.53 | 0.00 | 1.00 | 0.47 | 2.19 | 2.81 |
| ABCD2 | 0.00 | 0.00 | 0.00 | 0.00 | 1.00 | 0.46 | 3.82 | 3.87 |
| CAND1 | 0.93 | 1.16 | 1.25 | 1.09 | 0.64 | 0.75 | 0.95 | 1.49 |
| CDT1 | 0.87 | 0.83 | 0.81 | 0.86 | 0.42 | 1.36 | 0.93 | 1.45 |
| COPS3 | 0.83 | 1.11 | 1.07 | 0.87 | 0.37 | 1.31 | 1.07 | 1.49 |
| COPS5 | 0.99 | 1.17 | 1.32 | 1.08 | 0.78 | 1.12 | 1.00 | 1.37 |
| COPS8 | 1.02 | 1.22 | 1.34 | 1.10 | 0.47 | 0.98 | 1.03 | 1.63 |
| CUL1 | 1.00 | 1.15 | 1.32 | 1.16 | 0.60 | 1.16 | 1.00 | 1.41 |
| CUL2 | 1.23 | 1.39 | 1.48 | 1.44 | 0.45 | 1.54 | 0.98 | 1.45 |
| CUL3 | 0.99 | 1.14 | 1.29 | 1.11 | 0.52 | 1.13 | 0.98 | 1.27 |
| CUL4A | 1.04 | 1.20 | 1.39 | 1.18 | 0.88 | 1.14 | 1.34 | 1.65 |
| CUL4B | 1.08 | 1.17 | 1.16 | 1.08 | 0.27 | 0.88 | 1.06 | 1.66 |
| CUL5 | 0.94 | 0.89 | 1.11 | 0.96 | 0.67 | 0.92 | 1.04 | 1.49 |
| CUL7 | 1.05 | 0.84 | 1.26 | 0.60 | 1.08 | 0.96 | 0.96 | 1.38 |
| DCUN1D1 | 1.18 | 1.27 | 1.51 | 1.27 | 0.45 | 1.19 | 0.98 | 1.46 |
| NAE1 | 0.94 | 1.10 | 1.18 | 1.02 | 0.77 | 1.05 | 0.89 | 1.19 |
| NEDD8 | 0.98 | 1.10 | 1.18 | 0.93 | 0.57 | 1.24 | 0.96 | 1.32 |
| NFE2L2 | 0.81 | 0.94 | 1.10 | 0.85 | 0.40 | 1.02 | 1.02 | 1.43 |
| NFKB1 | 0.85 | 1.01 | 1.06 | 1.02 | 0.50 | 1.12 | 1.19 | 1.48 |
| SENP8 | 1.14 | 1.18 | 1.33 | 1.01 | 0.47 | 1.38 | 1.21 | 1.15 |
| UBA3 | 1.48 | 1.51 | 1.61 | 1.38 | 0.77 | 1.03 | 1.05 | 1.39 |
| UBE2F | 1.24 | 1.27 | 1.27 | 0.92 | 0.58 | 1.18 | 1.16 | 1.56 |
| UBE2M | 1.03 | 1.30 | 1.30 | 0.91 | 0.34 | 1.23 | 1.15 | 1.30 |

HCT-116, Calu-6 and NCI-H460 WT and resistant clones were subjected to quantitative RT-PCR analysis of genes. Data is represented as normalized to WT cell lines and indicates fold change in expression level of each mRNA.

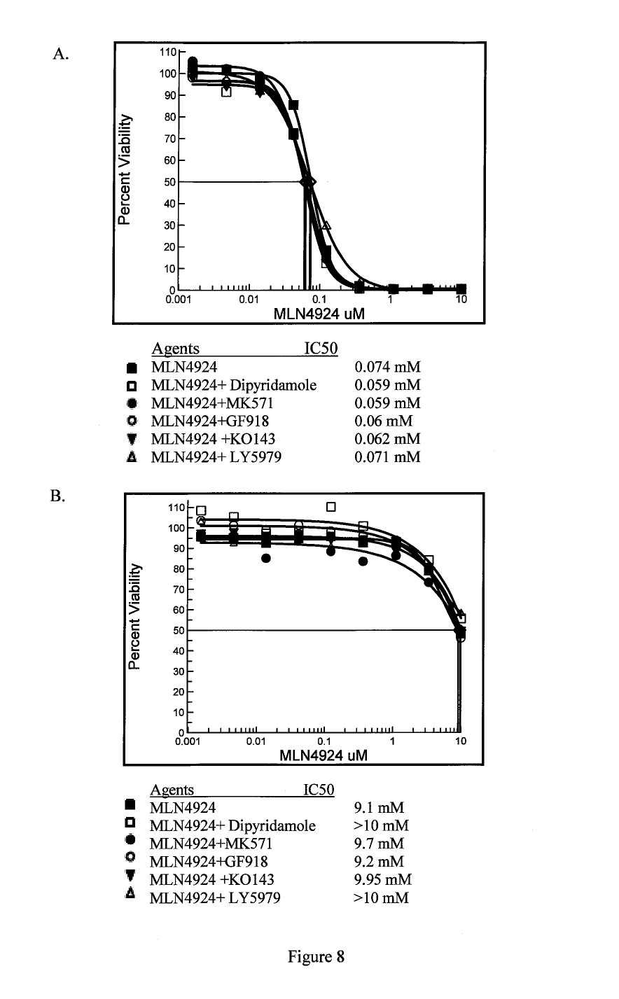

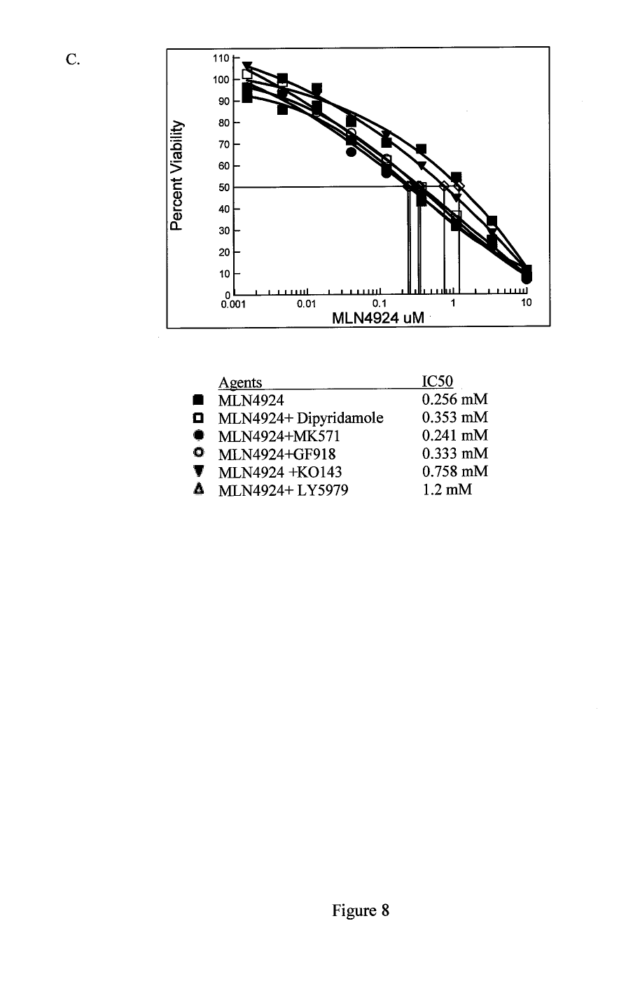

Changes were observed in mRNA levels of ABC-transporter proteins; however, MLN4924 activity was unaffected by co-incubation with drug efflux inhibitors (FIGS. 8A-C).

The resistant cell lines were selected following a short exposure (4 days) to high concentrations of MLN4924. All cell lines were isolated as clonal populations, shown to be resistant to MLN4924 and still sensitive to other chemotherapies including proteasome inhibition (bortezomib), an anthracycline (doxorubicin) and a topoisomerase I inhibitor (SN-38). RT-PCR analysis of the clones indicated an elevation of drug efflux mechanisms with increased detection of mRNA for PgP and BCRP in some cell lines. However, it appears that the elevation of drug efflux does not significantly contribute to the resistance as co-treating cells with a number of Pgp, BCRP and MRP2 inhibitors did not sensitize cells to MLN4924 (Dipyridamole (Shalinsky et al., 1993), MK519 (Gekeler et al., 1995), GF120918 (Hyafil et al., 1993), K0143 (Allen et al., 2002) and LY5979 (Dantzig et al., 1993). This data, in addition to the presence of mutations in NAEβ, indicate that the resistance is driven by mutations in the target enzyme.

### Example 2

### Tumor Cell Lines that are Resistant to MLN4924 Contain a Mutation in NAEβ

Isolation and Characterization of Resistance cDNA Sequences

Genomic Isolations and DNA Sequencing

DNA isolation of cells and xenografts was conducted using DNAeasy (Qiagen). RNA isolation was conducted using MegaMax (Ambion). Genomic isolations were conducted following manufacturer recommend protocols. Sequencing of NAE1, NAEβ, UBC12 and NEDD8 was performed as described below.

Sanger Sequencing Methodology

PCR amplifications (see below) were conducted using optimized cycling conditions per gene-exon. Primer extension sequencing was performed using Applied Biosystems BigDye version 3.1. The reactions were then run on Applied Biosystem's 3730x1 DNA Analyzer. Sequencing base calls were done using KB™ Basecaller (Applied Biosystems). Somatic Mutation calls were determined by Mutation Surveyor (SoftGenetics) and confirmed manually by aligning sequencing data with the corresponding reference sequence using Seqman (DNASTAR).

TABLE 2
Primers used for gene and exon amplification for Sanger sequencing

| Gene | Exon | | |
|---|---|---|---|
| Name | Number | Forward Primer Sequence | Reverse Primer Sequence |
| NAEβ | 1 | agcccagccgcagtcaacc | agcatcgcgctacacactgg |
| (UBA3) | 2 | ccagtgtgtagcgcgatgct | cgtcggggccaggctgtactgcc |
| | 3 | ATTTGAGCAGGTCTGGTGTG | CAAATATTCAAACAAAATCAGTTGTG |
| | 4 | CAAAGTCTCCTCTTTATCCTAGTGG | CTTGAAGTTAGGAGCGTTTTGC |
| | 5 | CAAGAGTCTTTCAGCCATTGAATAC | AAACTTTACCTACAATGGGAATGG |
| | 6 | GGACCATTAGCAAGGGTTGAC | AAATGTTACCAGCAGTTATCTCAGTAAT |
| | 7 | GCAGAGTGTGTTCCTTATGGC | CGTTCTCAGAGCTCCAATTCC |
| | 8 | TCCCACAAACACACTACCTTCC | CCTATGAGTCGGTTGTGCTATTG |
| | 9 | CCGACTCATAGGATTACTTGAAAGC | TCGGTTCATATCTTTCCTCAAATAG |
| | 10 | TTACAGGCAGTGCAGCCTAAG | TCATCTCCATCTAATGGAACCC |
| | 11 | CATCTATGCCCAGGCTACCAG | TGGACAGTTTGACTTGGATGA |
| | 12 | CATTGTGCCTGGAACATAATACTC | TCGACAGCTTAGTTACACAACCC |
| | 13 | tgaatcacacaaaacaatgtaaaa | gaaaatgtatgggtgactttgttc |
| | 14 | SAME AS ABOVE | SAME AS ABOVE |
| | 15 | AGCGAAGTAATTTACAAGAAATGGTC | GGCTGGAGATTTCATTTGCC |
| | 16 | tgtagccagcttcctcaaaat | gaaaaacatcaaaatccaatcctc |
| | 17 | SAME AS ABOVE | SAME AS ABOVE |
| | 18 | TCTTAATGCCTTGTATATGGTCAGG | AAAGACAAATCGTGGCAACAC |
| UBE2M | 1 | Not tested | Not tested |
| | 2 | CTAACAAACACCCGCCCTAA | AGCTGCTGCCTCCTCTGT |
| | 3 | TCTCTTCCCCTCCCTCTTTC | AGCTGCTGCCTCCTCTGTC |
| | 4 | tcccagggcttctacaagagt | gtcaggccatgaggaagatg |
| | 5-6 | cctctcacatgctcactctcc | ccgaccttaatcacatggtgt |
| NEDD8 | 1 | gacagtgacccggaagtagaa | gtcctccaggctagggaaag |
| | 2 | CCAAAGCAGTGCTGCTGAGAA | TAGGGGTAGGACCATGGAAAC |
| | 3 | CATGCCCAACTCCTCTTCAT | AAGGGGCTTAGAGGCTTCAC |
| | 4 | gcgagactctgtcaaaaacaac | aagcacacaggactgcaaact |
| NAE1 | 1 | ATTACTGCATGGACAGAGGGC | CTGGAAGAAGGCCTGAGGAAG |
| | 2-3 | gcaaggaaagtttaaagatcacc | tcaggccttgccttccttaac |
| | 4 | TCCAGGAAGGCCATTACAGTC | TCAAGATTTAGCTTTAATACAGGATGC |
| | 5 | ATGCTACAAACTGGGCAACAG | GAGGCAACAATTCCAATTCAAC |
| | 6 | TTGAATTGGAATTGTTGCCTC | GCCCAGCCATAAATCTGAAAC |
| | 7 | AGCCTGTGGAAATGCTTGC | TGATTGCCACTTTGAATCTGC |
| | 8 | CAGCCTGGGTAATACAGCAAG | ATCGGGATGACAGGAATATGG |
| | 9 | CCTTTCTTACTTAGGATTGGTGTCTG | AAGCATCCATTTGCCCTACC |
| | 10 | TCAAGTGAGCCTCTGATTTCAC | GCGTGGCTTCTTAACTTCTCC |
| | 11 | AAGTTAAGAAGCCACGCCTTG | CCCGGCCCTGTATTTATTTC |
| | 12-13 | tgaaaataaagatgggcgaat | cactgcagctggcctagttta |
| | 14 | GGTGCTGTTGATAGCATTTCC | CAAGCTATCCCATTAGGCAGG |
| | 15 | CGCTGTGGGTAATCATGTTG | CACTGAGGCCATGGAAAACT |
| | 16 | AGGCCATCACCTTACTTGCTT | CACACAATGGACTCAGTGACC |
| | 17 | TTCAGTAGAGATTGTGTGACTCCAG | AAAGGTGGTTCTTCACTGGG |
| | 18-19 | TCTGATTATAGGTTTGTGATATGTGC | CAACATCCTGCTTCACTGACC |
| | 20 | TTTGGTCAACCACACTACTGTTTAG | TTTACAGACTAAAGCACAACCCG |

Sequenom Sequencing Methodology

Sequenom NAEβ (UBA3) and NEDD8 assays were designed using TypePLEX® chemistry with single-base extension. This process consists of three steps: 1) A text file containing the SNPs of interest and flanking sequence is uploaded at mysequenom.com where it is run through a web based program ProxSNP, 2) The output of ProxSNP is run through PreXTEND and 3) the output of PreXTEND is run through Assay Design which determines the expected mass weight of the extend products to ensure separation between all potential peaks found within a multiplexed reaction.

15 nl of amplified and extended product is spotted on a 384 SpectroCHIP II using a Nanodispenser RS1000. A 3-point calibrant is added to every chip to ensure proper performance of the Sequenom Maldi-tof compact mass spectrometer.

The SpectroCHIP II is placed in the Sequenom MALDI-TOF compact mass spectrometer. The mass spectrometer is set to fire a maximum of 9 acquisitions for each spot on the 384 well spectroCHIP. TypePLEX Gold kit SpectroCHIP II from Sequenom (10142-2) is used following manufacturers recommended protocols. Analysis is performed using Sequenom analysis software, MassARRAY® Typer Analyzer v4.

TABLE 3
Primer sequences and context sequence of final species assayed for
NEDD8 and NAEβ (UBA3) mutation designs.

| UBA3 Mutation | | | |
|---|---|---|---|
| Assay | PCR primer 1 | PCR primer 2 | Extend primer |
| G201V | ACGTTGGATGGTGGT | ACGTTGGATGAGCCC | AAAACCTTCTGTCCCC |
| | GGATAAAGTTCCAGC | TTCAAATTGTTTTCC | |
| C324Y | ACGTTGGATGCACCT | ACGTTGGATGGAAAC | GCCCTCCCTTCAGCTGTGT |
| | GTATTTTCTCCTCCC | CTATTACCTTGTG | |
| A171T | ACGTTGGATGGAAAG | ACGTTGGATGTGAAG | GGACTGGACTCTATCATC |
| | ATCATCATGCAACTAC | CACAGCAGCAAGAAC | |
| A209K | ACGTTGGATGGCTTT | ACGTTGGATGGTTTT | CAGAATCACCCGGGC |
| | TTTGATTACCTGTGG | CCTAGATATCTCTTC | |
| E204K | ACGTTGGATGGTGGT | ACGTTGGATGAGCCC | tGGCATTTCCTTTAAAACCTT |
| | GGATAAAGTTCCAGC | TTCAAATTGTTTTCC | |
| Y228H | ACGTTGGATGGATTC | ACGTTGGATGGCAGG | TGCACGCTGGAACTT |
| | TGCCTGGAATGACTG | TGCTTTTTTGATTAC | |
| A171D | ACGTTGGATGGAAAG | ACGTTGGATGTGAAG | GACTGGACTCTATCATCG |
| | ATCATCATGCAACTAC | CACAGCAGCAAGAAC | |
| NEDD8 Mutation | | | |
| Assay | PCR primer 1 | PCR primer 2 | Extend primer |
| I144T | ACGTTGGATGTGTCT | ACGTTGGATGTGATC | AACAGCAGAGGCTCA |
| | CTCTAAAGGTGGAGC | CACCTCAGTACGTGC | |

Next Generation Sequencing (NGS) Methodology

Targeted NGS using the Illumina platform was used to confirm and identify low frequency mutations of NAEβ. Primer pairs were designed to amplify NAEβ coding exons 8, 9, 11, and 13. PCR products were quantified using a PicoGreen assay and combined in equal molar ratios for each sample. The purified products were end-repaired and concatenated by ligation. The concatenated products were used for Hi-Seq 2000 library preparation. The concatenated PCR products were sheared and used to make barcoded Hi-Seq 2000 libraries consisting of 12 bar-coded samples per multiplexed pool. The pooled Hi-Seq 2000 libraries underwent clonal amplification by cluster generation on eight lanes of a Hi-Seq 2000 flow cell and were sequenced using 1×100 single-end sequencing on a Hi-Seq 2000. Over 50,00× coverage was generated for the target bases of all the samples. Matching of primary sequencing reads to the human genome build Hg18, as well as SNP analysis were performed using Illumina's CASAVA software version 1.7.1.

Results

DNA sequencing revealed no treatment emergent DNA mutations in NAE1, UBC12 or NEDD8; however, heterozygous mutations in NAEβ were detected by Sanger sequencing in all resistant cell lines (Table 4).

TABLE 4
Mutational status of NAEβ, NAE1, NEDD8 and UBC12 in HCT-
116, Calu-6, NCI-H460 WT and resistant cell line clones

| | Genes Sequenced | | | | |
|---|---|---|---|---|---|
| | NAEb | | NAE | | |
| Cell line | Sanger | Sequenom | alpha | NEDD8 | UBC12 |
| HCT-116 | WT | WT | WT | I44T | WT |
| parental | | | | | |
| HCT-116.1 | A171T | A171T | WT | I44T | WT |
| HCT-116.2 | A171T | A171T | WT | I44T | WT |
| HCT-116.3 | A171T | A171T | WT | I44T | WT |
| HCT-116.4 | C324Y | C324Y | WT | I44T | WT |
| HCT-116.5 | G201V | G201V | WT | I44T | WT |
| HCT-116.6 | A171T | A171T | WT | I44T | WT |
| NCI-H460 | WT | WT | WT | WT | WT |
| parental | | | | | |
| NCI-H460.1 | E204K | E204K | WT | WT | WT |
| NCI-H460.2 | A171D | A171D | WT | WT | WT |
| Calu-6 parental | WT | WT | WT | WT | WT |
| Calu-6.1 | N209K | N209K | WT | WT | WT |

These mutations were confirmed using additional mass spectrometry based and Next Generation sequencing methods (Table 5).

TABLE 5
Mutational status of NAEβ in HCT-116, Calu-6,
NCI-H460 WT and resistant cell line clones.

| | | | | | | Mutant |
|---|---|---|---|---|---|---|
| Cell Line | Sanger | Sequenom | Next Gen Seq | Final Call | WT nt | nt |
| HCT116 WT | WT | WT | WT | WT | — | — |
| HCT116 clone1 | A171T | A171T (54%) | A171T (42%) | A171T | GCC | ACC |
| HCT116 clone 2 | A171T | A171T (52%) | A171T (43%) | A171T | GCC | ACC |
| HCT116 clone 3 | A171T | A171T (54%) | A171T (45%) | A171T | GCC | ACC |
| HCT116 clone 4 | C324Y | C324Y (54%) | C324Y (47%) | C324Y | TGT | TAT |
| HCT116 clone 5 | G201V | G201V (53%) | G201V (53%) | G201V | GGG | GTG |
| HCT116 clone 6 | A171T | A171T (51%) | A171T (50%) | A171T | GCC | ACC |
| H460 WT | WT | WT | WT | WT | — | — |
| H460 clone 1 | E204K | E204K (51%) | E204K (47%) | E204K | GAA | AAA |
| H460 clone 2 | A171D | A171D (58%) | A171D (45%) | A171D | GCC | GAC |
| CALU6 WT | WT | WT | WT | WT | — | — |
| CALU6 clone1 | N209K | N209K (36%) | N209K (26%) | N209K | AAT | AAA |

Isolated DNA was subjected to Sanger, Sequenom and Next Generation sequencing where indicated. Codon change and amino acid change is indicated.
nt = not tested.

The location of the mutations would be consistent with modification of MLN4924 binding in the nucleotide binding pocket (A171T and A171D) or in affecting the ability of NEDD8 to bind to NAEβ (G201V, E204K, N209K, C324Y). Of note, a heterozygous mutation in NEDD8 (I44T) was detected in wild type HCT-116 cells that is maintained in the resistant clones. However, the activity of this NEDD8 mutant was identical to wild type NEDD8 in biochemical assays (data not shown).

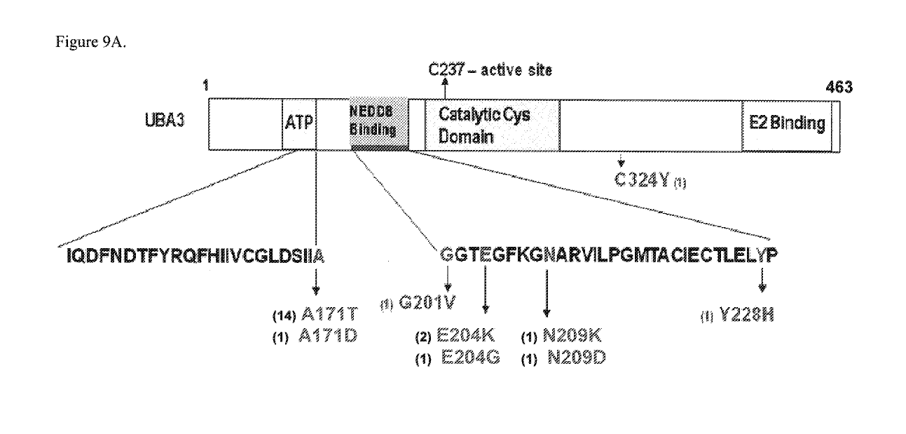

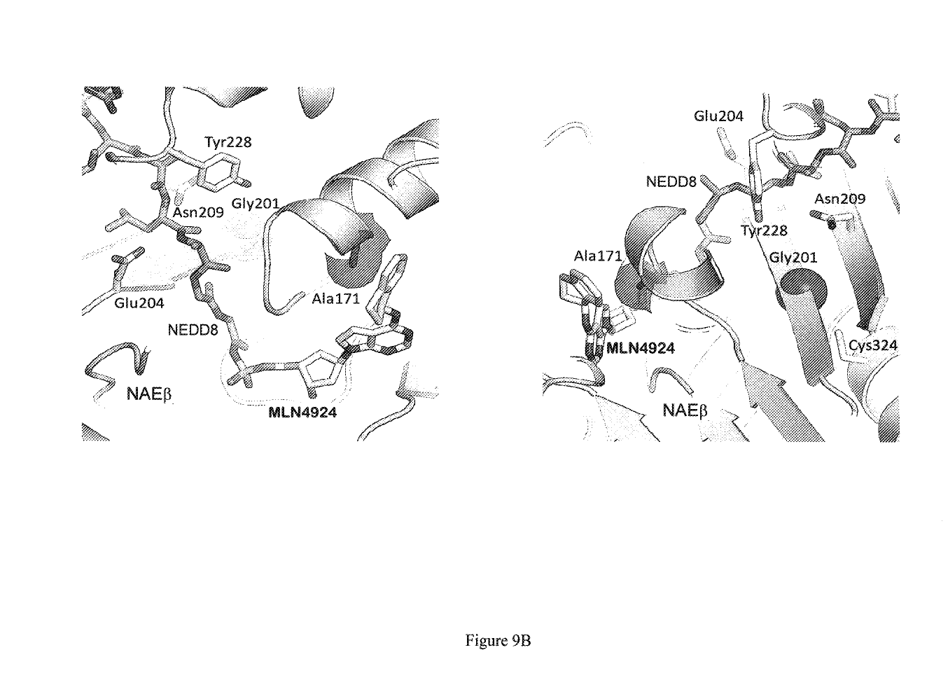

The Y228H mutation corresponds with a residue previously shown to be important for “clamping” the C-terminus of NEDD8 into the adenylation domain and mutation of Y228 has been previously shown to diminish NEDD8 adenylation (Walden et al., 2003). This would suggest that this mutant enzyme is inefficient for NEDD8-activation. In addition, the mutation detected at C324Y is in a region of NAEβ that may also impact NEDD8 binding through structural perturbation of the NEDD8 binding cleft (see FIG. 9B). Interestingly, Alanine at position 171 is conserved in most E1 activating enzymes including UBA1, UBA6 and Sumo-activating enzyme suggesting that selective inhibitors of these enzymes may also be susceptible to the same resistance mechanism.

### Example 3

### Pathway Analysis of Mutant Cell Lines

Western Blot Analysis

Whole cell extracts were prepared and immunoblotting assays performed as previously described (Soucy, 2009) with primary antibodies as follows: CDT1, NRF2, NEDD8, NAEβ, UBC12 and 4924-NEDD8-ADS (MIL22) (Millennium); UBC10 (Boston Biochem); K48 (Millipore); NAE1 (sigma) and tubulin (Santa Cruz). Secondary Alexa-680-labelled antibodies to rabbit/mouse IgG (Molecular Probes) were used as appropriate and blots were imaged using the Li-Cor Odyssey Infared Imaging system.

Cell Cycle Analysis

Cell cycle analysis was performed as previously described (Soucy, 2009).

Logarithmically growing cells were incubated with either MLN4924 or DMSO for the times indicated. Collected cells were fixed in 70% ethanol and stored overnight at 4° C. Fixed cells were centrifuged to remove ethanol, and the pellets were resuspended in propidium iodide and RNaseA in PBS for 1 h on ice protected from light. Cell-cycle distributions were determined using flow cytometry (FACS Calibur, Becton Dickinson) and analysed using Winlist software (Verity).

Results

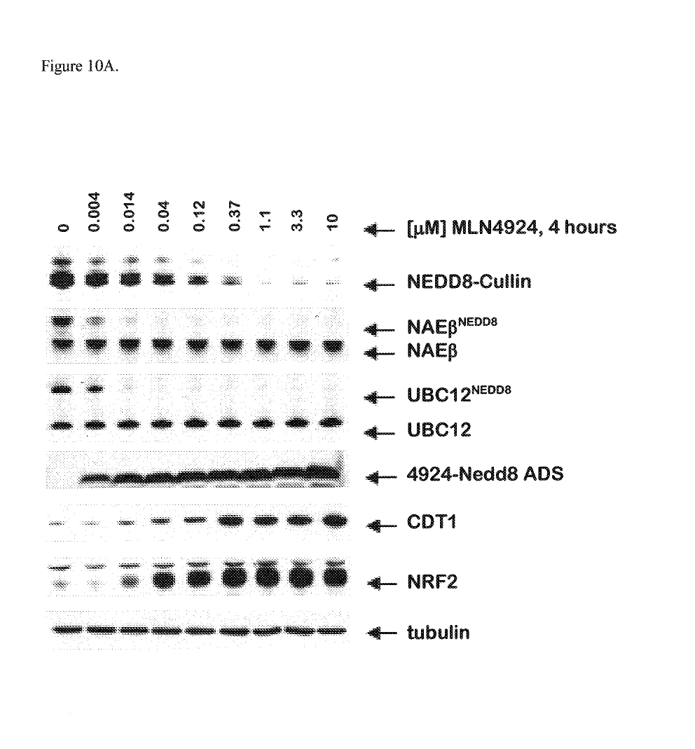

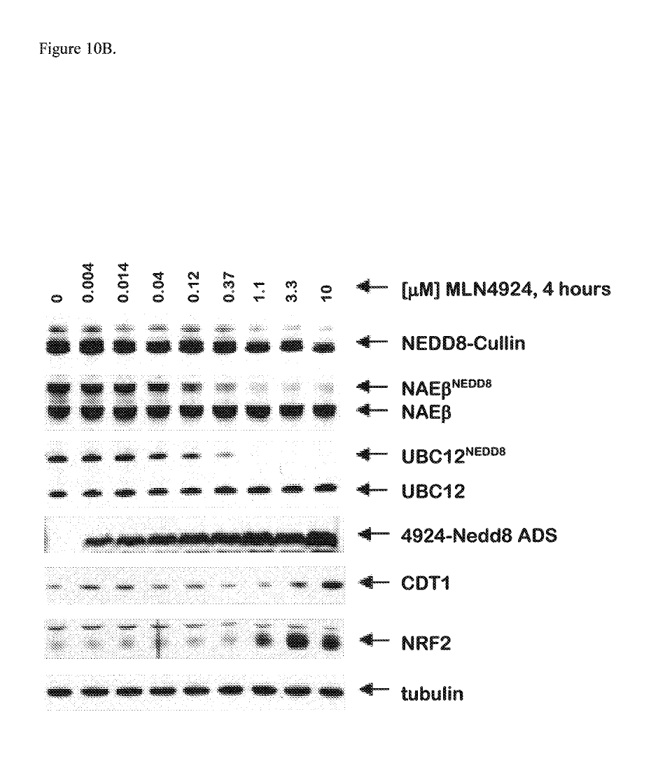

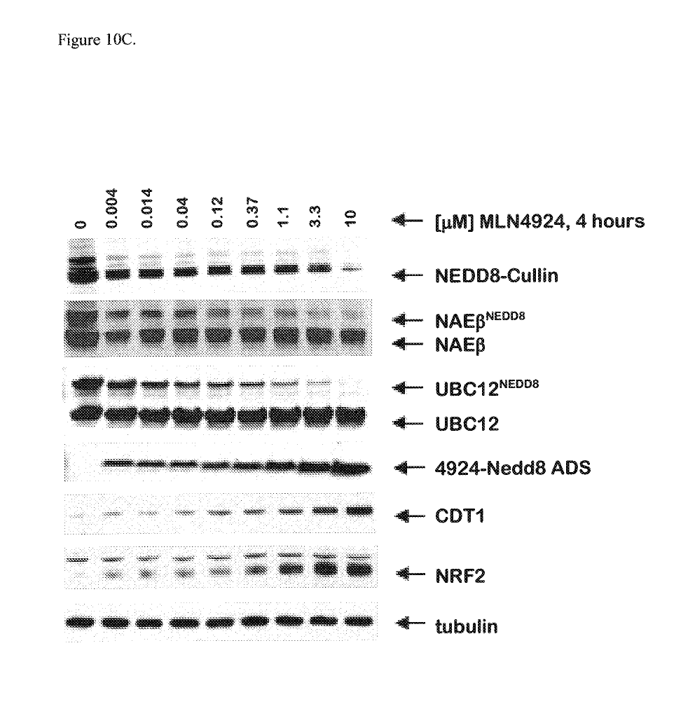

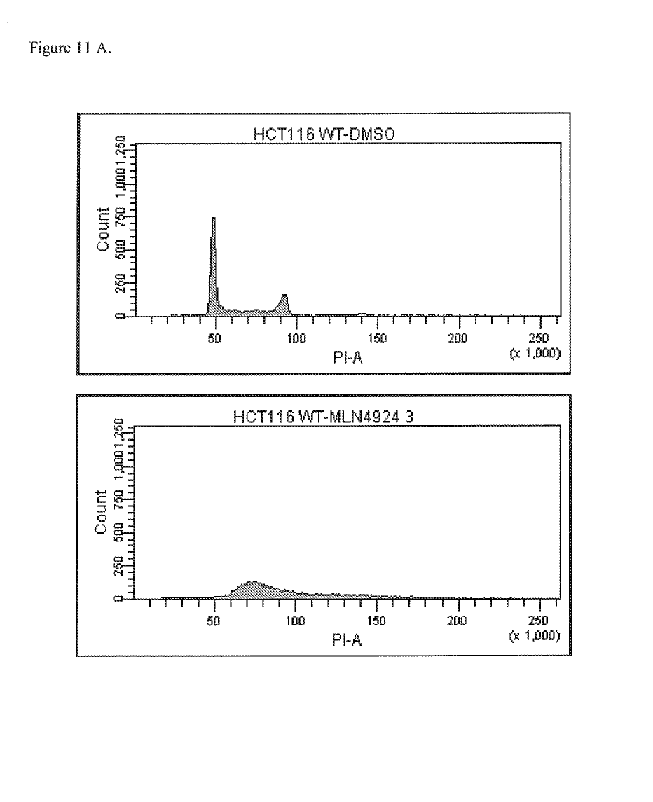

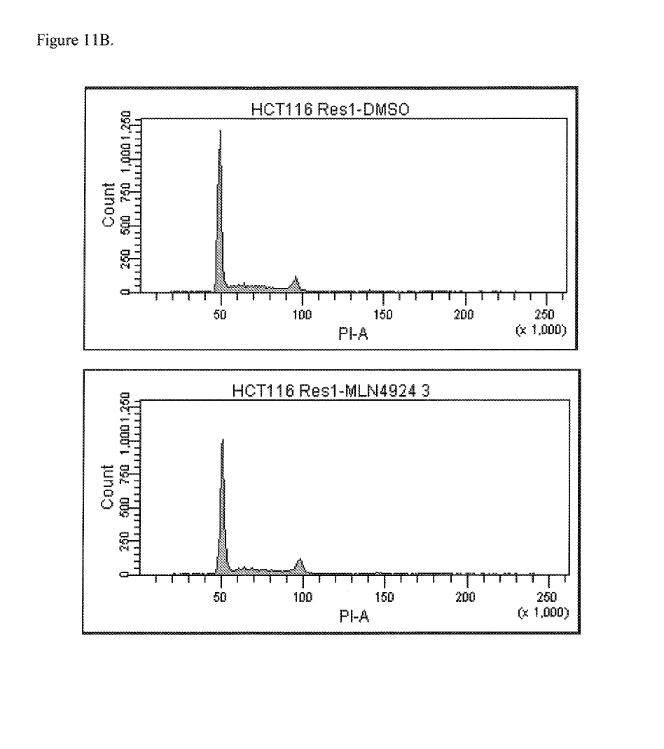

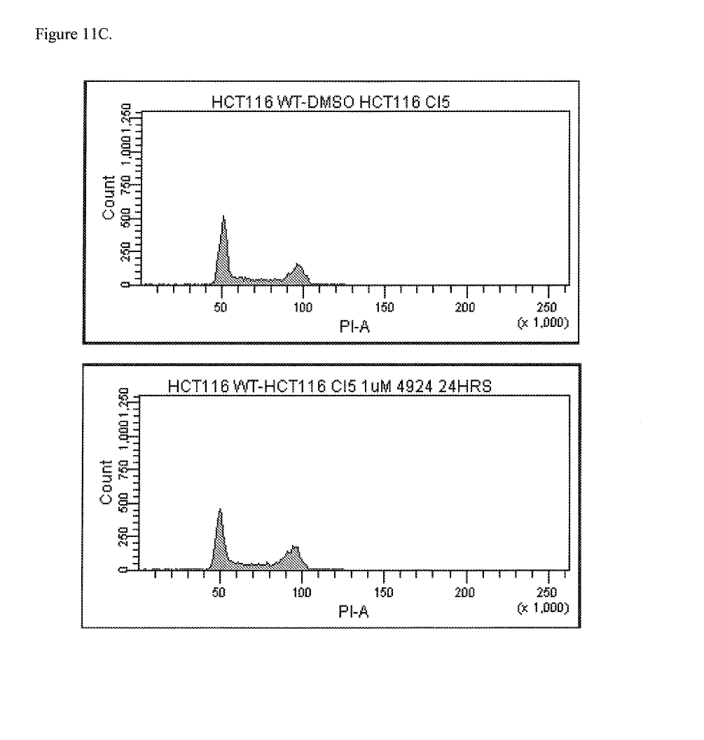

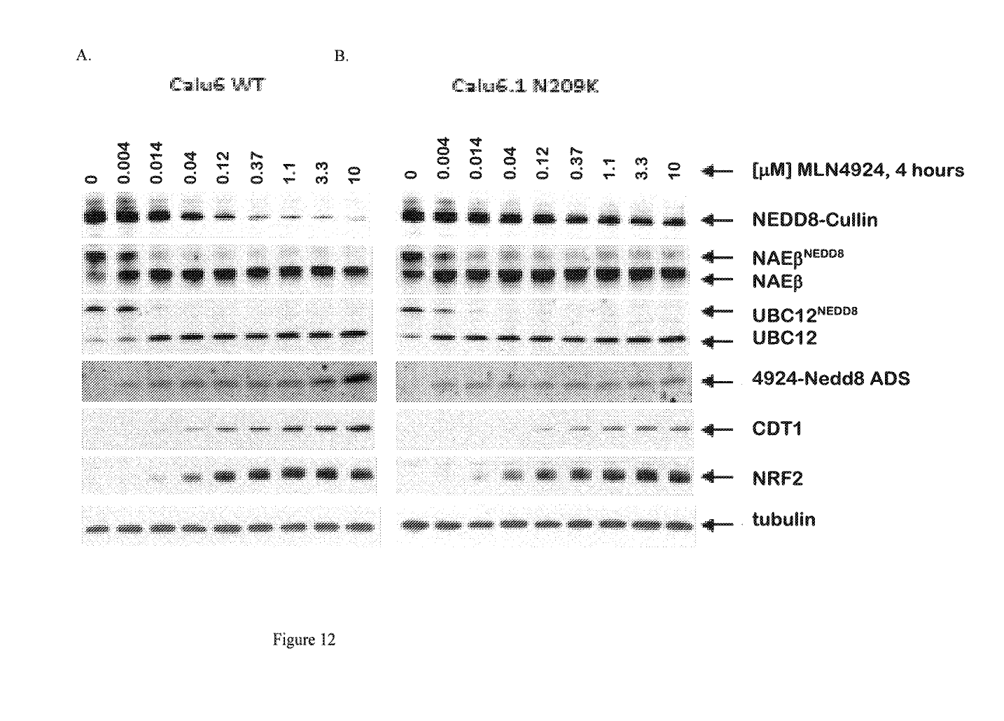

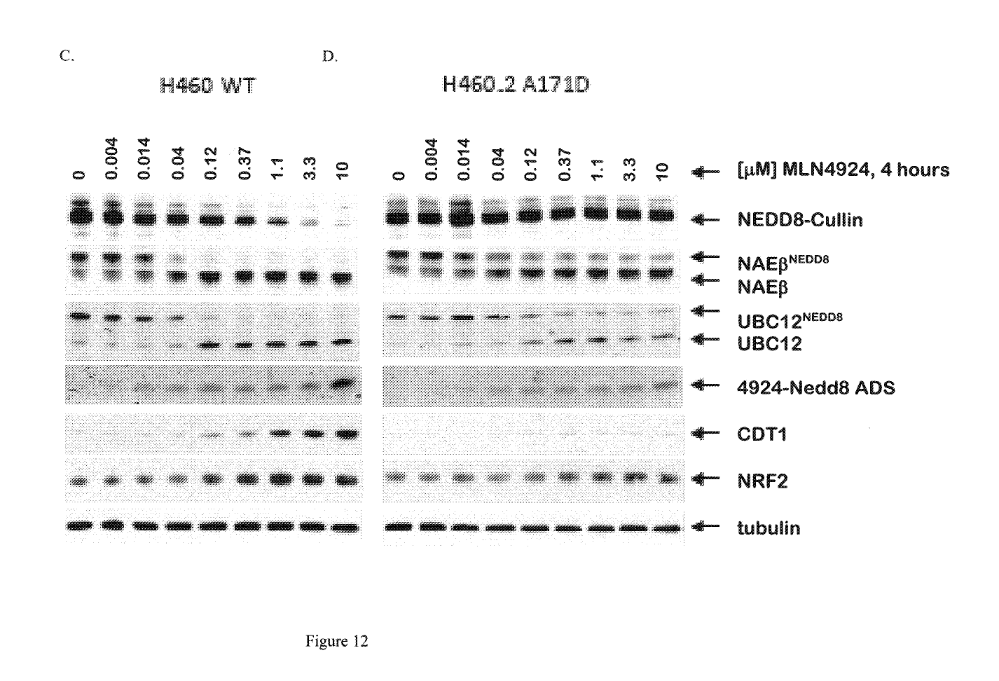

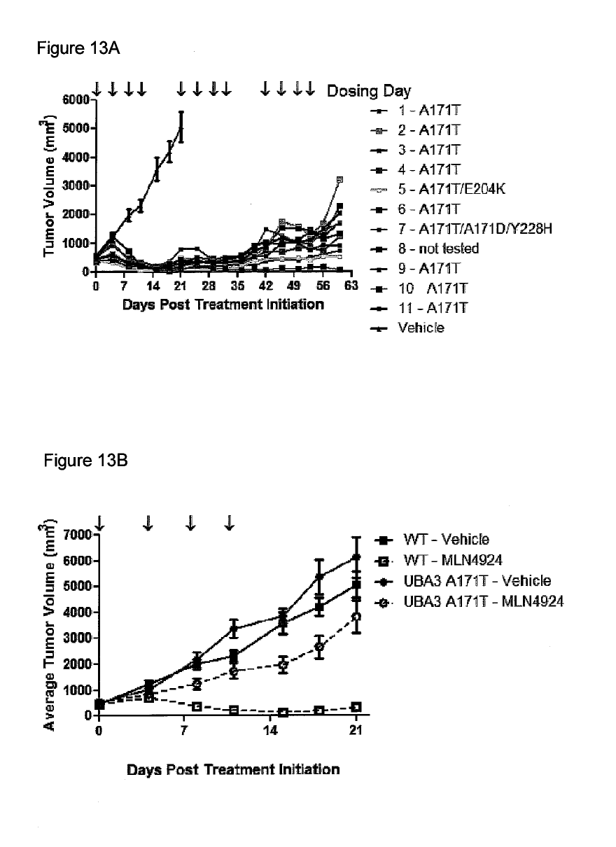

Pathway analysis by Western Blotting was performed following a 4 hour incubation of compound to assess the effect of MLN4924 on the NEDD8 pathway in the resistant clones. Two HCT-116 clones were selected for analysis (one A171T and G201V) and a reduced effect of inhibition of NEDD8 conjugation to NAEB, UBC12 and the cullin proteins was demonstrated when compared to WT cells (FIGS. 10A-C). The reduced effect on NAE inhibition was confirmed by a reduced accumulation of two CRL substrates, Nrf2 and Cdt1. As expected, NEDD8-MLN4924 adduct can still be detected in the resistant cell lines since there is still one wild type copy of NAEβ. MLN4924 induces DNA re-replication in HCT-116 cells (Milhollen et al., 2011), yet the cell cycle distribution of HCT-116 resistant A171T and G201V clones treated wth MLN4924 is not significantly altered consistent with their insensitivity to MLN4924 (FIGS. 11A-C). Similar effects of reduced potency of MLN4924 on the neddylation pathway and CRL substrate accumulation were observed in NCI-H460 A171D and Calu-6 N209K clones (FIGS. 12A-D).

These data demonstrate that in vitro derived MLN4924 resistant cell lines contain heterozygous mutations in NAEβ and as a result are less sensitive to NAE inhibition.

### Example 4

### HCT116 Xenografts Become Resistant to Antitumor Effects of MLN4924, Demonstrate Reduced Pharmacodynamic Effects and Contain Mutations in NAEβ

Immunocompromised Rat and Mouse Antitumor Studies

Female NCr Nude rats (Taconic Farms) aged 6-8 weeks were inoculated with 10×10⁶ HCT-116 cells subcutaneously in the right flank. Tumor growth was measured using digital vernier calipers. When mean tumor growth reached approximately 500 mm³ rats were assigned randomly to treatment groups and dosed subcutaneously with vehicle (20% hydroxypropyl-beta-cyclodextrin) or MLN4924. Rats received one dose per day biweekly for 2 weeks (days 1, 4, 8, and 11) of a 21-day therapy cycle. After three cycles of treatment refractory tumors were collected. Female CB-17 SCID mice (Charles River Laboratories) aged 6-8 weeks were inoculated with 2×10⁶ THP-1 or OCI-Ly10 cells with Matrigel™ support (1:1, v/v). When mean tumor growth reached 200 mm³, mice were assigned randomly to treatment groups and dosed subcutaneously with vehicle (20% hydroxypropyl-beta-cyclodextrin) or MLN4924. Mice received two doses per day bi-weekly (days 1, 4, 8, 11, 15, 18, 22 . . . ) until tumors reached approximately 500-800 mm³. Tumors were then collected and one 40-50 mg piece of tumor was subcutaneously implanted using a 13 gauge trocar needle into 6 to 8 naïve animals for further study. In addition tumor DNA was extracted for mutational analysis as described in supplementary information.

Isolation and Characterization of Resistance cDNA Sequences was performed as in Example 2.

Pharmacodynamic Marker Analysis

Mice and rats bearing HCT-116, THP-1 or OCI-Ly10 tumours were administered a single MLN4924 dose, and at the indicated times tumours were excised and extracts prepared. The relative levels of NEDD8-cullin and pIκBα were estimated by quantitative immunoblot analysis (Li-cor Odyssey system) using Alexa680-labelled anti-IgG (Molecular Probes) as the secondary antibody. For the analysis of CDT1 and cleaved caspase-3 levels in tumor sections, formalin-fixed, paraffin-embedded tumour sections were stained with the relevant antibodies, amplified with HRP-labelled secondary antibodies and detected with the ChromoMap DAB Kit (Ventana Medical Systems). Slides were counterstained with haematoxylin. Images were captured using an Eclipse E800 microscope (Nikon Instruments) and Retiga EXi colour digital camera (QImaging) and processed using Metamorph software (Molecular Devices). CDT1 and cleaved caspase-3 are expressed as a function of the DAB signal area.

Measurement of NEDD8-MLN4924 Adduct Levels in Tumor Xenografts

To quantify the absolute level of NEDD8-MLN4924 adduct in tumor xenografts, 30 μg total protein of each lysate sample was mixed with 0.1 pmol (0.9 ng) NEDD8*-MLN4924 followed by NuPAGE Bis-Tris 4-12% SDS gel separation (Invitrogen); NEDD8 gel fractions were excised and in-gel tryptic digestion was performed as described (Brownell et al., 2010). The digests were analyzed on a LC/MS/MS system as previously described (Brownell et al., 2010). The NEDD8-MLN4924 adduct amount in each sample was calculated from the ratio of the peak areas of Gly-Gly-MLN4924 to Gly*-Gly*-MLN4924 in the chromatogram.

Results

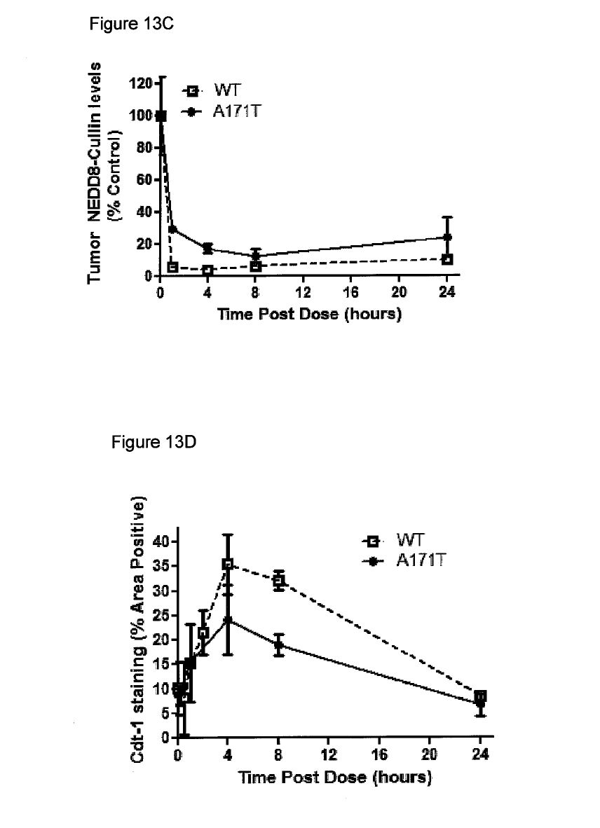

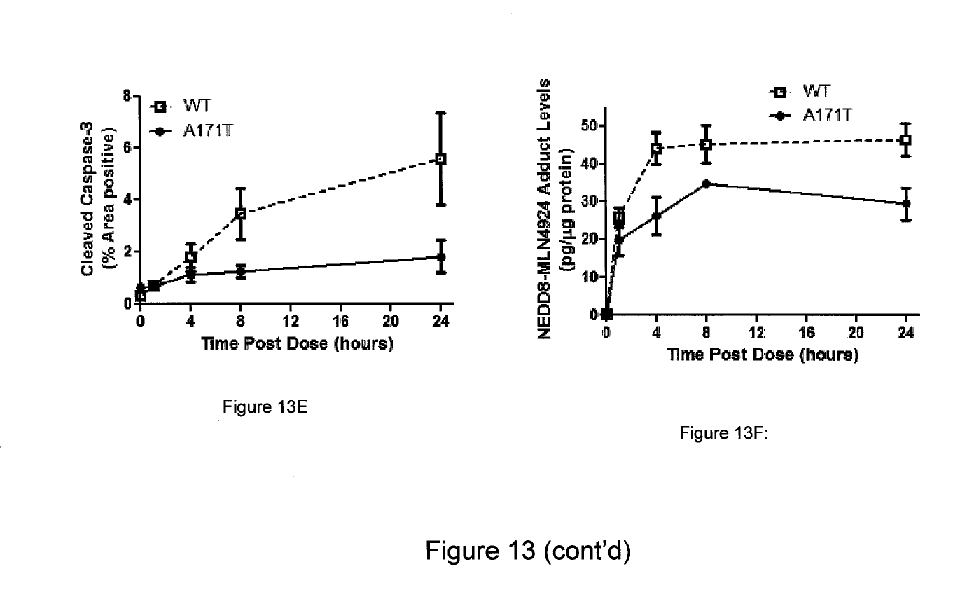

HCT-116 cells were grown as subcutaneous xenografts in immuncompromised rats then treated with the maximum tolerated dose of MLN4924 (150 mg/kg) subcutaneously on a dosing schedule of days 1, 4, 8 and 11 of a 21 day therapy cycle. Importantly, this regimen was chosen as it is currently being utilized in Phase I clinical studies of MLN4924 in solid and hematological malignancies. After the first cycle of MLN4924 treatment tumor regressions were observed that were maintained through most of the second cycle of treatment (FIG. 13A). However, during cycle 3 all but one tumor began to re-grow even in the presence of MLN4924 administration (FIG. 13A). Tumors were harvested at the end of treatment and the nucleic acid sequence of NAEβ was analyzed. Eight of ten tumors were found to contain a heterozygous mutation at A171T of NAEβ, one contained a heterozygous mutation at Y228H and the remainder was wild type for NAEB (FIG. 13A and Table 6). Interestingly, in two tumors more than one mutation was detected indicating that multiple clones may emerge within a tumor population. The xenograft with no detectable NAEβ mutations had the least re-growth consistent with the association of an NAEβ mutation and resistance. No mutations were detected in NAEα or UBC12 and the I44T mutation within NEDD8 was detected consistent with cells grown in vitro.

TABLE 6
Mutational status of resistant HCT-116, OCI-Ly10 and THP-1 xenografts.

| Animal | | | Next Gen | | | |
|---|---|---|---|---|---|---|
| Identification | Sanger | Sequenom | Sequencing | Final Call | WT nt | Mutant nt |
| HCT116 #5 | WT | A171T (23%) | A171T (13%) | A171T | GCC | ACC |
| HCT116 #5 | WT | E204K (24%) | E204K (23%) | E204K | GAA | AAA |
| HCT116 #2 | A171T | A171T (46%) | nt | A171T | GCC | ACC |
| HCT116 #10 | A171T | A171T (49%) | A171T (46%) | A171T | GCC | ACC |
| HCT116 #7 | Y228H | Y228H (40%) | Y228H (33%) | Y228H | TAT | CAT |
| HCT116 #7 | WT | A171D (21%) | A171D (10%) | A171D | GCC | GAC |
| HCT116 #7 | WT | A171T (13%) | A171T (7%) | A171T | GCC | ACC |
| HCT116 #3 | A171T | A171T (49%) | nt | A171T | GCC | ACC |
| HCT116 #1 | A171T | A171T (38%) | nt | A171T | GCC | ACC |
| HCT116 #9 | A171T | A171T (49%) | nt | A171T | GCC | ACC |
| HCT116 #11 | A171T | A171T (50%) | nt | A171T | GCC | ACC |
| HCT116 #6 | A171T | A171T (33%) | nt | A171T | GCC | ACC |
| HCT116 #4 | A171T | A171T (38%) | nt | A171T | GCC | ACC |
| THP-1 #6 | WT | WT | WT | WT | — | — |
| THP-1 #4 | WT | A171T (13%) | A171T (13%) | A171T | GCC | ACC |
| THP-1 #7 | WT | nt | N209D (25%) | N209D | AAT | GAT |
| THP-1 #8 | A171T | A171T (51%) | A171T (51%) | A171T | GCC | ACC |
| THP-1 #9 | A171T | A171T (54%) | A171T (50%) | A171T | GCC | ACC |
| THP-1 #10 | WT | WT | WT | WT | — | — |
| LY10 #5 | E204G | nt | E204G (50%) | E204G | GAA | GGA |

Isolated DNA was subjected to Sanger, Sequenom and Next Generation sequencing where indicated.
Codon change and amino acid change is indicated.
nt = not tested.

To confirm that the tumors were now stably resistant to MLN4924 one xenograft with an A171T mutation was re-transplanted into nude rats. The antitumor activity of MLN4924 was dramatically reduced compared to wild type xenografts, one cycle of 150 mg/kg MLN4924 (days 1, 4, 8, 11) inhibited tumor growth by only 38% compared to 94% (including tumor regressions) that were observed in wild type xenografts (FIG. 13B). The A171T NAEβ mutation was still present in this resistant xenograft model as confirmed by both Sanger and Sequenom methodologies. Pharmacodynamic analysis of NEDD8 pathway inhibition by MLN4924 in WT HCT-116 and A171T HCT-116 resistant xenografts was conducted following a single dose of 150 mg/kg MLN4924. Maximal inhibition of NEDD8-cullin levels occurred as early as 1 hour post dose in WT HCT-116 xenografts compared to 8 hours post-dose in the A171T resistant model (FIG. 13C). In agreement with reduced effects on NEDD8-cullin levels in A171T HCT-116 was a reduction in the levels of Cdt1 (FIG. 13D) and apoptosis as measured by cleaved caspase-3 (FIG. 13E) and NEDD8-MLN4924 adduct (FIG. 13F), compared to WT HCT-116 xenografts. These data demonstrate the tumor xenografts treated with a clinically relevant dosing schedule can acquire resistance to MLN4924 that is associated with mutations in NAEβ.

Since NAEβ mutations appear the most common cause of resistance in our studies, irrespective of the cellular outcome following NAE inhibition, this confirms the selectivity of MLN4924 for its target. We did observe the re-growth of THP-1 AML xenografts that do not contain a mutation in NAEβ suggesting that other mechanisms of resistance to MLN4924 are also likely to occur.

### Example 5

### Treatment Emergent Mutations in NAEβ are Observed in Acute Myelogenous Leukemia and Diffuse Large B-Cell Lymphoma Xenografts

Methods

See Example 4.

Results

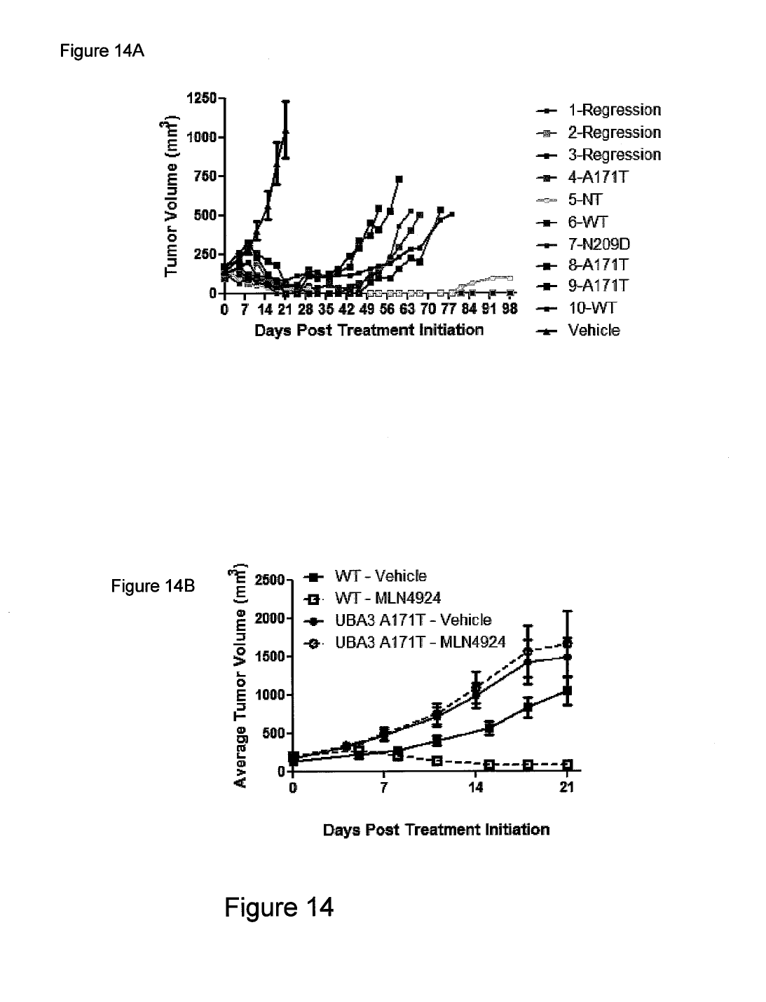

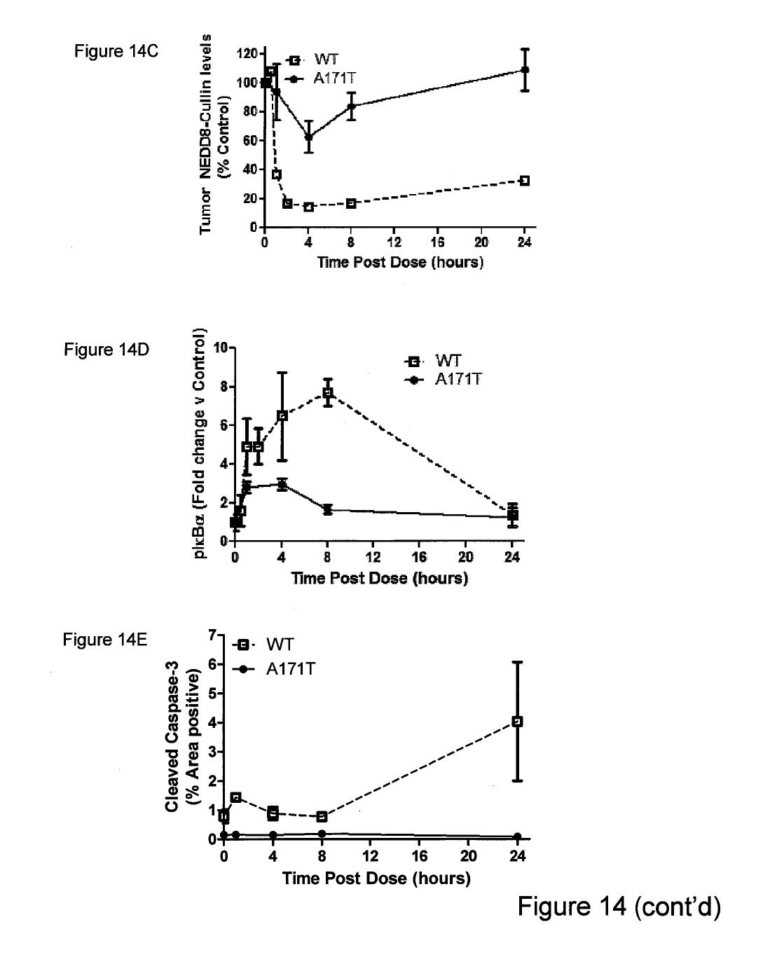

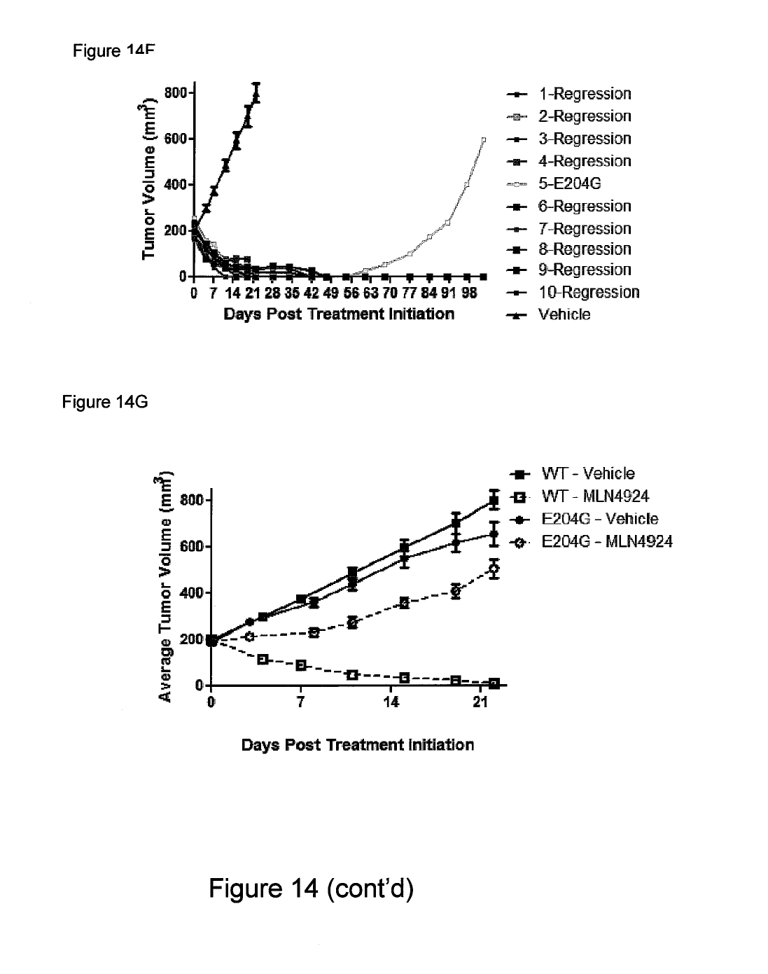

MLN4924 has shown clinical activity in patients with Acute Myelogenous Leukemia (AML) (Wang et al., 2011). Therefore we utilized THP-1 cells, a relevant subcutaneous AML pre-clinical model, to determine whether resistance to MLN4924 could be driven by NAEβ mutations. The subcutaneous model was utilized to facilitate harvesting tumors for subsequent analysis. MLN4924 was administered to SCID mice bearing THP-1 xenografts (90 mg/kg BID, biweekly) and uniform tumor regressions were observed (FIG. 14A). Six THP-1 xenografts re-grew during the MLN4924 treatment period and were harvested for analysis. Three THP-1 xenografts contained a heterozygous mutation in NAEB at A171T, one contained a heterozygous mutation in at N209D and the remaining two were wild type for NAEB and so may be refractory through an alternate mechanism (FIG. 14A and Table 6). Again no mutations were observed in NAE1, UBC12, or NEDD8. One A171T THP-1 xenograft was successfully re-established in SCID mice and shown to be resistant to MLN4924 when dosed at 90 mg/kg BID bi-weekly (FIG. 14B). Pharmacodynamic evaluation in A171T THP-1 xenografts showed that MLN4924 produced minimal inhibition of NEDD8-cullin levels (FIG. 14C) resulting in a reduced elevation of the CRL substrate pIκBα (FIG. 14D) and a failure to activate apoptosis (FIG. 14E) in comparison to WT THP-1 xenografts.

We have previously shown that Activated B-Cell like Diffuse Large B-cell lymphomas (ABC-DLBCL) may be particularly sensitive to MLN4924 through inhibition of constitutively active NF-κB signaling (Milhollen et al., 2010). To determine whether resistance to MLN4924 through NAEβ mutations occurs in models where re-replication does not drive the terminal outcome, we followed OCI-Ly10 xenograft bearing mice administered 90 mg/kg BID MLN4924 bi-weekly for over 100 days. Consistent with our previous findings MLN4924 induced tumor regressions in the OCI-Ly10 model (FIG. 14F). Only one tumor re-grew during therapy and subsequent analysis revealed a heterozygous mutation in NAEβ at E204G. The tumor was re-implanted and, as with the other re-introduced resistant tumors, was resistant to MLN4924 treatment at 90 mg/kg BID (FIG. 14G). These data demonstrate that regardless of the MLN4924-dependent terminal outcome or genetic background certain NAEβ mutations can drive resistance to MLN4924.

MLN4924 induces DNA damage in cells via re-replication (Milhollen et al., 2011) and this mechanism of inducing hyper-replication of DNA may aid in the development of resistance through increased random mutagenesis. HCT-116, H460 and Calu-6 cells all undergo DNA re-replication whereas OCI-Ly10 cells do not (Milhollen et al., 2010). We were able to detect an NAEβ mutation in the OCI-Ly10 model suggesting that resistant mutants can emerge irrespective of the mechanism of action of MLN4924. HCT-116 cells have a deficiency in the DNA mismatch repair protein MLH1 (Taverna et al., 2000) and this may make them more susceptible to resistance mutations by virtue of increased genomic instability. This assertion may be supported by the number of mutations detected in HCT-116 cell lines and xenografts compared to others used in these studies that do not possess the same defect in DNA repair.

### Example 6

### Mutations in the Nucleotide Binding Pocket and NEDD8 Binding Cleft of NAEβ Affect MLN4924 Adduct Formation and Dissociation from NAEβ

Biochemical Characterization of NAEβ and Mutant Enzymes

Materials

[32P]-PPi (Cat. No. NEX019), [α-32P]-ATP (Cat. No. BLU003H250UC), and [α-32P]-ATP (Cat. No. BLU002250UC) were obtained from Perkin Elmer (Boston, Mass., USA). Other chemicals were purchased from Sigma. N-terminally FLAG-tagged NEDD8 with the sequence of N-MDYKDDDDK-NEDD8 was expressed and purified as described (Soucy et al., 2009). Untagged NEDD8 and N15 and C13 labeled untagged NEDD8 (NEDD8*) was expressed and purified similarly. N-terminal GST-tagged UBC12 was expressed and purified as described (Soucy et al., 2009). His-tagged NAE proteins (NAE1 and His-tagged NAEβ WT and mutants) were cloned into Rosetta (DE3) cells and complexes were generated by co-expression into E. coli. Expressed proteins were purified by affinity (Ni-NTA agarose, Qiagen) or conventional chromatography. GST tagged NAE proteins (NAE1 and GST-tagged NAEβ WT and mutants) were generated by co-infection of Sf9 cells (Soucy et al., 2009). GST-NAE proteins were purified by affinity chromatography (Glutathione Sepharose 4B, GE Healthcare) followed bi HiTrap Q HP (GE Healthcare).

Assays

Biochemical characterization and IC₅₀ determinations were performed using an improved pyrophosphate exchange assay developed by Bruzzese, et al. ((2009) Anal. Biochem. 394:24-29). ATP-PPi exchange reactions were performed in buffer containing 50 mM HEPES (pH 7.5), 25 mM NaCl, 10 mM MgCl₂, 0.05% BSA, 0.01% Tween-20 and 1 mM DTT. NEDD8 K_{M}s were determined by serial diluting NEDD8 into a 96-well assay plate containing 10 nM NAE, 1 mM ATP and 0.2 mM PPi (50 cpm/pmol [32P] PPi). Assays were incubated for 30 minutes at 37° C. in a final volume of 50 μL and were stopped with the addition of 500 μl of 5% (w/v) trichloroacetic acid (TCA) containing 10 mM PPi. The quenched reactions were transferred to a dot-blot apparatus fitted with activated charcoal filter paper as described (Bruzzese, et al., 2009). ATP K_{M}s were determined by serial diluting ATP into a 96-well assay plate under similar assay conditions. This time reactions were initiated with addition of NEDD8 (0.16 μM for WT, A171T/D and 2.5 μM for N209K, E204K and G201V). Assays were incubated for 30 minutes at 37° C. PPi titrations were performed under similar conditions, except serial diluting PPi instead and using 1 mM ATP.

IC₅₀s were determine by serial diluting each compound into a 96-well assay plate containing 5 nM NAE, 1 mM ATP and 0.2 mM PPi (50 cpm/pmol [32P] PPi). Reactions were initiated with addition of NEDD8 (0.16 μM for WT, A171T/D and 2.5 μM for N209K, E204K and G201V). Assays were incubated for 60 minutes at 37° C. in a final volume of 50 μL and were stopped as previously described.

The enzyme reversibility assay was run in the FLAG-NEDD8-GST-UBC12 HTRF transthiolation assay described previously (Soucy et al., 2009, Brownell et al., 2010). Final concentrations of each enzyme after dilution were 10 pM WT NAEβ, 12.5 pM A171T NAEβ, 30 pM N209K NAEβ, and 33 pM E204K/G201V NAEβ.

SPR experiments were performed on a Biacore S51 instrument (GE Healthcare, Piscataway, N.J., USA). N-terminal GST-tagged NAE enzyme preps were immobilized on a sensor chip surface using the anti-GST antibody capturing method described (Chen, J., et al. (2011) J. Biol. Chem. 286:40867-40877). The SPR data were collected at 25° C. with a flow rate of 90 μL/min in a sample running buffer containing 10 mM HEPES, 150 mM NaCl, 0.005% P-20 (as surfactant), 0.1 mg/mL BSA, pH 7.5. All data acquisition (in duplicate) and subsequent analysis were performed with recombinant GST as the control. The kinetics of association and dissociation data was fit with a single exponential rise or decay equation. The equilibrium affinity binding data was fit using a one-site binding model.

Results

The NAEβ mutations that have been detected in MLN4924 resistant cells derived in tissue culture or in vivo occur in two areas of the gene, the nucleotide binding pocket at Alanine 171 and at various residues that are within or close to the NEDD8 binding cleft (FIG. 9A). Mutations at Alanine 171 appear to be a “hot-spot” since approximately two-thirds of the mutations detected lie at this residue. Structural renderings of the A171T and A171D mutants suggest that decreased potency of MLN4924 could occur through a clash with the indane group of MLN4924 and the bulkier threonine or aspartic acid residue in mutant NAEβ (FIG. 9B). In contrast, mutations found in the NEDD8 binding region of NAEβ are hypothesized to affect the affinity of NEDD8 and in turn the MLN4924-NEDD8 adduct (FIG. 9B). To understand the biochemical consequences of mutations in these regions recombinant enzymes expressing A171T, A171D, N209K, E204K or G201V were constructed as representative of the two classes of mutations. Structural modeling of A171T or A171D did not predict an effect on ATP binding, yet these mutations did result in weaker affinity for ATP. However, the affinity for NEDD8 was largely unaffected by mutations to A171. In contrast, mutations in the NEDD8 binding cleft (N209K and E204K) resulted in an increase in the K_{M} for NEDD8 with minimal affect on the K_{M} for ATP. Interestingly, a mutation at G201V results in an increase in both the K_{M} for ATP and NEDD8. The K_{M} for PPi was unaffected by any of these mutations. In addition, other than the A171D mutant, the catalytic rate (kcat) of the NAE reaction is not severely affected by any of these mutations, in fact it appears the G201V mutant is more catalytically active than wild type enzyme.

We have previously demonstrated that MLN4924 inhibits the NEDD8-NAEβ thioester form of NAE by occupying the nucleotide binding site and forming a covalent adduct between NEDD8 and MLN4924 (Brownell et al., 2010). To determine the effect of inhibitory potency of MLN4924 against WT and mutant enzymes the PPi-ATP assay was performed using a concentration of 1 mM ATP. Interestingly, the A171T mutant was still capable of being inhibited by MLN4924 with only a modest 2-fold decrease in potency compared to WT enzyme (Tables 7 and 8).

TABLE 7
Biochemical characterization of NAEβ mutants

| NAEβ | K_{M} ATP | K_{M} NEDD8 | K_{M} PPi | | Nedd8 K_{D} (SPR) |
|---|---|---|---|---|---|
| Enzyme | (μM) | (μM) | (μM) | k_{cat} (s⁻¹) | (μM) |
| wt | 88 ± 3 | 0.044 ± 0.024 | 22 ± 2 | 1.2 ± 0.2 | 0.23 ± 0.01 |
| A171T | 600 ± 30 | 0.080 ± 0.018 | 22 ± 2 | 1.5 ± 0.4 | 0.41 ± 0.03 |
| A171D | 1,900 ± 20 | 0.016 ± 0.0037 | 10 ± 2 | 0.33 ± 0.08 | 0.22 ± 0.01 |
| N209K | 31 ± 1 | 0.43 ± 0.081 | 24 ± 2 | 2.0 ± 0.1 | >>10 |
| E204K | 21 ± 1 | 0.48 ± 0.053 | 18 ± 1 | 1.3 ± 0.5 | |
| G201V | 820 ± 36 | 3.5 ± 0.22 | 10 ± 1 | 3.7 ± 0.5 | n.d. |

Several NAEβ mutants were characterized in the pyrophosphate exchange assay under saturating conditions at 37° C. and analyzed for parameters of K_{M} for ATP, NEDD8, PPi and catalytic activity. The K_{M} for each substrate was determine by titrating each substrate into the PPi-ATP assay and fitting using the standard Michaelis-Menten equation, y = Vₘₐₓ* [S]/ K_{M} + [S], where ‘y’ is PPi-ATP activity. The standard error was extrapolated from the fit. The k_{cat} for each enzyme was determined under optimal conditions (saturating ATP, PPi and peak [NEDD8]) and using a [α-32P]-ATP standard curve. Each k_{cat} value represents the average and standard deviations of duplicates experiments.

TABLE 8
Potency of E1 Enzyme Inhibitors on NAEβ Mutants

| NAEβ | MLN4924 | Compound 1 | Adenosine Sulfate |
|---|---|---|---|
| Enzyme | IC50 (μM) | IC50 (μM) | IC50 (μM) |
| wt | 0.049 ± 0.011 | 0.0041 ± 0.0002 | 0.0050 ± 0.0002 |
| A171T | 0.10 ± 0.026 | 0.030 ± 0.002 | 0.0080 ± 0.009 |
| A171D | >100 | 70 ± 20 | 0.034 ± 0.004 |
| N209K | 0.78 ± 0.16 | 0.076 ± 0.005 | 0.12 ± 0.013 |
| E204K | 1.6 ± 0.20 | 0.17 ± 0.005 | |
| G201V | 0.51 ± 0.057 | 0.0057 ± 0.0006 | 0.22 ± 0.006 |

IC₅₀ values were determined by titrating compounds into the pyrophosphate exchange assay containing 1 mMATP and 5 nM of wt or mutant NAE. IC₅₀ curves using the average of 4 replicates were fit using a sigmoidal logistics 3-parameter equation, y = a/(1 + ([I]/IC₅₀)^{b}, where ‘y’ is % Inhibition, ‘a’ is amplitude, and ‘b’ is hill slope. Standard errors were extrapolated from the fit.

MLN4924 did not inhibit A171D up to a concentration of 100 μM suggesting that the bulkier aspartic acid residue impedes MLN4924 binding in the nucleotide binding pocket. MLN4924 was approximately 10-fold less active against the NEDD8 binding cleft mutants compared to WT enzyme. Compound 1, a structurally similar N6-substituted adenosine sulfamate, was used for comparison with MLN4924 (Brownell et al., 2010). The potency of Compound 1 by the ATP-PPi assay decreased 7-fold versus A171T mutant and 17,000 fold versus the A171D mutant. A noticeable decrease in potency was also observed with Compound 1 in the NEDD8 binding pocket mutants.

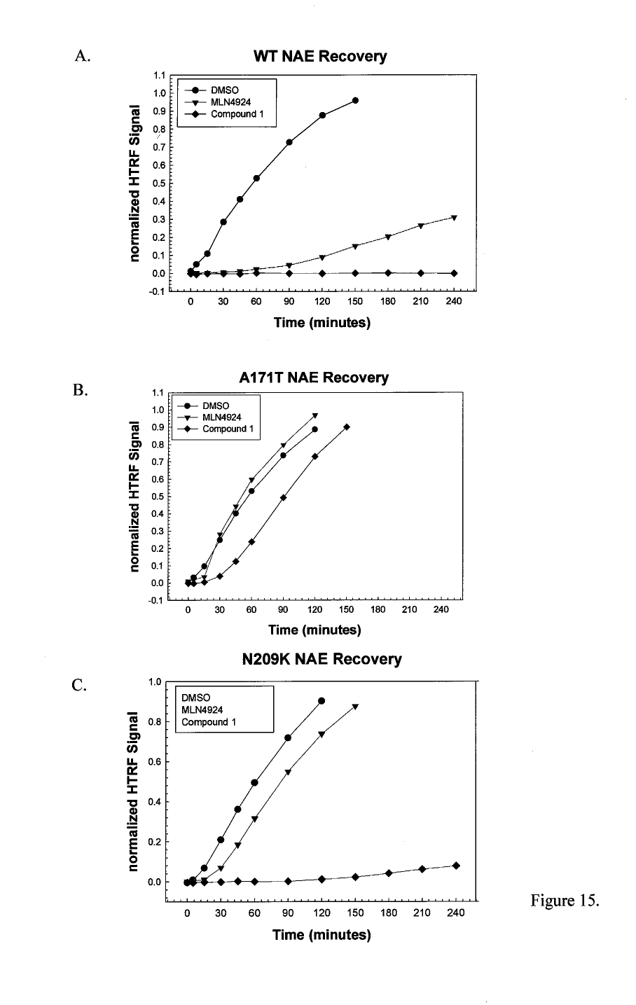

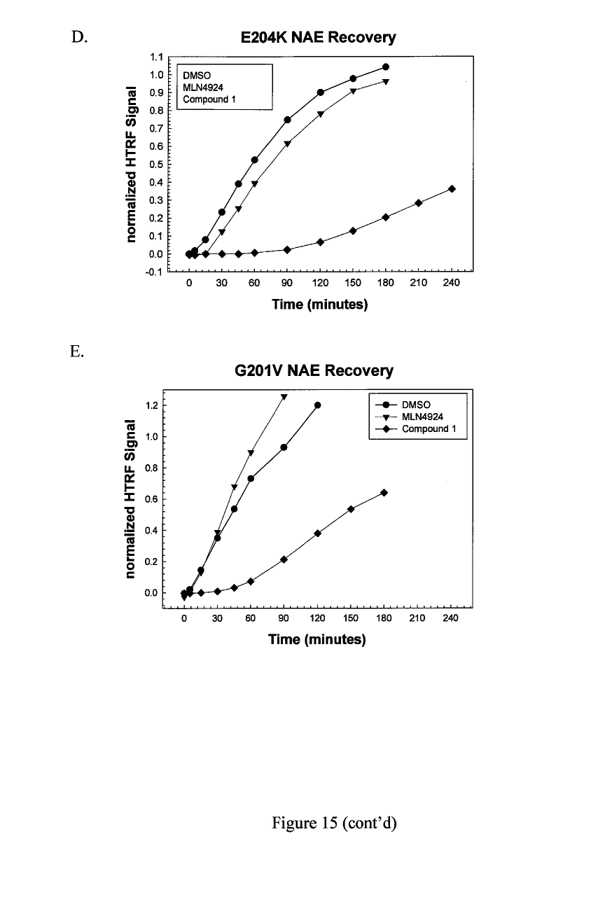

Since in vitro IC₅₀s of enzyme inhibition did not fully explain the resistance to MLN4924 observed, further compound characterization was performed to evaluate the rates of enzyme inactivation and the reversibility of compound inhibition. A171D NAEβ mutant was not used in these studies as it was completely insensitive to MLN4924 inhibition. Enzyme-Inhibitor complexes, NEDD8-MLN4924 or NEDD8-Cpd1 were pre-formed on the enzyme, purified and added to a UBC12 transthiolation reaction to measure the recovery of enzyme activity (FIGS. 15A-E). As previously reported MLN4924-NEDD8 is a tight binding inhibitor of WT NAEβ with enzyme activity recovering to approximately ˜30% of DMSO control levels by 240 mins. In contrast, the recovery of enzymatic activity following MLN4924 inhibition was similar to that of the DMSO control for A171T, N209K, E204K and G201V mutants. These data indicate that although the NEDD8-MLN4924 adduct is formed by the mutant enzymes, it no longer is a tight binding inhibitor. Compound 1-NEDD8 adduct binds more tightly to WT NAEβ than MLN4924-NEDD8 adduct with no discernable recovery of enzymatic activity at 240 minutes. In addition, Compound 1-NEDD8 adduct appears a tighter binder of all mutant NAE enzymes suggesting compound 1 may be a more potent inhibitor of mutant enzyme in cells compared to MLN4924. (see Table 9 for a summary of binding kinetics and Table 10 for half life of dissociation)

TABLE 9
Summary of Binding Kinetics of UBA3 mutants

| | NEDD8- | | NEDD8- | |
|---|---|---|---|---|
| | adenosine sulfamate | | MLN4924 | |
| | kₒₙ | | kₒₙ (×10⁶ | |
| Enzyme | (×10⁶ M⁻¹s⁻¹) | k_{off} (s⁻¹) | M⁻¹s⁻¹) | k_{off} (s⁻¹) |
| WT | To be measured | | 5.10 ± 0.04 | <1.4 × 10⁻⁵** |
| A171T | 5.12 ± 0.03 | <1.4 × 10⁻⁵** | 2.82 ± 0.10 | ~4 × 10⁻⁴ |
| A171D | 5.59 ± 0.10 | 0.023 ± 0.003 | 3.32 ± 0.04 | 0.15 ± 0.01 |
| E204K | 5.22 ± 0.15 | ~3 × 10⁻⁵ | 3.10 ± 0.07 | ~1 × 10⁻⁴ |
| N209K | 5.67 ± 0.15 | <1.4 × 10⁻⁵** | 1.22 ± 0.03 | ~6 × 10⁻⁵ |

**The detection limit for k_{off} measurement is about 1 Response Unit (RU) change in 1 h, or 1.4 × 10⁻⁵ s⁻¹ for NEDD8 with RU of ~20.

TABLE 10
Dissociation half-life analysis of purified NEDD8-adducts against
NAEβ mutants using SPR

| NAEβ | Nedd8-MLN4924 | | Nedd8-compound 2 | |
|---|---|---|---|---|
| samples | t_{1/2} | | t_{1/2} | |
| wild-type | >20 | h | >20 | h |
| A171T | 0.48 | h | >20 | h |
| A171D | 4.6 | s | 30 | s |
| N209K | 3.2 | h | >20 | h |

Assays performed in the absence of ATP. t_{1/2} calculated from k_{off} in Table 9.

The rate of enzyme inactivation by MLN4924 for both A171T and N209K mutants was also dramatically slower compared to WT as opposed to Compound 1 where the rate of WT and mutant enzyme inactivation was more rapid than MLN4924 (Table 11).

TABLE 11
Rate of inactivation of MLN4924, Compound 1 vs. recombinantly
purified NAEβ mutants

| | | | Adenosine |
|---|---|---|---|
| NAEβ | MLN4924 | Compound 1 k_{inact}/Kᵢ | sulfamate |
| Enzyme | k_{inact}/Kᵢ (M⁻¹ s⁻¹) | (M⁻¹ s⁻¹) | k_{inact}/Kᵢ (M⁻¹ s⁻¹) |
| wt | 7,500 ± 2000 | 56,000 ± 2200 | 25,800 ± 3000 |
| A171T | 180 ± 160 | 5,900 ± 500 | 20,900 ± 2500 |
| N209K | 1,600 ± 500 | 7,900 ± 2200 | 1,200 ± 730 |

The progress curves for the each inhibitor concentration were fitted using an equation for slow tight binding inhibitors:
P = a * (1−e^{-k}obs*^{t}) + v_{bkg}* Where ‘P’ is the pmol of PPi exchanged into ATP, ‘t’ is the time, and ‘a’ is the amplitude. A “background” correction factor was included to account for the rate of uninhibited activity which presumably is the fraction of enzyme that contains an oxidized cysteine and can not form the MLN4924-NEDD8 adduct. The 2^{nd} order rate constant for inactivation (≡rate of adduct formation) was estimated using the PPi-ATP exchange assay at 1 mM ATP from the slope of a linear regression of k_{obs} on [I]. The values are given with 95% confidence limit.

These data provide a rationale for explaining the resistance that is conferred by mutations in NAEβ, namely the slower rate of inactivation and faster off-rate of the NEDD8-MLN4294 adduct. MLN4924 and Compound 1 have large indane N6 substitutions on the adenosine group. Adenosine sulfamate, without the substitution, does not lose its potency on the A171T UBA3 variant. Mutations in the conserved alanine within the ATP binding pocket could serve as a mechanism of resistance against the N⁶-substituted andenosine sulfamate-like inhibitors.

### Example 7

### Cells Containing Mutations in NAEβ Form Lower Levels of NEDD8-MLN4924 Adduct and Show Increased Recovery of Pathway Activity

Cell Culture Washout and Immunoprecipitation Analysis

HCT-116 wild type, A171 and G201V cells were treated with either 1 or 10 μM MLN4924 or DMSO for 1 hour. Cells were washed with media to remove drug, replaced with fresh media and harvested at 0. 0.25, 2 and 5 hours post washout. Lysates were prepared as previously reported (Soucy et al., 2009). 100 ug washout lysate was incubated with 5 ug NAEβ antibody on ice for 1 hour. 50 uls of 50% slurry Protein G agarose (Upstate) was added and tumbled for 1 hour at 4° C. Samples were spun down and supernatant removed to fresh tube (unbound fraction) and beads were washed 3 times with buffer. 50 uls 2× sample buffer was added to beads (bound fraction) and samples fractionated on SDS PAGE gels and immunoblotted as indicated. To unbound fraction repeat above procedure using 2 ug 4924-NEDD8-ADS (MIL22) antibody and 50 uls of 50% slurry Protein A agarose (Pierce).

Results

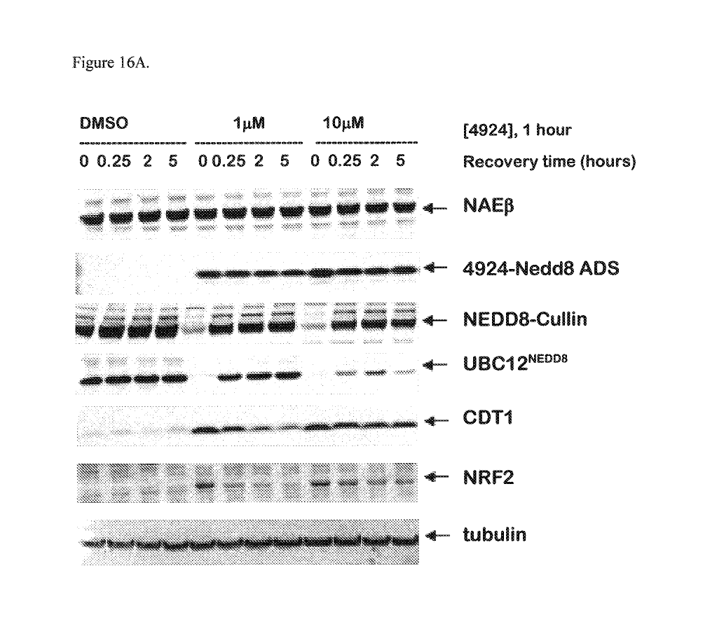

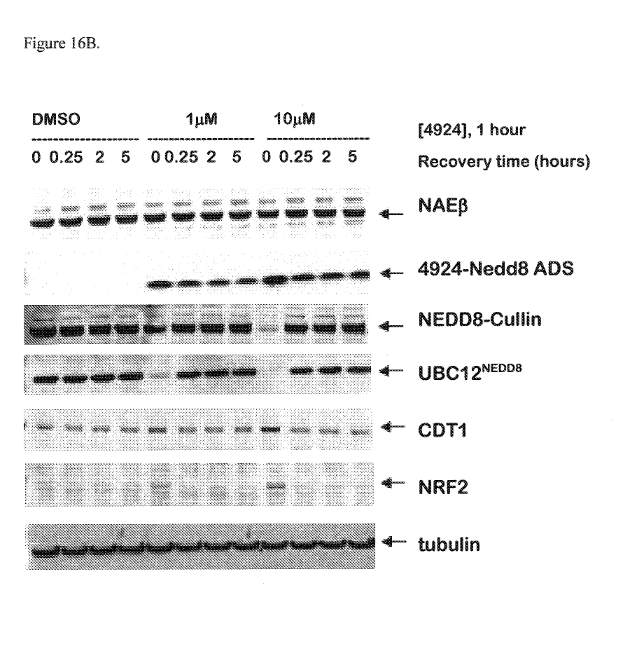

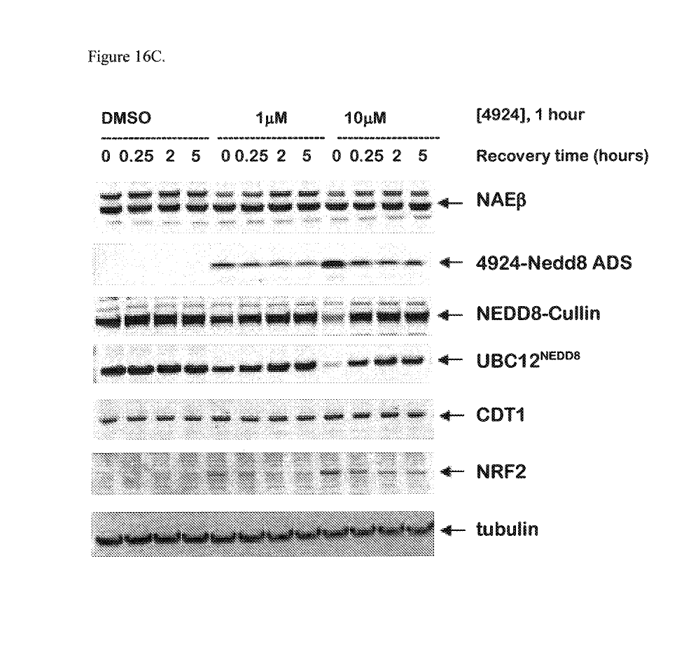

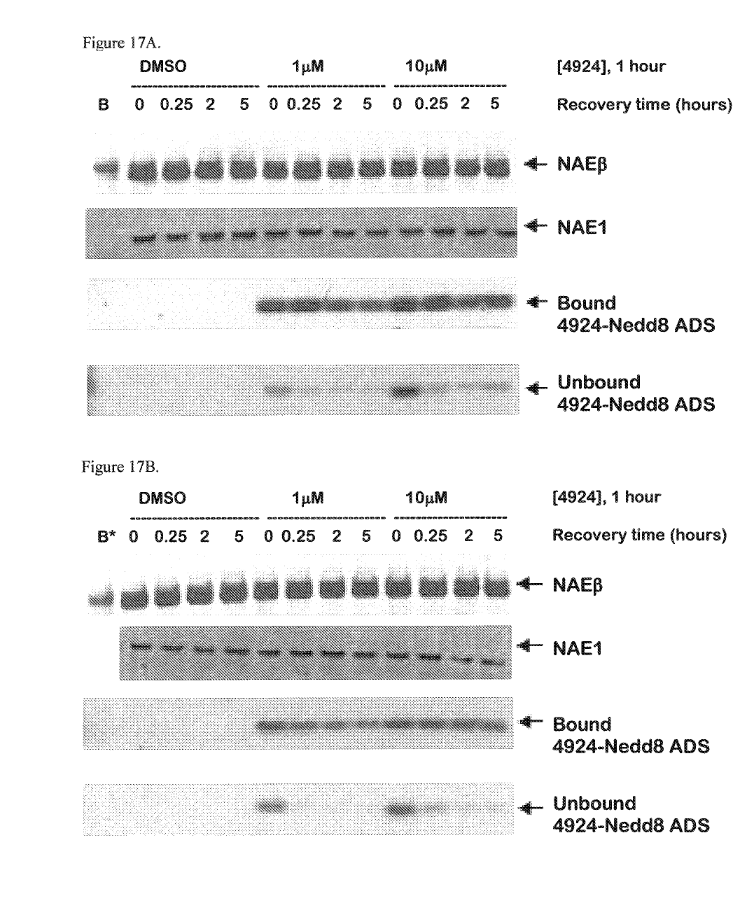

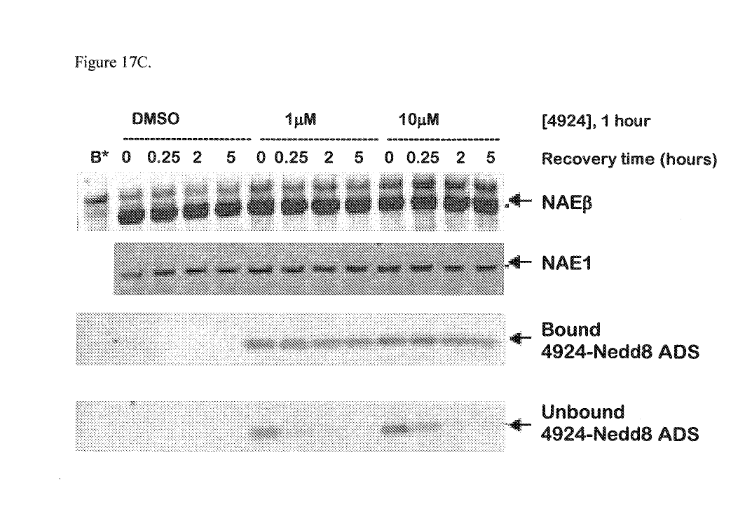

HCT116 mutant cells lines (A171T and G201V) were evaluated during and after MLN4924 treatment to determine the effect of these mutations on pathway activity recovery. Cells were treated with 1 or 10 μM MLN4924 for 1 hour after which compound was removed, replaced with drug free media and cells harvested at 0 min, 30 min, 2 hours or 5 hours post-drug washout (FIGS. 16A-C). In agreement with previous observations (Brownell et al., 2010), western blot analysis of WT cells indicated incomplete recovery of NEDD8-cullin, NEDD8-UBC12 and a persistence of NEDD8-MLN4924 adduct levels at both 1 and 10 μM for at least 5 hours post-washout. The prolonged pathway inhibition in the washout setting was corroborated by continued elevation of two CRL substrates NRF-2 and Cdt-1. In contrast, both A171T and G201V mutant cell lines showed almost complete recovery of pathway activity as early as 30 minutes post-washout. Interestingly, it appeared that the two mutant cell lines contained a reduced amount of NEDD8-MLN4924 adduct compared to WT cells, even though the levels of total NAEβ appeared similar. This would support the biochemical findings of slower rate of adduct formation and weaker binding of adduct in the mutant enzymes. To determine whether lower amounts of NEDD8-MLN4924 adduct were bound to NAE cells were treated under the same washout conditions but subjected to immunoprecipitation assays with NAEβ (FIGS. 17A-C). Similar levels of NAEβ and NAE1 were detected in immunoprecipitates indicating that the NAE heterodimer had been efficiently extracted from cell lysates. Immunoprecipitates were next probed with the MLN4924-NEDD8 antibody which detected lower levels bound to A171T and G201V mutants compared to WT. It is likely that the amount of NEDD8-MLN4294 adduct bound to NAEβ is comprised mostly of adduct bound to WT enzyme but not to mutant as the mutant enzyme can form adduct but not bind the adduct tightly. To determine if higher levels of unbound adduct is present in mutant versus WT cells, the flow-through from the immunoprecipitates was probed with the MLN4294-NEDD8 adduct antibody. However, there did not appear to be a difference in the amount of free adduct in WT versus mutant cells which may be due to proteolysis of adduct when it is released from NAEβ in cells. These data show that following inhibition of mutant NAEβ in cells pathway activity recovers quickly and correlates with lower amounts of NEDD8-MLN4924 adduct bound to the enzyme.

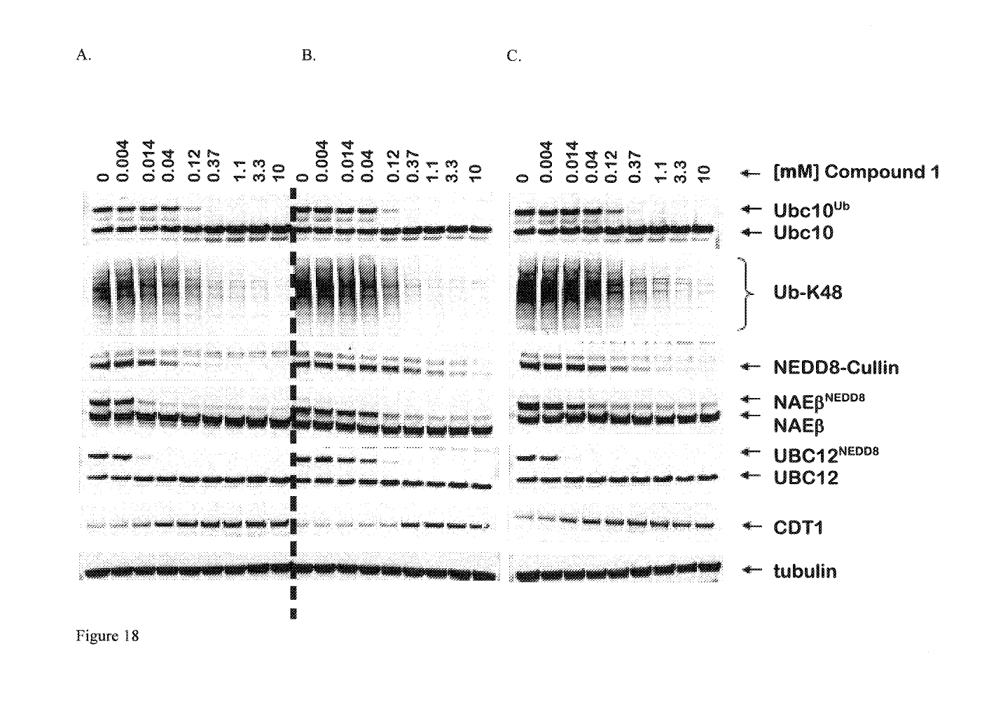

The data in biochemical and cell based assays would suggest that a NEDD8-Cpd adduct that was able to bind more tightly to the enzyme may overcome resistance to MLN4924 treatment emergent NAE mutations. To test this hypothesis we used Compound 1 which had faster rates of enzyme inhibition and slower rates of recovery compared to MLN4924 in biochemical assays. HCT-116 WT, A171T and G201V mutant cells were exposed to increasing concentrations of Compound 1 for four hours and assessed for pathway activity by Western blotting (FIGS. 18A-C). Since Compound 1 also inhibits UBA1 we could show comparable inhibition of ubiquitination of UBC10 (an E2 for ubiquitin) and polyubiquitination in all cells. In contrast to MLN4924 (see FIG. 10), Compound 1 was able to produce comparable inhibition of NAEβ-NEDD8, NEDD8-cullin and NEDD8-UBC12 in G201V mutant versus WT cells whereas there was still a modest decrease in A171T mutant cells compared to WT. This likely reflects the quicker recovery of enzyme activity seen with Compound 1 in A171T biochemical assays. These data suggest that NEDD8-compounds with improved binding affinities may overcome the resistance observed in the NAEβ mutations we have identified.

Treatment emergent mutations in NAEβ were detected in the ATP binding pocket and NEDD8 binding cleft with approximately ⅔ at Alanine 171 representing a potential hotspot. Whilst mutations in both areas generally lead to changes in affinities for ATP or NEDD8 this did not lead to a dramatic change in catalytic rate of NAE (except in the case of A171D and G201V). However, mutations in both areas of NAEβ lead to a slower rate of enzyme inhibition and a MLN4924-NEDD8 adduct that was no longer tightly bound. We have previously demonstrated that MLN4924-NEDD8 adduct formation is necessary for potent NAE inhibition by MLN4924 and that the tight binding nature of the adduct is required for in vitro and cellular potency (Brownell et al., 2010). In keeping with this notion we demonstrated reduced pathway inhibition in cells and a more rapid recovery from compound inhibition in cell washout experiments. We used a non-selective E1 inhibitor (Compound 1) to probe the effects of more potent enzyme inhibition in vitro and in cells. Compound 1 forms an adduct with NEDD8 in vitro and shows a slower rate of recovery from enzyme inhibition compared to MLN4924 in the WT and mutant enzymes tested. These data indicate that the Compound1-NEDD8 adduct is a tighter binder of WT and mutant NAEβ enzymes compared to MLN4924. Indeed, in A171T and G201V mutant HCT-116 cells Compound 1 was able to more potently inhibit NEDD8-cullin and NEDD8-UBC12 thioester levels than MLN4924. These data support our notion that a NAE selective compound-NEDD8 adduct with high affinity for mutant and WT enzyme should overcome resistance in cells and tumor xenografts. Unfortunately it was not possible to test this in cell viability or xenograft experiments as compound 1 is non-selective for other E1 enzymes whose inhibition can result in viability effects (Brownell et al., 2010).

### Example 8

### Sequencing of DNA from AML, Colon or Melanoma Tumor Samples does not Detect Pre-Existing Mutations in NAEβ

Clinical Human Tumor Testing

AML: 41 unique malignant Acute Myeloid Leukemia (AML) patient tumors (21 bone marrow aspirates and 20 bone marrow mononuclear cells) were genotyped for mutations found in preclinical models using the sequenom NAEβ assays and the full gene was sequenced by an Illumina Next Generation Sequencing assay. All samples were found to be wild type for NAEβ. The AML tumors represented newly diagnosed and relapse patients with M1 to M5 diagnosis classification. The blast tumor count ranged from 2 to 94 percent.

Matched PBMC were also sequenced and found to be wild type.

Colon: A collection of 50 unique mucinous and sigmoidal colon adenocarcinomas with representative histologies of poor, moderate and well differentiated and 10 to 100 percent tumor per tissue were genotyped for mutations found in preclinical models using the sequenom NAEβ assays and the full gene was sequenced by Sanger sequencing. All samples were found to be wild type for NAEβ.

Melanoma: A collection of 25 unique epithelioid and spindle cell type melanoma adenocarcinomas ranging from 25 to 100 percent tumor per tissue were genotyped for mutations found in preclinical models using the sequenom NAEβ assays and the full gene was sequenced by Sanger sequencing. All samples were found to be wild type for NAEβ.

Results

To determine whether mutations in NAEβ could be detected in cancer patients and therefore may exist prior to MLN4924 therapy, DNA from 50 colon and melanoma tumor samples and 41 AML samples were subjected to Sanger and Sequenom sequencing (Table 12).

TABLE 12
Mutational status of NAEβ from DNA samples isolated from
patients with AML, colorectal cancer or melanoma

| | | | Next Gen | NAE-beta |
|---|---|---|---|---|
| Tumor Type | Sanger | Sequenom | Sequencing | Status |
| AML (Bone Marrow | nt | 21 | 13 | wt |
| Aspirates) | | | | |
| AML (BMMC) | nt | 20 | 8 | wt |
| Colon | 50 | 50 | nt | wt |
| Melanoma | 25 | 25 | nt | wt |

DNA was assayed by Sanger, Sequenom and Next Generation sequencing where indicated. The number of samples tested is shown.
BMMC = Bone Marrow Mononuclear Cells,
nt = not tested.

No NAEβ mutations were detected by either method with the Sequenom method having a sensitivity limit of approximately 10%. To increase the sensitivity of detection we subjected 21 of the AML samples to Next generation sequencing of NAEβ using the illumina platform but did not detect a mutation in NAEβ. Similarly, “pre-exisiting’ mutations in NANi were not detected in WT HCT-116, Calu-6 or NCI-H460 cells by Next Generation sequencing. Interestingly, one heterozygous NAEB mutation (C249Y resulting from an amino acid change G>A) has been reported in an ovarian cancer patient in the Cosmic database (total 218 tumor samples tested, See Distribution of Somatic Mutations in UBA3 record in Cosmic Database maintained by Wellcome Trust Sanger Institute, Hinxton, Cambridge UK). This mutation is in a region of NAEβ that binds NAE1 (Walden et al., 2003) and so may interfere with heterodimer formation and enzyme activity. Thus we were not able to detect a pre-existing mutation in NAE in DNA isolated from patient tumors, leukemic blasts or cancer cell lines.

In these studies we have detected heterozygous mutations in NAEβ and thus cells maintain one wild type copy of NAEβ. An important model system to conclusively prove that mutations in NAEβ drive resistance would be an engineered cell line that only expresses mutant and not wild type enzyme. Thus far we have not been successful in developing such a model system. It is possible that a wild type enzyme is required to support growth and that the role of the mutant enzyme is to enable NEDD8 conjugation when the WT enzyme is under transient inhibition by MLN4924 in cells and xenografts. Since the mutation frequency of NAEβ in cell and xenograft populations is ˜50% we developed mass spectrometry and next generation sequencing methodologies that allowed us to detect NAEβ mutations to a frequency of ˜0.5%. We were not able to detect mutations in NAEβ that were pre-existing in cell line, xenograft or patient tumor DNA samples suggesting that the mutations are acquired during the selection process. A search of single-nucleotide polymorphism databases did not reveal the existence of the mutations reported in human populations but we cannot rule out the possibility that mutations do exist at a lower frequency that 0.5% in tumors. Indeed, a mutation in NAEβ has been reported in the COSMIC database in an ovarian cancer sample at C249Y. This residue is in a region of NAEβ that is involved in binding NAE1 (Walden et al., 2003) and so it is possible this would interfere with heterodimer formation and enzyme activity.

### Example 9

### Survey Comparing Activity of NAE Inhibitors on WT and A171T Mutant Enzyme

Method

IC50 measurements were performed in the HTRF assay described in Example 6. Assays for each enzyme were performed at their K_{M} for ATP (20 μM for wild type, 120 μM for the A171T mutant).

Results

The Table 13 below lists the IC50 measurements for each inhibitor. Inhibitors in this list that result in about less than 4-fold ratio of A271T/WT are about equipotent. Compounds that are identified reference the PCT and US patent application publication example in which they are disclosed.

TABLE 13
Comparison of activity of 1-substituted methyl sulfamates on UBA3 variant with A171T mutation Compared to wild type UBA3

| | | | NAE | NAE | | |
|---|---|---|---|---|---|---|
| PCT | US | | WT | Mutant | | |
| publi- | publi- | Example | IC50 | A171T | A171T/ | |
| cation | cation | number | (uM) | IC50 (uM) | WT | compound name |
| WO 2007/ | US 2007/ | I-52 | 0.013 | 0.013 | 1 | [(1S,2S,4R)-2-hydroxy-4-(4-{[(1R,2S)-2-methoxy-2,3-dihydro-1H-inden-1-yl]- |
| 092213 | 0191293 | | | | | amino}-7H-pyrrolo[2,3-d]pyrimidin-7-yl)cyclopentyl]methyl sulfamate |
| WO 2007/ | US 2007/ | I-71 | 0.0082 | 0.015 | 2 | [(1S,2S,4R)-2-hydroxy-4-(4-{[(1R,2S)-2-hydroxy-2,3-dihydro-1H-inden-1- |
| 092213 | 0191293 | | | | | yl]amino}-7H-pyrrolo[2,3-d]pyrimidin-7-yl)cyclopentyl]methyl sulfamate |
| WO 2008/ | US 2008/ | I-118 | 0.16 | 0.43 | 3 | ((1R,2R,3S,4R)-2,3-dihydroxy-4-{[6-(5,6,7,8-tetrahydronaphthalen-1- |
| 019124 | 0051404 | | | | | ylamino)pyrimidin-4-yl]amino}cyclopentyl)methyl sulfamate |
| WO 2007/ | US 2007/ | I-85 | 0.011 | 0.034 | 3 | [(1S,2S,4R)-2-hydroxy-4-(4-{[(1R,2S)-2-methoxy-1,2,3,4-tetrahydronaphthalen-1- |
| 092213 | 0191293 | | | | | yl]amino}-7H-pyrrolo[2,3-d]pyrimidin-7-yl)cyclopentyl]methyl sulfamate |
| WO 2007/ | US 2007/ | I-9 | 0.11 | 0.34 | 3 | {(1S,2S,4R)-4-[4-(acetylamino)-7H-pyrrolo[2,3-d]pyrimidin-7-yl]-2- |
| 092213 | 0191293 | | | | | hydroxycyclopentyl}methyl sulfamate |
| WO 2006/ | US 2006/ | I-14 | 0.0017 | 0.0053 | 3 | [(2R,3S,4R,5R)-3,4-dihydroxy-5-(6-{[(1R,2S)-2-hydroxy-2,3-dihydro-1H-inden-1- |
| 084281 | 0189636 | | | | | yl]amino}-9H-purin-9-yl)tetrahydrofuran-2-yl]methyl sulfamate |
| WO 2007/ | US 2007/ | I-14 | 0.0063 | 0.022 | 3 | [(1S,2S,4R)-4-(4-{[(1S)-3,3-dimethyl-2,3-dihydro-1H-inden-1-yl]amino}-7H- |
| 092213 | 0191293 | | | | | pyrrolo[2,3-d]pyrimidin-7-yl)-2-hydroxycyclopentyl]methyl sulfamate |
| WO 2007/ | US 2007/ | I-18 | 0.0031 | 0.012 | 4 | [(1S,2S,4R)-4-(4-amino-7W-pyrrolo[2,3-d]pyrimidin-7-yl)-2-hydroxycyclopentyl]- |
| 092213 | 0191293 | | | | | methyl sulfamate |
| WO 2007/ | US 2007/ | I-69 | 0.023 | 0.12 | 5 | [(1S,2S,4R)-4-(5-fluoro-4-{[1R,2S)-2-methoxy-2,3-dihydro-1H-inden-1-yl]amino}- |
| 092213 | 0191293 | | | | | 7H-pyrrolo[2,3-d]pyrimidin-7-yl)-2-hydroxycyclopentyl]methyl sulfamate |
| WO 2006/ | US 2006/ | I-48 | 0.0043 | 0.026 | 6 | ((2R,3S,4R,5R)-3,4-dihydroxy-5-{6-[(phenylsulfonyl)amino]-9H-purin-9-yl}- |
| 084281 | 0189636 | | | | | tetrahydrofuran-2-yl)methyl sulfamate |
| WO 2007/ | US 2007/ | I-35 | 0.0044 | 0.028 | 6 | ((1S,2S,4R)-4-(4-((1S)-2,3-dihydro-1H-inden-1-ylamino)-7H-pyrrolo[2,3- |
| 092213 | 0191293 | | | | | d]pyrimidin-7-yl)-2-hydroxycyclopentyl)methyl sulfamate |
| WO 2007/ | US 2007/ | I-46 | 0.037 | 0.25 | 7 | [(1S,2S,4R)-2-hydroxy-4-(4-methyl-7H-pyrrolo[2,3-d]pyrimidin-7-yl)cyclopentyl]- |
| 092213 | 0191293 | | | | | methyl sulfamate |
| WO 2007/ | US 2007/ | I-65 | 0.0081 | 0.055 | 7 | [(1S,2S,4R)-4-(4-{[(1S)-4-bromo-2,3-dihydro-1H-inden-1-yl]amino}-7H- |
| 092213 | 0191293 | | | | | pyrrolo[2,3-d]pyrimidin-7-yl)-2-hydroxycyclopentyl]methyl sulfamate |
| WO 2007/ | US 2007/ | I-82 | 0.0077 | 0.059 | 8 | [(1S,2S,4R)-2-hydroxy-4-(4-{[(1S,2S)-2-methyl-2,3-dihydro-1H-inden-1-yl]amino}- |
| 092213 | 0191293 | | | | | 7H-pyrrolo[2,3-d]pyrimidin-7-yl)cyclopentyl]methyl sulfamate |
| WO 2006/ | US 2006/ | I-26 | 0.019 | 0.16 | 8 | ((2R,3S,4R,5R)-3,4-dihydroxy-5-{6-[(piperidin-4-ylmethyl)amino]-9H-purin-9-yl}- |
| 084281 | 0189636 | | | | | tetrahydrofuran-2-yl)methyl sulfamate |
| WO 2007/ | US 2007/ | I-40 | 0.072 | 0.63 | 9 | {(1S,2S,4R)-2-hydroxy-4-[4-(methylamino)-7H-pyrrolo[2,3-d]pyrimidin-7-yl]- |
| 092213 | 0191293 | | | | | cyclopentyl}methyl sulfamate |
| WO 2007/ | US 2007/ | I-12 | 0.012 | 0.11 | 9 | ((1S,2S,4R)-4-{4-[(4S)-3,4-dihydro-2H-chromen-4-ylamino]-7H-pyrrolo[2,3-d]- |
| 092213 | 0191293 | | | | | pyrimidin-7-yl}-2-hydroxycyclopentyl)methyl sulfamate |
| WO 2007/ | US 2007/ | I-27 | 0.027 | 0.25 | 9 | [(1S,2S,4R)-4-(4-{[(1S)-5-chloro-2,3-dihydro-1H-inden-1-yl]amino}-7H-pyrrolo- |
| 092213 | 0191293 | | | | | [2,3-d]pyrimidin-7-yl)-2-hydroxycyclopentyl]methyl sulfamate |
| WO 2007/ | US 2007/ | I-84 | 0.0097 | 0.095 | 10 | [(1S,2S,4R)-4-(4-{[(1S,2S)-2-ethyl-2,3-dihydro-1H-inden-1-yl]amino}-7H- |
| 092213 | 0191293 | | | | | pyrrolo[2,3-d]pyrimidin-7-yl)-2-hydroxycyclopentyl]methyl sulfamate |
| WO 2006/ | US 2006/ | I-24 | 0.012 | 0.17 | 14 | [(2R,3S,4R,5R)-3,4-dihydroxy-5-(6-{[(1R)-2-hydroxy-1-phenylethyl]amino}-9H- |
| 084281 | 0189636 | | | | | purin-9-yl)tetrahydrofuran-2-yl]methyl sulfamate |
| WO 2006/ | US 2006/ | I-12 | 0.0012 | 0.017 | 14 | {(2R,3S,4R,5R)-5-[6-(benzoylamino)-9H-purin-9-yl]-3,4-dihydroxytetrahydro- |
| 084281 | 0189636 | | | | | furan-2-yl}methyl sulfamate |
| WO 2007/ | US 2007/ | I-33 | 0.02 | 0.29 | 15 | [(1S,2S,4R)-4-(4-{[(1S)-5-bromo-2,3-dihydro-1H-inden-1-yl]amino}-7H-pyrrolo- |
| 092213 | 0191293 | | | | | [2,3-d]pyrimidin-7-yl)-2-hydroxycyclopentyl]methyl sulfamate |
| WO 2006/ | US 2006/ | I-28 | 0.011 | 0.16 | 15 | [(2R,3S,4R,5R)-5-(6-{[2-(4-benzylpiperazin-1-yl)ethyl]amino}-9H-purin-9-yl)-3,4- |
| 084281 | 0189636 | | | | | dihydroxytetrahydrofuran-2-yl]methyl sulfamate |
| WO 2006/ | US 2006/ | I-23 | 0.0028 | 0.042 | 15 | [(2R,3S,4R,5R)-3,4-dihydroxy-5-(6-{[(1S)-2-hydroxy-1-phenylethyl]amino}-9H- |
| 084281 | 0189636 | | | | | purin-9-yl)tetrahydrofuran-2-yl]methyl sulfamate |
| WO 2007/ | US 2007/ | I-36 | 0.0095 | 0.16 | 17 | [(1S,2S,4R)-4-(4-{[(1S)-5-fluoro-2,3-dihydro-1H-inden-1-yl]amino}-7H-pyrrolo- |
| 092213 | 0191293 | | | | | [2,3-d]pyrimidin-7-yl)-2-hydroxycyclopentyl]methyl sulfamate |
| WO 2008/ | US 2008/ | I-55 | 0.0042 | 0.071 | 17 | {(1R,2R,3S,4R)-2,3-dihydroxy-4-[(4-{[(1R,2S)-2-methoxy-2,3-dihydro-1H-inden-1- |
| 019124 | 0051404 | | | | | yl]amino}-1,3,5-triazin-2-yl)amino]cyclopentyl}methyl sulfamate |
| WO 2008/ | US 2008/ | I-64 | 0.00039 | 0.0069 | 18 | [(1S,2S,4R)-4-({8-[4-(dimethylamino)-1-naphthyl]-9H-purin-6-yl}amino)-2- |
| 019124 | 0051404 | | | | | hydroxycyclopentyl]methyl sulfamate |
| WO 2006/ | US 2006/ | I-47 | 0.0022 | 0.04 | 18 | ((2R,3S,4R,5R)-5-{6-[(4-fluorobenzoyl)amino]-9H-purin-9-yl}-3,4- |
| 084281 | 0189636 | | | | | dihydroxytetrahydrofuran-2-yl)methyl sulfamate |
| WO 2007/ | US 2007/ | I-67 | 0.024 | 0.44 | 18 | [(1S,2S,4R)-4-(4-{[(1S)-5-chloro-3,3-dimethyl-2,3-dihydro-1H-inden-1-yl]amino}- |
| 092213 | 0191293 | | | | | 7H-pyrrolo[2,3-d]pyrimidin-7-yl)-2-hydroxycyclopentyl]methyl sulfamate |
| WO 2007/ | US 2007/ | I-2 | 0.0093 | 0.2 | 22 | [(1S,2S,4R)-4-(4-{[2-(difluoromethoxy)benzyl]amino}-7H-pyrrolo[2,3-d]pyrimidin- |
| 092213 | 0191293 | | | | | 7-yl)-2-hydroxycyclopentyl]methyl sulfamate |
| WO 2006/ | US 2006/ | I-2 | 0.00055 | 0.012 | 22 | ((2R,3S,4R,5R)-5-{6-[(1S)-2,3-dihydro-1H-inden-1-ylamino]-9H-purin-9-yl}-3,4- |
| 084281 | 0189636 | | | | | dihydroxytetrahydrofuran-2-yl)methyl sulfamate |
| WO 2007/ | US 2007/ | I-38 | 0.013 | 0.3 | 23 | ((1S,2S,4R)-2-hydroxy-4-{4-[(1-naphthylmethyl)amino]-7H-pyrrolo[2,3-d]- |
| 092213 | 0191293 | | | | | pyrimidin-7-yl}cyclopentyl)methyl sulfamate |
| WO 2006/ | US 2006/ | I-22 | 0.0051 | 0.12 | 24 | [(2R,3S,4R,5R)-3,4-dihydroxy-5-(6-{[(5-methylpyrazin-2-yl)methyl]amino}-9H- |
| 084281 | 0189636 | | | | | purin-9-yl)tetrahydrofuran-2-yl]methyl sulfamate |
| WO 2007/ | US 2007/ | I-63 | 0.008 | 0.2 | 25 | [(1S,2S,4R)-4-(4-{[(1R)-4-chloro-2,3-dihydro-1H-inden-1-yl]amino}-7H- |
| 092213 | 0191293 | | | | | pyrrolo[2,3-d]pyrimidin-7-yl)-2-hydroxycyclopentyl]methyl sulfamate |
| WO 2007/ | US 2007/ | I-49 | 0.012 | 0.36 | 30 | {(1S,2S,4R)-4-[4-(benzylamino)-7H-pyrrolo[2,3-d]pyrimidin-7-yl]-2- |
| 092213 | 0191293 | | | | | hydroxycyclopentyl}methyl sulfamate |
| WO 2008/ | US 2008/ | I-9 | 0.007 | 0.21 | 30 | {(1R,2R,3S,4R)-2,3-dihydroxy-4-[(8-phenyl-9H-purin-6-yl)amino]cyclopentyl}- |
| 019124 | 0051404 | | | | | methyl sulfamate |
| WO 2006/ | US 2006/ | I-89 | 0.0002 | 0.0061 | 31 | ((2R,3S,4R,5R)-3,4-dihydroxy-5-{6-[(2-trifluoromethylphenyl)ethynyl]-9H-purin- |
| 084281 | 0189636 | | | | | 9-yl}tetrahydrofuran-2-yl)methyl sulfamate |
| WO 2008/ | US 2008/ | I-139 | 0.00068 | 0.024 | 35 | ((1S,2S,4R)-2-hydroxy-4-{[8-(4-pyrrolidin-1-yl-1-naphthyl)-7H-purin-6- |
| 019124 | 0051404 | | | | | yl]oxy}cyclopentyl)methyl sulfamate |
| WO 2008/ | US 2008/ | I-39 | 0.0013 | 0.046 | 35 | {(1S,2S,4R)-4-[(8-dibenzo[b,d]furan-4-yl-9H-purin-6-yl)amino]-2- |
| 019124 | 0051404 | | | | | hydroxycyclopentyl}methyl sulfamate |
| WO 2007/ | US 2007/ | I-62 | 0.051 | 2 | 39 | [(1S,2S,4R)-4-(4-{[(1S)-4,7-difluoro-2,3-dihydro-1H-inden-1-yl]amino}-7H- |
| 092213 | 0191293 | | | | | pyrrolo[2,3-d]pyrimidin-7-yl)-2-hydroxycyclopentyl]methyl sulfamate |
| WO 2006/ | US 2006/ | I-91 | 0.0011 | 0.045 | 41 | ((2R,3S,4R,5R)-5-{6-[(4-chlorophenyl)ethynyl]-9H-purin-9-yl}-3,4- |
| 084281 | 0189636 | | | | | dihydroxytetrahydrofuran-2-yl)methyl sulfamate |
| WO 2008/ | US 2008/ | I-12 | 0.015 | 0.62 | 41 | [(1R,2R,3S,4R)-2,3-dihydroxy-4-({6-[(1S)-1,2,3,4-tetrahydronaphthalen-1- |
| 019124 | 0051404 | | | | | ylamino]pyrimidin-4-yl}amino)cyclopentyl]methyl sulfamate |
| WO 2007/ | US 2007/ | I-43 | 0.0031 | 0.13 | 42 | ((1S,2S,4R)-2-hydroxy-4-{4-[(2-methoxybenzyl)amino]-7H-pyrrolo[2,3-d]- |
| 092213 | 0191293 | | | | | pyrimidin-7-yl}cyclopentyl)methyl sulfamate |
| WO 2008/ | US 2008/ | I-100 | 0.0049 | 0.21 | 43 | ((1S,2S,4R)-4-{[8-(2,2-dimethyl-2,3-dihydro-1-benzofuran-7-yl)-9H-purin-6- |
| 019124 | 0051404 | | | | | yl]amino}-2-hydroxycyclopentyl)methyl sulfamate |
| WO 2007/ | US 2007/ | I-23 | 0.0095 | 0.41 | 43 | ((1S,2S,4R)-4-{6-[(4-chlorobenzyl)amino]-9H-purin-9-yl}-2- |
| 092213 | 0191293 | | | | | hydroxycyclopentyl)methyl sulfamate |
| WO 2008/ | US 2008/ | I-2 | 0.0063 | 0.28 | 44 | {(1R,2R,3S,4R)-2,3-dihydroxy-4-[(6-{[(1R,2S)-2-methoxy-2,3-dihydro-1H-inden-1- |
| 019124 | 0051404 | | | | | yl]amino}pyrimidin-4-yl)amino]cyclopentyl}methyl sulfamate |
| WO 2007/ | US 2007/ | I-66 | 0.0066 | 0.3 | 45 | [(1S,2S,4R)-4-(4-{[(1S)-7-fluoro-2,3-dihydro-1H-inden-1-yl]amino}-7H- |
| 092213 | 0191293 | | | | | pyrrolo[2,3-d]pyrimidin-7-yl)-2-hydroxycyclopentyl]methyl sulfamate |
| WO 2008/ | US 2008/ | I-47 | 0.0025 | 0.14 | 56 | {(1S,2S,4R)-2-hydroxy-4-[(6-{[(1R,2S)-2-methoxy-2,3-dihydro-1H-inden-1- |
| 019124 | 0051404 | | | | | yl]amino}pyrimidin-4-yl)amino]cyclopentyl}methyl sulfamate |
| WO 2006/ | US 2006/ | I-5 | 0.00032 | 0.018 | 56 | {(2R,3S,4R,5R)-3,4-dihydroxy-5-[6-(phenylethynyl)-9H-purin-9-yl]tetrahydro- |
| 084281 | 0189636 | | | | | furan-2-yl}methyl sulfamate |
| WO 2006/ | US 2006/ | I-10 | 0.0018 | 0.11 | 61 | ((2R,3S,4R,5R)-5-{6-[(4-chlorobenzyl)amino]-9H-purin-9-yl}-3,4- |
| 084281 | 0189636 | | | | | dihydroxytetrahydrofuran-2-yl)methyl sulfamate |
| WO 2007/ | US 2007/ | I-45 | 0.0093 | 0.58 | 62 | ((1S,2S,4R)-4-{4-[(4-chlorobenzyl)amino]-7H-pyrrolo[2,3-d]pyrimidin-7-yl}-2- |
| 092213 | 0191293 | | | | | hydroxycyclopentyl)methyl sulfamate |
| WO 2008/ | US 2008/ | I-146 | 0.0067 | 0.42 | 63 | ((1S,2S,4R)-4-{4-[(1S)-2,3-dihydro-1H-inden-1-ylamino]-5,6-dihydro-7H- |
| 019124 | 0051404 | | | | | pyrrolo[2,3-d]pyrimidin-7-yl}-2-hydroxycyclopentyl)methyl sulfamate |
| WO 2007/ | US 2007/ | I-24 | 0.011 | 0.7 | 64 | ((1S,2S,4R)-4-{4-[(3,4-dichlorobenzyl)amino]-7H-pyrrolo[2,3-d]pyrimidin-7-yl}-2- |
| 092213 | 0191293 | | | | | hydroxycyclopentyl)methyl sulfamate |
| WO 2007/ | US 2007/ | I-51 | 0.0085 | 0.55 | 65 | ((1S,2S,4R)-4-{4-[(3-chlorobenzyl)amino]-7H-pyrrolo[2,3-d]pyrimidin-7-yl}-2- |
| 092213 | 0191293 | | | | | hydroxycyclopentyl)methyl sulfamate |
| WO 2007/ | US 2007/ | I-64 | 0.0087 | 0.58 | 67 | [(1S,2S,4R)-4-(4-{[(1S)-4-chloro-2,3-dihydro-1H-inden-1-yl]amino}-7H- |
| 092213 | 0191293 | | | | | pyrrolo[2,3-d]pyrimidin-7-yl)-2-hydroxycyclopentyl]methyl sulfamate |
| WO 2006/ | US 2006/ | I-90 | 0.00025 | 0.017 | 68 | ((2R,3S,4R,5R)-3,4-dihydroxy-5-{6-[(2-methoxyphenyl)ethynyl]-9H-purin-9-yl}- |
| 084281 | 0189636 | | | | | tetrahydrofuran-2-yl)methyl sulfamate |
| WO 2007/ | US 2007/ | I-72 | 0.051 | 3.8 | 75 | [(1S,2S,4R)-2-hydroxy-4-(4-{[(1R,2R)-2-methoxy-2,3-dihydro-1H-inden-1- |
| 092213 | 0191293 | | | | | yl]amino}-7H-pyrrolo[2,3-d]pyrimidin-7-yl)cyclopentyl]methyl sulfamate |
| WO 2006/ | US 2006/ | I-11 | 0.0016 | 0.12 | 75 | ((2R,3S,4R,5R)-3,4-dihydroxy-5-{6-[(3-methoxybenzyl)amino]-9H-purin-9-yl}- |
| 084281 | 0189636 | | | | | tetrahydrofuran-2-yl)methyl sulfamate |
| WO 2008/ | US 2008/ | I-105 | 0.0028 | 0.21 | 75 | {(1S,2S,4R)-4-[(8-biphenyl-3-yl-9H-purin-6-yl)amino]-2- |
| 019124 | 0051404 | | | | | hydroxycyclopentyl}methyl sulfamate |
| WO 2008/ | US 2008/ | I-142 | 0.0053 | 0.45 | 85 | {(1S,2S,4R)-2-hydroxy-4-[(6-{[(1R,2S)-2-methoxy-1,2,3,4-tetrahydronaphthalen- |
| 019124 | 0051404 | | | | | 1-yl]amino}pyrimidin-4-yl)oxy]cyclopentyl}methyl sulfamate |
| WO 2008/ | US 2008/ | I-67 | 0.0027 | 0.23 | 85 | ((1S,2S,4R)-4-{[8-(2,3-dimethoxyphenyl)-9H-purin-6-yl]amino}-2- |
| 019124 | 0051404 | | | | | hydroxycyclopentyl)methyl sulfamate |
| WO 2006/ | US 2006/ | I-63 | 0.003 | 0.26 | 87 | ((2R,3S,4R,5R)-5-{6-[(1,3-benzodioxol-5-ylmethyl)amino]-9H-purin-9-yl}-3,4- |
| 084281 | 0189636 | | | | | dihydroxytetrahydrofuran-2-yl)methyl-sulfamate |
| WO 2006/ | US 2006/ | I-30 | 0.0053 | 0.46 | 87 | [(2R,3S,4R,5R)-3,4-dihydroxy-5-(6-{[4-(trifluoromethoxy)benzyl]amino}-9H- |
| 084281 | 0189636 | | | | | purin-9-yl)tetrahydrofuran-2-yl]methyl sulfamate |
| WO 2008/ | US 2008/ | I-68 | 0.0048 | 0.42 | 88 | [(1S,2S,4R)-4-({8-[2-(benzyloxy)phenyl]-9H-purin-6-yl}amino)-2- |
| 019124 | 0051404 | | | | | hydroxycyclopentyl]methyl sulfamate |
| WO 2008/ | US 2008/ | I-37 | 0.0046 | 0.44 | 96 | ((1S,2S,4R)-2-hydroxy-4-{[8-(2-phenoxyphenyl)-9H-purin-6- |
| 019124 | 0051404 | | | | | yl]amino}cyclopentyl)methyl sulfamate |
| WO 2006/ | US 2006/ | I-88 | 0.00026 | 0.025 | 96 | ((2R,3S,4R,5R)-5-{6-[(3-fluorophenyl)ethynyl]-9H-purin-9-yl}-3,4- |
| 084281 | 0189636 | | | | | dihydroxytetrahydrofuran-2-yl)methyl sulfamate |
| WO 2006/ | US 2006/ | I-8 | 0.00098 | 0.11 | 112 | ((2R,3S,4R,5R)-3,4-dihydroxy-5-{6-[(2-thienylmethyl)amino]-9H-purin-9-yl}- |
| 084281 | 0189636 | | | | | tetrahydrofuran-2-yl)methyl sulfamate |
| WO 2006/ | US 2006/ | I-65 | 0.0014 | 0.16 | 114 | ((2R,3S,4R,5R)-5-{6-[(4-fluorobenzyl)amino]-9H-purin-9-yl}-3,4- |
| 084281 | 0189636 | | | | | dihydroxytetrahydrofuran-2-yl)methyl-sulfamate |
| WO 2006/ | US 2006/ | I-6 | 0.00073 | 0.084 | 115 | {(2R,3S,4R,5R)-5-[6-(benzylamino)-9H-purin-9-yl]-3,4-dihydroxytetrahydrofuran- |
| 084281 | 0189636 | | | | | 2-yl}methyl sulfamate |
| WO 2008/ | US 2008/ | I-73 | 0.00092 | 0.11 | 120 | ((1S,2S,4R)-4-{[8-(7-chloroquinolin-4-yl)-7H-purin-6-yl]oxy}-2- |
| 019124 | 0051404 | | | | | hydroxycyclopentyl)methyl sulfamate |
| WO 2008/ | US 2008/ | I-111 | 0.011 | 1.5 | 136 | ((1S,2S,4R)-4-{[8-(4-chlorophenyl)-9H-purin-6-yl]amino}-2- |
| 019124 | 0051404 | | | | | hydroxycyclopentyl)methyl sulfamate |
| WO 2008/ | US 2008/ | I-74 | 0.00058 | 0.11 | 190 | ((1S,2S,4R)-2-hydroxy-4-{[6-(1-naphthyl)-7H-pyrrolo[2,3-d]pyrimidin-4- |
| 019124 | 0051404 | | | | | yl]amino}cyclopentyl)methyl sulfamate |
| WO 2008/ | US 2008/ | I-41 | 0.00089 | 0.17 | 191 | ((1S,2S,4R)-4-{[8-(2,3-dihydro-1,4-benzodioxin-5-yl)-9H-purin-6-yl]amino}-2- |
| 019124 | 0051404 | | | | | hydroxycyclopentyl)methyl sulfamate |
| WO 2008/ | US 2008/ | I-117 | 0.00099 | 0.22 | 222 | ((1S,2S,4R)-4-{[8-(2,3-dihydro-1-benzofuran-7-yl)-7H-purin-6-yl]amino}-2- |
| 019124 | 0051404 | | | | | hydroxycyclopentyl)methyl sulfamate |
| WO 2006/ | US 2006/ | I-1 | 0.0011 | 0.28 | 255 | ((2R,3S,5R)-5-{6-[(1S)-2,3-dihydro-1H-inden-1-ylamino]-9H-purin-9-yl}-3- |
| 084281 | 0189636 | | | | | hydroxytetrahydrofuran-2-yl)methyl sulfamate |
| WO 2006/ | US 2006/ | I-95 | 0.0015 | 0.39 | 260 | {(2R,3S,4R,5R)-5-[6-(cyclopropylethynyl)-9H-purin-9-yl]-3,4- |
| 084281 | 0189636 | | | | | dihydroxytetrahydrofuran-2-yl}methyl sulfamate |
| WO 2008/ | US 2008/ | I-26 | 0.008 | 2.2 | 275 | [(1R,2R,3S,4R)-4-({4-[(1S)-2,3-dihydro-1H-inden-1-ylamino]-1,3,5-triazin-2-yl}- |
| 019124 | 0051404 | | | | | amino)-2,3-dihydroxycyclopentyl]methyl sulfamate |
| WO 2008/ | US 2008/ | I-126 | 0.00097 | 0.53 | 546 | ((1S,2S,4R)-2-hydroxy-4-{[8-(5,6,7,8-tetrahydronaphthalen-1-yl)-9H-purin-6- |
| 019124 | 0051404 | | | | | yl]amino}cyclopentyl)methyl sulfamate |
| WO 2008/ | US 2008/ | I-121 | 0.0023 | 1.4 | 609 | ((1S,2S,4R)-2-hydroxy-4-{[8-(1,2,3,4-tetrahydronaphthalen-1-yl)-9H-purin-6- |
| 019124 | 0051404 | | | | | yl]amino}cyclopentyl)methyl sulfamate |
| WO 2008/ | US 2008/ | I-32 | 0.0055 | 3.9 | 709 | [(1S,2S,4R)-4-({6-[(1S)-2,3-dihydro-1H-inden-1-ylamino]-5-methylpyrimidin-4- |
| 019124 | 0051404 | | | | | yl}oxy)-2-hydroxycyclopentyl]methyl sulfamate |
| WO 2008/ | US 2008/ | I-5 | 0.0049 | 5.9 | 1204 | [(1S,2S,4R)-4-({6-[(1S)-2,3-dihydro-1H-inden-1-ylamino]pyrimidin-4-yl}amino)-2- |
| 019124 | 0051404 | | | | | hydroxycyclopentyl]methyl sulfamate |

### Example 10

### Additional E1 Enzyme Mutants

Bovine untagged ubiquitin (Cat. No. U6253) was purchased from Sigma (St. Louis, Mo., USA). Baculoviruses were generated with the Bac-to-Bac Expression System (Invitrogen). wild-type and A573D/T N-terminal His6-UBA6 was generated by single infection of Sf9 cells. Expression constructs for UAE A580T were generated both in baculovirus for biochemical studies and in a mammalian expression vector for cell culture studies. Expressed proteins were purified by affinity (Ni-NTA agarose, Qiagen) or conventional chromatography as described (Soucy et al. (2009) Nature 458:732-736). Mutant UAE enzyme was purified for biochemistry and the mammalian expression constructs were confirmed for their ability to express the mutant. Purified NEDD8-compound adduct was made and purified as described (Chen, J., et al. (2011) J. Biol. Chem. 286:40867-40877).

For Uba6 biochemical reactions, similar assay conditions as in Example 6 were used. For untagged ubiquitin titrations, reactions containing 15 nM Uba6, 1 mM ATP, 0.2 mM PPi (50 CPM/pmole [³²P PPi) in E1 buffer (described above) were incubated for 30 minutes at 30° C. before stopped with 5% (w/v) trichloroacetic acid (TCA) containing 10 mM PPi and processed as described (Bruzzese, et al., 2009). ATP and PPi titrations were run under similar condition, except ubiquitin was fixed at 4 μM. Since ubiquitin was inhibitory at hgher concentrations, inhibited top points were excluded from estimated K_{M} fits. k_{cat} values were calculated from the rates of the PPi-ATP exchange reactions at optimal conditions, 1 mM ATP, 0.2 mM ATP, 4 μM ubiquitin and using an [α-³²P] ATP standard curve.

IC₅₀s were determine by serial diluting each compound into a 96-well assay plate containing 5 nM Uba6, 1 mM ATP and 0.2 mM PPi (50 cpm/pmol [³²P] PPi). Reactions were initiated with addition of 4 μM ubiquitin. Assays were incubated for 60 minutes at 30° C. in a final volume of 50 μL and were stopped and processed as previously described (Bruzzese, et al., 2009).

The results of the biochemical characterization of the UBA6 mutants are summarized in Table 14. The results of assays for potency of E1 enzyme inhibitors on UBA6 mutants are summarized in Table 15.

TABLE 14
Kinetic characterization of the UBA6 mutants.

| UBA6 | | K_{M} Ub | | k_{cat} |
|---|---|---|---|---|
| Samples | K_{M} ATP (μM) | (μM) | K_{M} PPi (μM) | (s⁻¹) |
| wild-type | 30 ± 3.3 | 0.82 ± 0.01 | 12 ± 1.2 | 1.2 ± 0.07 |
| A573T | 57 ± 8.0 | 0.87 ± 0.09 | 15 ± 1.8 | 1.5 ± 0.11 |
| A573D | 1015 ± 162 | 1.6 ± 0.33 | 2 ± 0.4 | 0.15 ± 0.020 |

TABLE 15
Potency of E1 enzyme inhibitors on UBA6 mutants

| | | | IC₅₀ Adenosine |
|---|---|---|---|
| | IC₅₀ MLN4924 | IC₅₀ Compound 1 | sulfamate |
| UBA6 Samples | (μM) | (μM) | (μM) |
| wt | 6.2 ± 0.80 | 0.92 ± 0.13 | 0.042 ± 0.002 |
| A573T | >100 | 29 ± 4.0 | 0.027 ± 0.002 |
| A573D | >100 | >100 | 0.36 ± 0.02 |

As with the UBA3 A171 substitution, adenosine-sulfamate-like inhibitors with a large N6-substitution (i.e., a bulky group, e.g., indane, off an amino substituent of the heteroaryl (e.g., purine)), such as MLN4924 and Compound 1 lost potency in the variant UBA6 enzyme with a mutation at the analogous position, A573. Compounds without the large substitution (such as adenosine sulfamate) did not lose as much potency.

### Example 11

### General Procedures

Generation of E1 Enzymes

Following manufacturer instructions, baculoviruses are generated with the Bac-to-Bac Expression System (Invitrogen) for the following proteins: untagged NAEα (APPBP1; NP_{—}003896.1), N-terminally His-tagged NAEβ (UBE1C; NP_{—}003959.3), untagged SAEα (SAE1; NP_{—}005491.1), N-terminally His-tagged SAEβ (UBA2; NP_{—}005490.1), N-terminally His-tagged murine UAE (UBE1X; NP_{—}033483). NAEα/His-NAEβ and SAEα/His-SAEβ complexes are generated by co-infection of Sf9 cells, which are harvested after 48 hours. His-mUAE is generated by single infection of Sf9 cells and harvested after 72 hours. Expressed proteins are purified by affinity chromatography (Ni-NTA agarose, Qiagen) using standard buffers.

Generation of E2 Enzymes

UBC12 (UBE2M; NP_{—}003960.1), UBC9 (UBE2I; NP_{—}003336.1), UBC2 (UBE2A; NP_{—}003327.2) are subcloned into pGEX (Pharmacia) and expressed as N-terminally GST tagged fusion proteins in E. coli. Expressed proteins are purified by conventional affinity chromatography using standard buffers.

Generation of Ub1 Proteins

NEDD8 (NP_{—}006147), Sumo-1 (NP_{—}003343) and Ubiquitin (with optimized codons) are subcloned into pFLAG-2 (Sigma) and expressed as N-terminally Flag tagged fusion proteins in E. coli. Expressed proteins are purified by conventional chromatography using standard buffers.

E1 Enzyme Assays

NEDD8-Activating Enzyme (NAE) HTRF Assay

An NAE enzymatic reaction totals 50 μL and contains 50 mM HEPES (pH 7.5), 0.05% BSA, 5 mM MgCl₂, 20 μM ATP, 250 μM GSH, 0.01 μM UBC12-GST, 0.075 μM NEDD8-Flag and 0.28 nM recombinant human NAE enzyme. The enzymatic reaction mixture, with and without compound inhibitor, is incubated at 24° C. for 90 minutes in a 384-well plate before termination with 25 μL of Stop/Detection buffer (0.1M HEPES pH 7.5, 0.05% Tween20, 20 mM EDTA, 410 mM KF, 0.53 nM Europium-Cryptate labeled monoclonal anti-FLAG M2 antibody (CisBio International) and 8.125 μg/mL PHYCOLINK goat anti-GST allophycocyanin (XL-APC) antibody (Prozyme)). After incubation for 3 hours at 24° C., quantification of the FRET is performed on the Analyst HT 96.384 (Molecular Devices).

An SAE enzymatic reaction is conducted as outlined above for NAE except that UBC12-GST and NEDD8-Flag are replaced by 0.01 μM UBC9-GST and 0.125 μM Sumo-Flag respectively and the concentration of ATP is 0.5 μM. Recombinant human SAE (0.11 nM) is the source of enzyme.

An UAE enzymatic reaction is conducted as outlined above for NAE except that UBC12-GST and NEDD8-Flag are replaced by 0.005 μM UBC2-GST and 0.125 μM Ubiquitin-Flag respectively and the concentration of ATP is 0.1 μM. Recombinant mouse UAE (0.3 nM) is the source of enzyme.

Anti-Proliferation Assay (WST)

Calu-6 (2400/well) or other tumor cells in 80 μL of appropriate cell culture medium (MEM for Calu6, Invitrogen) supplemented with 10% fetal bovine serum (Invitrogen) are seeded in wells of a 96-well cell culture plate and incubated for 24 hours in a tissue culture incubator. Compound inhibitors are added in 20 μL culture media to the wells and the plates are incubated for 72 hours at 37° C. 10% final concentration of WST-1 reagent (Roche) is added to each well and incubated for 3.5 hours (for Calu6) at 37° C. The optical density for each well is read at 450 nm using a spectrophotometer (Molecular Devices). Percent inhibition is calculated using the values from a DMSO control set to 100% viability.

Anti-Proliferation Assay (ATPLite)

Calu-6 (1500 cells/well) or other tumor cells are seeded in 72 μL of appropriate cell culture medium (MEM for Calu6, Invitrogen) supplemented with 10% fetal bovine serum (Invitrogen) in wells of a 384-well Poly-D-Lysine coated cell culture plate. Compound inhibitors are added in 8 μL 10% DMSO/PBS to the wells and the plates are incubated for 72 hours at 37° C. Cell culture medium is aspirated, leaving 25 μL in each well. 25 μL of ATPlite 1Step™ reagent (Perkin Elmer) is added to each well. The luminescence for each well is read using the LeadSeeker Microplate Reader (Molecular Devices). Percent inhibition is calculated using the values from a DMSO control set to 100% viability.

In Vivo Assays

In Vivo Tumor Efficacy Model

Calu6 (5×10⁶ cells), HCT116 (2×10⁶ cells) or other tumor cells in 100 μL phosphate buffered saline are aseptically injected into the subcutaneous space in the right dorsal flank of female Ncr nude mice (age 5-8 weeks, Charles River) using a 26-gauge needle. Beginning on day 7 after inoculation, tumors are measured twice weekly using a vernier caliper. Tumor volumes are calculated using standard procedures (0.5×(length×width²)). When the tumors reach a volume of approximately 200 mm³ mice are randomized into groups and injected intravenously in the tail vein with compound inhibitor (100 μL) at various doses and schedules. Alternatively, compound inhibitor may be delivered to mice by intraperitoneal or subcutaneous injection or oral administration. All control groups receive vehicle alone. Tumor size and body weight is measured twice a week and the study is terminated when the control tumors reach approximately 2000 mm³.

The patent and scientific literature referred to herein establishes knowledge that is available to those with skill in the art. Unless otherwise defined, all technical and scientific terms used herein have the same meaning as commonly understood by one of ordinary skill in the art to which this invention belongs. The issued patents, applications, and references that are cited herein are hereby incorporated by reference to the same extent as if each was specifically and individually indicated to be incorporated by reference. In the case of inconsistencies, the present disclosure, including definitions, will control.

While a number of embodiments of this invention have been described, it is apparent that the provided basic examples may be altered to convey other embodiments, which utilize the compounds and methods of this invention. It will thus be appreciated that the scope of this invention has been represented herein by way of example and is not intended to be limited by the specific embodiments, rather is defined by the appended claims.

1.-32. (canceled)

33. An isolated E1 enzyme variant polypeptide selected from the group consisting of:
a) a UBA3 variant polypeptide comprising a variant of the amino acid sequence of SEQ ID NO:2, wherein the variant has a mutation of the amino acid selected from the group consisting of amino acid residue 171, 201, 204, 205, 209, 211, 228, 229, 249, 305, 311, 314 and 324;
b) a UBA3 variant polypeptide comprising a fragment of a variant of the amino acid sequence of SEQ ID NO:2, wherein the fragment comprises at least 15 contiguous amino acids of SEQ ID NO: 2, and wherein the fragment has a mutation of the amino acid selected from the group consisting of amino acid residue 171, 201, 204, 205, 209, 211, 228, 229, 249, 305, 311, 314 and 324;
c) a polypeptide comprising an amino acid sequence selected from the group consisting of: an SAE2 variant of SEQ ID NO:27 with a mutation at residue 121, a UBA1 variant of SEQ ID NO:28 with a mutation at residue 548, a UBA1 variant of SEQ ID NO:29, with a mutation at residue 580, a UBA4 variant of SEQ ID NO:30 with a mutation at residue 185, a UBA5 variant of SEQ ID NO:31 with a mutation at residue 187, a UBA6 variant of SEQ ID NO:32 with a mutation at residue 573, a UBA7 variant of SEQ ID NO:33 with a mutation at residue 544, an ATG7 variant of SEQ ID NO:34 with a mutation at residue 480, a moeb variant of SEQ ID NO:35 with a mutation at residue 134 and a thif variant of SEQ ID NO:36 with a mutation at residue 131; and
d) a polypeptide comprising a fragment of the variant E1 enzyme in c), wherein the fragment comprises at least 15 contiguous amino acids of the E1 enzyme comprising the mutated amino acid;
wherein the E1 enzyme variant has a decreased sensitivity to an E1 enzyme inhibitor.

34. A method for identifying a compound which modulates the activity of a polypeptide of claim 33, comprising:
a) contacting a polypeptide of claim 33 with a test compound; and
b) determining the effect of the test compound on the activity of the polypeptide to thereby identify a compound which modulates the activity of the polypeptide.

35. (canceled)

36. An isolated variant nucleic acid molecule which encodes the E1 enzyme variant polypeptide of claim 33.

37. The isolated nucleic acid molecule of claim 36, comprising a nucleic acid selected from the group consisting of:
a) a variant of SEQ ID NO:1 wherein the variant has a mutation of a base selected from the group consisting of nucleotide 531, 532, 533, 621, 622, 623, 630, 631, 632, 633, 634, 635, 645, 646, 647, 651, 652, 653, 702, 703, 704, 705, 706, 707, 765, 766, 767, 933, 934, 935, 951, 952, 953, 960, 961, 962, 989, 990, and 991; and
b) a nucleic acid molecule comprising a fragment of at least 20 nucleotides of the nucleotide sequence of SEQ ID NO: 1, wherein the fragment has a mutation of a base selected from the group consisting of nucleotide 531, 532, 533, 621, 622, 623, 630, 631, 632, 633, 634, 635, 645, 646, 647, 651, 652, 653, 702, 703, 704, 705, 706, 707, 765, 766, 767, 933, 934, 935, 951, 952, 953, 960, 961, 962, 989, 990, and 991.

38. A reagent that binds specifically to the variant nucleic acid molecule of claim 36 and does not bind to the wild type nucleic acid molecule.

39. The nucleic acid molecule of claim 36 further comprising vector nucleic acid sequences.

40. The nucleic acid molecule of claim 36 further comprising nucleic acid sequences encoding a heterologous polypeptide.

41. A cultured host cell which comprises the nucleic acid molecule of claim 36.

42. A cultured mammalian host cell that expresses the nucleic acid molecule of claim 36.

43. A method for producing an E1 enzyme variant polypeptide comprising culturing the host cell of claim 41 under conditions in which the nucleic acid molecule is expressed.

44. A method for detecting the presence of a polypeptide of claim 33 in a sample, comprising:
a) contacting the sample with a compound which selectively binds to the polypeptide; and
b) determining whether the compound binds to the polypeptide in the sample.

45. The method of claim 44, wherein the compound which binds to the polypeptide is an antibody.

46. A kit comprising a compound which selectively binds to a polypeptide of claim 33 and instructions for use.

47. A method for detecting the presence of a nucleic acid molecule of claim 36 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

48. The method of claim 47, wherein the sample comprises mRNA molecules and is contacted with the nucleic acid probe.

49. A kit comprising the reagent of claim 38 and instructions for use.

50. A method for identifying a compound which binds to a polypeptide of claim 33 comprising the steps of:
a) contacting a polypeptide, or a cell expressing the polypeptide, with a test compound; and
b) determining whether the polypeptide binds to the test compound.

51. The method of claim 50, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for NAE pathway activity.

52. An antibody that specifically binds to the polypeptide of claim 33.

53. A method for modulating the activity of a polypeptide of claim 33 comprising contacting a polypeptide or a cell expressing the polypeptide with a compound which binds to the polypeptide in a sufficient concentration to modulate the activity of the polypeptide.

54. A method for determining whether a test compound modulates the resistance of a cell to an E1 enzyme inhibitor, the method comprising:
a) measuring the level of expression or activity of a polypeptide of claim 33 in a cell in the presence of a test compound;
b) measuring the level of expression or activity of the polypeptide in a cell in the absence of the test compound; and
c) identifying the compound as a modulator of resistance of the cell to an E1 enzyme inhibitor if the level of expression or activity of the polypeptide in the cell in the presence of the test compound differs from the level of expression of the polypeptide in the cell in the absence of the test compound.

55. A method for determining whether a test cell has a drug-resistant phenotype, the method comprising:
a) measuring the expression or activity of an E1 enzyme variant sequence in the test cell;
b) comparing the expression or activity of the E1 enzyme variant sequence measured in step a) to the expression or activity of the E1 enzyme sequence in a control cell not having a drug-resistant phenotype; and
c) determining that the test cell has a drug resistant phenotype if the expression or activity of the E1 enzyme variant sequence in the test cell differs compared to the expression or activity of the E1 enzyme variant sequence in the control cell,
wherein the drug is an E1 enzyme inhibitor.

56. The method of claim 55, wherein the E1 enzyme variant sequence is a UBA3 variant sequence selected from the group consisting of
i) a nucleic acid molecule comprising a variant of SEQ ID NO:1 wherein the variant has a mutation of a base selected from the group consisting of nucleotide 531, 532, 533, 621, 622, 623, 630, 631, 632, 633, 634, 635, 645, 646, 647, 651, 652, 653, 702, 703, 704, 705, 706, 707, 765, 766, 767, 933, 934, 935, 951, 952, 953, 960, 961, 962, 989, 990, and 991; and
ii) a polypeptide comprising a variant of the amino acid sequence of SEQ ID NO:2, wherein the variant has a mutation of the amino acid selected from the group consisting of amino acid residue 171, 201, 204, 205, 209, 211, 228, 229, 249, 305, 311, 314 and 324.

57. A method for determining whether a subject has or is at risk of developing a drug resistant tumor, the method comprising:
a) measuring the expression of an E1 enzyme variant of claim 33 in a biological sample comprising tumor cells obtained from the subject;
b) comparing the expression of the E1 enzyme variant measured in step a) to the expression of the E1 enzyme variant in a control biological sample that is not drug resistant; and
c) determining that the patient has or is at risk of developing a drug resistant tumor if the expression of the E1 enzyme variant in the biological sample obtained from the patient is up-regulated, wherein the drug is an E1 enzyme inhibitor.

58. The method of claim 57, wherein the mRNA amount of the E1 enzyme variant is measured.

59. A method for treating a subject with a drug therapy comprising an E1 enzyme inhibitor, the method comprising:
a. obtaining from the subject a pre-administration biological sample and a post-administration biological sample;
b. measuring the expression level of a E1 enzyme variant sequence in the two samples; and
c. providing the drug therapy when the expression level of the E1 enzyme variant sequence indicates that the patient will benefit from the treatment.

60. A method for treating a drug resistant tumor in a patient, the method comprising administering to the patient a therapeutically effective amount of a compound that overcomes the resistance of the E1 enzyme variant polypeptide of claim 33 to an E1 enzyme inhibitor.
